SEQUENCE LISTING (133 sequences)

SEQ ID NO: 1 - protein, 401 aa, synthetic construct
  VAR_SEQ 277..282: note = x = any amino acid
YTYEDYKNTAEWLLSHTKHRPQVAIICGSGLGGLTDKLTQAQIFDYGEIPNFPRSTVPGHAGRLVFGFLNGRACVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAECRVLQKLGADAVGMSTVPEVIVARHCGLRVFGFSLITNKVIMDYESLEKANHEEVLAAGKQAAQKLEQFVSILMAXXXXXXDLKRFLYKKLPSVEGLHAIVVSDRDGVPVIKVANDNAPEHALRPGFLSTFALATDQGSKLGLSKNKSIICYYNTYQVVQFNRLPLVVSFIASSSANTGLIVSLEKELAPLFEELRQVVE

SEQ ID NO: 2 - protein, 672 aa, synthetic construct
YTYEDYKNTAEWLLSHTKHRPQVAIICGSGLGGLTDKLTQAQIFDYGEIPNFPRSTVPGHAGRLVFGFLNGRACVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAECRVLQKLGADAVGMSTVPEVIVARHCGLRVFGFSLITNKVIMDYESLEKANHEEVLAAGKQAAQKLEQFVSILMARETLQAWKERVGQELDRVVAFWMEHSHDQEHGGFFTCLGREGRVYDDLKYVWLQGRQVWMYCRLYRTFERFRHAQLLDAAKAGGEFLLRYARVAPPGKKCAFVLTRDGRPVKVQRTIFSECFYTMAMNELWRATGEVRYQTEAVEMMDQIVHWVQEDASGLGRPQLQGAPAAEPMAVPMMLLNLVEQLGEADEELAGKYAELGDWCARRILQHVQRDGQAVLENVSEGGKELPGCLGRQQNPGHTLEAGWFLLRHCIRKGDPELRAHVIDKFLLLPFHSGWDPDHGGLFYFQDADNFCPTQLEWAMKLWWPHSEAMIAFLMGYSDSGDPVLLRLFYQVAEYTFRQFRDPEYGEWFGYLSREGKVALSIKGGPFKGCFHVPRCLAMCEEMLGALLSR

SEQ ID NO: 3 - protein, 604 aa, synthetic construct
  VAR_SEQ 258..262: note = x = any amino acid
RFGSQRVLVEPDAGAGVAVMKFKNPPVNSLSLEFLTELVISLEKLENDKSFRGVILTSDRPGVFSAGLDLTEMCGRSPAHYAGYWKAVQELWLRLYQSNLVLVSAINGACPAGGCLVALTCDYRILADNPRYCIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLXXXXXYAQEKQDFVQHFSQIVRVLTEDEMGHPEIGDAIARLKEVLEYNAIGGKYNRGLTVVVAFRELVEPRKQDADSLQRAWTVGWCVELLQAFFLVADDIMDSSLTRRGQICWYQKPGVGLDAINDANLLEACIYRLLKLYCREQPYYLNLIELFLQSSYQTEIGQTLDLLTAPQGNVDLVRFTEKRYKSIVKYKTAFYSFYLPIAAAMYMAGIDGEKEHANAKKILLEMGEFFQIQDDYLDLFGDPSVTGKIGTDIQDNKCSWLVVQCLQRATPEQYQILKENYGQKEAEKVARVKALYEELDLPAVFLQYEEDSYSHIMALIEQYAAPLPPAVFLGLARKIYKR

SEQ ID NO: 4 - protein, 832 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGCFIDFPKPLIAVVNGPAVGISVTLLGLFDAVYASDRATFHTPFSHLGQSPEGCSSYTFPKIMSPAKATEMLIFGKKLTAGEACAQGLVTEVFPDSTFQKEVWTRLKAFAKLPPNALRISKEVIRKREREKLHAVNAEECNVLQGRWLSDECTNAVVNFLEQELSRRLQRLYPAVNQQETPLPRSWSPKDKYNYIGLSQGNLRVHYKGHGKNHKDAASVRATHPIPAACGIYYFEVKIVSKGRDGYMGIGLSAQGVNMNRLPGWDKHSYGYHGDDGHSFCSSGTGQPYGPTFTTGDVIGCCVNLINGTCFYTKNGHSLGIAFTDLPANLYPTVGLQTPGEIVDANFGQQPFLFDIEDYMREWRAKVQGTVHCFPISARLGEWQAVLQNMVSSYLVHHGYCATATAFARMTETPIQEEQASIKNRQKIQKLVLEGRVGEAIETTQRFYPGLLEHNPNLLFMLKCRQFVEMVNGTDSEVRSLSSRSPKSQDSYPGSPSLSPRHGPSSSHMHNTGADSPSCSNGVASTKSKQNHSKYPAPSSSSSSSSSSSSSSPSSVNYSESNSTDSTKSQHHSSTSNQETSDSEMEMEAEHYPNGVLGSMSTRIVNGAYKHEDLQTDESSMDDRHPRRQLCGGNQAATERIILFGRELQALSEQLGREYGKNLAHTEMLQDAFSLLAYSDPWSCPVGQQLDPIQREPVCAALNSAILESQNLPKQPPLMLALGQASECLRLMARAGLGSCSFA

SEQ ID NO: 5 - protein, 432 aa, synthetic construct
  VAR_SEQ 298..302: note = x = any amino acid
KYIPRRAVLYVPGNDEKKIKKIPSLNVDCAVLDCEDGVAANKKNEARLRIVKTLEDIDLGPTEKCVRVNSVSSGLAEEDLETLLQSRVLPSSLMLPKVESPEEIQWFADKFSFHLKGRKLEQPMNLIPFVETAMGLLNFKAVCEETLKVGPQVGLFLDAVVFGGEDFRASIGATSSKETLDILYARQKIVVIAKAFGLQAIDLVYIDFRDGAGLLRQSREGAAMGFTGKQVIHPNQIAVVQEQFSPSPEKIKWAEELIAAFKEHQQLGKGAFTFQGSMIDMPLLKQAQNTVTLATSIXXXXXKTYVDQACRAAEEFVNVYYTTMDKRRRLLSRLYMGTATLVWNGNAVSGQESLSEFFEMLPSSEFQISVVDCQPVHDEATPSQTTVLVVICGSVKFEGNKQRDFNQNFILTAQASPSNTVWKIASDCFRFQ

SEQ ID NO: 6 - protein, 372 aa, synthetic construct
  VAR_SEQ 258: note = x = any amino acid
RFGSQRVLVEPDAGAGVAVMKFKNPPVNSLSLEFLTELVISLEKLENDKSFRGVILTSDRPGVFSAGLDLTEMCGRSPAHYAGYWKAVQELWLRLYQSNLVLVSAINGACPAGGCLVALTCDYRILADNPRYCIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLXEAETEEQQRFSYQQRLKAAVHYTVGCLCEEVALDKEMQFSKQTIAAISELTFRQCENFAKDLEMFARHAKRTTINTEDVKLLARRSNSLLKYITDKSEEIAQINLERKAQKKKK

SEQ ID NO: 7 - protein, 606 aa, synthetic construct
  VAR_SEQ 251: note = x = any amino acid
FETLVVTSEDGITKIMFNRPKKKNAINTEMYHEIMRALKAASKDDSIITVLTGNGDYYSSGNDLTNFTDIPPGGVEEKAKNNAVLLREFVGCFIDFPKPLIAVVNGPAVGISVTLLGLFDAVYASDRATFHTPFSHLGQSPEGCSSYTFPKIMSPAKATEMLIFGKKLTAGEACAQGLVTEVFPDSTFQKEVWTRLKAFAKLPPNALRISKEVIRKREREKLHAVNAEECNVLQGRWLSDECTNAVVNFLXQEQKKELSEIAQSIVANGKGILAADESVGTMGNRLQRIKVENTEENRRQFREILFSVDSSINQSIGGVILFHETLYQKDSQGKLFRNILKEKGIVVGIKLDQGGAPLAGTNKETTIQGLDGLSERCAQYKKDGVDFGKWRAVLRIADQCPSSLAIQENANALARYASICQQNGLVPIVEPEVIPDGDHDLEHCQYVTEKVLAAVYKALNDHHVYLEGTLLKPNMVTAGHACTKKYTPEQVAMATVTALHRTVPAAVPGICFLSGGMSEEDATLNLNAINLCPLPKPWKLSFSYGRALQASALAAWGGKAANKEATQEAFMKRAMANCQAAKGQYVHTGSSGAASTQSLFTACYTY

SEQ ID NO: 8 - protein, 348 aa, synthetic construct
  VAR_SEQ 258..263: note = x = any amino acid
RFGSQRVLVEPDAGAGVAVMKFKNPPVNSLSLEFLTELVISLEKLENDKSFRGVILTSDRPGVFSAGLDLTEMCGRSPAHYAGYWKAVQELWLRLYQSNLVLVSAINGACPAGGCLVALTCDYRILADNPRYCIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLXXXXXXARLRAFLSEPIGEKDVCWVDGISHELAINLVTKGINKAYILLGQFLLMHKNEAEFQRWLICCFGATECEAQQTSHCLKEWCACFL

SEQ ID NO: 9 - protein, 364 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGCFIDFPKPLIAVVNGPAVGISVTLLGLFDAVYASDRATFHTPFSHLGQSPEGCSSYTFPKIMSPAKATEMLIFGKKLTAGEACAQGLVTEVFPDSTFQKEVWTRLKAFAKLPPNALRISKEVIRKREREKLHAVNAEECNVLQGRWLSDECTNAVVNFLEAETEEQQRFSYQQRLKAAVHYTVGCLCEEVALDKEMQFSKQTIAAISELTFRQCENFAKDLEMFARHAKRTTINTEDVKLLARRSNSLLKYITDKSEEIAQINLERKAQKKKK

SEQ ID NO: 10 - protein, 588 aa, synthetic construct
  VAR_SEQ 258..259: note = x = any amino acid
RFGSQRVLVEPDAGAGVAVMKFKNPPVNSLSLEFLTELVISLEKLENDKSFRGVILTSDRPGVFSAGLDLTEMCGRSPAHYAGYWKAVQELWLRLYQSNLVLVSAINGACPAGGCLVALTCDYRILADNPRYCIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLXXVVEFFVVTFKVLWAFVLAAARWLVRPKEKSVAGQVCLITGAGSGLGRLFALEFARRRALLVLWDINTQSNEETAGMVRHIYRDLEAADAAALQAGNGEEEILPHCNLQVFTYTCDVGKRENVYLTAERVRKEVGEVSVLVNNAGVVSGHHLLECPDELIERTMMVNCHAHFWTTKAFLPTMLEINHGHIVTVASSLGLFSTAGVEDYCASKFGVVGFHESLSHELKAAEKDGIKTTLVCPYLVDTGMFRGCRIRKEIEPFLPPLKPDYCVKQAMKAILTDQPMICTPRLMYIVTFMKSILPFEAVVCMYRFLGADKCMYPFIAQRKQAT

SEQ ID NO: 11 - protein, 700 aa, synthetic construct
  VAR_SEQ 258..262: note = x =any amino acid
RFGSQRVLVEPDAGAGVAVMKFKNPPVNSLSLEFLTELVISLEKLENDKSFRGVILTSDRPGVFSAGLDLTEMCGRSPAHYAGYWKAVQELWLRLYQSNLVLVSAINGACPAGGCLVALTCDYRILADNPRYCIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLXXXXXLAAQKARGLFKDFFPETGTKIELPELFDRGTASFPQTIYCGFDPTADSLHVGHLLALLGLFHLQRAGHNVIALVGGATARLGDPSGRTKEREALETERVRANARALRLGLEALAANHQQLFTDGRSWGSFTVLDNSAWYQKQHLVDFLAAVGGHFRMGTLLSRQSVQLRLKSPEGMSLAEFFYQVLQAYDFYYLFQRYGCRVQLGGSDQLGNIMSGYEFINKLTGEDVFGITVPLITSTTGAKLGKSAGNAVWLNRDKTSPFELYQFFVRQPDDSVERYLKLFTFLPLPEIDHIMQLHVKEPERRGPQKRLAAEVTKLVHGREGLDSAKRCTQALYHSSIDALEVMSDQELKELFKEAPFSEFFLDPGTSVLDTCRKANAIPDGPRGYRMITEGGVSINHQQVTNPESVLIVGQHILKNGLSLLKIGKRNFYIIKWLQ

SEQ ID NO: 12 - protein, 610 aa, synthetic construct
RFGSQRVLVEPDAGAGVAVMKFKNPPVNSLSLEFLTELVISLEKLENDKSFRGVILTSDRPGVFSAGLDLTEMCGRSPAHYAGYWKAVQELWLRLYQSNLVLVSAINGACPAGGCLVALTCDYRILADNPRYCIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLQKKELSEIAQSIVANGKGILAADESVGTMGNRLQRIKVENTEENRRQFREILFSVDSSINQSIGGVILFHETLYQKDSQGKLFRNILKEKGIVVGIKLDQGGAPLAGTNKETTIQGLDGLSERCAQYKKDGVDFGKWRAVLRIADQCPSSLAIQENANALARYASICQQNGLVPIVEPEVIPDGDHDLEHCQYVTEKVLAAVYKALNDHHVYLEGTLLKPNMVTAGHACTKKYTPEQVAMATVTALHRTVPAAVPGICFLSGGMSEEDATLNLNAINLCPLPKPWKLSFSYGRALQASALAAWGGKAANKEATQEAFMKRAMANCQAAKGQYVHTGSSGAASTQSLFTACYTY

SEQ ID NO: 13 - protein, 642 aa, synthetic construct
  VAR_SEQ 298..300: note = x = any amino acid
KYIPRRAVLYVPGNDEKKIKKIPSLNVDCAVLDCEDGVAANKKNEARLRIVKTLEDIDLGPTEKCVRVNSVSSGLAEEDLETLLQSRVLPSSLMLPKVESPEEIQWFADKFSFHLKGRKLEQPMNLIPFVETAMGLLNFKAVCEETLKVGPQVGLFLDAVVFGGEDFRASIGATSSKETLDILYARQKIVVIAKAFGLQAIDLVYIDFRDGAGLLRQSREGAAMGFTGKQVIHPNQIAVVQEQFSPSPEKIKWAEELIAAFKEHQQLGKGAFTFQGSMIDMPLLKQAQNTVTLATSIXXXRAYQWCREYLGGAWRRVQPEELRVYPVSGGLSNLLFRCSLPDHLPSVGEEPREVLLRLYGAILQGVDSLVLESVMFAILAERSLGPQLYGVFPEGRLEQYIPSRPLKTQELREPVLSAAIATKMAQFHGMEMPFTKEPHWLFGTMERYLKQIQDLPPTGLPEMNLLEMYSLKDEMGNLRKLLESTPSPVVFCHNDIQEGNILLLSEPENADSLMLVDFEYSSYNYRGFDIGNHFCEWVYDYTHEEWPFYKARPTDYPTQEQQLHFIRHYLAEAKKGETLSQEEQRKLEEDLLVEVSRYALASHFFWGLWSILQASMSTIEFGYLDYAQSRFQFYFQQKGQLT

SEQ ID NO: 14 - protein, 369 aa, synthetic construct
  VAR_SEQ 277..282: note = x = any amino acid
YTYEDYKNTAEWLLSHTKHRPQVAIICGSGLGGLTDKLTQAQIFDYGEIPNFPRSTVPGHAGRLVFGFLNGRACVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAECRVLQKLGADAVGMSTVPEVIVARHCGLRVFGFSLITNKVIMDYESLEKANHEEVLAAGKQAAQKLEQFVSILMAXXXXXXTLEQHLEDTMKNPSIVGVLCTDSQGLNLGCRGTLSDEHAGVISVLAQQAAKLTSDPTDIPVVCLESDNGNIMIQKHDGITVAVHKMA

SEQ ID NO: 15 - protein, 368 aa, synthetic construct
  VAR_SEQ 251..254: note = x = any amino acid
FETLVVTSEDGITKIMFNRPKKKNAINTEMYHEIMRALKAASKDDSIITVLTGNGDYYSSGNDLTNFTDIPPGGVEEKAKNNAVLLREFVGCFIDFPKPLIAVVNGPAVGISVTLLGLFDAVYASDRATFHTPFSHLGQSPEGCSSYTFPKIMSPAKATEMLIFGKKLTAGEACAQGLVTEVFPDSTFQKEVWTRLKAFAKLPPNALRISKEVIRKREREKLHAVNAEECNVLQGRWLSDECTNAVVNFLXXXXEAETEEQQRFSYQQRLKAAVHYTVGCLCEEVALDKEMQFSKQTIAAISELTFRQCENFAKDLEMFARHAKRTTINTEDVKLLARRSNSLLKYITDKSEEIAQINLERKAQKKKK

SEQ ID NO: 16 - protein, 717 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGCFIDFPKPLIAVVNGPAVGISVTLLGLFDAVYASDRATFHTPFSHLGQSPEGCSSYTFPKIMSPAKATEMLIFGKKLTAGEACAQGLVTEVFPDSTFQKEVWTRLKAFAKLPPNALRISKEVIRKREREKLHAVNAEECNVLQGRWLSDECTNAVVNFLWSSHDKMLAQPLKDSDVEVYNIIKKESNRQRVGLELIASENFASRAVLEALGSCLNNKYSEGYPGQRYYGGTEFIDELETLCQKRALQAYKLDPQCWGVNVQPYSGSPANFAVYTALVEPHGRIMGLDLPDGGHLTHGFMTDKKKISATSIFFESMPYKVNPDTGYINYDQLEENARLFHPKLIIAGTSCYSRNLEYARLRKIADENGAYLMADMAHISGLVAAGVVPSPFEHCHVVTTTTHKTLRGCRAGMIFYRKGVKSVDPKTGKEILYNLESLINSAVFPGLQGGPHNHAIAGVAVALKQAMTLEFKVYQHQVVANCRALSEALTELGYKIVTGGSDNHLILVDLRSKGTDGGRAEKVLEACSIACNKNTCPGDRSALRPSGLRLGTPALTSRGLLEKDFQKVAHFIHRGIELTLQIQSDTGVRATLKEFKERLAGDKYQAAVQALREEVESFASLFPLPGLP

SEQ ID NO: 17 - protein, 366 aa, synthetic construct
  VAR_SEQ 251..252: note = x = any amino acid
FETLVVTSEDGITKIMFNRPKKKNAINTEMYHEIMRALKAASKDDSIITVLTGNGDYYSSGNDLTNFTDIPPGGVEEKAKNNAVLLREFVGCFIDFPKPLIAVVNGPAVGISVTLLGLFDAVYASDRATFHTPFSHLGQSPEGCSSYTFPKIMSPAKATEMLIFGKKLTAGEACAQGLVTEVFPDSTFQKEVWTRLKAFAKLPPNALRISKEVIRKREREKLHAVNAEECNVLQGRWLSDECTNAVVNFLXXEAETEEQQRFSYQQRLKAAVHYTVGCLCEEVALDKEMQFSKQTIAAISELTFRQCENFAKDLEMFARHAKRTTINTEDVKLLARRSNSLLKYITDKSEEIAQINLERKAQKKKK

SEQ ID NO: 18 - protein, 708 aa, synthetic construct
RFGSQRVLVEPDAGAGVAVMKFKNPPVNSLSLEFLTELVISLEKLENDKSFRGVILTSDRPGVFSAGLDLTEMCGRSPAHYAGYWKAVQELWLRLYQSNLVLVSAINGACPAGGCLVALTCDYRILADNPRYCIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLAMDELLRRAVPPTPAYELREKTPAPAEGQCADFVSFYGGLAETAQRAELLGRLARGFGVDHGQVAEQSAGVLHLRQQQREAAVLLQAEDRLRYALVPRYRGLFHHISKLDGGVRFLVQLRADLLEAQALKLVEGPDVREMNGVLKGMLSEWFSSGFLNLERVTWHSPCEVLQKISEAEAVHPVKNWMDMKRRVGPYRRCYFFSHCSTPGEPLVVLHVALTGDISSNIQAIVKEHPPSETEEKNKITAAIFYSISLTQQGLQGVELGTFLIKRVVKELQREFPHLGVFSSLSPIPGFTKWLLGLLNSQTKEHGRNELFTDSECKEISEITGGPINETLKLLLSSSEWVQSEKLVRALQTPLMRLCAWYLYGEKHRGYALNPVANFHLQNGAVLWRINWMADVSLRGITGSCGLMANYRYFLEETGPNSTSYLGSKIIKASEQVLSLVAQFQK

SEQ ID NO: 19 - protein, 348 aa, synthetic construct
  VAR_SEQ 258..263: note = x = any amino acid
RFGSQRVLVEPDAGAGVAVMKFKNPPVNSLSLEFLTELVISLEKLENDKSFRGVILTSDRPGVFSAGLDLTEMCGRSPAHYAGYWKAVQELWLRLYQSNLVLVSAINGACPAGGCLVALTCDYRILADNPRYCIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLXXXXXXQKHRDFVAEPMGEKPVGSLAGIGEVLGKKLEERGFDKAYVVLGQFLVLKKDEDLFREWLKDTCGANAKQSRDCFGCLREWCDAFL

SEQ ID NO: 20 - protein, 525 aa, synthetic construct
  VAR_SEQ 258: note = x = any amino acid
RFGSQRVLVEPDAGAGVAVMKFKNPPVNSLSLEFLTELVISLEKLENDKSFRGVILTSDRPGVFSAGLDLTEMCGRSPAHYAGYWKAVQELWLRLYQSNLVLVSAINGACPAGGCLVALTCDYRILADNPRYCIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLXIEEIKEKMKTVKHKILVLSGKGGVGKSTFSAHLAHGLAEDENTQIALLDIDICGPSIPKIMGLEGEQVHQSGSGWSPVYVEDNLGVMSVGFLLSSPDDAVIWRGPKKNGMIKQFLRDVDWGEVDYLIVDTPPGTSDEHLSVVRYLATAHIDGAVIITTPQEVSLQDVRKEINFCRKVKLPIIGVVENMSGFICPKCKKESQIFPPTTGGAELMCQDLEVPLLGRVPLDPLIGKNCDKGQSFFIDAPDSPATLAYRSIIQRIQEFCNL

SEQ ID NO: 21 - protein, 527 aa, synthetic construct
  VAR_SEQ 251..256: note = x = any amino acid
FETLVVTSEDGITKIMFNRPKKKNAINTEMYHEIMRALKAASKDDSIITVLTGNGDYYSSGNDLTNFTDIPPGGVEEKAKNNAVLLREFVGCFIDFPKPLIAVVNGPAVGISVTLLGLFDAVYASDRATFHTPFSHLGQSPEGCSSYTFPKIMSPAKATEMLIFGKKLTAGEACAQGLVTEVFPDSTFQKEVWTRLKAFAKLPPNALRISKEVIRKREREKLHAVNAEECNVLQGRWLSDECTNAVVNFLXXXXXXQECMDYAVTLARQAGEVVCEAIKNEMNVMLKSSPVDLVTATDQKVEKMLISSIKEKYPSHSFIGEESVAAGEKSILTDNPTWIIDPIDGTTNFVHRFPFVAVSIGFAVNKKIEFGVVYSCVEGKMYTARKGKGAFCNGQKLQVSQQEDITKSLLVTELGSSRTPETVRMVLSNMEKLFCIPVHGIRSVGTAAVNMCLVATGGADAYYEMGIHCWDVAGAGIIVTEAGGVLMDVTGGPFDLMSRRVIAANNRILAERIAKEIQVIPLQRDDE

SEQ ID NO: 22 - protein, 401 aa, synthetic construct
  VAR_SEQ 277..287: note = x = any amino acid
YTYEDYKNTAEWLLSHTKHRPQVAIICGSGLGGLTDKLTQAQIFDYSEIPNFPQSTVPGHVGRLVFGFLNGRACVIMQGRFHMYEGYSLWKVTFPVRVFHLLGVDTLVVTNAAGGLNPEFEVGDIMLIRDHINLPGFSGQNPLRGPNDERFGVRFPAMSDAYDRTMRQRALSMWKQMGEQRELQEGTYVMLAGPSFETVAESRLLQKLGADAVGMSTVPEVIVARHCGLRVFGFSLITNKVIMDYESLEKANHEEVLAAGKQAAQKLEQLVSILMAXXXXXXDLKRFLYKKLPSVEGLHAIVVSDRDGVPVIKVANDNAPEHALRPGFLSTFALATDQGSKLGLSKNKSIICYYNTYQVVQFNRLPLVVSFIASSSANTGLIVSLEKELAPLFEELRQVVE

SEQ ID NO: 23 - protein, 401 aa, synthetic construct
  VAR_SEQ 277..282: note = x = any amino acid
FTYEDYETTAKWLLQHTEYRPQVAVICGSGLGGLTAHLKEAQIFDYNEIPNFPQSTVQGHAGRLVFGLLNGRCCVMMQGRFHMYEGYSLSKVTFPVRVFHLLGVETLVVTNAAGGLNPNFEVGDIMLIRDHINLPGFCGQNPLRGPNDERFGVRFPAMSDAYDRDMRQKAFSAWKQMGEQRKLQEGTYVMLAGPNFETVAESRLLKMLGADAVGMSTVPEVIVARHCGLRVFGFSLITNKVVMDYENLEKANHMEVLDAGKAAAQTLERFVSILMEXXXXXXDLKRFLYKKLPSVEGLHAIVVSDRDGVPVIKVANDSAPEHALRPGFLSTFALATDQGSKLGLSKNKSIICYYNTYQVVQFNRLPLVVSFIASSSANTGLIVSLEKELAPLFEELIKVVE

SEQ ID NO: 24 - protein, 672 aa, synthetic construct
YTYEDYKNTAEWLLSHTKHRPQVAIICGSGLGGLTDKLTQAQIFDYSEIPNFPQSTVPGHVGRLVFGFLNGRACVIMQGRFHMYEGYSLWKVTFPVRVFHLLGVDTLVVTNAAGGLNPEFEVGDIMLIRDHINLPGFSGQNPLRGPNDERFGVRFPAMSDAYDRTMRQRALSMWKQMGEQRELQEGTYVMLAGPSFETVAESRLLQKLGADAVGMSTVPEVIVARHCGLRVFGFSLITNKVIMDYESLEKANHEEVLAAGKQAAQKLEQLVSILMARETLQAWKERVAQELDRVVAFWMEHSHDQEHGGFFTCLGREGRVYDDLKYVWLQGRQVWMYCRLYRTFQRFHHSQLLNAAKAGGEFLLRYARLAPPGKKCAFVLTRDGRPVKVQRTIFSECFYTMAMNELWRATGEVQYQTEAVEMMDQIVHWVREDASGLGRPQLQGAPTAEPMAVPMMLLNLVEQLGEADEELAGKYTELGDWCAQRILQHVQRDGQAVLENVSEDGKELSGCLGRQQNPGHALEAGWFLLRHCNRKGDPELRAHVVDKFLLLPFRSGWDPDHGGLFYFQDADDFCPTQLEWAMKLWWPHSEAMIAFLMGYSESGDPVLLRLFYQVAEYTFRQFRDPEYGEWFGYLSREGKVALSIKGGPFKGCFHVPRCLAMCEEMLGALLSR

SEQ ID NO: 25 - protein, 672 aa, synthetic construct
FTYEDYETTAKWLLQHTEYRPQVAVICGSGLGGLTAHLKEAQIFDYNEIPNFPQSTVQGHAGRLVFGLLNGRCCVMMQGRFHMYEGYSLSKVTFPVRVFHLLGVETLVVTNAAGGLNPNFEVGDIMLIRDHINLPGFCGQNPLRGPNDERFGVRFPAMSDAYDRDMRQKAFSAWKQMGEQRKLQEGTYVMLAGPNFETVAESRLLKMLGADAVGMSTVPEVIVARHCGLRVFGFSLITNKVVMDYENLEKANHMEVLDAGKAAAQTLERFVSILMERETLQVWKKRVEQELDRVIAFWMEHSHDQEHGGFFTCLGRDGKVYDHLKYVWLQGRQVWMYCRLYRSFERFRRVELLDAARAGGEFLLRYARVAPPGKKCAFVLTRDGRPVKVQRTIFSECFYTMAMNELWKVTGEVRYQSEAIEMMDQIIHWVREDPAGLGRPQLSGALATEPMAVPMMLLSLVEQLGEEDEELTNMYAELGDWCVHRILQHVQRDGQVVLENVSEDGKELPGCLGRHQNPGHTLEAGWFLLQYALRKGDPKLRMHIIDKFLLLPFHSGWDPEHGGLFYFQDADGLCPTQLEWNMKLWWPHSEAMIAFLMGYSDSGDPALLHLFYKVAEYTFRQFRDPEYGEWFGYLNQEGKVALTIKGGPFKGCFHVPRCLAMCEQILGALLQR

SEQ ID NO: 26 - protein, 604 aa, synthetic construct
  VAR_SEQ 258..262: note = x = any amino acid
RFGSQRVLVEPDAGAGVAVMKLKNPPVNTLSLEFLTELVICLEKLENDKSFRGVVLTSDCPGVFSAGLDLTEMCGKSPAHYAEYWKAVQELWLQFYQSNLVLVSAINGACPAGGCLMTLTCDYRVLADNPRYCIGLNETQLGIVAPFWFKDTLENVIGHRAAERALQLGLLFPPAEALQVGIVDQVVPEEQVQSTALSAIAQWMTIPDHARQLTKAMMRKATASRLITQRDADVQNFVSFISKDSIQKSLQLYLERLXXXXXYAQEKQDFVQHFSQIVRVLTEDEMGHPETGDAIARLKEVLEYNAIGGKYHRGLTVVVAFRELVEPRKQDADSLQRAWTVGWCVELLQAFFLVTDDIMDSSLTRRGQICWYQKPGVGLDAINDAILLEACIYRLLKLYCREQPYYLNLIELFLQSSYQTEIGQTLDLITAPQGNVDLGRFTEKRYKSIVKYKTAFYSFYLPIAAAMYMAGIDGEKEHANAKKILLEMGEFFQIQDDYLDLFGDPSVTGKVGTDIQDNKCSWLVVQCLQRATPEQYQILKENYGQKEAEKVARVKALYEELDLQAVFLQYEEDSYSHIMALIEQYAAPLPPAIFLGLARKIYKR

SEQ ID NO: 27 - protein, 604 aa, synthetic construct
  VAR_SEQ 258..262: note = x = any amino acid
RFANKRVLVETEGPAGVAVMKLRNPPVNSLSLECLTEFTISLEKLENDKSIRGVILTSECPGIFSAGLDLLEMYGRNPAHYAEYWKNVQELWLRLYTSNMILVSAINGASPAGGCLLALCCDYRVMADNPKYTIGLNESLLGIVAPFWFKDMYVNTIGHREAERALQLGTLFSPAEALKVGVVDEVVPEDQVHSKARSVMTKWLAIPDHSRQLTKNMMRKATADNLIKQREADIQNFTSFISKDSIQKSLHMYLEKLXXXXXYNQEKQNFIQHFSQIVKVLTEKELGHPEIGDAIARLKEVLEYNALGGKYNRGLTVVQAFQELVEPKKQDAESLQRALTVGWCVELLQAFFLVSDDIMDSSLTRRGQICWYQKPGIGLDAINDALLLEASIYRLLKFYCREQPYYLNLLELFLQSSYQTEIGQTLDLMTAPQGHVDLGRYTEKRYKSIVKYKTAFYSFYLPIAAAMYMAGIDGEKEHANALKILMEMGEFFQVQDDYLDLFGDPSVTGKVGTDIQDNKCSWLVVQCLLRASPQQRQILEENYGQKDPEKVARVKALYEALDLQSAFFKYEEDSYNRLKSLIEQCSAPLPPSIFMELANKIYKR

SEQ ID NO: 28 - protein, 521 aa, synthetic construct
THPIPAACGIYYFEVKIVSKGRDGYMGIGLSAQGVNMNRLPGWDKHSYGYHGDDGHSFCSSGTGQPYGPTFTTGDVIGCCVNLINGTCFYTKNGHSLGIAFTDLPANLYPTVGLQTPGEIVDANFGQQPFLFDIEDYMREWRAKVQGTVHCFPISARLGEWQAVLQNMVSSYLVHHGYCATATAFARMTETPIQEEQASIKNRQKIQKLVLEGRVGEAIETTQRFYPGLLEHNPNLLFMLKCRQFVEMVNGTDSEVRSLSSRSPKSQDSYPGSPSLSPRHGPSSSHMHNTGADSPSCSNGVVSTKSKQNHSKYPAPSSSSSSSSSSSSSSPSSINYSESNSTDSTKSQPHSSTSNQETSDSEMEMEAEHYPNGVLGSMSTRIVNGAYKHEDLQTDESSMDDGHPRRQLCGGNQAATERIILFGRELQALSEQLGREYGNNLAHTEMLQDAFSLLAYSDPWSCPVGQQLDPIQREPVCAALNSAILESQNLPKQPPLMLALGQASECLRLMARAGLGSCSFA

SEQ ID NO: 29 - protein, 535 aa, synthetic construct
GHGKNHKDAASVRATHPIPAACGIYYFEVKIVSKGRDGYMGIGLSAQGVNMNRLPGWDKHSYGYHGDDGHSFCSSGTGQPYGPTFTTGDVIGCCVNLINGTCFYTKNGHSLGIAFTDLPANLYPTVGLQTPGEIVDANFGQQPFLFDIEDYMREWRAKVQGTVHGFPISARLGEWQAVLQNMVSSYLVHHGYCSTATAFARMTETPIQEEQASIKNRQKIQKLVLEGRVGEAIETTQRFYPGLLEHNPNLLFMLKCRQFVEMVNGTDSEVRSLSSRSPKSQDSYPGSPSLSPRHGPSSSHIHNTGADSPSCSNGVASTKNKQNHSKYPAPSSSSSSSSSSSSSSPSSVNYSESNSTDSTKSQPHSSTSNQETSDSEMEMEAEHYPNGVLESVSTRIVNGAYKHDDLQTDESSMDDGHPRRQLCGGNQAATERIILFGRELQALSEQLGREYGKNLAHTEMLQDAFSLLAYSDPWSCPVGHQLDPIQREPVCAALNSAILESQNLPKQPPLMLALGQASECLRLMARAGLGSCSFA

SEQ ID NO: 30 - protein, 432 aa, synthetic construct
  VAR_SEQ 298..302: note = x = any amino acid
KYIPRRAVLYVPGNDEKKIKKIPSLNVDCAVLDCEDGVAANKKNEARLRIVKTLEDFDLGPTEKCVRVNSVSSGLAEEDLETLLQSRVLPSSLMLPKVESPEEIQWFADKFSFHLKGRKLEQPMNLIPFVETAMGLLNFKAVCEETLKIGPQVGLFLDAVVFGGEDFRASIGATSSKETLDILYARQKIVVIAKAFGLQAIDLVYIDFRDGAGLLRQSREGAAMGFTGKQVIHPNQIAVVQEQFSPSPEKIKWAEELIAAFKEHQQLGKGAFTFQGSMIDMPLLKQAQNIVTLATSIXXXXXKTYVDQACRAAEEFVNVYYTTMDKRRRLLSRLYMGTATLVWNGNAVSGQESLSEFFEMLPSSEFQISVVDCQPVHDEATPSQTTVLVVICGSVKFEGNKQRDFNQNFILTAQASPSNTVWKIASDCFRFQ

SEQ ID NO: 31 - protein, 432 aa, synthetic construct
  VAR_SEQ 298..302: note = x = any amino acid
KYVPRRAVLYVPGNDEKKIRKIPSLKVDCAVLDCEDGVAENKKNEARLRIAKTLEDFDLGTTEKCVRINSVSSGLAEVDLETFLQARVLPSSLMLPKVEGPEEIRWFSDKFSLHLKGRKLEQPMNLIPFVETAMGLLNFKAVCEETLKTGPQVGLCLDAVVFGGEDFRASIGATSNKDTQDILYARQKVVVTAKAFGLQAIDLVYIDFRDEDGLLRQSREAAAMGFTGKQVIHPNQIAVVQEQFTPTPEKIQWAEELIAAFKEHQQLGKGAFTFRGSMIDMPLLKQAQNIVTLATSIXXXXXKTYVDQACRAAEEFVNVYYTTMDKRRRLLSRLYMGTATLVWNGNAVSGQESLSEFFEMLPSSEFQISVVDCQPVHDDATPSQTTVLVVICGTVKFEGNKQRDFNQNFILTAQASPSNTVWKIASDCFRFQ

SEQ ID NO: 32 - protein, 342 aa, synthetic construct
  VAR_SEQ 228: note = x = any amino acid
SLEFLTELVICLEKLENDKSFRGVVLTSDCPGVFSAGLDLTEMCGKSPAHYAEYWKAVQELWLQFYQSNLVLVSAINGACPAGGCLMTLTCDYRVLADNPRYCIGLNETQLGIVAPFWFKDTLENVIGHRAAERALQLGLLFPPAEALQVGIVDQVVPEEQVQSTALSAIAQWMTIPDHARQLTKAMMRKATASRLITQRDADVQNFVSFISKDSIQKSLQLYLERLXEAETEDQQRFSYQQRLKAAVHYTVGCLCEEVALDKEMQFSKQTIAAISELTFRQCENFAKDLEMFARHAKRTTINTEDVKLLARRSNSLLKYITDKSEEIAQINLERKAQKKKK

SEQ ID NO: 33 - protein, 370 aa, synthetic construct
  VAR_SEQ 230: note = x = any amino acid
SLSLECLTEFTISLEKLENDKSIRGVILTSECPGIFSAGLDLLEMYGRNPAHYAEYWKNVQELWLRLYTSNMILVSAINGASPAGGCLLALCCDYRVMADNPKYTIGLNESLLGIVAPFWFKDMYVNTIGHREAERALQLGTLFSPAEALKVGVVDEVVPEDQVHSKARSVMTKWLAIPDHSRQLTKNMMRKATADNLIKQREADIQNFTSFISKDSIQKSLHMYLEKLXEVEAEEPQEFSHRQRLKAAVHYTVGCLCQEVTLNKQVNFSKQTIAAISEVTFRQCENFAKDLEMFARHAKRSTVTTEDVKLLARRNNSLLKYITEKNEEIAQLNLKGKAKKKRKPEDESRSSRESMAEELDGAEELQSES

SEQ ID NO: 34 - protein, 606 aa, synthetic construct
  VAR_SEQ 251: note = x = any amino acid
YETLMVTSEDGITKIMLNRPTKKNALNVEMYHEIMHALKAASKDDSAITVLTGNGDYYSSGNDLTNFTDIPPGGVEEKAKNGAILLREFVGCFIDFPKPLIAVVNGPAVGISVTLLGLFDAVYASDRATFHTPFSHLGQSPEGCSSYTFPKIMSPAKATEMLVFGKKLTAGEACAQGLVTEVFPDSTFQKEVWTRLKAFAKLPPNAMRISKEIIRNREKEKLHAVNAEECSVLQGRWLSDECMNAVVNFLXQEQKKELSEIAHSIVANGKGILAADESVGTMGNRLQRIKVENTEENRRQFREILFSVDSSINQSIGGVILFHETLYQKDSQGKLFRNILKEKGIVVGIKLDQGGAPLAGTNKETTIQGLDGLSERCAQYKKDGVDFGKWRAVLRIADQCPSSLAIQENANALARYASICQQNGLVPIVEPEVIPDGDHDLEHCQYVTEKVLAAVYKALNDHHVYLEGTLLKPNMVTAGHACTKKYTPEQVAMATVTALHRTVPAAVPGICFLSGGMSEEDATLNLNAINLCPLPKPWKLSFSYGRALQASALAAWGGKAANKEATQEAFMKRAVANCQAAKGQYIHTGSSGAASTQSLFTACYTY

SEQ ID NO: 35 - protein, 602 aa, synthetic construct
  VAR_SEQ 247..248: note = x = any amino acid
ILVTSEDGITKITFNRPTKKNAISFQMYRDIILALKNASTDNTVMAVFTGTGDYYCSGNDLTNFTSATGGIEEAASNGAVLLRDFVNSFIDFPKPLVAVVNGPAVGISVTLLGLFDAVFASDRATFHTPFSQLGQSPEACSSYTFPKMMGSAKAAEMLLFGKKLTAREAWAQGLVTEVFPESTFETEVWTRLKTYAKLPPNAMRISKELIRKNEKEKLYAVNAEECTTLQARWLSEECMNAIMSFVXXEQKKELSEIAQRIVANGKGILAADESVGTMGNRLQRIKVENTEENRRQFRELLFSVDNSISQSIGGVILFHETLYQKDSQGNLFRNVLKEKGIVVGIKLDQGGAPLAGTNKETTIQGLDGLSERCAQYKKDGVDFGKWRAVLRIADQCPSSLAIQENANALARYASICQQNGLVPIVEPEVLPDGDHDLEHCQYVSEKVLAAVYKALNDHHVYLEGTLLKPNMVTAGHACTKKYTPEQVAMATVTALHRTVPAAVPGICFLSGGMSEEDATLNLNAINRCPLPRPWKLSFSYGRALQASALAAWGGKAANKKATQEAFMKRAMANCQAAQGQYVHTGSSGAAATQSLFTASYTY

SEQ ID NO: 36 - protein, 348 aa, synthetic construct
  VAR_SEQ 258..264: note = x = any amino acid
RFGSQRVLVEPDAGAGVAVMKLKNPPVNTLSLEFLTELVICLEKLENDKSFRGVVLTSDCPGVFSAGLDLTEMCGKSPAHYAEYWKAVQELWLQFYQSNLVLVSAINGACPAGGCLMTLTCDYRVLADNPRYCIGLNETQLGIVAPFWFKDTLENVIGHRAAERALQLGLLFPPAEALQVGIVDQVVPEEQVQSTALSAIAQWMTIPDHARQLTKAMMRKATASRLITQRDADVQNFVSFISKDSIQKSLQLYLERLXXXXXXXRLRAFLSEPIGEKDVCWVDGISHELAINLVTKGINKAYILLGQFLLMHKNEAEFQRWLICCCGATECEAQQSSNCLKEWCACFL

SEQ ID NO: 37 - protein, 348 aa, synthetic construct
  VAR_SEQ 258..264: note = x = any amino acid
RFANKRVLVETEGPAGVAVMKLRNPPVNSLSLECLTEFTISLEKLENDKSIRGVILTSECPGIFSAGLDLLEMYGRNPAHYAEYWKNVQELWLRLYTSNMILVSAINGASPAGGCLLALCCDYRVMADNPKYTIGLNESLLGIVAPFWFKDMYVNTIGHREAERALQLGTLFSPAEALKVGVVDEVVPEDQVHSKARSVMTKWLAIPDHSRQLTKNMMRKATADNLIKQREADIQNFTSFISKDSIQKSLHMYLEKLXXXXXXXRLRAFLSEPIGEKDVAWVDGISRELAINLVTKGFNKAYVLLGQFLLMHKNEAEFQRWIICCCGATECEARQSSTCLKEWCSCFL

SEQ ID NO: 38 - protein, 364 aa, synthetic construct
YETLMVTSEDGITKIMLNRPTKKNALNVEMYHEIMHALKAASKDDSAITVLTGNGDYYSSGNDLTNFTDIPPGGVEEKAKNGAILLREFVGCFIDFPKPLIAVVNGPAVGISVTLLGLFDAVYASDRATFHTPFSHLGQSPEGCSSYTFPKIMSPAKATEMLVFGKKLTAGEACAQGLVTEVFPDSTFQKEVWTRLKAFAKLPPNAMRISKEIIRNREKEKLHAVNAEECSVLQGRWLSDECMNAVVNFLEAETEDQQRFSYQQRLKAAVHYTVGCLCEEVALDKEMQFSKQTIAAISELTFRQCENFAKDLEMFARHAKRTTINTEDVKLLARRSNSLLKYITDKSEEIAQINLERKAQKKKK

SEQ ID NO: 39 - protein, 386 aa, synthetic construct
ILVTSEDGITKITFNRPTKKNAISFQMYRDIILALKNASTDNTVMAVFTGTGDYYCSGNDLTNFTSATGGIEEAASNGAVLLRDFVNSFIDFPKPLVAVVNGPAVGISVTLLGLFDAVFASDRATFHTPFSQLGQSPEACSSYTFPKMMGSAKAAEMLLFGKKLTAREAWAQGLVTEVFPESTFETEVWTRLKTYAKLPPNAMRISKELIRKNEKEKLYAVNAEECTTLQARWLSEECMNAIMSFVEVEAEEPQEFSHRQRLKAAVHYTVGCLCQEVTLNKQVNFSKQTIAAISEVTFRQCENFAKDLEMFARHAKRSTVTTEDVKLLARRNNSLLKYITEKNEEIAQLNLKGKAKKKRKPEDESRSSRESMAEELDGAEELQSES

SEQ ID NO: 40 - protein, 588 aa, synthetic construct
  VAR_SEQ 258..259: note = x = any amino acid
RFGSQRVLVEPDAGAGVAVMKLKNPPVNTLSLEFLTELVICLEKLENDKSFRGVVLTSDCPGVFSAGLDLTEMCGKSPAHYAEYWKAVQELWLQFYQSNLVLVSAINGACPAGGCLMTLTCDYRVLADNPRYCIGLNETQLGIVAPFWFKDTLENVIGHRAAERALQLGLLFPPAEALQVGIVDQVVPEEQVQSTALSAIAQWMTIPDHARQLTKAMMRKATASRLITQRDADVQNFVSFISKDSIQKSLQLYLERLXXVVEFFVVTFKVLWAFVLAAARWLVRPKEKSVAGQVCLITGAGSGLGRLFALEFARRRALLVLWDINTQSNEETAGMVRHIYRDLEAADAAALQAGNGEEEILPHCNLQVFTYTCDVGKRENVYLTAERVRKEVGEVSVLVNNAGVVSGHHLLECPDELIERTMMVNCHAHFWTTKAFLPTMLEINHGHIVTVASSLGLFSTAGVEDYCASKFGVVGFHESLSHELKAAEKDGIKTTLVCPYLVDTGMFRGCRIRKEIEPFLPPLKPDYCVKQAMKAILTDQPMICTPRLMYIVTFMKSILPFEAVVCMYRFLGADKCMYPFIAQRKQAT

SEQ ID NO: 41 - protein, 588 aa, synthetic construct
  VAR_SEQ 258..259: note = x = any amino acid
RFANKRVLVETEGPAGVAVMKLRNPPVNSLSLECLTEFTISLEKLENDKSIRGVILTSECPGIFSAGLDLLEMYGRNPAHYAEYWKNVQELWLRLYTSNMILVSAINGASPAGGCLLALCCDYRVMADNPKYTIGLNESLLGIVAPFWFKDMYVNTIGHREAERALQLGTLFSPAEALKVGVVDEVVPEDQVHSKARSVMTKWLAIPDHSRQLTKNMMRKATADNLIKQREADIQNFTSFISKDSIQKSLHMYLEKLXXVVEFFVVTFKVLWAFVLAAARWLVRPKEKSVAGQVCLITGAGSGLGRLFALEFARRRALLVLWDINTQSNEETAGMVRHIYRDLEAADAAALQAGKGEEEILPPCNLQVFTYTCDVGKRENVYLTAERVRKEVGEVSVLVNNAGVVSGHHLLECPDELIERTMMVNCHAHFWTTKAFLPTMLEINHGHIVTVASSLGLFSTAGVEDYCASKFGVVGFHESLSHELKAAEKDGIKTTLVCPYLVDTGMFRGCRIRKEIEPFLPPLKPDYCVKQAMRAILTDQPMVCTPRLMYIVTFMKSILPFEAVVCMYRFLGADKCMYPFIAQRKQAT

SEQ ID NO: 42 - protein, 700 aa, synthetic construct
  VAR_SEQ 258..262: note = x = any amino acid
RFGSQRVLVEPDAGAGVAVMKLKNPPVNTLSLEFLTELVICLEKLENDKSFRGVVLTSDCPGVFSAGLDLTEMCGKSPAHYAEYWKAVQELWLQFYQSNLVLVSAINGACPAGGCLMTLTCDYRVLADNPRYCIGLNETQLGIVAPFWFKDTLENVIGHRAAERALQLGLLFPPAEALQVGIVDQVVPEEQVQSTALSAIAQWMTIPDHARQLTKAMMRKATASRLITQRDADVQNFVSFISKDSIQKSLQLYLERLXXXXXLAAQKARGLFKDFFPETGTKIELPELFDRGTASFPQTIYCGFDPTADSLHVGHLLALLGLFHLQRAGHNVIALVGGATARLGDPSGRTKEREALETERVRANARALRLGLEALAANHQQLFTDGRSWGSFTVLDNSAWYQKQHLVDFLAAVGGHFRMGTLLSRQSVQLRLKSAEGMSLAEFFYQVLQAYDFYYLFQRYGCRVQLGGSDQLGNIMSGYEFINKLTGEDVFGITVPLITSTTGAKLGKSAGNAVWLNRDKTSPFELYQFFVRQPDDSVERYLKLFTFLPLPEIDHIMQLHVKEPERRGAQKRLAAEVTKLVHGREGLDSAKRCTQALYHSSIDALEVMSDQELKELFKEAPFSELFLDPGTSVLDTCRKANAIPDGPRGYRMITEGGVSINHRQVTNPESVLVVGQHILKNGLSLLKIGKRNFYIIKWLQ

SEQ ID NO: 43 - protein, 701 aa, synthetic construct
  VAR_SEQ 258..262: note = x = any amino acid
RFANKRVLVETEGPAGVAVMKLRNPPVNSLSLECLTEFTISLEKLENDKSIRGVILTSECPGIFSAGLDLLEMYGRNPAHYAEYWKNVQELWLRLYTSNMILVSAINGASPAGGCLLALCCDYRVMADNPKYTIGLNESLLGIVAPFWFKDMYVNTIGHREAERALQLGTLFSPAEALKVGVVDEVVPEDQVHSKARSVMTKWLAIPDHSRQLTKNMMRKATADNLIKQREADIQNFTSFISKDSIQKSLHMYLEKLXXXXXLVAPRARGLFKEFFPESGTKTELPELFDRRRAGSSPQTVYCGFDPTGDSLHVGHLLTLLGLFHFQRAGHNVIALVGGSTALLGDPSGRTKGREALSAECVRANAHALRRGLEALAANHARLFADGRPWGSFTVLDNAAWFQEQHLVDFLATVGGHFRMGTLLSRLSVQSRLKSPEGMSLAEFFYQVLQAYDFYYLFQHYGCRVQLGGSDQLGNIMSGYEFIHKLTGEDVFGITVPLITSTTGAKLGKSAGNAVWLNREKTSPFELYQFFVRQQDDSVERYLKLFTFLPLPEIDHIMQLHVKEPEKRVAQKRLAAEVTKLVHGQEGLDSAKRCTQALYHSSIEALEVMSDQELKELFKEASFSELVLDPGTSVIDTCRKANAIPDGPRGYRMITEGGVSINHRQVTNPESVLVIGQHILKNGLSLLKIGKRNFYIIKWLQ

SEQ ID NO: 44 - protein, 610 aa, synthetic construct
RFGSQRVLVEPDAGAGVAVMKLKNPPVNTLSLEFLTELVICLEKLENDKSFRGVVLTSDCPGVFSAGLDLTEMCGKSPAHYAEYWKAVQELWLQFYQSNLVLVSAINGACPAGGCLMTLTCDYRVLADNPRYCIGLNETQLGIVAPFWFKDTLENVIGHRAAERALQLGLLFPPAEALQVGIVDQVVPEEQVQSTALSAIAQWMTIPDHARQLTKAMMRKATASRLITQRDADVQNFVSFISKDSIQKSLQLYLERLQKKELSEIAHSIVANGKGILAADESVGTMGNRLQRIKVENTEENRRQFREILFSVDSSINQSIGGVILFHETLYQKDSQGKLFRNILKEKGIVVGIKLDQGGAPLAGTNKETTIQGLDGLSERCAQYKKDGVDFGKWRAVLRIADQCPSSLAIQENANALARYASICQQNGLVPIVEPEVIPDGDHDLEHCQYVTEKVLAAVYKALNDHHVYLEGTLLKPNMVTAGHACTKKYTPEQVAMATVTALHRTVPAAVPGICFLSGGMSEEDATLNLNAINLCPLPKPWKLSFSYGRALQASALAAWGGKAANKEATQEAFMKRAVANCQAAKGQYIHTGSSGAASTQSLFTACYTY

SEQ ID NO: 45 - protein, 610 aa, synthetic construct
RFANKRVLVETEGPAGVAVMKLRNPPVNSLSLECLTEFTISLEKLENDKSIRGVILTSECPGIFSAGLDLLEMYGRNPAHYAEYWKNVQELWLRLYTSNMILVSAINGASPAGGCLLALCCDYRVMADNPKYTIGLNESLLGIVAPFWFKDMYVNTIGHREAERALQLGTLFSPAEALKVGVVDEVVPEDQVHSKARSVMTKWLAIPDHSRQLTKNMMRKATADNLIKQREADIQNFTSFISKDSIQKSLHMYLEKLQKKELSEIAQRIVANGKGILAADESVGTMGNRLQRIKVENTEENRRQFRELLFSVDNSISQSIGGVILFHETLYQKDSQGNLFRNVLKEKGIVVGIKLDQGGAPLAGTNKETTIQGLDGLSERCAQYKKDGVDFGKWRAVLRIADQCPSSLAIQENANALARYASICQQNGLVPIVEPEVLPDGDHDLEHCQYVSEKVLAAVYKALNDHHVYLEGTLLKPNMVTAGHACTKKYTPEQVAMATVTALHRTVPAAVPGICFLSGGMSEEDATLNLNAINRCPLPRPWKLSFSYGRALQASALAAWGGKAANKKATQEAFMKRAMANCQAAQGQYVHTGSSGAAATQSLFTASYTY

SEQ ID NO: 46 - protein, 642 aa, synthetic construct
  VAR_SEQ 298..300: note = x = any amino acid
KYIPRRAVLYVPGNDEKKIKKIPSLNVDCAVLDCEDGVAANKKNEARLRIVKTLEDFDLGPTEKCVRVNSVSSGLAEEDLETLLQSRVLPSSLMLPKVESPEEIQWFADKFSFHLKGRKLEQPMNLIPFVETAMGLLNFKAVCEETLKIGPQVGLFLDAVVFGGEDFRASIGATSSKETLDILYARQKIVVIAKAFGLQAIDLVYIDFRDGAGLLRQSREGAAMGFTGKQVIHPNQIAVVQEQFSPSPEKIKWAEELIAAFKEHQQLGKGAFTFQGSMIDMPLLKQAQNIVTLATSIXXXRAYQWCREYLGGAWRRVQPEELRVYPVSGGLSNLLFRCSLPDHLPSVGEEPREVLLRLYGAILQGVDSLVLESVMFAILAERSLGPQLYGVFPEGRLEQYIPSRPLKTQELREPVLSAAIATKMAQFHGMEMPFTKEPHWLFGTMERYLKQIQDLPPTGLPEMNLLEMYSLKDEMGNLRKLLESTPSPVVFCHNDIQEGNILLLSEPENADSLMLVDFEYSSYNYRGFDIGNHFCEWVYDYTHEEWPFYKARPTDYPTQGQQLHFIRHYLAEAKKGETLSQEEQRKLEEDLLVEVSRYALASHFFWGLWSILQASMSTIEFGYLDYAQSRFQFYFQQKGQLT

SEQ ID NO: 47 - protein, 642 aa, synthetic construct
  VAR_SEQ 298..300: note = x = any amino acid
KYVPRRAVLYVPGNDEKKIRKIPSLKVDCAVLDCEDGVAENKKNEARLRIAKTLEDFDLGTTEKCVRINSVSSGLAEVDLETFLQARVLPSSLMLPKVEGPEEIRWFSDKFSLHLKGRKLEQPMNLIPFVETAMGLLNFKAVCEETLKTGPQVGLCLDAVVFGGEDFRASIGATSNKDTQDILYARQKVVVTAKAFGLQAIDLVYIDFRDEDGLLRQSREAAAMGFTGKQVIHPNQIAVVQEQFTPTPEKIQWAEELIAAFKEHQQLGKGAFTFRGSMIDMPLLKQAQNIVTLATSIXXXRAYQWCREYLGGAWRRARPEELSVCPVSGGLSNLLFRCSLPNHVPSVGGEPREVLLRLYGAILQGVDSLVLESVMFAILAERSLGPQLYGVFPEGRLEQYLPSRPLKTQELRDPVLSGAIATRMARFHGMEMPFTKEPRWLFGTMERYLKQIQDLPSTSLPQMNLVEMYSLKDEMNSLRKLLDDTPSPVVFCHNDIQEGNILLLSEPDSDDNLMLVDFEYSSYNYRGFDIGNHFCEWVYDYTYEEWPFYKARPTDYPTREQQLHFIRHYLAEVQKGEILSEEEQKKREEELLLEISRYSLASHFFWGLWSTLQASMSTIEFGYLEYAQSRFQFYFQQKGQLT

SEQ ID NO: 48 - protein, 369 aa, synthetic construct
  VAR_SEQ 277..282: note = x = any amino acid
YTYEDYKNTAEWLLSHTKHRPQVAIICGSGLGGLTDKLTQAQIFDYSEIPNFPQSTVPGHVGRLVFGFLNGRACVIMQGRFHMYEGYSLWKVTFPVRVFHLLGVDTLVVTNAAGGLNPEFEVGDIMLIRDHINLPGFSGQNPLRGPNDERFGVRFPAMSDAYDRTMRQRALSMWKQMGEQRELQEGTYVMLAGPSFETVAESRLLQKLGADAVGMSTVPEVIVARHCGLRVFGFSLITNKVIMDYESLEKANHEEVLAAGKQAAQKLEQLVSILMAXXXXXXTLEQHLEDTMKNPSIVGVLCTDSQGLNLGCRGTLSDEHAGVISVLAQQAAKLTSDPTDIPVVCLESDNGNIMIQKHDGITVAVHKMA

SEQ ID NO: 49 - protein, 369 aa, synthetic construct
  VAR_SEQ 277..282: note = x = any amino acid
FTYEDYETTAKWLLQHTEYRPQVAVICGSGLGGLTAHLKEAQIFDYNEIPNFPQSTVQGHAGRLVFGLLNGRCCVMMQGRFHMYEGYSLSKVTFPVRVFHLLGVETLVVTNAAGGLNPNFEVGDIMLIRDHINLPGFCGQNPLRGPNDERFGVRFPAMSDAYDRDMRQKAFSAWKQMGEQRKLQEGTYVMLAGPNFETVAESRLLKMLGADAVGMSTVPEVIVARHCGLRVFGFSLITNKVVMDYENLEKANHMEVLDAGKAAAQTLERFVSILMEXXXXXXTLEQHLEDTMKNPSIVGVLCTDSQGLNLGCRGTLSDEHAGVISVLAQQAARLTSDPTDIPVVCLESDNGNIMIQKHDGITVAVHKMA

SEQ ID NO: 50 - protein, 368 aa, synthetic construct
  VAR_SEQ 251..254: note = x = any amino acid
YETLMVTSEDGITKIMLNRPTKKNALNVEMYHEIMHALKAASKDDSAITVLTGNGDYYSSGNDLTNFTDIPPGGVEEKAKNGAILLREFVGCFIDFPKPLIAVVNGPAVGISVTLLGLFDAVYASDRATFHTPFSHLGQSPEGCSSYTFPKIMSPAKATEMLVFGKKLTAGEACAQGLVTEVFPDSTFQKEVWTRLKAFAKLPPNAMRISKEIIRNREKEKLHAVNAEECSVLQGRWLSDECMNAVVNFLXXXXEAETEDQQRFSYQQRLKAAVHYTVGCLCEEVALDKEMQFSKQTIAAISELTFRQCENFAKDLEMFARHAKRTTINTEDVKLLARRSNSLLKYITDKSEEIAQINLERKAQKKKK

SEQ ID NO: 51 - protein, 390 aa, synthetic construct
  VAR_SEQ 247..250: note = x = any amino acid
ILVTSEDGITKITFNRPTKKNAISFQMYRDIILALKNASTDNTVMAVFTGTGDYYCSGNDLTNFTSATGGIEEAASNGAVLLRDFVNSFIDFPKPLVAVVNGPAVGISVTLLGLFDAVFASDRATFHTPFSQLGQSPEACSSYTFPKMMGSAKAAEMLLFGKKLTAREAWAQGLVTEVFPESTFETEVWTRLKTYAKLPPNAMRISKELIRKNEKEKLYAVNAEECTTLQARWLSEECMNAIMSFVXXXXEVEAEEPQEFSHRQRLKAAVHYTVGCLCQEVTLNKQVNFSKQTIAAISEVTFRQCENFAKDLEMFARHAKRSTVTTEDVKLLARRNNSLLKYITEKNEEIAQLNLKGKAKKKRKPEDESRSSRESMAEELDGAEELQSES

SEQ ID NO: 52 - protein, 717 aa, synthetic construct
YETLMVTSEDGITKIMLNRPTKKNALNVEMYHEIMHALKAASKDDSAITVLTGNGDYYSSGNDLTNFTDIPPGGVEEKAKNGAILLREFVGCFIDFPKPLIAVVNGPAVGISVTLLGLFDAVYASDRATFHTPFSHLGQSPEGCSSYTFPKIMSPAKATEMLVFGKKLTAGEACAQGLVTEVFPDSTFQKEVWTRLKAFAKLPPNAMRISKEIIRNREKEKLHAVNAEECSVLQGRWLSDECMNAVVNFLWSSHDKMLAQPLKDSDVEVYNIIKKESNRQRVGLELIASENFASRAVLEALGSCLNNKYSEGYPGQRYYGGTEFIDELETLCQKRALQAYKLDPQCWGVNVQPYSGSPANFAVYTALVEPHGRIMGLDLPDGGHLTHGFMTDKKKISATSIFFESMPYKVNPDTGYINYDQLEENARLFHPKLIIAGTSCYSRNLDYARLRKIADENGAYLMADMAHISGLVVAGVVPSPFEHCHVVTTTTHKTLRGCRAGMIFYRKGVKNVDPKTGKEILYNLESLINSAVFPGLQGGPHNHAIAGVAVALKQAMTLEFKVYQHQVVANCRALSEALMELGYKIVTGGSDNHLILVDLRSKGTDGGRAEKVLEACSIACNKNTCPGDRSALRPSGLRLGTPALTSRGLLEKDFQKVAQFIHRGIELTLQIQSDIGVRATLKEFKERLAGDKYQGAVQALREEVESFASLFPLPGLP

SEQ ID NO: 53 - protein, 714 aa, synthetic construct
ILVTSEDGITKITFNRPTKKNAISFQMYRDIILALKNASTDNTVMAVFTGTGDYYCSGNDLTNFTSATGGIEEAASNGAVLLRDFVNSFIDFPKPLVAVVNGPAVGISVTLLGLFDAVFASDRATFHTPFSQLGQSPEACSSYTFPKMMGSAKAAEMLLFGKKLTAREAWAQGLVTEVFPESTFETEVWTRLKTYAKLPPNAMRISKELIRKNEKEKLYAVNAEECTTLQARWLSEECMNAIMSFVWASHEKMLSQPLKDSDAEVYSIIKKESNRQRVGLELIASENFASRAVLEALGSCLNNKYSEGYPGQRYYGGTEFIDELEMLCQKRALQAYHLDPQCWGVNVQPYSGSPANFAVYTALVEPHGRIMGLDLPDGGHLTHGFMTDKKKISATSIFFESMPYKVYPETGYINYDQLEENASLFHPKLIIAGTSCYSRNLDYARLRKIADDNGAYLMADMAHISGLVAAGVVPSPFEHCHVVTTTTHKTLRGCRAGMIFYRKGVRSVDPKTGKETYYELESLINSAVFPGLQGGPHNHAIAGVAVALKQAMTTEFKIYQLQVLANCRALSDALTELGYKIVTGGSDNHLILMDLRSKGTDGGRAEKVLEACSIACNKNTCPGDKSALRPSGLRLGTPALTSRGLLEEDFQKVAHFIHRGIELTLQIQSHMATKATLKEFKEKLAGDEKIQSAVATLREEVENFASNFSLPGLP

SEQ ID NO: 54 - protein, 366 aa, synthetic construct
  VAR_SEQ 251..252: note = x = any amino acid
YETLMVTSEDGITKIMLNRPTKKNALNVEMYHEIMHALKAASKDDSAITVLTGNGDYYSSGNDLTNFTDIPPGGVEEKAKNGAILLREFVGCFIDFPKPLIAVVNGPAVGISVTLLGLFDAVYASDRATFHTPFSHLGQSPEGCSSYTFPKIMSPAKATEMLVFGKKLTAGEACAQGLVTEVFPDSTFQKEVWTRLKAFAKLPPNAMRISKEIIRNREKEKLHAVNAEECSVLQGRWLSDECMNAVVNFLXXEAETEDQQRFSYQQRLKAAVHYTVGCLCEEVALDKEMQFSKQTIAAISELTFRQCENFAKDLEMFARHAKRTTINTEDVKLLARRSNSLLKYITDKSEEIAQINLERKAQKKKK

SEQ ID NO: 55 - protein, 388 aa, synthetic construct
  VAR_SEQ 247..248: note = x = any amino acid
ILVTSEDGITKITFNRPTKKNAISFQMYRDIILALKNASTDNTVMAVFTGTGDYYCSGNDLTNFTSATGGIEEAASNGAVLLRDFVNSFIDFPKPLVAVVNGPAVGISVTLLGLFDAVFASDRATFHTPFSQLGQSPEACSSYTFPKMMGSAKAAEMLLFGKKLTAREAWAQGLVTEVFPESTFETEVWTRLKTYAKLPPNAMRISKELIRKNEKEKLYAVNAEECTTLQARWLSEECMNAIMSFVXXEVEAEEPQEFSHRQRLKAAVHYTVGCLCQEVTLNKQVNFSKQTIAAISEVTFRQCENFAKDLEMFARHAKRSTVTTEDVKLLARRNNSLLKYITEKNEEIAQLNLKGKAKKKRKPEDESRSSRESMAEELDGAEELQSES

SEQ ID NO: 56 - protein, 708 aa, synthetic construct
RFGSQRVLVEPDAGAGVAVMKLKNPPVNTLSLEFLTELVICLEKLENDKSFRGVVLTSDCPGVFSAGLDLTEMCGKSPAHYAEYWKAVQELWLQFYQSNLVLVSAINGACPAGGCLMTLTCDYRVLADNPRYCIGLNETQLGIVAPFWFKDTLENVIGHRAAERALQLGLLFPPAEALQVGIVDQVVPEEQVQSTALSAIAQWMTIPDHARQLTKAMMRKATASRLITQRDADVQNFVSFISKDSIQKSLQLYLERLAMDELLRRAVPPTPAYELREKTPAPAEGQCADFVSFYGGLAETAERAELLGRLARGFGVDHGQVAEQSAGVLQLRQQQREAAVLLQAEDRLRYALVPRYRGLFHHISKLDGGVRFLVQLRADLLEAQALKLVEGPDVREMNGVLKGMLSEWFSSGFLNLERVTWHSPCEVLQKISEAEAVHPVKNWMDMKRRVGPYRRCYFFSHCSTPGEPLVVLHVALTGDISSNIQAIVKECPPSETEEKNTIAAAIFYSISLTQQGLQGVELGTFLIKRVVKELQREFPHLGVFSSLSPIPGFTKWLLGLLNSQVKDHGRNELFTDSECKEISEITGGPTNETLKLLLSSSEWVQSETLVRALQTPLMRLCAWYLYGEKHRGYALNPVANFHLQNGAVLWRINWMADVSLKGITGSCGLMANYRYFLEETGPNSTSYLSSKNIKASEQVLSLVAQFQK

SEQ ID NO: 57 - protein, 708 aa, synthetic construct
RFANKRVLVETEGPAGVAVMKLRNPPVNSLSLECLTEFTISLEKLENDKSIRGVILTSECPGIFSAGLDLLEMYGRNPAHYAEYWKNVQELWLRLYTSNMILVSAINGASPAGGCLLALCCDYRVMADNPKYTIGLNESLLGIVAPFWFKDMYVNTIGHREAERALQLGTLFSPAEALKVGVVDEVVPEDQVHSKARSVMTKWLAIPDHSRQLTKNMMRKATADNLIKQREADIQNFTSFISKDSIQKSLHMYLEKLAMDELLRRAVPPTPAYELREKTPAPAEGQCADFVSFYGGLAEASQRAELLGRLAQGFGVDHGQVAEQSAGVLQLRQQAREAAVLLQAEDRLRYALVPRYRGLFHHISKLDGGVRFLVQLRADLLEAQALKLVEGPHVREMNGVLKSMLSEWFSSGFLNLERVTWHSPCEVLQKISECEAVHPVKNWMDMKRRVGPYRRCYFFSHCSTPGEPLVVLHVALTGDISNNIQGIVKECPPTETEERNRIAAAIFYSISLTQQGLQGVELGTFLIKRVVKELQKEFPQLGAFSSLSPIPGFTKWLLGLLNVQGKEHGRNELFTDSECQEISAVTGNPVHESLKGFLSSGEWVKSEKLTQALQGPLMRLCAWYLYGEKHRGYALNPVANFHLQNGAVMWRINWMADSSLKGLTSSCGLMVNYRYYLEETGPNSISYLGSKNIKASEQILSLVAQFQN

SEQ ID NO: 58 - protein, 348 aa, synthetic construct
  VAR_SEQ 258..263: note = x = any amino acid
RFGSQRVLVEPDAGAGVAVMKLKNPPVNTLSLEFLTELVICLEKLENDKSFRGVVLTSDCPGVFSAGLDLTEMCGKSPAHYAEYWKAVQELWLQFYQSNLVLVSAINGACPAGGCLMTLTCDYRVLADNPRYCIGLNETQLGIVAPFWFKDTLENVIGHRAAERALQLGLLFPPAEALQVGIVDQVVPEEQVQSTALSAIAQWMTIPDHARQLTKAMMRKATASRLITQRDADVQNFVSFISKDSIQKSLQLYLERLXXXXXXQKHRDFVAEPMGEKPVGSLAGIGEVLGKKLEERGFDKAYVVLGQFLVLKKDEDLFREWLKDTCGANAKQSRDCFGCLREWCDAFL

SEQ ID NO: 59 - protein, 348 aa, synthetic construct
  VAR_SEQ 258..263: note = x = any amino acid
RFANKRVLVETEGPAGVAVMKLRNPPVNSLSLECLTEFTISLEKLENDKSIRGVILTSECPGIFSAGLDLLEMYGRNPAHYAEYWKNVQELWLRLYTSNMILVSAINGASPAGGCLLALCCDYRVMADNPKYTIGLNESLLGIVAPFWFKDMYVNTIGHREAERALQLGTLFSPAEALKVGVVDEVVPEDQVHSKARSVMTKWLAIPDHSRQLTKNMMRKATADNLIKQREADIQNFTSFISKDSIQKSLHMYLEKLXXXXXXQKHRDFVAEPMGEKPVGSLAGIGDVLSKRLEERGFDKAYVVLGQFLVLKKDEDLFREWLKDTCGANAKQSRDCFGCLREWCDAFL

SEQ ID NO: 60 - protein, 525 aa, synthetic construct
  VAR_SEQ 258: note = x = any amino acid
RFGSQRVLVEPDAGAGVAVMKLKNPPVNTLSLEFLTELVICLEKLENDKSFRGVVLTSDCPGVFSAGLDLTEMCGKSPAHYAEYWKAVQELWLQFYQSNLVLVSAINGACPAGGCLMTLTCDYRVLADNPRYCIGLNETQLGIVAPFWFKDTLENVIGHRAAERALQLGLLFPPAEALQVGIVDQVVPEEQVQSTALSAIAQWMTIPDHARQLTKAMMRKATASRLITQRDADVQNFVSFISKDSIQKSLQLYLERLXIEEIKEKMKTVKHKILVLSGKGGVGKSTFSAHLAHGLAEDENTQIALLDIDICGPSIPKIMGLEGEQVHQSGSGWSPVYVEDNLGVMSVGFLLSSPDDAVIWRGPKKNGMIKQFLRDVDWGEVDYLIVDTPPGTSDEHLSVVQYLAAARIDGAVIITTPQEVSLQDVRKEINFCRKVKLPIIGVVENMSGFICPKCKKESQIFPPTTGGAELMCQDLEVPLLGRVPLDPLIGKNCDKGQSFFIDAPDSPATLAYRSIIQRIQEFCDL

SEQ ID NO: 61 - protein, 523 aa, synthetic construct
  VAR_SEQ 257: note = x = any amino acid
RFANKRVLVETEGPAGVAVMKLRNPPVNSLSLECLTEFTISLEKLENDKSIRGVILTSECPGIFSAGLDLLEMYGRNPAHYAEYWKNVQELWLRLYTSNMILVSAINGASPAGGCLLALCCDYRVMADNPKYTIGLNESLLGIVAPFWFKDMYVNTIGHREAERALQLGTLFSPAEALKVGVVDEVVPEDQVHSKARSVMTKWLAIPDHSRQLTKNMMRKATADNLIKQREADIQNFTSFISKDSIQKSLHMYLEKXVEEIREKMKTVRHKLLVLSGKGGVGKSTFSAHLAHGLAEDGDTQVALLDIDICGPSIPKIMGLEGEQVHQSGSGWSPVYVDDNLGVMSVGFLLSSPDDAVIWRGPKKNGMIKQFLRDVDWGDVDYLIVDTPPGTSDEHLSVVQYLAAAHIDGAVILTTPQEVALQDVRKEISFCHKVKLPIIGVVENMSGFICPKCKKESQIFPPTTGGAEAMCQDLRIPLLGKVPLDPHIGKSCDKGQSFFVEAPDSPATAAYRSIIQRIRDFCN

SEQ ID NO: 62 - protein, 527 aa, synthetic construct
  VAR_SEQ 251..256: note = x = any amino acid
YETLMVTSEDGITKIMLNRPTKKNALNVEMYHEIMHALKAASKDDSAITVLTGNGDYYSSGNDLTNFTDIPPGGVEEKAKNGAILLREFVGCFIDFPKPLIAVVNGPAVGISVTLLGLFDAVYASDRATFHTPFSHLGQSPEGCSSYTFPKIMSPAKATEMLVFGKKLTAGEACAQGLVTEVFPDSTFQKEVWTRLKAFAKLPPNAMRISKEIIRNREKEKLHAVNAEECSVLQGRWLSDECMNAVVNFLXXXXXXQECMDYAVTLARQAGEVVCEALKNEMNVMLKSSPVDLVTATDQKVEKMLISSIKEKYPSHSFIGEESVAAGEKSILTNNPTWIIDPIDGTTNFVHRFPFVAVSIGFAVNKKIEFGVVYSCVEDKMYTARKGKGAFCNGQKLHVSQQEDITKSLLVTELGSSRTPETVRIVLSNMEKLFCIPVHGIRSVGTAAVNMCLVATGGADAYYEMGIHCWDVAGAGIIVTEAGGVLMDVTGGPFDLMSRRVIAANNRILAERIAKEIQVIPLQRDDE

SEQ ID NO: 63 - protein, 523 aa, synthetic construct
  VAR_SEQ 247..252: note = x = any amino acid
ILVTSEDGITKITFNRPTKKNAISFQMYRDIILALKNASTDNTVMAVFTGTGDYYCSGNDLTNFTSATGGIEEAASNGAVLLRDFVNSFIDFPKPLVAVVNGPAVGISVTLLGLFDAVFASDRATFHTPFSQLGQSPEACSSYTFPKMMGSAKAAEMLLFGKKLTAREAWAQGLVTEVFPESTFETEVWTRLKTYAKLPPNAMRISKELIRKNEKEKLYAVNAEECTTLQARWLSEECMNAIMSFVXXXXXXQECMDYAVILARQAGEMIREALKNEMDVMIKSSPADLVTVTDQKVEKMLMSSIKEKYPCHSFIGEESVAAGEKTVFTEQPTWVIDPIDGTTNFVHRFPFVAVSIGFLVNKEMEFGIVYSCVEDKMYTGRKGKGAFCNGQKLQVSQQEDITKSLLVTELGSSRKPETLRIVLSNMEKLCSIPIHGIRSVGTAAVNMCLVATGGADAYYEMGIHCWDMAGAGIIVTEAGGVLMDVTGGPFDLMSRRIIAANSITLAKRIAKEIEIIPLQRDDE

SEQ ID NO: 64 - protein, 366 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSPEFQAAVAAKLAALAAKAAEAAAAARQARLAAVREAVTALLEKLAKEKEMQFSKQTIAAISELTFRQAENFAKDLEMFARHAKRTTINTEDVKLLARRSNSLLKYITDKSEEIAQINLERKAQKKKK

SEQ ID NO: 65 - protein, 366 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSQEFKDAVAKVLAKIKAEEEKKKEEERKKKLEEVLKKVEEKLKKLAKEKEMQFSKQTIAAISELTFRQAENFAKDLEMFARHAKRTTINTEDVKLLARRSNSLLKYITDKSEEIAQINLERKAQKKKK

SEQ ID NO: 66 - protein, 366 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVNFLKKQKETEEQQRFSYQQRLKAAVHYTVGKLMEEVALDKEMQFSKQTIAAISELTFRQAENFAKDLEMFARHAKRTTINTEDVKLLARRSNSLLKYITDKSEEIAQINLERKAQKKKK

SEQ ID NO: 67 - protein, 366 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVNFLKKIKETEEQQRFSYQQRLKAAVHYTVGKLMEEVALDKEMQFSKQTIAAISELTFRQAENFAKDLEMFARHAKRTTINTEDVKLLARRSNSLLKYITDKSEEIAQINLERKAQKKKK

SEQ ID NO: 68 - protein, 364 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSEEFKNAIAKLFEKIEKKKKEEEELEKRKKKVLEEVTKLLKKLAKEKEMQFSKQTIAAISELTFRQAENFAKDLEMFARHAKRTTINTEDVKLLARRSNSLLKYITDKSEEIAQINLERKAQKKKK

SEQ ID NO: 69 - protein, 364 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSKEFKDAVAALFKKIEEEKKKEEERKKKYEEVLEKVTKELEKLAKEKEMQFSKQTIAAISELTFRQAENFAKDLEMFARHAKRTTINTEDVKLLARRSNSLLKYITDKSEEIAQINLERKAQKKKK

SEQ ID NO: 70 - protein, 364 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVAKLKKETEEQQRFSYQQRLKAAVHYTVGKLMEEVALDKEMQFSKQTIAAISELTFRQAENFAKDLEMFARHAKRTTINTEDVKLLARRSNSLLKYITDKSEEIAQINLERKAQKKKK

SEQ ID NO: 71 - protein, 364 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVALKKKETEEQQRFSYQQRLKAAVHYTVGKLMEEVALDKEMQFSKQTIAAISELTFRQAENFAKDLEMFARHAKRTTINTEDVKLLARRSNSLLKYITDKSEEIAQINLERKAQKKKK

SEQ ID NO: 72 - protein, 372 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQARLAAFLAAVAAKAAAAAAAAAARQKELEAVRAKVEEKMKKIAKEKEMQFSKQTIAAISELTFRQAENFAKDLEMFARHAKRTTINTEDVKLLARRSNSLLKYITDKSEEIAQINLERKAQKKKK

SEQ ID NO: 73 - protein, 372 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQAALAAFLAAVAAARAAAAAAAAAREARKEAVLAAVTALLEKLAKEKEMQFSKQTIAAISELTFRQAENFAKDLEMFARHAKRTTINTEDVKLLARRSNSLLKYITDKSEEIAQINLERKAQKKKK

SEQ ID NO: 74 - protein, 372 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLEELKKKETEEQQRFSYQQRLKAAVHYTVGKLMEEVALDKEMQFSKQTIAAISELTFRQAENFAKDLEMFARHAKRTTINTEDVKLLARRSNSLLKYITDKSEEIAQINLERKAQKKKK

SEQ ID NO: 75 - protein, 372 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLEKVKQKETEEQQRFSYQQRLKAAVHYTVGKLMEEVALDKEMQFSKQTIAAISELTFRQAENFAKDLEMFARHAKRTTINTEDVKLLARRSNSLLKYITDKSEEIAQINLERKAQKKKK

SEQ ID NO: 76 - protein, 717 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSPEFQAAVAKVLAKLEKLKAEMPLALRDPEAYAALQAARAARRVGLELIASENFASRAVLEALGSDLNNKYSEGYPGQRYYGGTEFIDELETLAQKRALQAYKLDPQKWGVNVQPYSGSPANFAVYTALVEPHGRIMGLDLPDGGHLTHGFMTDKKKISATSIFFESMPYKVNPDTGYINYDQLEENARLFHPKLIIAGTSSYSRNLEYARLRKIADENGAYLMADMAHISGLVAAGVVPSPFEHAHVVTTTTHKTLRGPRAGMIFYRKGVKSVDPKTGKEILYNLESLINSAVFPGLQGGPHNHAIAGVAVALKQAMTLEFKVYQHQVVANTRALSEALTELGYKIVTGGSDNHLILVDLRSKGTDGGRAEKVLEACSIACNKNTVPGDRSALRPSGLRLGTPALTSRGLLEKDFQKVAHFIHRGIELTLQIQSDTGVRATLKEFKERLAGDKYQAAVQALREEVESFASLFPLPGLP

SEQ ID NO: 77 - protein, 717 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFKAAVAKVLAKAAAAAAAAPLAVRDPEAAAALQAARERRRVGLELIASENFASRAVLEALGSDLNNKYSEGYPGQRYYGGTEFIDELETLAQKRALQAYKLDPQKWGVNVQPYSGSPANFAVYTALVEPHGRIMGLDLPDGGHLTHGFMTDKKKISATSIFFESMPYKVNPDTGYINYDQLEENARLFHPKLIIAGTSSYSRNLEYARLRKIADENGAYLMADMAHISGLVAAGVVPSPFEHAHVVTTTTHKTLRGPRAGMIFYRKGVKSVDPKTGKEILYNLESLINSAVFPGLQGGPHNHAIAGVAVALKQAMTLEFKVYQHQVVANTRALSEALTELGYKIVTGGSDNHLILVDLRSKGTDGGRAEKVLEACSIACNKNTVPGDRSALRPSGLRLGTPALTSRGLLEKDFQKVAHFIHRGIELTLQIQSDTGVRATLKEFKERLAGDKYQAAVQALREEVESFASLFPLPGLP

SEQ ID NO: 78 - protein, 717 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVKVLKKSHDKMLAQPLKDSDVEVYNIIKKESNRQRVGLELIASENFASRAVLEALGSDLNNKYSEGYPGQRYYGGTEFIDELETLAQKRALQAYKLDPQKWGVNVQPYSGSPANFAVYTALVEPHGRIMGLDLPDGGHLTHGFMTDKKKISATSIFFESMPYKVNPDTGYINYDQLEENARLFHPKLIIAGTSSYSRNLEYARLRKIADENGAYLMADMAHISGLVAAGVVPSPFEHAHVVTTTTHKTLRGPRAGMIFYRKGVKSVDPKTGKEILYNLESLINSAVFPGLQGGPHNHAIAGVAVALKQAMTLEFKVYQHQVVANTRALSEALTELGYKIVTGGSDNHLILVDLRSKGTDGGRAEKVLEACSIACNKNTVPGDRSALRPSGLRLGTPALTSRGLLEKDFQKVAHFIHRGIELTLQIQSDTGVRATLKEFKERLAGDKYQAAVQALREEVESFASLFPLPGLP

SEQ ID NO: 79 - protein, 717 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVGVLKKSHDKMLAQPLKDSDVEVYNIIKKESNRQRVGLELIASENFASRAVLEALGSDLNNKYSEGYPGQRYYGGTEFIDELETLAQKRALQAYKLDPQKWGVNVQPYSGSPANFAVYTALVEPHGRIMGLDLPDGGHLTHGFMTDKKKISATSIFFESMPYKVNPDTGYINYDQLEENARLFHPKLIIAGTSSYSRNLEYARLRKIADENGAYLMADMAHISGLVAAGVVPSPFEHAHVVTTTTHKTLRGPRAGMIFYRKGVKSVDPKTGKEILYNLESLINSAVFPGLQGGPHNHAIAGVAVALKQAMTLEFKVYQHQVVANTRALSEALTELGYKIVTGGSDNHLILVDLRSKGTDGGRAEKVLEACSIACNKNTVPGDRSALRPSGLRLGTPALTSRGLLEKDFQKVAHFIHRGIELTLQIQSDTGVRATLKEFKERLAGDKYQAAVQALREEVESFASLFPLPGLP

SEQ ID NO: 80 - protein, 606 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSEEFKKAVAAKFEEAKKKELAAIANKIVANGKGILAADESVGTMGNRLQRIKVENTEENRRQFREILFSVDSSISQSIGGVILFHETLYQKDSQGKLFRNILKEKGIVVGIKLDQGGAPLAGTNKETTIQGLDGLSERCAQYKKDGVDFGKWRAVLRIADQSPSSLAIQENANALARYASICQQNGLVPIVEPEVIPDGDHDLEHHQYVTEKVLAAVYKALNDHHVYLEGTLLKPNMVTAGHAHTKKYTPEQVAMATVTALHRTVPAAVPGIAFLSGGMSEEDATLNLNAINLNPLPKPWKLSFSYGRALQASALAAWGGKAANKEATQEAFMKRAMANSQAAKGQYVHTGSSGAASTQSLFTACYTY

SEQ ID NO: 81 - protein, 606 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSEEHKKAIAALKEQAKKDELAAIANKIVANGKGILAADESVGTMGNRLQRIKVENTEENRRQFREILFSVDSSISQSIGGVILFHETLYQKDSQGKLFRNILKEKGIVVGIKLDQGGAPLAGTNKETTIQGLDGLSERCAQYKKDGVDFGKWRAVLRIADQSPSSLAIQENANALARYASICQQNGLVPIVEPEVIPDGDHDLEHHQYVTEKVLAAVYKALNDHHVYLEGTLLKPNMVTAGHAHTKKYTPEQVAMATVTALHRTVPAAVPGIAFLSGGMSEEDATLNLNAINLNPLPKPWKLSFSYGRALQASALAAWGGKAANKEATQEAFMKRAMANSQAAKGQYVHTGSSGAASTQSLFTACYTY

SEQ ID NO: 82 - protein, 606 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVNLFKEQQKKELSEIAQSIVANGKGILAADESVGTMGNRLQRIKVENTEENRRQFREILFSVDSSISQSIGGVILFHETLYQKDSQGKLFRNILKEKGIVVGIKLDQGGAPLAGTNKETTIQGLDGLSERCAQYKKDGVDFGKWRAVLRIADQSPSSLAIQENANALARYASICQQNGLVPIVEPEVIPDGDHDLEHHQYVTEKVLAAVYKALNDHHVYLEGTLLKPNMVTAGHAHTKKYTPEQVAMATVTALHRTVPAAVPGIAFLSGGMSEEDATLNLNAINLNPLPKPWKLSFSYGRALQASALAAWGGKAANKEATQEAFMKRAMANSQAAKGQYVHTGSSGAASTQSLFTACYTY

SEQ ID NO: 83 - protein, 606 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVNAFKEAQKKELSEIAQSIVANGKGILAADESVGTMGNRLQRIKVENTEENRRQFREILFSVDSSISQSIGGVILFHETLYQKDSQGKLFRNILKEKGIVVGIKLDQGGAPLAGTNKETTIQGLDGLSERCAQYKKDGVDFGKWRAVLRIADQSPSSLAIQENANALARYASICQQNGLVPIVEPEVIPDGDHDLEHHQYVTEKVLAAVYKALNDHHVYLEGTLLKPNMVTAGHAHTKKYTPEQVAMATVTALHRTVPAAVPGIAFLSGGMSEEDATLNLNAINLNPLPKPWKLSFSYGRALQASALAAWGGKAANKEATQEAFMKRAMANSQAAKGQYVHTGSSGAASTQSLFTACYTY

SEQ ID NO: 84 - protein, 708 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQAALAAYLRGLEIDALLARAVPPTPAYELREKTPAPAEGQAADFVSFYGGLAETAQRAELLGRLARGFGVDHGQVAEQSAGVLHLRQQQREAAVLLQAEDRLRYALVPRYRGLFHHISKLDGGVRFLVQLRADLLEAQALKLVEGPDVREMNGVLKGMLSEWFSSGFLNLERVTWHSPREVLQKISEAEAVHPVKNWMDMKRRVGPYRRCYFFSHRSTPGEPLVVLHVALTGDISSNIQAIVKEHPPSETEEKNKITAAIFYSISLTQQGLQGVELGTFLIKRVVKELQREFPHLGVFSSLSPIPGFTKWLLGLLNSQTKEHGRNELFTDSERKEISEITGGPIRETLKLLLSSSEWVQSEKLVRALQTPLMRLAAWYLYGEKHRGYALNPVANFHLQNGAVLWRINWMADVSLRGITGSAGLMANYRYFLEETGPNSTSYLGSKIIKASEQVLSLVAQFQK

SEQ ID NO: 85 - protein, 708 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQAALAAFLRGLEIDALLAAAVPPTPAYELREKTPAPAEGQAADFVSFYGGLAETAQRAELLGRLARGFGVDHGQVAEQSAGVLHLRQQQREAAVLLQAEDRLRYALVPRYRGLFHHISKLDGGVRFLVQLRADLLEAQALKLVEGPDVREMNGVLKGMLSEWFSSGFLNLERVTWHSPREVLQKISEAEAVHPVKNWMDMKRRVGPYRRCYFFSHRSTPGEPLVVLHVALTGDISSNIQAIVKEHPPSETEEKNKITAAIFYSISLTQQGLQGVELGTFLIKRVVKELQREFPHLGVFSSLSPIPGFTKWLLGLLNSQTKEHGRNELFTDSERKEISEITGGPIRETLKLLLSSSEWVQSEKLVRALQTPLMRLAAWYLYGEKHRGYALNPVANFHLQNGAVLWRINWMADVSLRGITGSAGLMANYRYFLEETGPNSTSYLGSKIIKASEQVLSLVAQFQK

SEQ ID NO: 86 - protein, 708 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLFGLEADELLRRAVPPTPAYELREKTPAPAEGQAADFVSFYGGLAETAQRAELLGRLARGFGVDHGQVAEQSAGVLHLRQQQREAAVLLQAEDRLRYALVPRYRGLFHHISKLDGGVRFLVQLRADLLEAQALKLVEGPDVREMNGVLKGMLSEWFSSGFLNLERVTWHSPREVLQKISEAEAVHPVKNWMDMKRRVGPYRRCYFFSHRSTPGEPLVVLHVALTGDISSNIQAIVKEHPPSETEEKNKITAAIFYSISLTQQGLQGVELGTFLIKRVVKELQREFPHLGVFSSLSPIPGFTKWLLGLLNSQTKEHGRNELFTDSERKEISEITGGPIRETLKLLLSSSEWVQSEKLVRALQTPLMRLAAWYLYGEKHRGYALNPVANFHLQNGAVLWRINWMADVSLRGITGSAGLMANYRYFLEETGPNSTSYLGSKIIKASEQVLSLVAQFQK

SEQ ID NO: 87 - protein, 708 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLRGLEIDELLRRAVPPTPAYELREKTPAPAEGQAADFVSFYGGLAETAQRAELLGRLARGFGVDHGQVAEQSAGVLHLRQQQREAAVLLQAEDRLRYALVPRYRGLFHHISKLDGGVRFLVQLRADLLEAQALKLVEGPDVREMNGVLKGMLSEWFSSGFLNLERVTWHSPREVLQKISEAEAVHPVKNWMDMKRRVGPYRRCYFFSHRSTPGEPLVVLHVALTGDISSNIQAIVKEHPPSETEEKNKITAAIFYSISLTQQGLQGVELGTFLIKRVVKELQREFPHLGVFSSLSPIPGFTKWLLGLLNSQTKEHGRNELFTDSERKEISEITGGPIRETLKLLLSSSEWVQSEKLVRALQTPLMRLAAWYLYGEKHRGYALNPVANFHLQNGAVLWRINWMADVSLRGITGSAGLMANYRYFLEETGPNSTSYLGSKIIKASEQVLSLVAQFQK

SEQ ID NO: 88 - protein, 610 aa, synthetic construct
RFGSQRVLVEPDAGAGVAVMKFKNPPVNSLSLEFLTELVISLEKLENDKSFRGVILTSDRPGVFSAGLDLTEMVGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQARLAAYLEELKKKELKAIAEKIVANGKGILAADESVGTMGNRLQRIKVENTEENRRQFREILFSVDSSISQSIGGVILFHETLYQKDSQGKLFRNILKEKGIVVGIKLDQGGAPLAGTNKETTIQGLDGLSERCAQYKKDGVDFGKWRAVLRIADQSPSSLAIQENANALARYASICQQNGLVPIVEPEVIPDGDHDLEHHQYVTEKVLAAVYKALNDHHVYLEGTLLKPNMVTAGHAHTKKYTPEQVAMATVTALHRTVPAAVPGIAFLSGGMSEEDATLNLNAINLNPLPKPWKLSFSYGRALQASALAAWGGKAANKEATQEAFMKRAMANSQAAKGQYVHTGSSGAASTQSLFTACYTY

SEQ ID NO: 89 - protein, 610 aa, synthetic construct
RFGSQRVLVEPDAGAGVAVMKFKNPPVNSLSLEFLTELVISLEKLENDKSFRGVILTSDRPGVFSAGLDLTEMVGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQAALKAFLEQLKKKELKEIANKIVANGKGILAADESVGTMGNRLQRIKVENTEENRRQFREILFSVDSSISQSIGGVILFHETLYQKDSQGKLFRNILKEKGIVVGIKLDQGGAPLAGTNKETTIQGLDGLSERCAQYKKDGVDFGKWRAVLRIADQSPSSLAIQENANALARYASICQQNGLVPIVEPEVIPDGDHDLEHHQYVTEKVLAAVYKALNDHHVYLEGTLLKPNMVTAGHAHTKKYTPEQVAMATVTALHRTVPAAVPGIAFLSGGMSEEDATLNLNAINLNPLPKPWKLSFSYGRALQASALAAWGGKAANKEATQEAFMKRAMANSQAAKGQYVHTGSSGAASTQSLFTACYTY

SEQ ID NO: 90 - protein, 610 aa, synthetic construct
RFGSQRVLVEPDAGAGVAVMKFKNPPVNSLSLEFLTELVISLEKLENDKSFRGVILTSDRPGVFSAGLDLTEMVGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQARLAAFLEELRKKELAAIANKIVANGKGILAADESVGTMGNRLQRIKVENTEENRRQFREILFSVDSSISQSIGGVILFHETLYQKDSQGKLFRNILKEKGIVVGIKLDQGGAPLAGTNKETTIQGLDGLSERCAQYKKDGVDFGKWRAVLRIADQSPSSLAIQENANALARYASICQQNGLVPIVEPEVIPDGDHDLEHHQYVTEKVLAAVYKALNDHHVYLEGTLLKPNMVTAGHAHTKKYTPEQVAMATVTALHRTVPAAVPGIAFLSGGMSEEDATLNLNAINLNPLPKPWKLSFSYGRALQASALAAWGGKAANKEATQEAFMKRAMANSQAAKGQYVHTGSSGAASTQSLFTACYTY

SEQ ID NO: 91 - protein, 610 aa, synthetic construct
RFGSQRVLVEPDAGAGVAVMKFKNPPVNSLSLEFLTELVISLEKLENDKSFRGVILTSDRPGVFSAGLDLTEMVGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLEELKKKELSEIAQSIVANGKGILAADESVGTMGNRLQRIKVENTEENRRQFREILFSVDSSISQSIGGVILFHETLYQKDSQGKLFRNILKEKGIVVGIKLDQGGAPLAGTNKETTIQGLDGLSERCAQYKKDGVDFGKWRAVLRIADQSPSSLAIQENANALARYASICQQNGLVPIVEPEVIPDGDHDLEHHQYVTEKVLAAVYKALNDHHVYLEGTLLKPNMVTAGHAHTKKYTPEQVAMATVTALHRTVPAAVPGIAFLSGGMSEEDATLNLNAINLNPLPKPWKLSFSYGRALQASALAAWGGKAANKEATQEAFMKRAMANSQAAKGQYVHTGSSGAASTQSLFTACYTY

SEQ ID NO: 92 - protein, 610 aa, synthetic construct
RFGSQRVLVEPDAGAGVAVMKFKNPPVNSLSLEFLTELVISLEKLENDKSFRGVILTSDRPGVFSAGLDLTEMVGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLEELRKKELSEIAQSIVANGKGILAADESVGTMGNRLQRIKVENTEENRRQFREILFSVDSSISQSIGGVILFHETLYQKDSQGKLFRNILKEKGIVVGIKLDQGGAPLAGTNKETTIQGLDGLSERCAQYKKDGVDFGKWRAVLRIADQSPSSLAIQENANALARYASICQQNGLVPIVEPEVIPDGDHDLEHHQYVTEKVLAAVYKALNDHHVYLEGTLLKPNMVTAGHAHTKKYTPEQVAMATVTALHRTVPAAVPGIAFLSGGMSEEDATLNLNAINLNPLPKPWKLSFSYGRALQASALAAWGGKAANKEATQEAFMKRAMANSQAAKGQYVHTGSSGAASTQSLFTACYTY

SEQ ID NO: 93 - protein, 832 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSKEFQDAVKAAFQKQYDARLKALYPAVNQQETPLPRSWSPKDKYNYIGLSQGNLRVHYKGHGKNHKDAASVRATHPIPAACGIYYFEVKIVSKGRDGYMGIGLSAQGVNMNRLPGWDKHSYGYHGDDGHSFDSSGTGQPYGPTFTTGDVIGVVVNLIAGTLFYTKNGHSLGIAFTDLPANLYPTVGLQTPGEIVDANFGQQPFLFDIEDYMREWRAKVQGTVHLFPISARLGEWQAVLQNMVSSYLVHHGYVATATAFARMTETPIQEEQASIKNRQKIQKLVLEGRVGEAIETTQRFYPGLLEHNPNLLFMLKSRQFVEMVNGTDSEVRSLSSRSPKSQDSYPGSPSLSPRHGPSSSHMHNTGADSPSASNGVASTKSKQNHSKYPAPSSSSSSSSSSSSSSPSSVNYSESNSTDSTKSQHHSSTSNQETSDSEMEMEAEHYPNGVLGSMSTRIVNGAYKHEDLQTDESSMDDRHPRRQLLGGNQAATERIILFGRELQALSEQLGREYGKNLAHTEMLQDAFSLLAYSDPWSAPVGQQLDPIQREPVCAALNSAILESQNLPKQPPLMLALGQASECLRLMARAGLGSCSFA

SEQ ID NO: 94 - protein, 832 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSPEHQAAIAALFQKQYDARLKKLYPAVNQQETPLPRSWSPKDKYNYIGLSQGNLRVHYKGHGKNHKDAASVRATHPIPAACGIYYFEVKIVSKGRDGYMGIGLSAQGVNMNRLPGWDKHSYGYHGDDGHSFDSSGTGQPYGPTFTTGDVIGVVVNLIAGTLFYTKNGHSLGIAFTDLPANLYPTVGLQTPGEIVDANFGQQPFLFDIEDYMREWRAKVQGTVHLFPISARLGEWQAVLQNMVSSYLVHHGYVATATAFARMTETPIQEEQASIKNRQKIQKLVLEGRVGEAIETTQRFYPGLLEHNPNLLFMLKSRQFVEMVNGTDSEVRSLSSRSPKSQDSYPGSPSLSPRHGPSSSHMHNTGADSPSASNGVASTKSKQNHSKYPAPSSSSSSSSSSSSSSPSSVNYSESNSTDSTKSQHHSSTSNQETSDSEMEMEAEHYPNGVLGSMSTRIVNGAYKHEDLQTDESSMDDRHPRRQLLGGNQAATERIILFGRELQALSEQLGREYGKNLAHTEMLQDAFSLLAYSDPWSAPVGQQLDPIQREPVCAALNSAILESQNLPKQPPLMLALGQASECLRLMARAGLGSCSFA

SEQ ID NO: 95 - protein, 832 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVALFEQELSRRLQRLYPAVNQQETPLPRSWSPKDKYNYIGLSQGNLRVHYKGHGKNHKDAASVRATHPIPAACGIYYFEVKIVSKGRDGYMGIGLSAQGVNMNRLPGWDKHSYGYHGDDGHSFDSSGTGQPYGPTFTTGDVIGVVVNLIAGTLFYTKNGHSLGIAFTDLPANLYPTVGLQTPGEIVDANFGQQPFLFDIEDYMREWRAKVQGTVHLFPISARLGEWQAVLQNMVSSYLVHHGYVATATAFARMTETPIQEEQASIKNRQKIQKLVLEGRVGEAIETTQRFYPGLLEHNPNLLFMLKSRQFVEMVNGTDSEVRSLSSRSPKSQDSYPGSPSLSPRHGPSSSHMHNTGADSPSASNGVASTKSKQNHSKYPAPSSSSSSSSSSSSSSPSSVNYSESNSTDSTKSQHHSSTSNQETSDSEMEMEAEHYPNGVLGSMSTRIVNGAYKHEDLQTDESSMDDRHPRRQLLGGNQAATERIILFGRELQALSEQLGREYGKNLAHTEMLQDAFSLLAYSDPWSAPVGQQLDPIQREPVCAALNSAILESQNLPKQPPLMLALGQASECLRLMARAGLGSCSFA

SEQ ID NO: 96 - protein, 832 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVAKFEEELSRRLQRLYPAVNQQETPLPRSWSPKDKYNYIGLSQGNLRVHYKGHGKNHKDAASVRATHPIPAACGIYYFEVKIVSKGRDGYMGIGLSAQGVNMNRLPGWDKHSYGYHGDDGHSFDSSGTGQPYGPTFTTGDVIGVVVNLIAGTLFYTKNGHSLGIAFTDLPANLYPTVGLQTPGEIVDANFGQQPFLFDIEDYMREWRAKVQGTVHLFPISARLGEWQAVLQNMVSSYLVHHGYVATATAFARMTETPIQEEQASIKNRQKIQKLVLEGRVGEAIETTQRFYPGLLEHNPNLLFMLKSRQFVEMVNGTDSEVRSLSSRSPKSQDSYPGSPSLSPRHGPSSSHMHNTGADSPSASNGVASTKSKQNHSKYPAPSSSSSSSSSSSSSSPSSVNYSESNSTDSTKSQHHSSTSNQETSDSEMEMEAEHYPNGVLGSMSTRIVNGAYKHEDLQTDESSMDDRHPRRQLLGGNQAATERIILFGRELQALSEQLGREYGKNLAHTEMLQDAFSLLAYSDPWSAPVGQQLDPIQREPVCAALNSAILESQNLPKQPPLMLALGQASECLRLMARAGLGSCSFA

SEQ ID NO: 97 - protein, 527 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVKKLLEKEKYIEERLEESIKLAKEAGEIVREAMKNEMNVMLKSSPVDLVTATDQKVEKMLISSIKEKYPSHSFIGEESVAAGEKSILTDNPTWIIDPIDGTTNFVHRFPFVAVSIGFAVNKKIEFGVVYSVVEGKMYTARKGKGAFLNGQKLQVSQQEDITKSLLVTELGSSRTPETVRMVLSNMEKLFKIPVHGIRSVGTAAVNMCLVATGGADAYYEMGIHSWDVAGAGIIVTEAGGVLMDVTGGPFDLMSRRVIAANNRILAERIAKEIQVIPLQRDDE

SEQ ID NO: 98 - protein, 527 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVKKKLAEEKYLEERLEDAIKLAKEAGEIVREAMKNEMNVMLKSSPVDLVTATDQKVEKMLISSIKEKYPSHSFIGEESVAAGEKSILTDNPTWIIDPIDGTTNFVHRFPFVAVSIGFAVNKKIEFGVVYSVVEGKMYTARKGKGAFLNGQKLQVSQQEDITKSLLVTELGSSRTPETVRMVLSNMEKLFKIPVHGIRSVGTAAVNMCLVATGGADAYYEMGIHSWDVAGAGIIVTEAGGVLMDVTGGPFDLMSRRVIAANNRILAERIAKEIQVIPLQRDDE

SEQ ID NO: 99 - protein, 527 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVKKKLAKEKELEERYELAVKLAKEAGEIVREAMKNEMNVMLKSSPVDLVTATDQKVEKMLISSIKEKYPSHSFIGEESVAAGEKSILTDNPTWIIDPIDGTTNFVHRFPFVAVSIGFAVNKKIEFGVVYSVVEGKMYTARKGKGAFLNGQKLQVSQQEDITKSLLVTELGSSRTPETVRMVLSNMEKLFKIPVHGIRSVGTAAVNMCLVATGGADAYYEMGIHSWDVAGAGIIVTEAGGVLMDVTGGPFDLMSRRVIAANNRILAERIAKEIQVIPLQRDDE

SEQ ID NO: 100 - protein, 527 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVNFLKREKYLQERMDYAVTLARQAGEVVREAIKNEMNVMLKSSPVDLVTATDQKVEKMLISSIKEKYPSHSFIGEESVAAGEKSILTDNPTWIIDPIDGTTNFVHRFPFVAVSIGFAVNKKIEFGVVYSVVEGKMYTARKGKGAFLNGQKLQVSQQEDITKSLLVTELGSSRTPETVRMVLSNMEKLFKIPVHGIRSVGTAAVNMCLVATGGADAYYEMGIHSWDVAGAGIIVTEAGGVLMDVTGGPFDLMSRRVIAANNRILAERIAKEIQVIPLQRDDE

SEQ ID NO: 101 - protein, 527 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVNFLKEEKYLQERMDYAVTLARQAGEVVREAIKNEMNVMLKSSPVDLVTATDQKVEKMLISSIKEKYPSHSFIGEESVAAGEKSILTDNPTWIIDPIDGTTNFVHRFPFVAVSIGFAVNKKIEFGVVYSVVEGKMYTARKGKGAFLNGQKLQVSQQEDITKSLLVTELGSSRTPETVRMVLSNMEKLFKIPVHGIRSVGTAAVNMCLVATGGADAYYEMGIHSWDVAGAGIIVTEAGGVLMDVTGGPFDLMSRRVIAANNRILAERIAKEIQVIPLQRDDE

SEQ ID NO: 102 - protein, 527 aa, synthetic construct
FETLVVTSEDGITKIMFNRPKKKNAINTEMYHEIMRALKAASKDDSIITVLTGNGDYYSSGNDLTNFTDIPPGGVEEKAKNNAVLLREFVGAFIDFPKPLIAVVNGPAVGISVTLLGLFDAVYASDRATFHTPFSHLGQSPEGCSSYTFPKIMSPAKATEMLIFGKKLTAGEAQAQGLVTEVFPDSTFQKEVWTRLKAFAKLPPNALRISKEVIRKREREKLHAVNAEEANVLQGRWLSDEFTNAVVNFLKREKFLQERMDYAVTLARQAGEVVREAIKNEMNVMLKSSPVDLVTATDQKVEKMLISSIKEKYPSHSFIGEESVAAGEKSILTDNPTWIIDPIDGTTNFVHRFPFVAVSIGFAVNKKIEFGVVYSVVEGKMYTARKGKGAFLNGQKLQVSQQEDITKSLLVTELGSSRTPETVRMVLSNMEKLFKIPVHGIRSVGTAAVNMCLVATGGADAYYEMGIHSWDVAGAGIIVTEAGGVLMDVTGGPFDLMSRRVIAANNRILAERIAKEIQVIPLQRDDE

SEQ ID NO: 103 - protein, 348 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQAALARFLAALRAAAAEARLKKFLSAPIGDRDVIWVDGISHELAINLVTKGINKAYILLGQFLLMHKNEAEFQRWLIENFGATEEEAQQTSHALKEWCAKFL

SEQ ID NO: 104 - protein, 348 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQARLAAFLAALAERRRQERLEKFLSEPIGDRDVIWVDGISHELAINLVTKGINKAYILLGQFLLMHKNEAEFQRWLIENFGATEEEAQQTSHALKEWCAKFL

SEQ ID NO: 105 - protein, 348 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLKEAAREARLRAFLSEPIGEKDVIWVDGISHELAINLVTKGINKAYILLGQFLLMHKNEAEFQRWLIENFGATEEEAQQTSHALKEWCAKFL

SEQ ID NO: 106 - protein, 348 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLAQARREARLRAFLSEPIGEKDVIWVDGISHELAINLVTKGINKAYILLGQFLLMHKNEAEFQRWLIENFGATEEEAQQTSHALKEWCAKFL

SEQ ID NO: 107 - protein, 401 aa, synthetic construct
YTYEDYKNTAEWLLSHTKHRPQVAIIAGSGLGGLTDKLTQAQIFDYGEIPNFPRSTVPGHAGRLVFGFLNGRAVVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAEARVLQKLGADAVGMSTVPEVIVARHCGLRVFGFSLITNKVIMDYESLEKADFASVLEAGKAALPRVTEFLTALLADEAELDEIRAYLASKLPSVEGLHAIVVSDRDGVPVIKVANDNAPEHALRPGFLSTFALATDQGSKLGLSKVKSIISYYNTYQVVQFNRLPLVVSFIASSSANTGLIVSLEKELAPLFEELRQVVE

SEQ ID NO: 108 - protein, 401 aa, synthetic construct
YTYEDYKNTAEWLLSHTKHRPQVAIIAGSGLGGLTDKLTQAQIFDYGEIPNFPRSTVPGHAGRLVFGFLNGRAVVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAEARVLQKLGADAVGMSTVPEVIVARHCGLRVFGFSLITNKVIMDYESLEKANFAEVLAAGAAALPRISRFLGQLLADIAREDEIRAYLSSKLPSVEGLHAIVVSDRDGVPVIKVANDNAPEHALRPGFLSTFALATDQGSKLGLSKVKSIISYYNTYQVVQFNRLPLVVSFIASSSANTGLIVSLEKELAPLFEELRQVVE

SEQ ID NO: 109 - protein, 401 aa, synthetic construct
YTYEDYKNTAEWLLSHTKHRPQVAIIAGSGLGGLTDKLTQAQIFDYGEIPNFPRSTVPGHAGRLVFGFLNGRAVVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAEARVLQKLGADAVGMSTVPEVIVARHCGLRVFGFSLITNKVIMDYESLEKANHEEVLAAGKQAAQKLEQFVSILMADIARDDDLKRFLYKKLPSVEGLHAIVVSDRDGVPVIKVANDNAPEHALRPGFLSTFALATDQGSKLGLSKVKSIISYYNTYQVVQFNRLPLVVSFIASSSANTGLIVSLEKELAPLFEELRQVVE

SEQ ID NO: 110 - protein, 401 aa, synthetic construct
YTYEDYKNTAEWLLSHTKHRPQVAIIAGSGLGGLTDKLTQAQIFDYGEIPNFPRSTVPGHAGRLVFGFLNGRAVVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAEARVLQKLGADAVGMSTVPEVIVARHCGLRVFGFSLITNKVIMDYESLEKANHEEVLAAGKQAAQKLEQFVSILMADWAEEEDLKRFLYKKLPSVEGLHAIVVSDRDGVPVIKVANDNAPEHALRPGFLSTFALATDQGSKLGLSKVKSIISYYNTYQVVQFNRLPLVVSFIASSSANTGLIVSLEKELAPLFEELRQVVE

SEQ ID NO: 111 - protein, 432 aa, synthetic construct
KYIPRRAVLYVPGNDEKKIKKIPSLNVDVAVLDTEDGVAANKKNEARLRIVKTLEDIDLGPTEKMVRVNSVSSGLAEEDLETLLQSRVLPSSLMLPKVESPEEIQWFADKFSFHLKGRKLEQPMNLIPFVETAMGLLNFKAVCEETLKVGPQVGLFLDAVVFGGEDFRASIGATSSKETLDILYARQKIVVIAKAFGLQAIDLVYIDFRDGAGLLRQSREGAAMGFTGKQVIHPNQIAVVQEQFSPSPEKIKWAEELIAAFKEHQQLGKGAFTFQGSMINQADIDEAKAIVEEAKAIQERKEKRAQALAEKVAQEFVETYFTTFDKRRRLLSRLYMGTATLVWNGNAVSGQESLSEFFEMLPSSEFQISVVDSQPVHDEATPSQTTVLVVIKGSVKFEGNKQRDFNQNFILTAQASPSNTVWKIASDDFRFQ

SEQ ID NO: 112 - protein, 432 aa, synthetic construct
KYIPRRAVLYVPGNDEKKIKKIPSLNVDVAVLDTEDGVAANKKNEARLRIVKTLEDIDLGPTEKMVRVNSVSSGLAEEDLETLLQSRVLPSSLMLPKVESPEEIQWFADKFSFHLKGRKLEQPMNLIPFVETAMGLLNFKAVCEETLKVGPQVGLFLDAVVFGGEDFRASIGATSSKETLDILYARQKIVVIAKAFGLQAIDLVYIDFRDGAGLLRQSREGAAMGFTGKQVIHPNQIAVVQEQFSPSPEKIKWAEELIAAFKEHQQLGKGAFTFQGSMIDDAKIAEAEAIVKEAKAIKEREEARKQALAEQAAAAFVEEYFTTFDKRRRLLSRLYMGTATLVWNGNAVSGQESLSEFFEMLPSSEFQISVVDSQPVHDEATPSQTTVLVVIKGSVKFEGNKQRDFNQNFILTAQASPSNTVWKIASDDFRFQ

SEQ ID NO: 113 - protein, 432 aa, synthetic construct
KYIPRRAVLYVPGNDEKKIKKIPSLNVDVAVLDTEDGVAANKKNEARLRIVKTLEDIDLGPTEKMVRVNSVSSGLAEEDLETLLQSRVLPSSLMLPKVESPEEIQWFADKFSFHLKGRKLEQPMNLIPFVETAMGLLNFKAVCEETLKVGPQVGLFLDAVVFGGEDFRASIGATSSKETLDILYARQKIVVIAKAFGLQAIDLVYIDFRDGAGLLRQSREGAAMGFTGKQVIHPNQIAVVQEQFSPSPEKIKWAEELIAAFKEHQQLGKGAFTFQGSMIDMPLLKQAQNTVTLATSIKERKEKTYVDQAKRAAEEFVNVYYTTMDKRRRLLSRLYMGTATLVWNGNAVSGQESLSEFFEMLPSSEFQISVVDSQPVHDEATPSQTTVLVVIKGSVKFEGNKQRDFNQNFILTAQASPSNTVWKIASDDFRFQ

SEQ ID NO: 114 - protein, 525 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQARLAAFLEGLEIAEIAERMKTVKHKILVLSGKGGVGKSTFSAHLAHGLAEDENTQIALLDIDICGPSIPKIMGLEGEQVHQSGSGWSPVYVEDNLGVMSVGFLLSSPDDAVIWRGPKKNGMIKQFLRDVDWGEVDYLIVDTPPGTSDEHLSVVRYLATAHIDGAVIITTPQEVSLQDVRKEINFDRKVKLPIIGVVENMSGFITPKCKKESQIFPPTTGGAELMNQDLEVPLLGRVPLDPLIGKNCDKGQSFFIDAPDSPATLAYRSIIQRIQEFCNL

SEQ ID NO: 115 - protein, 525 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQARLKAFLEEQEIAKIAERMKTVKHKILVLSGKGGVGKSTFSAHLAHGLAEDENTQIALLDIDICGPSIPKIMGLEGEQVHQSGSGWSPVYVEDNLGVMSVGFLLSSPDDAVIWRGPKKNGMIKQFLRDVDWGEVDYLIVDTPPGTSDEHLSVVRYLATAHIDGAVIITTPQEVSLQDVRKEINFDRKVKLPIIGVVENMSGFITPKCKKESQIFPPTTGGAELMNQDLEVPLLGRVPLDPLIGKNCDKGQSFFIDAPDSPATLAYRSIIQRIQEFCNL

SEQ ID NO: 116 - protein, 525 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQAALAAFLEGLEIAEIAERMKTVKHKILVLSGKGGVGKSTFSAHLAHGLAEDENTQIALLDIDICGPSIPKIMGLEGEQVHQSGSGWSPVYVEDNLGVMSVGFLLSSPDDAVIWRGPKKNGMIKQFLRDVDWGEVDYLIVDTPPGTSDEHLSVVRYLATAHIDGAVIITTPQEVSLQDVRKEINFDRKVKLPIIGVVENMSGFITPKCKKESQIFPPTTGGAELMNQDLEVPLLGRVPLDPLIGKNCDKGQSFFIDAPDSPATLAYRSIIQRIQEFCNL

SEQ ID NO: 117 - protein, 525 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLEQLEIEEIKEKMKTVKHKILVLSGKGGVGKSTFSAHLAHGLAEDENTQIALLDIDICGPSIPKIMGLEGEQVHQSGSGWSPVYVEDNLGVMSVGFLLSSPDDAVIWRGPKKNGMIKQFLRDVDWGEVDYLIVDTPPGTSDEHLSVVRYLATAHIDGAVIITTPQEVSLQDVRKEINFDRKVKLPIIGVVENMSGFITPKCKKESQIFPPTTGGAELMNQDLEVPLLGRVPLDPLIGKNCDKGQSFFIDAPDSPATLAYRSIIQRIQEFCNL

SEQ ID NO: 118 - protein, 525 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLEGLEIAEIKEKMKTVKHKILVLSGKGGVGKSTFSAHLAHGLAEDENTQIALLDIDICGPSIPKIMGLEGEQVHQSGSGWSPVYVEDNLGVMSVGFLLSSPDDAVIWRGPKKNGMIKQFLRDVDWGEVDYLIVDTPPGTSDEHLSVVRYLATAHIDGAVIITTPQEVSLQDVRKEINFDRKVKLPIIGVVENMSGFITPKCKKESQIFPPTTGGAELMNQDLEVPLLGRVPLDPLIGKNCDKGQSFFIDAPDSPATLAYRSIIQRIQEFCNL

SEQ ID NO: 119 - protein, 642 aa, synthetic construct
KYIPRRAVLYVPGNDEKKIKKIPSLNVDVAVLDLEDGVAANKKNEARLRIVKTLEDIDLGPTEKMVRVNSVSSGLAEEDLETLLQSRVLPSSLMLPKVESPEEIQWFADKFSFHLKGRKLEQPMNLIPFVETAMGLLNFKAVCEETLKVGPQVGLFLDAVVFGGEDFRASIGATSSKETLDILYARQKIVVIAKAFGLQAIDLVYIDFRDGAGLLRQSREGAAMGFTGKQVIHPNQIAVVQEQFSPSPEKIKWAEELIAAFKEHQQLGKGAFTFQGSMVTQADIDKAKAIIEQAEAELRRFAARWCSELLGGAWRRVQPEELRVYPVSGGLSNLLFRVSLPDHLPSVGEEPREVLLRLYGAILQGVDSLVLESVMFAILAERSLGPQLYGVFPEGRLEQYIPSRPLKTQELREPVLSAAIATKMAQFHGMEMPFTKEPHWLFGTMERYLKQIQDLPPTGLPEMNLLEMYSLKDEMGNLRKLLESTPSPVVFCHNDIQEGNILLLSEPENADSLMLVDFEYSSYNYRGFDIGNHFNEWVYDYTHEEWPFYKARPTDYPTQEQQLHFIRHYLAEAKKGETLSQEEQRKLEEDLLVEVSRYALASHFFWGLWSILQASMSTIEFGYLDYAQSRFQFYFQQKGQLT

SEQ ID NO: 120 - protein, 642 aa, synthetic construct
KYIPRRAVLYVPGNDEKKIKKIPSLNVDVAVLDLEDGVAANKKNEARLRIVKTLEDIDLGPTEKMVRVNSVSSGLAEEDLETLLQSRVLPSSLMLPKVESPEEIQWFADKFSFHLKGRKLEQPMNLIPFVETAMGLLNFKAVCEETLKVGPQVGLFLDAVVFGGEDFRASIGATSSKETLDILYARQKIVVIAKAFGLQAIDLVYIDFRDGAGLLRQSREGAAMGFTGKQVIHPNQIAVVQEQFSPSPEKIKWAEELIAAFKEHQQLGKGAFTFQGSMVDEAKIAEAKAIVEEAEALLRRFAARWCSEHLGGAWRRVQPEELRVYPVSGGLSNLLFRVSLPDHLPSVGEEPREVLLRLYGAILQGVDSLVLESVMFAILAERSLGPQLYGVFPEGRLEQYIPSRPLKTQELREPVLSAAIATKMAQFHGMEMPFTKEPHWLFGTMERYLKQIQDLPPTGLPEMNLLEMYSLKDEMGNLRKLLESTPSPVVFCHNDIQEGNILLLSEPENADSLMLVDFEYSSYNYRGFDIGNHFNEWVYDYTHEEWPFYKARPTDYPTQEQQLHFIRHYLAEAKKGETLSQEEQRKLEEDLLVEVSRYALASHFFWGLWSILQASMSTIEFGYLDYAQSRFQFYFQQKGQLT

SEQ ID NO: 121 - protein, 642 aa, synthetic construct
KYIPRRAVLYVPGNDEKKIKKIPSLNVDVAVLDLEDGVAANKKNEARLRIVKTLEDIDLGPTEKMVRVNSVSSGLAEEDLETLLQSRVLPSSLMLPKVESPEEIQWFADKFSFHLKGRKLEQPMNLIPFVETAMGLLNFKAVCEETLKVGPQVGLFLDAVVFGGEDFRASIGATSSKETLDILYARQKIVVIAKAFGLQAIDLVYIDFRDGAGLLRQSREGAAMGFTGKQVIHPNQIAVVQEQFSPSPEKIKWAEELIAAFKEHQQLGKGAFTFQGSMIDMPLLKQAQNTVTLATSLLELKAYQWAREYLGGAWRRVQPEELRVYPVSGGLSNLLFRVSLPDHLPSVGEEPREVLLRLYGAILQGVDSLVLESVMFAILAERSLGPQLYGVFPEGRLEQYIPSRPLKTQELREPVLSAAIATKMAQFHGMEMPFTKEPHWLFGTMERYLKQIQDLPPTGLPEMNLLEMYSLKDEMGNLRKLLESTPSPVVFCHNDIQEGNILLLSEPENADSLMLVDFEYSSYNYRGFDIGNHFNEWVYDYTHEEWPFYKARPTDYPTQEQQLHFIRHYLAEAKKGETLSQEEQRKLEEDLLVEVSRYALASHFFWGLWSILQASMSTIEFGYLDYAQSRFQFYFQQKGQLT

SEQ ID NO: 122 - protein, 642 aa, synthetic construct
KYIPRRAVLYVPGNDEKKIKKIPSLNVDVAVLDLEDGVAANKKNEARLRIVKTLEDIDLGPTEKMVRVNSVSSGLAEEDLETLLQSRVLPSSLMLPKVESPEEIQWFADKFSFHLKGRKLEQPMNLIPFVETAMGLLNFKAVCEETLKVGPQVGLFLDAVVFGGEDFRASIGATSSKETLDILYARQKIVVIAKAFGLQAIDLVYIDFRDGAGLLRQSREGAAMGFTGKQVIHPNQIAVVQEQFSPSPEKIKWAEELIAAFKEHQQLGKGAFTFQGSMIDMPLLKQAQNTVTLATSLLERKAYQWAREYLGGAWRRVQPEELRVYPVSGGLSNLLFRVSLPDHLPSVGEEPREVLLRLYGAILQGVDSLVLESVMFAILAERSLGPQLYGVFPEGRLEQYIPSRPLKTQELREPVLSAAIATKMAQFHGMEMPFTKEPHWLFGTMERYLKQIQDLPPTGLPEMNLLEMYSLKDEMGNLRKLLESTPSPVVFCHNDIQEGNILLLSEPENADSLMLVDFEYSSYNYRGFDIGNHFNEWVYDYTHEEWPFYKARPTDYPTQEQQLHFIRHYLAEAKKGETLSQEEQRKLEEDLLVEVSRYALASHFFWGLWSILQASMSTIEFGYLDYAQSRFQFYFQQKGQLT

SEQ ID NO: 123 - protein, 642 aa, synthetic construct
KYIPRRAVLYVPGNDEKKIKKIPSLNVDVAVLDLEDGVAANKKNEARLRIVKTLEDIDLGPTEKMVRVNSVSSGLAEEDLETLLQSRVLPSSLMLPKVESPEEIQWFADKFSFHLKGRKLEQPMNLIPFVETAMGLLNFKAVCEETLKVGPQVGLFLDAVVFGGEDFRASIGATSSKETLDILYARQKIVVIAKAFGLQAIDLVYIDFRDGAGLLRQSREGAAMGFTGKQVIHPNQIAVVQEQFSPSPEKIKWAEELIAAFKEHQQLGKGAFTFQGSMIDMPLLKQAQNTVTLATSLLERRAYQWAREYLGGAWRRVQPEELRVYPVSGGLSNLLFRVSLPDHLPSVGEEPREVLLRLYGAILQGVDSLVLESVMFAILAERSLGPQLYGVFPEGRLEQYIPSRPLKTQELREPVLSAAIATKMAQFHGMEMPFTKEPHWLFGTMERYLKQIQDLPPTGLPEMNLLEMYSLKDEMGNLRKLLESTPSPVVFCHNDIQEGNILLLSEPENADSLMLVDFEYSSYNYRGFDIGNHFNEWVYDYTHEEWPFYKARPTDYPTQEQQLHFIRHYLAEAKKGETLSQEEQRKLEEDLLVEVSRYALASHFFWGLWSILQASMSTIEFGYLDYAQSRFQFYFQQKGQLT

SEQ ID NO: 124 - protein, 604 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQAALAAFLAAAAAAAERAALLAEFLAHFEQVIRDLTEDEMGHPEIGDAIARLKEVLEYNAIGGKYNRGLTVVVAFRELVEPRKQDADSLQRAWTVGWCVELLQAFFLVADDIMDSSLTRRGQIAWYQKPGVGLDAINDANLLEASIYRLLKLYCREQPYYLNLIELFLQSSYQTEIGQTLDLLTAPQGNVDLVRFTEKRYKSIVKYKTAFYSFYLPIAAAMYMAGIDGEKEHANAKKILLEMGEFFQIQDDYLDLFGDPSVTGKIGTDIQDNKLSWLVVQALQRATPEQYQILKENYGQKEAEKVARVKALYEELDLPAVFLQYEEDSYSHIMALIEQYAAPLPPAVFLGLARKIYKR

SEQ ID NO: 125 - protein, 604 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISQPEVQAALAAFLAALAAAAEEAALLAEFLAHFETIIKDLTEDEMGHPEIGDAIARLKEVLEYNAIGGKYNRGLTVVVAFRELVEPRKQDADSLQRAWTVGWCVELLQAFFLVADDIMDSSLTRRGQIAWYQKPGVGLDAINDANLLEASIYRLLKLYCREQPYYLNLIELFLQSSYQTEIGQTLDLLTAPQGNVDLVRFTEKRYKSIVKYKTAFYSFYLPIAAAMYMAGIDGEKEHANAKKILLEMGEFFQIQDDYLDLFGDPSVTGKIGTDIQDNKLSWLVVQALQRATPEQYQILKENYGQKEAEKVARVKALYEELDLPAVFLQYEEDSYSHIMALIEQYAAPLPPAVFLGLARKIYKR

SEQ ID NO: 126 - protein, 604 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLAAEAAYAQEKQDFVQHFSQIVRVLTEDEMGHPEIGDAIARLKEVLEYNAIGGKYNRGLTVVVAFRELVEPRKQDADSLQRAWTVGWCVELLQAFFLVADDIMDSSLTRRGQIAWYQKPGVGLDAINDANLLEASIYRLLKLYCREQPYYLNLIELFLQSSYQTEIGQTLDLLTAPQGNVDLVRFTEKRYKSIVKYKTAFYSFYLPIAAAMYMAGIDGEKEHANAKKILLEMGEFFQIQDDYLDLFGDPSVTGKIGTDIQDNKLSWLVVQALQRATPEQYQILKENYGQKEAEKVARVKALYEELDLPAVFLQYEEDSYSHIMALIEQYAAPLPPAVFLGLARKIYKR

SEQ ID NO: 127 - protein, 604 aa, synthetic construct
RFGSQRVLVEPDAGAGVAVMKFKNPPVNSLSLEFLTELVISLEKLENDKSFRGVILTSDRPGVFSAGLDLTEMTGRSPAHYAGYWKAVQELWLRLYQSNLVLVSAINGACPAGGALVALTCDYRILADNPRYPIGLNETQLGIIAPFWLKDTLENTIGHRAAERALQLGLLFPPAEALQVGIVDQVVPEEQVQSTALSAIAQWMAIPDHARQLTKAMMRKATASRLVTQRDADVQNFVSFISKDSIQKSLQMYLERLAAAAAYAQEKQDFVQHFSQIVRVLTEDEMGHPEIGDAIARLKEVLEYNAIGGKYNRGLTVVVAFRELVEPRKQDADSLQRAWTVGWCVELLQAFFLVADDIMDSSLTRRGQIAWYQKPGVGLDAINDANLLEASIYRLLKLYCREQPYYLNLIELFLQSSYQTEIGQTLDLLTAPQGNVDLVRFTEKRYKSIVKYKTAFYSFYLPIAAAMYMAGIDGEKEHANAKKILLEMGEFFQIQDDYLDLFGDPSVTGKIGTDIQDNKLSWLVVQALQRATPEQYQILKENYGQKEAEKVARVKALYEELDLPAVFLQYEEDSYSHIMALIEQYAAPLPPAVFLGLARKIYKR

SEQ ID NO: 128 - protein, 659 aa, synthetic construct
MGWSCIILFLVATATGVHSHHHHHHGSSAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTGGGSGGSGYTYEDYKNTAEWLLSHTKHRPQVAIIAGSGLGGLTDKLTQAQIFDYGEIPNFPRSTVPGHAGRLVFGFLNGRAVVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAEARVLQKLGADAVGMSTVPEVIVARHCGLRVFGFSLITNKVIMDYESLEKANFAEVLAAGAAALPRISRFLGQLLADIAREDEIRAYLSSKLPSVEGLHAIVVSDRDGVPVIKVANDNAPEHALRPGFLSTFALATDQGSKLGLSKVKSIISYYNTYQVVQFNRLPLVVSFIASSSANTGLIVSLEKELAPLFEELRQVVE

SEQ ID NO: 129 - protein, 659 aa, synthetic construct
MGWSCIILFLVATATGVHSHHHHHHGSSAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTGGGSGGSGYTYEDYKNTAEWLLSHTKHRPQVAIIAGSGLGGLTDKLTQAQIFDYGEIPNFPRSTVPGHAGRLVFGFLNGRAVVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAEARVLQKLGADAVGMSTVPEVIVARHCGLRVFGFSLITNKVIMDYESLEKANHEEVLAAGKQAAQKLEQFVSILMADIARDDDLKRFLYKKLPSVEGLHAIVVSDRDGVPVIKVANDNAPEHALRPGFLSTFALATDQGSKLGLSKVKSIISYYNTYQVVQFNRLPLVVSFIASSSANTGLIVSLEKELAPLFEELRQVVE

SEQ ID NO: 130 - protein, 913 aa, synthetic construct
MGWSCIILFLVATATGVHSHHHHHHGSSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALSGGGSGGSGYTYEDYKNTAEWLLSHTKHRPQVAIIAGSGLGGLTDKLTQAQIFDYGEIPNFPRSTVPGHAGRLVFGFLNGRAVVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAEARVLQKLGADAVGMSTVPEVIVARHCGLRVFGFSLITNKVIMDYESLEKANFAEVLAAGAAALPRISRFLGQLLADIAREDEIRAYLSSKLPSVEGLHAIVVSDRDGVPVIKVANDNAPEHALRPGFLSTFALATDQGSKLGLSKVKSIISYYNTYQVVQFNRLPLVVSFIASSSANTGLIVSLEKELAPLFEELRQVVE

SEQ ID NO: 131 - protein, 913 aa, synthetic construct
MGWSCIILFLVATATGVHSHHHHHHGSSVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALGGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLNFRESGQVYFGIIALSGGGSGGSGYTYEDYKNTAEWLLSHTKHRPQVAIIAGSGLGGLTDKLTQAQIFDYGEIPNFPRSTVPGHAGRLVFGFLNGRAVVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAEARVLQKLGADAVGMSTVPEVIVARHCGLRVFGFSLITNKVIMDYESLEKANHEEVLAAGKQAAQKLEQFVSILMADIARDDDLKRFLYKKLPSVEGLHAIVVSDRDGVPVIKVANDNAPEHALRPGFLSTFALATDQGSKLGLSKVKSIISYYNTYQVVQFNRLPLVVSFIASSSANTGLIVSLEKELAPLFEELRQVVE

SEQ ID NO: 132 - protein, 677 aa, synthetic construct
MGWSCIILFLVATATGVHSHHHHHHGSSDIVLTQSPATLSVTPGNSVSLSCRASQSIGNNLHWYQQKSHESPRLLIKYASQSISGIPSRFSGSGSGTDFTLSINSVETEDFGMYFCQQSNSWPYTFGGGTKLEIKRGGGGSGGGGSGGGGSGGGGSDVQLQESGPSLVKPSQTLSLTCSVTGDSITSDYWSWIRKFPGNRLEYMGYVSYSGSTYYNPSLKSRISITRDTSKNQYYLDLNSVTTEDTATYYCANWDGDYWGQGTLVTVSGGGSGGSGYTYEDYKNTAEWLLSHTKHRPQVAIIAGSGLGGLTDKLTQAQIFDYGEIPNFPRSTVPGHAGRLVFGFLNGRAVVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAEARVLQKLGADAVGMSTVPEVIVARHCGLRVFGFSLITNKVIMDYESLEKANFAEVLAAGAAALPRISRFLGQLLADIAREDEIRAYLSSKLPSVEGLHAIVVSDRDGVPVIKVANDNAPEHALRPGFLSTFALATDQGSKLGLSKVKSIISYYNTYQVVQFNRLPLVVSFIASSSANTGLIVSLEKELAPLFEELRQVVE

SEQ ID NO: 133 - protein, 13 aa, synthetic construct
DCAWHLGELVWCT

(19) United States
(12) Patent Application Publication
(10) Pub. No.: US 2026/0357224 A1
(43) Pub. Date: Nov. 12, 2026

(54) DESIGNED PROTEIN CAGE NANOPARTICLES BASED ON HUMAN AND MAMMALIAN PROTEIN SEQUENCES

(71) Applicant: Avimer Bio Corp., San Diego, CA (US)

(72) Inventors: Todd O. Yeates, Agoura Hills, CA (US); Peter Bowers, San Diego, CA (US); Nika Gladkov, Santa Cruz, CA (US); Robin Aglietti, South San Francisco, CA (US)

(73) Assignee: Avimer Bio Corp., San Diego, CA (US)

(21) Appl. No.: 19/536,940

(22) Filed: Feb. 11, 2026

Related U.S. Application Data
(60) Provisional application No. 63/757,279, filed on Feb. 11, 2025.

(51) Int. Cl. C12N 9/10; C07K 14/47; C07K 14/705; C12N 9/88; C12N 9/90

(52) U.S. Cl. CPC C12N 9/1077; C07K 14/47; C07K 14/4702; C07K 14/70532; C12N 9/88; C12N 9/90; C12Y 204/02001; C12Y 401/03025; C12Y 503/03008

(57) ABSTRACT

Provided herein are fusion protein subunits and polypeptides. The fusion protein subunit and polypeptides comprise an N-terminal domain comprising a first mammalian or human homo-oligomeric protein or portion thereof and a C-terminal domain comprising a second mammalian or human homo-oligomeric protein or portion thereof. The fusion protein subunit and polypeptides are capable of self-assembling into a three-dimensional multi-subunit structure. Also provided are methods of producing the polypeptides and fusion protein subunits.

### CROSS-REFERENCE TO RELATED APPLICATIONS

This application claims priority to U.S. Provisional Application No. 63/757,279, filed Feb. 11, 2025, the entire disclosure of which is hereby incorporated by reference herein.

### REFERENCE TO A SEQUENCE LISTING

The contents of the electronic sequence listing (AVB0001-00US_ST26_2026-06-25.xml; Size: 203,692 bytes; Date of Creation: Jun. 25, 2026) is incorporated herein by reference.

### BACKGROUND

Many proteins found in nature can self-assemble from multiple copies of the same subunit to give highly complex architectures. Examples include viral capsids, ferritin, clathrin, microtubules, and the like. The formation of such molecular assemblies from smaller protein subunits confers various functional capabilities and consequent selective advantages. The diverse utilities provided by large natural protein assemblies have motivated the creation of protein assemblies that are distinct from any known in nature. Protein cages are useful in medical applications, for example, to present multiple copies of viral antigens to be used as a vaccine (PMID: 33160446) or to present antibodies to bind cell surfaces (PMID: 33795432).

One type of protein assembly architecture of interest includes protein nanoparticles in the form of capsids or cages, with three-dimensional shapes reminiscent of the Platonic solids (e.g., tetrahedron, cube, icosahedron). And while naturally occurring protein assemblies have been studied previously, protein cages can also be designed based on novel (e.g., non-naturally occurring) protein sequences. Such diverse protein cages can be created by engineering a novel protein subunit formed from a fusion or concatenation of two specifically chosen sequences derived from homo-oligomeric proteins (e.g. dimers or trimers). Padilla et al. (2001) (PMID: 11226219); Lai et al. (2012. PMID: 22654051).

To date, however, such engineered fusion proteins are limited in number. Only a small number of protein cages have been successfully designed, limiting available choices for the size and shape of the cage and the disposition of (e.g. the spacing between) effector domains on the surface. Further, the small number of protein cages have been designed using non-human/non-mammalian protein components such as bacterial and viral protein components (Lai et al. (2014, PMID: 25411884)) or from components with entirely de novo (i.e., non-natural) protein sequences. The importance of using human, or humanized, protein sequences in therapeutic settings is well-understood. Owing to the generally high immunogenic properties of foreign proteins when introduced in to a human host, including those engineered by purely computational de novo design methods, it would be desirable to generate novel protein cages comprised of human or other closely related mammalian sequences to reduce the immunogenicity and potential adverse reactions for therapeutic uses.

### BRIEF DESCRIPTION OF THE DRAWINGS

FIG. 1 shows a 3D rendering of an individual human fusion protein subunit (Subunit 1, left panel) and the self-assembling human protein cage formed by 12 copies of Subunit 1 in two different orientations (Designed Human Protein Cage No. 1 or “DHPC-1”, middle and right panels) according to one embodiment. In this illustration, Subunit 1 is a fusion formed from portions of proteins Purine nucleoside phosphorylase (N-domain) and Regulator complex protein LAMTOR3 (C-domain), and having an amino acid sequence given by SEQ ID NO:1.

FIG. 2 shows a 3D rendering of an individual human fusion protein subunit (Subunit 2, left panel) and the self-assembling human protein cage formed by 12 copies of Subunit 2 in two different orientations (Designed Human Protein Cage No. 2 or “DHPC-2”, middle and right panels) according to one embodiment. In this illustration, Subunit 2 is a fusion formed from portions of proteins purine nucleoside phosphorylase (N-domain) and N-acylglucosamine 2-epimerase (C-domain), and having an amino acid sequence given by SEQ ID NO:2.

FIG. 3 shows a 3D rendering of an individual human fusion protein subunit (Subunit 3, left panel) and the self-assembling human protein cage formed by 12 copies of Subunit 3 in two different orientations (Designed Human Protein Cage No. 3 or “DHPC-3”, middle and right panels) according to one embodiment. In this illustration, Subunit 3 is a fusion formed from portions of proteins Enoyl-CoA delta isomerase 1—mitochondrial (N-domain) and Farnesyl pyrophosphate synthase (C-domain), and having an amino acid sequence given by SEQ ID NO:3.

FIG. 4 shows a 3D rendering of an individual human fusion protein subunit (Subunit 4, left panel) and the self-assembling human protein cage formed by 12 copies of Subunit 4 in two different orientations (Designed Human Protein Cage No. 4 or “DHPC-4”, middle and right panels) according to one embodiment. In this illustration, Subunit 4 is a fusion formed from portions of proteins Enoyl-CoA delta isomerase 2 (N-domain) and Ran-binding protein 10 (C-domain), and having an amino acid sequence given by SEQ ID NO:4.

FIG. 5 shows a 3D rendering of an individual human fusion protein subunit (Subunit 5, left panel) and the self-assembling human protein cage formed by 12 copies of Subunit 5 in two different orientations (Designed Human Protein Cage No. 5 or “DHPC-5”, middle and right panels) according to one embodiment. In this illustration, Subunit 5 is a fusion formed from portions of proteins Citramalyl-CoA lyase—mitochondrial (N-domain) and NTF2-related export protein 1 (C-domain), and having an amino acid sequence given by SEQ ID NO:5.

FIG. 6 shows a 3D rendering of an individual human fusion protein subunit (Subunit 6, left panel) and the self-assembling human protein cage formed by 12 copies of Subunit 6 in two different orientations (Designed Human Protein Cage No. 6 or “DHPC-6”, middle and right panels) according to one embodiment. In this illustration, Subunit 6 is a fusion formed from portions of proteins Enoyl-CoA delta isomerase 1—mitochondrial (N-domain) and Centromere protein S (C-domain), and having an amino acid sequence given by SEQ ID NO:6.

FIG. 7 shows a 3D rendering of an individual human fusion protein subunit (Subunit 7, left panel) and the self-assembling human protein cage formed by 12 copies of Subunit 7 in two different orientations (Designed Human Protein Cage No. 7 or “DHPC-7”, middle and right panels) according to one embodiment. In this illustration, Subunit 7 is a fusion formed from portions of proteins Enoyl-CoA delta isomerase 2 (N-domain) and Fructose-bisphosphate aldolase B (C-domain), and having an amino acid sequence given by SEQ ID NO:7.

FIG. 8 shows a 3D rendering of an individual human fusion protein subunit (Subunit 8, left panel) and the self-assembling human protein cage formed by 12 copies of Subunit 8 in two different orientations (Designed Human Protein Cage No. 8 or “DHPC-8”, middle and right panels) according to one embodiment. In this illustration, Subunit 8 is a fusion formed from portions of proteins Enoyl-CoA delta isomerase 1 mitochondrial (N-domain) and barrier-to-autointegration factor-like protein (C-domain), and having an amino acid sequence given by SEQ ID NO:8.

FIG. 9 shows a 3D rendering of an individual human fusion protein subunit (Subunit 9, left panel) and the self-assembling human protein cage formed by 12 copies of Subunit 9 in two different orientations (Designed Human Protein Cage No. 9 or “DHPC-9”, middle and right panels) according to one embodiment. In this illustration, Subunit 9 is a fusion formed from portions of proteins Enoyl-CoA delta isomerase 2 (N-domain) and Centromere protein S (C-domain), and having an amino acid sequence given by SEQ ID NO:9.

FIG. 10 shows a 3D rendering of an individual human fusion protein subunit (Subunit 10, left panel) and the self-assembling human protein cage formed by 12 copies of Subunit 10 in two different orientations (Designed Human Protein Cage No. 10 or “DHPC-10”, middle and right panels) according to one embodiment. In this illustration, Subunit 10 is a fusion formed from portions of proteins Enoyl-CoA delta isomerase 1—mitochondrial (N-domain) and Retinol dehydrogenase 10 (C-domain), and having an amino acid sequence given by SEQ ID NO:10.

FIG. 11 shows a 3D rendering of an individual human fusion protein subunit (Subunit 11, left panel) and the self-assembling human protein cage formed by 12 copies of Subunit 11 in two different orientations (Designed Human Protein Cage No. 11 or “DHPC-11”, middle and right panels) according to one embodiment. In this illustration, Subunit 11 is a fusion formed from portions of proteins Enoyl-CoA delta isomerase 1—mitochondrial (N-domain) and Tyrosine-tRNA ligase—mitochondrial (C-domain), and having an amino acid sequence given by SEQ ID NO:11.

FIG. 12 shows a 3D rendering of an individual human fusion protein subunit (Subunit 12, left panel) and the self-assembling human protein cage formed by 12 copies of Subunit 12 in two different orientations (Designed Human Protein Cage No. 12 or “DHPC-12”, middle and right panels) according to one embodiment. In this illustration, Subunit 12 is a fusion formed from portions of proteins Enoyl-CoA delta isomerase 1—mitochondrial (N-domain) and Fructose-bisphosphate aldolase B (C-domain), and having an amino acid sequence given by SEQ ID NO:12.

FIG. 13 shows a 3D rendering of an individual human fusion protein subunit (Subunit 13, left panel) and the self-assembling human protein cage formed by 12 copies of Subunit 13 in two different orientations (Designed Human Protein Cage No. 13 or “DHPC-13”, middle and right panels) according to one embodiment. In this illustration, Subunit 13 is a fusion formed from portions of proteins Citramalyl-CoA lyase—mitochondrial (N-domain) and Choline/ethanolamine kinase (C-domain), and having an amino acid sequence given by SEQ ID NO:13.

FIG. 14 shows a 3D rendering of an individual human fusion protein subunit (Subunit 14, left panel) and the self-assembling human protein cage formed by 12 copies of Subunit 14 in two different orientations (Designed Human Protein Cage No. 14 or “DHPC-14”, middle and right panels) according to one embodiment. In this illustration, Subunit 14 is a fusion formed from portions of proteins Purine nucleoside phosphorylase (N-domain) and Ragulator complex protein LAMTOR5 (C-domain), and having an amino acid sequence given by SEQ ID NO:14.

FIG. 15 shows a 3D rendering of an individual human fusion protein subunit (Subunit 15, left panel) and the self-assembling human protein cage formed by 12 copies of Subunit 15 in two different orientations (Designed Human Protein Cage No. 15 or “DHPC-15”, middle and right panels) according to one embodiment. In this illustration, Subunit 15 is a fusion formed from portions of proteins Enoyl-CoA delta isomerase 2 (N-domain) and Centromere protein S (C-domain), and having an amino acid sequence given by SEQ ID NO:15.

FIG. 16 shows a 3D rendering of an individual human fusion protein subunit (Subunit 16, left panel) and the self-assembling human protein cage formed by 12 copies of Subunit 16 in two different orientations (Designed Human Protein Cage No. 16 or “DHPC-16”, middle and right panels) according to one embodiment. In this illustration, Subunit 16 is a fusion formed from portions of proteins Enoyl-CoA delta isomerase 2 (N-domain) and Serine hydroxymethyltransferase—cytosolic (C-domain), and having an amino acid sequence given by SEQ ID NO:16.

FIG. 17 shows a 3D rendering of an individual human fusion protein subunit (Subunit 17, left panel) and the self-assembling human protein cage formed by 12 copies of Subunit 17 in two different orientations (Designed Human Protein Cage No. 17 or “DHPC-17”, middle and right panels) according to one embodiment. In this illustration, Subunit 17 is a fusion formed from portions of proteins Enoyl-CoA delta isomerase 2 (N-domain) and Centromere protein S (C-domain), and having an amino acid sequence given by SEQ ID NO:17.

FIG. 18 shows a 3D rendering of an individual human fusion protein subunit (Subunit 18, left panel) and the self-assembling human protein cage formed by 12 copies of Subunit 18 in two different orientations (Designed Human Protein Cage No. 18 or “DHPC-18”, middle and right panels) according to one embodiment. In this illustration, Subunit 18 is a fusion formed from portions of proteins Enoyl-CoA delta isomerase 1—mitochondrial (N-domain) and Malonyl-CoA decarboxylase—mitochondrial (C-domain), and having an amino acid sequence given by SEQ ID NO:18.

FIG. 19 shows a 3D rendering of an individual human fusion protein subunit (Subunit 19, left panel) and the self-assembling human protein cage formed by 12 copies of Subunit 19 in two different orientations (Designed Human Protein Cage No. 19 or “DHPC-19”, middle and right panels) according to one embodiment. In this illustration, Subunit 19 is a fusion formed from portions of proteins Enoyl-CoA delta isomerase 1—mitochondrial (N-domain) and Barrier-to-autointegration factor (C-domain), and having an amino acid sequence given by SEQ ID NO:19.

FIG. 20 shows a 3D rendering of an individual human fusion protein subunit (Subunit 20, left panel) and the self-assembling human protein cage formed by 12 copies of Subunit 20 in two different orientations (Designed Human Protein Cage No. 20 or “DHPC-20”, middle and right panels) according to one embodiment. In this illustration, Subunit 20 is a fusion formed from portions of proteins Enoyl-CoA delta isomerase 1—mitochondrial (N-domain) and Cytosolic Fe-S cluster assembly factor NUBP1 (C-domain), and having an amino acid sequence given by SEQ ID NO:20.

FIG. 21 shows a 3D rendering of an individual human fusion protein subunit (Subunit 21, left panel) and the self-assembling human protein cage formed by 12 copies of Subunit 21 in two different orientations (Designed Human Protein Cage No. 21 or “DHPC-21”, middle and right panels) according to one embodiment. In this illustration, Subunit 21 is a fusion formed from portions of proteins Enoyl-CoA delta isomerase 2 (N-domain) and C-domain: Inositol monophosphatase (C-domain), and having an amino acid sequence given by SEQ ID NO:21.

FIG. 22 shows an MSA highlighting sequence variations employed in the design of fusion proteins for production and testing with regards to SEQ ID NO: 107, 108, 109, and 110.

FIG. 23 shows the SDS Gel Analysis of scaled up preparations of human protein cages with SEQ ID NO: 107, 108, and 109.

FIG. 24 shows the Size Exclusion Chromatography (SEC) chromatograms of human protein cages with SEQ ID NO: 107, 108, and 109.

FIG. 25 shows the SEC and SDS of human protein cage with SEQ ID NO: 109 that is rerun after a first SEC step.

FIG. 26 shows the negative stain imaging of human protein cage with SEQ ID NO: 109.

FIG. 27 shows the cryo-EM imaging of human protein cage with SEQ ID NO: 109.

FIG. 28 shows the SEC chromatograms of human protein cage fused to effector protein payloads (SEQ ID NO: 128-132).

FIG. 29A shows the biolayer interferometry (BLI) binding curve for the Fc tagged antigen immobilized binding to analyte nanocages with SEQ ID NO: 129 and 130. FIG. 29B shows the BLI binding curve for the His tag immobilized nanocages binding to Fc tagged antigens for SEQ ID NO: 129 and 130.

### DETAILED DESCRIPTION

The field of the invention concerns engineered human or mammalian fusion protein subunits that are capable of self-assembling into mammalian or human protein nanoparticles- or protein cages-from multiple copies of the engineered human or mammalian fusion protein subunits. These designed human or mammalian protein cages provide a platform that lend to diverse medical therapeutic applications.

### DEFINITIONS

As used herein, the term “antibody” includes intact antibodies and any antigen-binding fragment (i.e. “antigen-binding fragment”) or single chains thereof. The antibody may be monoclonal, monospecific, multispecific (e.g., bispecific, trispecific, and so on), multifunctional, and/or humanized, and may be made by any method known in the art, including using hybridoma or recombinant methods.

As used herein, the term “antigen-binding fragment” of an antibody (or simply “antibody fragment”) refers to one or more fragments of an antibody that retain the ability to specifically bind to an antigen on the therapeutic target. Examples of binding fragments encompassed within the term “antigen-binding fragment” of an antibody include a Fab fragment, a Fab′ fragment, a F(ab′)₂ fragment, an Fd fragment; an Fv fragment, an scFv fragment, diabodies, minibodies, linear antibodies, single-chain antibody molecules, and multispecific antibodies formed from antibody fragment(s) or proteins, domains and engineered fragments of cytokines, chemokines or other functional binding proteins that may be attached to the nanocages.

As used herein, a “protein cage” refers to a plurality of polypeptides (“subunits” or “oligomeric protein units”) which collectively form a multi-subunit three-dimensional structure having an outer surface, which may have an interior cavity.

As used herein, a “designed protein cage” or “designed protein nanoparticle” refers to a protein cage formed by a plurality of fusion protein subunits derived from natural or endogenous proteins that self-assemble into a multi-subunit three-dimensional structure that can be used to display or deliver effector proteins.

As used herein, a “designed human protein cage” or “designed human protein nanoparticle” refers to a protein cage formed by a plurality of human fusion protein subunits derived from endogenous human proteins that self-assemble into a multi-subunit three-dimensional structure that can be used to display or deliver effector proteins.

As used herein, a “designed mammalian protein cage” or “designed mammalian protein nanoparticle” refers to a protein cage formed by a plurality of mammalian fusion protein subunits derived from endogenous mammalian proteins that self-assemble into a multi-subunit three-dimensional structure that can be used to display or deliver effector proteins.

As used herein, a mammalian or human “fusion protein subunit” is a fusion protein that is produced by fusing all or a portion of two oligomeric human or mammalian proteins or polypeptides that can self-assemble into a protein cage (e.g., a designed human protein cage or a designed mammalian protein cage). Said proteins or polypeptides may be fused together directly or via a linker.

As used herein, the terms “protein”, “polypeptide” and “peptide” are used interchangeably to refer to two or more amino acids linked together. Groups or strings of amino acid abbreviations (e.g., 1 letter abbreviations or 3 letter abbreviations known by those skilled in the art) are used to represent peptides. An “X” abbreviation used herein represents any amino acid. A protein, polypeptide, or peptide is encoded by a nucleotide sequence in accordance with the genetic code, and thus the nucleotide sequence used to generate the proteins, polypeptides, or peptides disclosed herein may vary based on the genetic code that may encode each amino acid in a protein, polypeptide, or peptide sequence. Except when specifically indicated, peptides are indicated with the N-terminus on the left and the sequence is written from the N-terminus to the C-terminus. Except when specifically indicated, peptides are indicated with the N-terminus on the left and the sequences are written from the N-terminus to the C-terminus. Similarly, except when specifically indicated, nucleic acid sequences are indicated with the 5′ end on the left and the sequences are written from 5′ to 3′.

As used herein, a “nucleic acid molecule” or “polynucleotide” refers to a polymeric compound including covalently linked nucleotides comprising natural subunits (e.g., purine or pyrimidine bases). Purine bases include adenine (A) and guanine (G), and pyrimidine bases include uracil (U), thymine (T), and cytosine (C). Nucleic acid molecules include polyribonucleic acid (RNA) and polydeoxyribonucleic acid (DNA), which includes cDNA, genomic DNA, and synthetic DNA, either of which may be single or double-stranded. A nucleic acid molecule encoding an amino acid sequence includes all nucleotide sequences that encode the same amino acid sequence. In addition to the single letter codes for the nucleotides A, G, U, T, C, nucleotide sequences may use IUPAC single letter codes to designate more than one nucleotide alternative/ambiguous sequence, e.g., R (A or G); Y (C or T/U); M (A or C); K (G or T); S (C or G); W (A or T); H (A or C or T); B (C or G or T); V (A or C or G); D (A or G or T); or N (A or C or G or T).

The terms “nucleotide” and “nucleic acid” are used interchangeably and refer to any nucleoside linked to a phosphate group. Nucleic acids in accordance with the embodiments described herein may include nucleotides entirely of the types found in naturally occurring nucleic acids, or may instead include one or more nucleotide analogs or have a structure that otherwise differs from that of a naturally occurring nucleic acid.

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., (1990) J Mol Biol 215:403-410. Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared. For purposes herein, where a given comparison window (e.g., over 80% of the given sequence) is not provided, the recited sequence identity is over 100% of the given sequence. Additionally, for the percentages of sequence identity of the proteins provided herein, the percentages are determined using BLASTP 2.8.0+, scoring matrix BLOSUM62, and the default parameters available at blast.ncbi.nlm.nih.gov/Blast.cgi. See also Altschul, et al., (1997) Nucleic Acids Res 25:3389-3402; and Altschul, et al., (2005) FEBS J 272:5101-5109.

Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection.

The term “pharmaceutical composition” refers to a composition suitable for pharmaceutical use in a subject. Pharmaceutical compositions may be formulated for the intended route of delivery and administered to subjects accordingly using methods in the art. Pharmaceutical compositions may include one or more of the following: a pharmaceutically acceptable vehicle, pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art. See, e.g., Carra et al., (2007) Vaccine 25:4149-4158.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

As provided herein, a sequence that falls within a genus sequence means that the sequence must have amino acid residues that are identical to those specifically enumerated for the genus sequence when optimally aligned thereto. That is, the sequence must comprise, when optimally aligned with the genus sequence, the same amino acid residues as the genus sequence except for Xaa's that are “any amino acid”.

To the extent necessary to understand or complete the disclosure of the present invention, all publications, patents, and patent applications mentioned herein are expressly incorporated by reference therein to the same extent as though each were individually so incorporated.

### Polypeptide Constructs

Various embodiments of the present invention provide novel polypeptides such as the designed human and mammalian protein cages and fusion proteins subunits described herein. In one or more embodiments, the polypeptide has at least 90%, at least 91%, at least 92%, at least 93%, at least 94%, at least 95%, at least 96%, at least 97%, at least 98%, at least 99%, or 100% sequence identity to SEQ ID NO: 1-21. The polypeptide may contain deletions, substitution, and/or insertions relative to any of SEQ ID NO: 1-21, such as having 1, 2, 3, 4, 5, 6, 7, 8, 9, 10, 11, 12, 13, 14, 15, or more deletions, substitutions and/or insertions to the amino acid sequence described by SEQ ID NO: 1-21. These sequences are discussed in detail below in Example 1.

In some embodiments, the polypeptide has at least 90%, at least 91%, at least 92%, at least 93%, at least 94%, at least 95%, at least 96%, at least 97%, at least 98%, at least 99%, or 100% sequence identity to SEQ ID NO: 22-63. The polypeptide may contain deletions, substitution, and/or insertions relative to any of SEQ ID NO: 22-63, such as having 1, 2, 3, 4, 5, 6, 7, 8, 9, 10, 11, 12, 13, 14, 15, or more deletions, substitutions and/or insertions to the amino acid sequence described by SEQ ID NO: 22-63. These sequences are discussed in detail below in Example 2.

In some embodiments, the polypeptide has at least 90%, at least 91%, at least 92%, at least 93%, at least 94%, at least 95%, at least 96%, at least 97%, at least 98%, at least 99%, or 100% sequence identity to SEQ ID NO: 64-127. The polypeptide may contain deletions, substitution, and/or insertions relative to any of SEQ ID NO: 64-127, such as having 1, 2, 3, 4, 5, 6, 7, 8, 9, 10, 11, 12, 13, 14, 15, or more deletions, substitutions and/or insertions to the amino acid sequence described by SEQ ID NO: 64-127. These sequences are discussed in detail below in Example 3.

In some embodiments, the polypeptide has at least 90%, at least 91%, at least 92%, at least 93%, at least 94%, at least 95%, at least 96%, at least 97%, at least 98%, at least 99%, or 100% sequence identity to SEQ ID NO: 107-109. The polypeptide may contain deletions, substitution, and/or insertions relative to any of SEQ ID NO: 107-109, such as having 1, 2, 3, 4, 5, 6, 7, 8, 9, 10, 11, 12, 13, 14, 15, or more deletions, substitutions and/or insertions to the amino acid sequence described by SEQ ID NO: 107-109.

In some embodiments, the polypeptide has at least 90%, at least 91%, at least 92%, at least 93%, at least 94%, at last 95%, at least 96%, at least 97%, at least 98%, at least 99%, or 100% sequence identity to SEQ ID NO: 128-131. The polypeptide may contain deletions, substitution, and/or insertions relative to any of SEQ ID NO: 128-131, such as having 1, 2, 3, 4, 5, 6, 7, 8, 9, 10, 11, 12, 13, 14, 15, or more deletions, substitutions and/or insertions to the amino acid sequence described by SEQ ID NO: 128-131. These sequences are discussed in detail below in Example 9.

### Designed Human and Mammalian Protein Cages and Fusion Protein Subunits

According to the embodiments disclosed herein, certain protein molecules and their amino acid sequences are provided, wherein such molecules are derived as combinations (or fusions) of natural human or other mammalian sequences-referred to herein as human or mammalian fusion protein subunits-that are designed so that the self-assembling properties of said human or mammalian fusion protein subunit sequences produce a protein cage (also known as a protein nanoparticle) composed of multiple copies of the human or mammalian fusion protein subunit. In some embodiments, the human protein cages disclosed herein are composed of 12 identical or substantially identical copies of the human or mammalian fusion protein subunits. The resulting designed protein cages (DPCs), also referred to as designed protein nanoparticles (DPNs), are based on the three-dimensional structure of protein sequences that can be designed and modeled into tetrahedrally-shaped protein cages that have termini (e.g., N-termini or C-termini) accessible on their outer surface. The termini suitable for the further fusion and presentation of diverse effector proteins in multiple copy numbers. In some embodiments, the DPCs/DPNs disclosed herein may be produced from two or more endogenous human or mammalian proteins or portions thereof. Such DPCs/DPNs may also be referred to as designed human protein cages (DHPCs) (or designed human protein nanoparticles (DHPNs) or designed mammalian protein cages (DMPCs) (or designed mammalian protein nanoparticles (DMPNs).

In some embodiments, the symmetric protein-based nanoparticles or cages that assemble from identical or substantially identical copies of a protein subunit, wherein the sequences of the protein subunits are based on the sequences of human proteins. The sizes, geometric shapes, and polyvalent nature of the nanoparticles endow them with advantageous properties in diverse nanotechnology applications, while their composition from human protein sequences endows them with advantageous clinical properties (e.g. low immunogenicity) for in vivo use.

According to the embodiments disclosed herein, a DPC/DPN is produced from a plurality of fusion protein subunits that are capable of self-assembling into the DPC/DPN. In some embodiments, the DPC/DPN is produced from a plurality of such fusion protein subunits that are derived or engineered from two endogenous human proteins. Such fusion protein subunits are also referred to herein as human fusion protein subunits. In some embodiments, the DPC/DPN is produced from a plurality of such fusion protein subunits that are derived or engineered from two endogenous mammalian proteins. Such fusion protein subunits are also referred to herein as mammalian fusion protein subunits.

In certain embodiments, a fusion protein subunit that is capable of self-assembling into a multi-subunit three-dimensional complex is provided herein. According to some embodiments, the fusion protein subunit has two protein domains: an N-terminal domain and a C-terminal domain. The N-terminal domain and C-terminal domain may be directly fused to each other. In such embodiments, there is no linker. In other embodiments, the N-terminal domain and C-terminal domain are fused to each other via a linker.

In some embodiments, the linker is one or more amino acids, for example the linker may be n amino acids in length, wherein n is 1, 2, 3, 4, 5, 6, or greater than 6. As such, in some embodiments, the linker is 1 amino acid in length, 2 amino acids in length, 3 amino acids in length. 4 amino acids in length, 5 amino acids in length, 6 amino acids in length, or greater than 6 amino acids in length. In some embodiments, the linker is composed of all polyalanine residues. In other embodiments, the linker is composed of a combination of alanine, glutamate and lysine.

In some embodiments, the linker is 1, 2, or 3 amino acids in length. In some such embodiments, each amino acid that forms the linker is an amino acid that prefers to adopt an alpha-helical conformation, including Ala, Arg, Asp, Glu, Gln, His, Ile, Leu, Lys, Met, Phe, Trp, and Val. In other embodiments, the linker sequence is obtained by a computer algorithm (e.g. MPNN or other sequence-optimization program) used to optimize the fit of the sequence to the designed structure.

In some embodiments, the linker is 4 amino acids in length. In some such embodiments, each amino acid that forms the linker is an amino acid that prefers to adopt an alpha-helical conformation, including Ala, Arg, Asp, Glu, Gln, His, Ile, Leu, Lys, Met, Phe, Trp, and Val. In other embodiments, the linker sequence is obtained by a computer algorithm (e.g. MPNN or other sequence-optimization program) used to optimize the fit of the sequence to the designed structure. In other such embodiments, the linker has an amino acid sequence of EAAK or KAAE.

In some embodiments, the linker is 5 amino acids in length. In some such embodiments, each amino acid that forms the linker is an amino acid that prefers to adopt an alpha-helical conformation, including Ala, Arg, Asp, Glu, Gln, His, Ile, Leu, Lys, Met, Phe, Trp, and Val. In other embodiments, the linker sequence is obtained by a computer algorithm (e.g. MPNN or other sequence-optimization program) used to optimize the fit of the sequence to the designed structure. In other such embodiments, the linker has an amino acid sequence of AEAAK, EAAKA, EEAAK, EAAKK, AKAAE, KAAEA, KKAAE, or KAAEE.

In some embodiments, the linker is 6 amino acids in length. In some such embodiments, each amino acid that forms the linker is an amino acid that prefers to adopt an alpha-helical conformation, including Ala, Arg, Asp, Glu, Gln, His, Ile, Leu, Lys, Met, Phe, Trp, and Val. In other embodiments, the linker sequence is obtained by a computer algorithm (e.g. MPNN or other sequence-optimization program) used to optimize the fit of the sequence to the designed structure. In other such embodiments, the linker has an amino acid sequence of AAEAAK, AEAAKA, AEEAAK, AEAAKK, AAKAAE, AKAAEA, AKKAAE, AKAAEE, AEAAKA, EAAKAA, EEAAKA, EAAKKA, AKAAEA, KAAEAA, KKAAEA, KAAEEA, AEAAAK, EAAAKA, EEAAAK, EAAAKK, AKAAAE, KAAAEA, KKAAAE, or KAAAEE.

In some embodiments, the linker is greater than 6 amino acids in length. In some such embodiments, the linker has an amino acid sequence that is one of those discussed above with respect to a linker with 6 amino acids in length, but is preceded or followed by one or two residues that prefer to adopt an alpha-helical conformation, including Ala, Arg, Asp, Glu, Gln, His, Ile, Leu, Lys, Met, Phe, Trp, and Val.

In some embodiments, the N-terminal domain includes a first mammalian homo-oligomeric protein or portion thereof. In certain embodiments, the first mammalian homo-oligomeric protein is a naturally dimeric or trimeric. In some embodiments, the N-terminal domain comprises a portion of Purine nucleoside phosphorylase (UniProtKB/Swiss-Prot No. P00491), Enoyl-CoA delta isomerase 1—mitochondrial (UniProtKB/Swiss-Prot No. P42126), Enoyl-CoA delta isomerase 2 (UniProtKB/Swiss-Prot No. 075521), Citramalyl-CoA lyase—mitochondrial (UniProtKB/Swiss-Prot No. Q8NOX4).

In some embodiments, the C-terminal domain includes a second mammalian homo-oligomeric protein or portion thereof. In certain embodiments, the second mammalian homo-oligomeric protein is a naturally dimeric or trimeric. In some embodiments, C-terminal domain comprises a portion of Ragulator complex protein LAMTOR3 (UniProtKB/Swiss-Prot No. Q9UHA4), N-acylglucosamine 2-epimerase (Renin Binding Protein) (UniProtKB/Swiss-Prot No. P51606), Farnesyl pyrophosphate synthase (UniProtKB/Swiss-Prot No. P14324), Ran-binding protein 10 (UniProtKB/Swiss-Prot No. Q6VN20), NTF2-related export protein 1 (UniProtKB/Swiss-Prot No. Q9UKK6), Centromere protein S (UniProtKB/Swiss-Prot No. Q8N2Z9), Fructose-bisphosphate aldolase B (UniProtKB/Swiss-Prot No. P05062), Barrier-to-autointegration factor-like protein (UniProtKB/Swiss-Prot No. Q9H503), Retinol dehydrogenase 10 (UniProtKB/Swiss-Prot No. Q8IZV5), Tyrosine—tRNA ligase—mitochondrial (UniProtKB/Swiss-Prot No. Q9Y2Z4), Fructose-bisphosphate aldolase B (UniProtKB/Swiss-Prot No. P05062), Choline/ethanolamine kinase (UniProtKB/Swiss-Prot No. Q9Y259), Serine hydroxymethyltransferase—cytosolic (UniProtKB/Swiss-Prot No. P34896), Malonyl-CoA decarboxylase—mitochondrial (UniProtKB/Swiss-Prot No. 095822), Barrier-to-autointegration factor (UniProtKB/Swiss-Prot No. 075531), Cytosolic Fe—S cluster assembly factor NUBP1 (UniProtKB/Swiss-Prot No. P53384), Inositol monophosphatase 1 (UniProtKB/Swiss-Prot No. P29218)

Numerous embodiments of human and mammalian fusion protein subunits (and their sequences) that can be used to form DHPCs and DMPCs are disclosed herein. In each embodiment, the self-assembling protein subunit comprises a genetic fusion—i.e. a linear joining of two amino acid sequences—of portions of two distinct human proteins, chosen according to highly specific engineering criteria (Padilla, Colovos, and Yeates in 2001. PMID: 11226219). In some embodiments, the two component human or mammalian protein domains that are fused are each homooligomeric in their natural forms. In some embodiments, one of the component domains is a natural dimeric human protein and the other component domain is a natural trimeric human protein. Furthermore, both natural protein components have alpha-helical conformations at or near their termini. Some particular combinations of two such proteins (or portions thereof), when fused through their alpha helical termini, will self-assemble in a predictable fashion to form symmetric cage-like or capsid-like architectures. Only rare choices for the combined oligomeric components meet the underlying geometric requirements, and the present invention specifies a select set of human protein fusion combinations that meet those exacting requirements.

In some embodiments, the two-component human or mammalian protein domains are homo-oligomeric variants of their natural (or wild type) forms. For example, in some embodiments, the first mammalian or human homo-oligomeric protein or portion thereof and/or the second mammalian or human homo-oligomeric protein or portion thereof comprise one or more amino acid substitutions to prevent or minimize chemical modifications. For example, some amino acid residues found in the natural or wild type sequence of the protein components (e.g., cysteine or asparagine) may sometime compromise protein expression in eukaryotic cell-based expression systems and/or may affect the stability of the product. Such amino acid residues may be subject to chemical modification, e.g., glycosylation, which can compromise stability of the product. Thus, in some embodiments, one or more amino acid substitutions can be made to prevent or minimize such effects including substituting one or more cysteine residues with a less reactive amino acid such as alanine, serine or threonine and/or substituting one or more asparagine residues with a less reactive amino acid such as alanine, glutamine or aspartic acid.

In other embodiments, the first mammalian or human homo-oligomeric protein or portion thereof and/or the second mammalian or human homo-oligomeric protein or portion thereof comprise one or more amino acid substitutions to attenuate endogenous enzymatic activity. In such embodiments, an amino acid in the native/wild type sequence that is required for enzymatic activity is changed to another amino acid residue such as a glycine, alanine, valine, leucine, or isoleucine.

Also disclosed herein are protein sequence compositions wherein the DHPN is further extended by genetic fusion to another protein domain, referred to herein as an “effector polypeptide”. According to the construction and self-assembling properties of a DHPN, the assembled nanoparticle presents 12 copies of the effector polypeptide as protrusions from its outer surface. Described herein are compositions wherein the DHPN effector polypeptide is one of: various cytokines, Fc antibody-binding domains of protein A or protein G or polypeptide mimics thereof, various viral antigens, collagen-binding polypeptides, and various polypeptides that stimulate bone or tissue growth. These are example cases, not intended to limit the broad use or claims of DHPNs.

In some embodiments, the fusion protein subunit is a human fusion protein subunit and has or comprises a sequence having at least 90%, at least 91%, at least 92%, at least 93%, at least 94%, at least 95%, at least 96%, at least 97%, at least 98%, at least 99% or 100% sequence identity to sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO: 12, SEQ ID NO: 13, SEQ ID NO:14, SEQ ID NO:15, SEQ ID NO: 16, SEQ ID NO: 17, SEQ ID NO: 18, SEQ ID NO:19, SEQ ID NO:20, or SEQ ID NO:21. These sequences are discussed in detail below in Example 1.

In some embodiments, the fusion protein subunit is a mammalian fusion protein subunit and has or comprises a mouse or monkey amino acid sequence having at least 90%, at least 91%, at least 92%, at least 93%, at least 94%, at least 95%, at least 96%, at least, 97%, at least 98%, at least 99% or 100% sequence identity to sequence identity to SEQ ID NO:22, SEQ ID NO:23, SEQ ID NO: 24, SEQ ID NO:25, SEQ ID NO:26, SEQ ID NO:27, SEQ ID NO:28, SEQ ID NO:29, SEQ ID NO: 30, SEQ ID NO:31, SEQ ID NO:32, SEQ ID NO:33, SEQ ID NO:34, SEQ ID NO:35, SEQ ID NO: 36, SEQ ID NO:37, SEQ ID NO:38, SEQ ID NO:39, SEQ ID NO:40, SEQ ID NO:41, SEQ ID NO: 42, SEQ ID NO:43, SEQ ID NO:44, SEQ ID NO:45, SEQ ID NO:46, SEQ ID NO:47, SEQ ID NO: 48, SEQ ID NO:49, SEQ ID NO:50, SEQ ID NO:51, SEQ ID NO:52, SEQ ID NO:53, SEQ ID NO: 54, SEQ ID NO:55, SEQ ID NO:56, SEQ ID NO:57, SEQ ID NO:58, SEQ ID NO:59, SEQ ID NO: 60, SEQ ID NO:61, SEQ ID NO:62, SEQ ID NO:63. These sequences are discussed in detail below in Example 2.

In some embodiments, the linker in SEQ ID NOs 1-63 is represented by one or more “X”. In some aspects, the “X” represents any amino acid. In some embodiments, the “X” represents an alpha-helix-preferring amino acid (e.g., A, E, L, M, K). In other embodiments, the “X” represents any of the other linkers disclosed herein.

In some embodiments, the fusion protein subunit is a human fusion protein subunit and has or comprises a sequence having at least 90%, at least 91%, at least 92%, at least 93%, at least 94%, at least 95%, at least 96%, at least 97%, at least 98%, at least 99% or 100% sequence identity to SEQ ID NO: 64-127. The sequence contains deletions, substitutions, and/or insertions relative to a corresponding sequence having a SEQ ID NO: 1-21. These sequences are discussed in detail below in Example 3.

In some embodiments, the fusion protein subunit is a human fusion protein subunit fused to effector proteins or payloads and has or comprises a sequence having at least 90%, at least 91%, at least 92%, at least 93%, at least 94%, at least 95%, at least 96%, at least 98%, at least 98%, at least 99%, or 100% sequence identity to SEQ ID NO: 128-131. These sequences are discussed in detail below in Example 9.

### Methods for Designing DPC/DPNs

In other embodiments, the component human or orthologous mammalian proteins that form the basis for the genetic fusion constructs disclosed herein may be designed from an endogenously expressed human or mammalian protein homo-oligomeric in nature. In accordance with the embodiments disclosed herein, the component proteins are either homo-dimeric or homo-trimeric, and their three-dimensional structures are known or can be modeled from known data. In some embodiments, the protein sequences and structures are obtained from the Protein Data Bank (PDB), which serves as the world-wide repository for three-dimensional protein structure data (H. M. Berman, J. Westbrook, Z. Feng, G. Gilliland, T. N. Bhat, H. Weissig, I. N. Shindyalov, P. E. Bourne (2000) Nucleic Acids Research, 28:235-242. https://doi.org/10.1093/nar/28.1.235). In addition, the three-dimensional structures of many human proteins can be modeled based on known structures of closely similar protein sequences (e.g. from related organisms) using computational tools known in the art (e.g. BLAST, AlphaFold-Oligomer). Each exemplary DPN/DPC disclosed herein is a fusion between a trimeric human protein sequence and a dimeric human protein sequence. In some embodiments, the trimeric component becomes the N-terminal domain and the dimeric component becomes the C-terminal domain of the larger fusion construct, while in other embodiments, the dimeric component becomes the N-terminal domain and the trimeric component becomes the C-terminal domain of the larger fusion construct. In certain aspects, the component proteins employed conform to a further restriction: they have alpha helical conformations at their termini. Specifically, in some embodiments, the trimeric (N-terminal) component of the fusion construct is alpha helical at its C-terminus, while the dimeric (C-terminal) component of the fusion construct is alpha helical at its N-terminus. Information about the alpha helical nature of the proteins may be discernible from the known structural data and computational tools known in the art (e.g. STRIDE or DSSP). Creating a fusion construct between two component proteins when their termini are both alpha helical produces a protein molecule wherein the geometry and spatial relationship of the two joined components may be designed according to the properties of a protein alpha helix. That is, given known structures for both components and their alpha helical termini, the three-dimensional structure of the entire fusion construct is designed with reasonable precision, subject in typical cases to some potential flexibility at the alpha helical joint.

Geometric considerations are critical to the designing DPNs/DPCs in accordance with the embodiments disclosed herein. The two protein components (or portions thereof) that are fused together are homo-oligomeric, as previously noted, meaning that each component confers its own self-associating properties to the fusion construct. Among the vast number of potential fusion constructs that could be created between all possible known homo-oligomeric proteins, only a very small unknown fraction may be used to form regular nanoparticle shapes as desired upon self-assembly. The only suitable constructs are those for which the symmetry axes conferred by the two components obey prescribed rules of symmetry dictated by the geometry of the Platonic solids. Those rules of symmetry have been articulated in the context of designed protein cages in prior work (Padilla et al, 2001. PMID: 11226219). Specifically, for the DHPNs disclosed here, the symmetry axes conferred by the fused dimeric and trimeric components must intersect in three dimensional space, and must form an angle of 54.7 degrees.

As described above, according to some embodiments, the geometric arrangement of a fusion between a first protein component relative to a second protein component can be calculated or modeled based on the separately known structures if they can be connected by way of a continuous alpha helix. The inventive process therefore relies on a systematic modeling of all possible fusion combinations between known dimeric and trimeric structures, selecting only those wherein the resulting fusion conforms to the necessary geometric requirements, i.e. the symmetry axes of the two components intersect at 54.7 degrees.

In some embodiments, a further design element concerns whether a small intervening alpha helical segment is introduced between the two components to be fused. As noted above, when two proteins bearing alpha helical termini are fused, a continuous alpha helical conformation can be anticipated to span between the two parts of the protein in the fused form. This expectation remains true if a small number of amino acids are inserted between the two components being fused, as long as the inserted amino acids are among those that prefer to adopt an alpha helical conformation. The tendency of different amino acid types to adopt specific conformations, e.g. alpha helix vs. beta sheet, has been established as a principle of protein science (Chou and Fasman. Biochemistry. 1974 January; 13 (2): pp 211-222). In some embodiments, a protein cage can be designed with up to six alpha helix-preferring amino acids inserted between the two oligomeric components (Padilla et al., 2001. PMID: 11226219; Lai et al. 2014. PMID: 25411884). The protocol for designing DPCs/DPNs therefore incorporates analysis of the geometry of fusion between pairs of oligomeric human proteins wherein up to 6 additional amino acids are inserted between their natural alpha helical termini. Owing to the geometry of the alpha helix—i.e. a rotation of approximately 100 degrees for each additional amino acid residue—each choice of length for the inserted segment provides for a different geometric arrangement of the two fused components. Each such consideration of linker length therefore provides an additional chance that the candidate fusion construct will satisfy the angular requirements that must be obeyed by the symmetry axes carried by the oligomeric components.

Note that no candidate construct can be expected to give a modeled geometry (i.e. an angle between symmetry axes) that matches exactly to the required value. But some candidates will have modeled geometries that are close to the desired values, and the known flexibility of an alpha helical linker is sufficient to allow the correct geometric angle to be adopted by the fusion construct upon self-assembly. The flexibility of an alpha helical linker in the context of protein fusions has been studied (Castells-Graells, PNAS 2023. PMID: 37669364). In some embodiments, it is also allowable in some design cases to make a fusion wherein one or a few amino acids are removed at the helical fusion junction. This amounts to overlapping the two components slightly at their ends, and is conceptually the opposite of inserting a few additional amino acids.

With the considerations above, the process for design of DPCs/DPNs disclosed herein entails the following in accordance with some embodiments: (1) gathering of known human oligomeric structures, here sufficing to consider dimeric and trimeric forms; (2) ascertaining the subset of those oligomeric structures that have an alpha helical conformation at a terminus, to support the underlying design requirement that two component proteins will be fused between their alpha helical termini; (3) considering all possible pairs of oligomeric candidates above as potential fusions; (4) considering a range of alpha helical residues to be inserted, up to a length of 6, while also considering removal of up to 2 residues for an overlapping construction; (5) evaluating the resulting geometry, specifically how close the angle between the symmetry axes carried by the two components agrees with the desired angle of intersection; and optionally, (6) based on the known bending modes established for an alpha helical linker, evaluate whether a small degree of allowable bending can bring the fusion construct into agreement with the desired angle. Finally, among the many thousands of candidate constructions that are examined, (7) select those that meet the requirements for a designed protein cage.

Fusing two separate oligomeric components can create a subunit that inherits the self-associating properties of the separate components, and the symmetry properties of the separate components are effectively conjoined in the engineered fusion protein. The two components must be joined together in a specific way in order for the resulting subunit to self-assemble (from multiple copies) into a complex architecture like those exemplified by a cube or tetrahedron. Specifically, the angle between the joined components—or more accurately the angle between the virtual symmetry axes they confer—must match a value specific for the symmetry of the underlying Platonic solid. Sets of specific angular requirements for engineering novel protein cages have been discussed previously (Padilla et al. (2001)). As an example relevant for the invention described here, a tetrahedral cage comprised of 12 subunits is obtained by self-assembly if a naturally dimeric human protein can be fused to a naturally trimeric protein in such a way that their axes of symmetry intersect at an angle of 54.7 degrees.

Connecting two protein components together in a geometrically precise fashion is a significant engineering challenge, and a solution to that problem was also introduced by Padilla et al, (2001). There it was established that geometrically defined outcomes from fusing two separate protein components could be obtained by relying on a specific structural feature of proteins, namely alpha helices. Alpha helices are segments of secondary structure with well-defined characteristics—e.g. defined twist and rise. It has been established that, if two protein components have alpha helical termini, then a fusion between them could create a subunit where a continuous and unbroken alpha helix connects the two parts. In that case, owing to the defined and predictable geometry of the joining helix, the relative geometry (e.g. the angle between) the two components can be anticipated on the basis of straightforward modeling of the fusion construct, assuming the three-dimensional structures of the components are known in advance. It is important to note that for the vast majority of known dimeric and trimeric proteins that might be considered for pairwise fusion, the resulting geometry would not meet the specific angular requirements for forming an ordered architecture like a protein cage. Only a very small fraction of potential choices for construction meet the requirements even approximately. The identification of suitable pairs of components to fuse together requires computational analysis of a large set of candidate models obtained from pairwise combinations of known oligomeric proteins (dimers and trimers in the case of interest here).

Owing to their composition from several copies of the same subunit, designed protein cages provide a facile route for displaying multiple copies of various effector proteins on their surface. According to some embodiments, if the protein subunit has a terminus disposed towards the exterior surface, an effector protein can be fused to the protein cage subunit. In other embodiments, the protein cage assembles from 12 copies of the subunit, then 12 copies of the fused effector protein will be presented on the exterior. Various mechanisms make it possible to present more than one type of effector on the exterior according to some embodiments. For example, if the cage protein subunit has both termini exposed, then two distinct effectors can be fused to the exterior, both in multiple copy number. Alternatively, the effector protein can be one with the ability to bind noncovalently to yet another protein, or a set of protein variants, facilitating the presentation of multiple different proteins. Polyvalency provides for several important effects. Binding events involving the association of multiple partner molecules generally exhibit higher affinity than similar associations between a single pair of molecules. That phenomenon is sometimes described as avidity. Tight binding is a major advantage in therapeutic applications where specificity for certain targets is critical. Presenting multiple distinct effectors provides further specificity, e.g. binding only to cells that present receptors for both effector molecules presented according to some embodiments.

The designed protein cages disclosed herein also provide opportunities to present multiple effectors in specific geometries and avidities, with the goal of affecting the co-localization of the receptors to which the effectors bind. Numerous critical cellular processes are regulated not simply by the binding of molecules to receptors, but by binding events that drive multiple receptors to associate or aggregate. Designed protein cages also provide the capacity for molecular delivery and controlled release of interior contents.

In accordance with the embodiments disclosed herein, methods for designing human/mammalian protein cages are provided as set forth below. Two distinct design approaches based on oligomeric proteins have been explored for creating protein cages and nanoparticles. In the first “fusion-type” approach, two oligomeric proteins are genetically fused by a linker of defined structure and favorable rigidity (e.g. an alpha helix). In the second “interface design” approach, a series of amino acid mutations are made to the surfaces of two oligomeric proteins with the effect of binding the two oligomeric protein components together. The designed interface in the second approach effectively replaces the fusion linker in the first approach. The first (fusion-type) method makes it possible to create protein cages whose sequences are derived essentially entirely from human protein sequences, conferring the benefits discussed herein. In contrast, the second (interface design) approach generally requires many amino acid changes relative to a natural sequence, making the sequence of the designed protein subunit divergent from any natural human sequence, for example. The designs and embodiments provided here employ the fusion-based approach.

### Effector Proteins or Polypeptides

According to the embodiments disclosed herein, a designed protein cage may display effector proteins of various types. In certain embodiments, the protein cage can display multiple copies of a single effector protein or peptide. In other embodiments, the protein cage can display more than one effector protein or peptide. For example, in certain embodiments, the protein cage can display an effector protein by attaching the same effector protein to each of the 12 fusion protein subunits on its exposed N- and/or C-terminal. In other embodiments, the protein cage can display two or more proteins by attaching each of the two or more proteins to an exposed N- or C-terminal of each of the 12 fusion protein subunits. The ability to display multiple copies of the same effector protein as well as distinct effector proteins simultaneously confers significant advantages for binding avidity and targeting specificity.

In some embodiments, the effector protein may include, but is not limited to, a viral antigen, a cellulolytic enzyme, a nanobody, a cytokine, a growth factor, a protein or polypeptide that binds to a tissue component such as collagen, a protein or polypeptide that binds to a protein on the surface of a cell such as an immune cell or a cancer cell, and a protein or polypeptide that binds the Fc region of an antibody.

In some embodiments, the effector protein is an Fc-binding protein including, but not limited to, Protein A, Protein B, or an Fc-III synthetic polypeptide sequence (Fc-III: DCAWHLGELVWCT) (SEQ ID NO:133). It is understood in the field that short polypeptides such as this can typically be fused to protein subunits or assemblies of multiple protein subunits without disrupting the structure or integrity of the latter.

In some embodiments, the effector protein is a cytokine, a cytokine receptor ligand, a chemokine, a chemokine receptor ligand, a growth factor, a hormone, a neuropeptide, a gastrointestinal or metabolic peptide, a cardiovascular peptide, or any other molecule that acts to modulate a therapeutic target, such as a receptor or cell surface protein on a target cell. In some embodiments, the effector protein is a cytokine. In some embodiments, the cytokine is an interleukin selected from those listed in Table 1 below.

TABLE 1
Interleukin Cytokine Effector Proteins

| | | NCBI | |
|---|---|---|---|
| Effector Protein Name | Abbreviation | Gene ID | UniProt ID |
| Interleukin 1 Alpha | IL-1A | 3552 | P01583 |
| Interleukin 1 Beta | IL-1B | 3553 | P01584 |
| Interleukin 2 | IL-2 | 3558 | P60568 |
| Interleukin 3 | IL-3 | 3562 | P08700 |
| Interleukin 4 | IL-4 | 3565 | P05112 |
| Interleukin 5 | IL-5 | 3567 | P05113 |
| Interleukin 6 | IL-6 | 3569 | P05231 |
| Interleukin 7 | IL-7 | 3574 | P13232 |
| Interleukin 8 | IL-8 | 3576 | P10145 |
| Interleukin 9 | IL-9 | 3578 | P15248 |
| Interleukin 10 | IL-10 | 3586 | P22301 |
| Interleukin 11 | IL-11 | 3589 | P20809 |
| Interleukin 12A | IL-12A | 3592 | P29459 |
| Interleukin 12B | IL-12B | 3593 | P29460 |
| Interleukin 13 | IL-13 | 3596 | P35225 |
| Interleukin 14 | IL-14 | 200081 | P40222 |
| (Taxilin Alpha) | (TXLNA) | | |
| Interleukin 15 | IL-15 | 3600 | P40933 |
| Interleukin 16 | IL-16 | 3603 | Q14005 |
| Interleukin 17A | IL-17A | 3605 | Q16552 |
| Interleukin 17B | IL-17B | 27190 | Q9UHF5 |
| Interleukin 17C | IL-17C | 27189 | Q9P0M4 |
| Interleukin 17D | IL-17D | 53342 | Q8TAD2 |
| Interleukin 17F | IL-17F | 112744 | Q96PD4 |
| Interleukin 18 | IL-18 | 3606 | Q14116 |
| Interleukin 19 | IL-19 | 29949 | Q9UHD0 |
| Interleukin 20 | IL-20 | 50604 | Q9NYY1 |
| Interleukin 21 | IL-21 | 59067 | Q9HBE4 |
| Interleukin 22 | IL-22 | 50616 | Q9GZX6 |
| Interleukin 23A | IL-23A | 51561 | Q9NPF7 |
| Interleukin 24 | IL-24 | 11009 | Q13007 |
| Interleukin 25 | IL-25 | 64806 | Q9H293 |
| (Interleukin 17E) | (IL-17E) | | |
| Interleukin 26 | IL-26 | 55801 | Q9NPH9 |
| Interleukin 27 | IL-27 | 246778 | Q8NEV9 |
| Interleukin 28A | IL-28A | 282616 | Q8IZJ0 |
| (Interferon Lambda 2) | (IFNL2) | | |
| Interleukin 28B | IL-28B | 282617 | Q8IZI9 |
| (Interferon Lambda 3) | (IFNL3) | | |
| Interleukin 29 | IL-29 | 282618 | Q8IU54 |
| (Interferon Lambda 1) | (IFNL1) | | |
| Interleukin 31 | IL-31 | 386653 | Q6EBC2 |
| Interleukin 32 | IL-32 | 9235 | P24001 |
| Interleukin 33 | IL-33 | 90865 | O95760 |
| Interleukin 34 | IL-34 | 146433 | Q6ZMJ4 |
| Interleukin 35 | IL-35 | 3592 | P29459 |
| (Dimer comprising | | (IL-12A) | (IL-12A) |
| IL-12A & EBI3)¹ | | & 10148 | & Q14213 |
| | | (EBI3) | (EBI3) |
| Interleukin 36A | IL-36A | 27179 | Q9UHA7 |
| Interleukin 36B | IL-36B | 27177 | Q9NZH7 |
| Interleukin 36G | IL-36G | 56300 | Q9NZH8 |

¹IL-35 is a dimeric protein composed of IL-12α and IL-27β chains, which are encoded by two separate genes called IL12A and EBI3 (Epstein-Barr virus-induced gene 3), respectively

In other embodiments, the effector protein is a cytokine receptor ligand. In some embodiments, the cytokine receptor ligand is a tumor necrosis factor ligand selected from those listed in Table 2 below.

TABLE 2
Tumor Necrosis Factor Receptor Ligand Effector Proteins

| | | NCBI | UniProt |
|---|---|---|---|
| Effector Protein Name | Abbreviation | Gene ID | ID |
| Tumor Necrosis Factor | TNF | 7124 | P01375 |
| TNF Superfamily Member 4 | TNFSF4 | 7292 | P23510 |
| (OX-40 ligand) | (OX40L) | | |
| TNF Superfamily Member 6 | TNFSF6 | 356 | P48023 |
| (Fas Ligand) | (FASLG) | | |
| TNF Superfamily Member 9 | TNFSF9 | 8744 | P41273 |
| (4-1BB ligand) | 4-1BBL | | |
| TNF Superfamily Member 10 | TNFSF10 | 8743 | P50591 |
| (Tumor Necrosis Factor-Related | (TRAIL) | | |
| Apoptosis-Inducing Ligand) | | | |
| TNF Superfamily Member 11 | TNFSF11 | 8600 | O14788 |
| | (RANKL) | | |
| TNF Superfamily Member 12 | TNFSF12 | 8742 | O43508 |
| (TNF-related weak inducer | (TWEAK) | | |
| of apoptosis) | | | |
| TNF Superfamily Member 13 | TNFSF13 | 8741 | O75888 |
| (A proliferation inducing ligand) | (APRIL) | | |
| TNF Superfamily Member 13b | TNFSF13B | 10673 | Q9Y275 |
| (B-cell activating factor, TNF | (BAFF, | | |
| Superfamily Member 20) | TNFSF20) | | |
| TNF Superfamily Member 14 | TNFSF14 | 8740 | O43557 |
| | (LIGHT) | | |
| TNF Superfamily Member 15 | TNFSF15 | 9966 | O95150 |
| | (TL1A) | | |
| TNF Superfamily Member 18 | TNFSF18 | 8995 | Q9UNG2 |
| | (GITR ligand) | | |
| TNF Superfamily Member 19 | TNFSF19 | 8742 | O43508 |
| | (TWEAK) | | |
| TNF Superfamily Member 20 | TNFSF20 | 7124 | P01375 |
| (Nerve Growth Factor) | (NGF) | | |
| TNF Superfamily Member 21 | TNFSF21 | 356 | P48023 |
| | (Fas ligand) | | |
| TNF Superfamily Member 22 | TNFSF22 | 8743 | P50591 |
| (Tumor Necrosis Factor-Related | (TRAIL) | | |
| Apoptosis-Inducing Ligand) | | | |
| TNF Superfamily Member 25 | TNFSF25 | 8718 | Q93038 |
| | (DR3 ligand) | | |

In other embodiments, the effector protein is a chemokine. In some embodiments, the chemokine is a C-C or C-X-C chemokine. In other embodiments, the effector protein is a chemokine receptor ligand. In some embodiments, the chemokine receptor ligand is a C-C or C-X-C ligand selected from those listed in Table 3 below.

TABLE 3
C-C or C-X-C Ligand Effector Proteins

| | | NCBI | |
|---|---|---|---|
| | | Gene | UniProt |
| Effector Protein Name | Abbreviation | ID | ID |
| C-C Motif Chemokine Ligand 1 | CCL1 | 6346 | P22362 |
| C-C Motif Chemokine Ligand 2 | CCL2 | 6347 | P13500 |
| C-C Motif Chemokine Ligand 3 | CCL3 | 6348 | P10147 |
| C-C Motif Chemokine Ligand 4 | CCL4 | 6351 | P13236 |
| C-C Motif Chemokine Ligand 5 | CCL5 | 6352 | P13501 |
| C-C Motif Chemokine Ligand 7 | CCL7 | 6356 | P51671 |
| C-C Motif Chemokine Ligand 8 | CCL8 | 6355 | P80075 |
| C-C Motif Chemokine Ligand 11 | CCL11 | 6356 | P51671 |
| C-C Motif Chemokine Ligand 13 | CCL13 | 6357 | Q99616 |
| C-C Motif Chemokine Ligand 17 | CCL17 | 6361 | Q92583 |
| C-C Motif Chemokine Ligand 18 | CCL18 | 6362 | P55774 |
| C-C Motif Chemokine Ligand 19 | CCL19 | 6363 | Q99731 |
| C-C Motif Chemokine Ligand 20 | CCL20 | 6364 | P78556 |
| C-C Motif Chemokine Ligand 21 | CCL21 | 6366 | O00585 |
| C-C Motif Chemokine Ligand 22 | CCL22 | 6367 | O00626 |
| C-C Motif Chemokine Ligand 24 | CCL24 | 6369 | O00175 |
| C-C Motif Chemokine Ligand 25 | CCL25 | 6370 | O15444 |
| C-C Motif Chemokine Ligand 26 | CCL26 | 10344 | Q9Y258 |
| C-C Motif Chemokine Ligand 27 | CCL27 | 10850 | Q9Y4X3 |
| C-C Motif Chemokine Ligand 28 | CCL28 | 56477 | Q9NRJ3 |
| C-X-C Motif Chemokine Ligand 1 | CXCL1 | 6376 | P78423 |
| C-X-C Motif Chemokine Ligand 2 | CXCL2 | 3579 | P25025 |
| C-X-C Motif Chemokine Ligand 3 | CXCL3 | 2921 | P19876 |
| C-X-C Motif Chemokine Ligand 4 | CXCL4 | 5196 | P02776 |
| (Platelet Factor 4) | (PF4) | | |
| C-X-C Motif Chemokine Ligand 5 | CXCL5 | 6374 | P42830 |
| C-X-C Motif Chemokine Ligand 6 | CXCL6 | 6372 | P80162 |
| C-X-C Motif Chemokine Ligand 7 | CXCL7 | 5473 | P02775 |
| (Pro-Platelet Basic Protein) | (PPBP) | | |
| C-X-C Motif Chemokine Ligand 8 | CXCL8 | 3576 | P10145 |
| C-X-C Motif Chemokine Ligand 9 | CXCL9 | 4283 | Q07325 |
| C-X-C Motif Chemokine Ligand 10 | CXCL10 | 3627 | P02778 |
| C-X-C Motif Chemokine Ligand 11 | CXCL11 | 6373 | O14625 |
| C-X-C Motif Chemokine Ligand 12 | CXCL12 | 6387 | P48061 |
| C-X-C Motif Chemokine Ligand 13 | CXCL13 | 10563 | O43927 |
| C-X-C Motif Chemokine Ligand 14 | CXCL14 | 9547 | O95715 |
| C-X-C Motif Chemokine Ligand 16 | CXCL16 | 58191 | Q9H2A7 |
| C-X-C Motif Chemokine Ligand 17 | CXCL17 | 284340 | Q6UXB2 |

In other embodiments, the effector protein is a growth factor selected from those listed in Table 4 below.

TABLE 4
Growth Factor Effector Proteins

| | | NCBI | UniProt |
|---|---|---|---|
| Effector Protein Name | Abbreviation | Gene ID | ID |
| Epidermal Growth Factor | EGF | 1950 | P01133 |
| Fibroblast Growth Factor 1 | FGF | 2246 | P05230 |
| Fibroblast Growth Factor 2 | FGF2 | 2247 | P09038 |
| Fibroblast Growth Factor 3 | FGF3 | 2248 | P11487 |
| Fibroblast Growth Factor 4 | FGF4 | 2249 | P08620 |
| Fibroblast Growth Factor 5 | FGF5 | 2250 | P12034 |
| Fibroblast Growth Factor 7 | FGF7 | 2252 | P21781 |
| Fibroblast Growth Factor 10 | FGF10 | 2255 | O15520 |
| Fibroblast Growth Factor 23 | FGF23 | 8074 | Q9GZV9 |
| Platelet-Derived Growth | PDGFA | 5154 | P04085 |
| Factor A | | | |
| Platelet-Derived Growth | PDGFB | 5155 | P01127 |
| Factor B | | | |
| Vascular Endothelial Growth | VEGFA | 7422 | P15692 |
| Factor A | | | |
| Vascular Endothelial Growth | VEGFB | 7423 | P49765 |
| Factor B | | | |
| Hepatocyte Growth Factor | HGF | 3082 | P14210 |
| Insulin-Like Growth Factor 1 | IGF1 | 3479 | P05019 |
| Insulin-Like Growth Factor 2 | IGF2 | 3481 | P01344 |
| Nerve Growth Factor | NGF | 4803 | P01138 |
| Transforming Growth Factor | TGFA | 7039 | P01135 |
| Alpha | | | |
| Transforming Growth Factor | TGFB1 | 7040 | P01137 |
| Beta 1 | | | |
| Transforming Growth Factor | TGFB2 | 7042 | P61812 |
| Beta 2 | | | |
| Transforming Growth Factor | TGFB3 | 7043 | P10600 |
| Beta 3 | | | |
| Brain-Derived Neurotrophic | BDNF | 627 | P23560 |
| Factor | | | |
| Glial Cell-Derived | GDNF | 2668 | P39905 |
| Neurotrophic Factor | | | |

In other embodiments, the effector protein is a hormone selected from those listed in Table 5 below.

TABLE 5
Hormone Effector Proteins

| | | NCBI | UniProt |
|---|---|---|---|
| Effector Protein Name | Abbreviation | Gene ID | ID |
| Insulin | INS | 3630 | P01308 |
| Glucagon | GCG | 2641 | P01275 |
| Growth Hormone | GH | 2688 | P01241 |
| Prolactin | PRL | 5617 | P01236 |
| Thyroid-Stimulating Hormone² | TSH | 1081 (TSHA) + | P01215 (TSHA) + |
| (TSH-Alpha + TSH-Beta) | (TSHA + TSHB) | 7252 (TSHB) | P01222 (TSHB) |
| Adrenocorticotropic Hormone | ACTH | 5443 | P01189 |
| Luteinizing Hormone | LH | 1081 (LHA) + | P01215 (LHA) + |
| (LH-Alpha + LH-Beta) | (LHA + LHB) | 3972 (LHB) | P01229 (LHB) |
| Follicle-Stimulating Hormone | FSH | 1081 (FSHA) + | P01215 (FSHA) + |
| (FSH-Alpha + FSH-Beta) | (FSHA + FSHB) | 2488 (FSHB) | P01225 (FSHB) |
| Parathyroid Hormone | PTH | 5741 | P01270 |
| Calcitonin | CT | 796 | P01258 |
| Oxytocin | OXT | 5020 | P01178 |
| Vasopressin | ADH | 551 | P01185 |
| Erythropoietin | EPO | 2056 | P01588 |
| Leptin | LEP | 3952 | P41159 |
| Ghrelin | GHRL | 51738 | Q9UBU3 |
| Gastrin | GAST | 2520 | P01350 |
| Cholecystokinin | CCK | 885 | P06307 |
| Secretin | SCT | 6343 | P09683 |

²The four human glycoprotein hormones chorionic gonadotropin (CG), luteinizing hormone (LH), follicle stimulating hormone (FSH), and thyroid stimulating hormone (TSH) are dimers consisting of alpha and beta subunits that are associated noncovalently. The alpha subunits of these hormones are identical, however, their beta chains are unique and confer biological specificity. The protein encoded by this gene is the alpha subunit and belongs to the glycoprotein hormones alpha chain family.

In other embodiments, the effector protein is a hypothalamic or pituitary hormone that can bind and/or modulate a corticotropin releasing hormone receptor (e.g., CRHR1, CRHR2), a melanocortin receptor (e.g., MC2R), a thyrotropin releasing hormone receptor (e.g., TRHR), a gonadotropin releasing hormone receptor (e.g., GNRHR), a luteinizing hormone/choriogonadotropin receptor (e.g., LHCGR), a follicle stimulating hormone receptor (e.g., FSHR). In certain embodiments, the effector protein is a hypothalamic or pituitary hormone selected from those listed in Table 6 below.

TABLE 6
Hypothalamic or Pituitary Hormone Effector Proteins

| Effector Protein Name | Abbreviation | NCBI Gene ID | UniProt ID |
|---|---|---|---|
| Corticotropin-Releasing Hormone | CRH | 1392 | P06850 |
| Adrenocorticotropic Hormone | ACTH | 5443 | P01189 |
| Thyrotropin-Releasing Hormone | TRH | 7200 | P20396 |
| Gonadotropin-Releasing Hormone | GnRH | 2796 | P01148 |
| Luteinizing Hormone (LH) | LH | 1081 (LHA) + | P01215 (LHA) + |
| (LH-Alpha + LH-Beta) | (LHA + LHB) | 3972 (LHB) | P01229 (LHB) |
| Follicle-Stimulating Hormone | FSH | 1081 (FSHA) + | P01215 (FSHA) + |
| (FSH-Alpha + FSH-Beta) | (FSHA + FSHB) | 2488 (FSHB) | P01225 (FSHB) |

In other embodiments, the effector protein is a neuropeptide that can bind and/or modulate an Opioid Receptor (e.g., OPRM1, OPRD1, OPRK1, OPRL1) an NPY Receptor, A tachykinin receptor (e.g., NK1R (TACR1)), a calcitonin receptor-like receptor (e.g., CALCRL), a calcitonin receptor (e.g., CALCR), a somatostatin receptor (e.g., SSTR1-5), a growth hormone or ghrelin receptor (e.g., GHSR), a neuromedin U receptor (e.g., NMUR1, NMUR2), a neuromedin B receptor (NMBR). In some embodiments, the effector protein is an opioid peptide. Non-limiting examples of opioid peptides include, but are not limiting to, enkephalins (e.g., leu-enkephalin, met-enkephalin, metorphamide, peptide E), endorphins (e.g., α-endorphin, β-endorphin, γ-endorphin), dynorphins (e.g., dynorphin A, dynorphin A1-8, dynorphin B, big dynorphin, α-neoendorphin), nociceptin, endomorphins (e.g., endomorphin-1, endomorphin-2). In certain embodiments, the effector protein is a neuropeptide selected from those listed in Table 7 below.

TABLE 7
Neuropeptide Effector Proteins

| | | NCBI | UniProt |
|---|---|---|---|
| Effector Protein Name | Abbreviation | Gene ID | ID |
| Neuropeptide Y | NPY | 4852 | P01303 |
| Substance P | TAC1 | 6863 | P20366 |
| (Tachykinin Precursor 1) | | | |
| Calcitonin Gene-Related Peptide | CGRP | 796 | P01258 |
| Somatostatin | SST | 6750 | P61278 |
| Ghrelin | GHRL | 51738 | Q9UBU3 |
| Neuromedin U | NMU | 7859 | P48645 |
| Neuromedin B | NMB | 4828 | P08949 |

In other embodiments, the effector protein is a gastrointestinal or metabolic peptide that can bind and/or modulate a glucagon receptor (e.g., GCGR), a glucagon-like peptide 1 receptor (e.g., GLP-1R), a gastric inhibitory polypeptide receptor (e.g., GIPR), a CCK receptor (e.g., CCKAR, CCKBR), a glucagon-VIP-secretin receptor (e.g., SCTR), or a motilin receptor (e.g., MLNR). In certain embodiments, the effector protein is a gastrointestinal or metabolic peptide selected from those listed in Table 8 below.

TABLE 8
Gastrointestinal or Metabolic Peptide Effector Proteins

| | | NCBI | UniProt |
|---|---|---|---|
| Effector Protein Name | Abbreviation | Gene ID | ID |
| Cholecystokinin | CCK | 885 | P06307 |
| Gastric Inhibitory Polypeptide | GIP | 2695 | P09681 |
| Glucagon-Like Peptide-1 | GLP-1 | 2641 | P01275 |
| Gastrin | GAST | 2520 | P01350 |
| Secretin | SCT | 6343 | P09683 |
| Motilin | MLN | 4295 | P12872 |

In other embodiments, the effector protein is a cardiovascular peptide that can bind and/or modulate an angiotensin II receptor (e.g., AGTR1, AGTR2), an endothelin receptor (e.g., EDNRA, EDNRB), a bradykinin receptor (e.g., BDKRB1, BDKRB2), or an apelin receptor (e.g., APLNR). In certain embodiments, the effector protein is a cardiovascular peptide selected from those listed in Table 9 below.

TABLE 9
Cardiovascular Peptide Effector Proteins

| Effector | | NCBI | UniProt |
|---|---|---|---|
| Protein Name | Abbreviation | Gene ID | ID |
| Angiotensin II | AGT | 183 | P01019 |
| (Angiotensinogen) | | | |
| Endothelin-1 | EDN1 | 1906 | P05305 |
| Bradykinin | KNG1 | 3827 | P01042 |
| (Kininogen 1) | | | |
| Apelin | APLN | 8862 | Q9ULZ1 |

In some embodiments, the effector protein (or polypeptide) is a targeting moiety such as an antibody or antigenic fragment thereof that is capable of binding a therapeutic target to block or stimulate the target's activity, act as a antagonist, agonist, or allosteric modulator of a therapeutic target. In some embodiments, the antibody or antigenic fragment thereof has an antigenic region or regions that are designed to target and bind one or more of a cytokine, a cytokine receptor ligand, a chemokine, a chemokine receptor ligand, a growth factor, a hormone, a neuropeptide, a gastrointestinal or metabolic peptide, a cardiovascular peptide, or any other molecule that acts to modulate a therapeutic target, such as a receptor or cell surface protein on a target cell. In some embodiments, the antibody or antigenic fragment thereof has an antigenic region or regions that are designed to target and bind one or more of the therapeutic targets selected from those listed in Tables 1-9 above. In other embodiments, the antibody or antigenic fragment thereof has an antigenic region or regions that are designed to target and bind one or more receptors or cell surface proteins including those disclosed herein.

In some embodiments, the effector protein (or polypeptide) is a receptor moiety that comprises a receptor, or any binding portion thereof, for any of the ligands described above. In some embodiments, the receptor moiety is a receptor, a binding protein, or any binding portion thereof, for any of the ligands disclosed in Tables 1 to 9 above. For example, the receptor can be an interleukin receptor (or binding portion thereof) or interleukin binding protein (or binding portion thereof) that is capable of binding an interleukin selected from those listed in Table 1 (e.g., interleukin receptors such as interleukin 1-36 receptors, chemokine receptors, or serpentine receptors). In other examples, the receptor can be a Tumor Necrosis Factor or TNF Superfamily Member receptor (or binding portion thereof) or Tumor Necrosis Factor or TNF Superfamily Member binding protein (or binding portion thereof) that is capable of binding Tumor Necrosis Factor or a TNF Superfamily Member selected from those listed in Table 2 (e.g., TNF receptors, TNF receptor superfamily members 1-27). In other examples, the receptor can be a C-C or C-X-C Ligand receptor (or binding portion thereof) or C-C or C-X-C Ligand binding protein (or binding portion thereof) that is capable of binding a C-C or C-X-C Ligand selected from those listed in Table 3 (e.g., chemokine receptors such as C-C chemokine receptors 1-28 or C-X-C chemokine receptors 1-17). In other examples, the receptor can be a growth factor receptor (or binding portion thereof) or growth factor binding protein (or binding portion thereof) that is capable of binding a growth factor selected from those listed in Table 4 (e.g., TGF receptors, EGF receptors, VEGF receptors, hepatocyte growth factor receptors, IGF receptors, BDNF receptors, GDNF receptors, FGF receptors, PDGF receptors). In other examples, the receptor can be a hormone receptor (or binding portion thereof) or hormone binding protein (or binding portion thereof) that is capable of binding a hormone_selected from those listed in Table 5 (e.g., insulin receptors, glucagon receptors, growth hormone receptors, prolactin receptors, calcitonin receptors, TSH receptors, parathyroid receptors, vasopressin receptors, erythropoietin receptors, leptin receptors, ghrelin receptors, CCK receptors, secretin receptors, FSH receptors, LH receptors). In other examples, the receptor can be a hypothalamic or pituitary hormone receptor (or binding portion thereof) or hypothalamic or pituitary hormone binding protein (or binding portion thereof) that is capable of binding a hypothalamic or pituitary hormone selected from those listed in Table 6 (e.g., LH receptor, corticotropin-releasing factor receptors, melanocortin receptors (i.e., ACTH receptors), thyrotropin-releasing hormone receptor, gonadotropin-releasing hormone receptors, follicle-stimulating hormone receptors). In other examples, the receptor can be a neuropeptide receptor (or binding portion thereof) or neuropeptide binding protein (or binding portion thereof) that is capable of binding a neuropeptide selected from those listed in Table 7 (e.g., NPY receptors, tachykinin receptors, ghrelin receptors, neuromedin receptors, calcitonin receptor-like receptor). In other examples, the receptor can be a gastrointestinal or metabolic peptide receptor (or binding portion thereof) or gastrointestinal or metabolic peptide binding protein (or binding portion thereof) that is capable of binding a gastrointestinal or metabolic peptide selected from those listed in Table 8 (e.g., CCK receptors, gastrin receptors, secretin receptors, glucagon receptors, Gastric inhibitory polypeptide receptors). In other examples, the receptor can be a cardiovascular peptide receptor (or binding portion thereof) or cardiovascular peptide binding protein (or binding portion thereof) that is capable of binding a cardiovascular peptide selected from those listed in Table 9 (e.g., antiotensin II receptor, endothelin-1 receptor, bradykinin receptor). The receptor moiety may bind to its ligand to inhibit, attenuate, or otherwise prevent or modulate the ligand from binding its target receptor in vivo.

In some embodiments, the effector protein is fused to the N-terminal of the first mammalian or human homo-oligomeric protein or portion thereof directly or via a linker. In certain such embodiments, the human fusion protein subunit has or comprises a sequence having at least 90%, at least 91%, at least 92%, at least 93%, at least 94%, at least 95%, at least 96%, at least, 97%, at least 98%, at least 99% or 100% sequence identity to sequence identity to SEQ ID NO:1, SEQ ID NO: 3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, or SEQ ID NO:21.

In other embodiments, the effector protein is fused to the C-terminal of the second mammalian or human homo-oligomeric protein or portion thereof directly or via a linker. In certain such embodiments, the human fusion protein subunit has or comprises a sequence having at least 90%, at least 91%, at least 92%, at least 93%, at least 94%, at least 95%, at least 96%, at least, 97%, at least 98%, at least 99% or 100% sequence identity to sequence identity to SEQ ID NO:2, SEQ ID NO: 5, SEQ ID NO:6, SEQ ID NO:9, SEQ ID NO:10, SEQ ID NO:11, SEQ ID NO:12, SEQ ID NO: 13, SEQ ID NO:14, SEQ ID NO:17, and SEQ ID NO:20.

### Compositions and Methods of Production

The polypeptides, protein cages and their subunit chains disclosed herein may be produced using suitable methods known in the art including chemical synthesis, biosynthesis (in vitro and in vivo synthesis) using recombinant DNA methods, and solid phase synthesis. See, e.g., Kelly & Winkler (1990) Genetic Engineering Principles and Methods, vol. 12, J. K. Setlow ed., Plenum Press, NY, pp 1-19; Merrifield (1964) J Amer Chem Soc 85:2149; Houghten (1985) PNAS USA 82:5131-5135; and Stewart & Young (1984) Solid Phase Peptide Synthesis, 2ed., Pierce, Rockford, IL, which are herein incorporated by reference. The proteins may be purified using suitable protein purification techniques known in the art such as reverse phase high-performance liquid chromatography (HPLC), ion-exchange, affinity, or immunoaffinity chromatography, filtration or size exclusion, or electrophoresis. See, e.g., Olsnes and Pihl (1973) Biochem 12 (16): 3121-3126; and Scopes (1982) Protein Purification, Springer-Verlag, NY, which are herein incorporated by reference. Alternatively, the proteins may be made by recombinant DNA techniques known in the art.

In some embodiments, the polypeptides or proteins can be expressed from host cells using appropriate vectors. Mammalian cells, such as CHO cells, can be used. Those of skill in the art can select alternative vectors suitable for transforming, transfecting, or transducing cells to produce the proteins described herein.

In some embodiments, the polypeptides or proteins is purified using a Ni affinity chromatography. In some embodiments, the polypeptides or proteins is further purified by size exclusion chromatography (SEC), including high-performance liquid chromatography-based SEC (SEC-HPLC).

Compositions, including pharmaceutical compositions, comprising, consisting essentially of, or consisting of one or more protein cages are contemplated herein. For example, such compositions comprise one or more one or more protein cages as described herein and a pharmaceutically acceptable vehicle.

In some embodiments, the present invention provides kits comprising one or more protein cages described herein packaged together with one or more reagents for using the protein cages, e.g., buffers and reagents for labeling and detecting the protein cages, and/or devices for using the protein cages, e.g., syringes pre-filled with the protein cages. In some embodiments, the kits comprise the one or more protein cages, a carrier, package, or container for receiving and/or mixing the protein cages. In some embodiments, the kits optionally include an identifying description or label or instructions relating to its use. In some embodiments, the kits include information prescribed by a governmental agency that regulates the manufacture, use, or sale of compounds and compositions as contemplated herein.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

### EXAMPLES

According to the principles and protocols articulated above, the sequences of several DPCs/DPNs are disclosed below. In addition to the protein sequences and specifications of the length of any inserted linkers (if present), the human proteins (or human protein domains) from which the two components are derived are indicated. Information is also provided concerning sequence position designations in the originating proteins and the fusion constructs. In addition, information is provided concerning whether the (new) termini of the fused protein subunit are accessible on the outer surface of the DHPN, as this governs how effector proteins can be fused to the exterior for display and biological effect.

### Example 1: Exemplary Self-Assembling Human Protein Cages

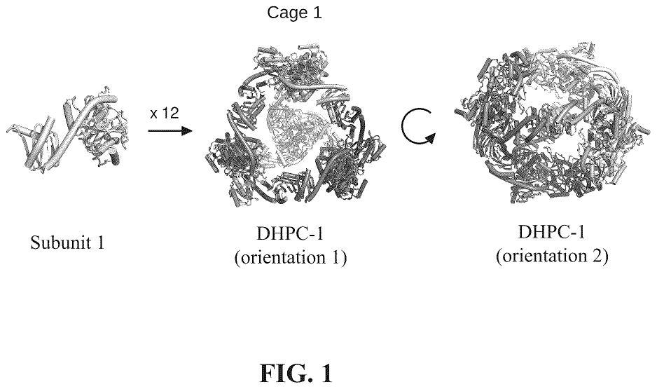

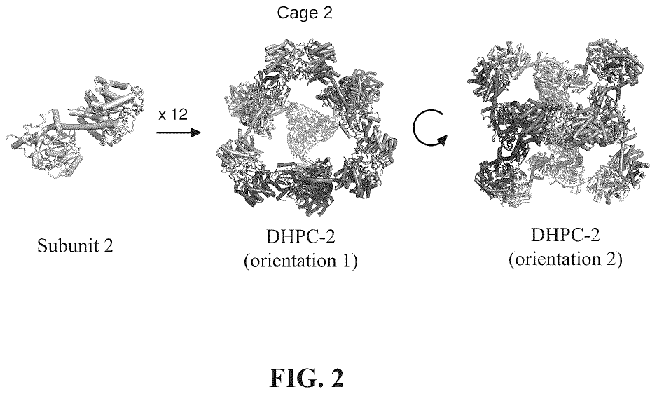

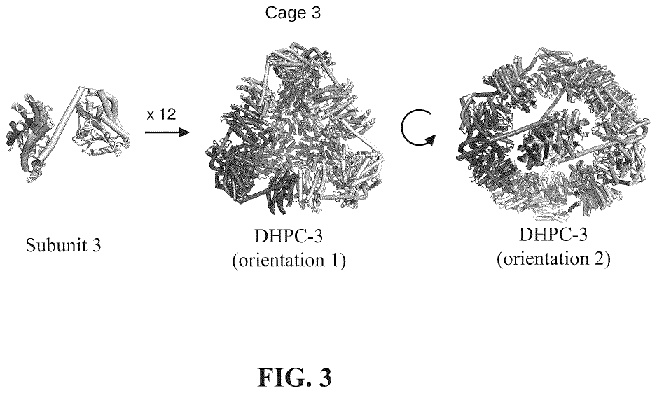

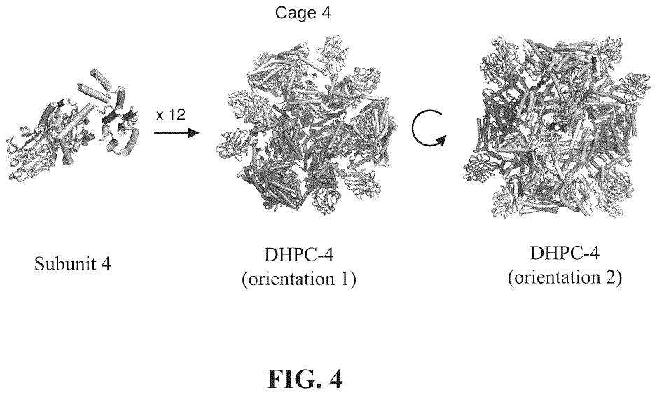

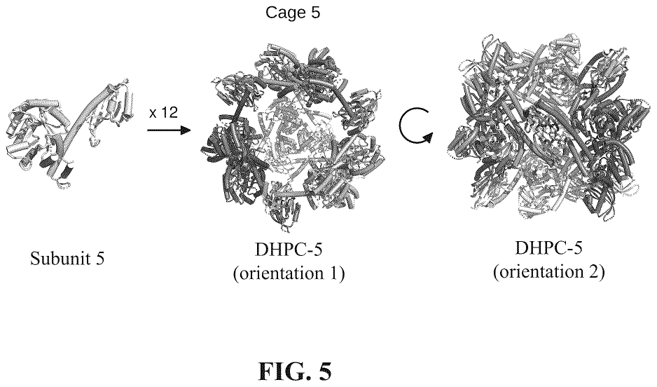

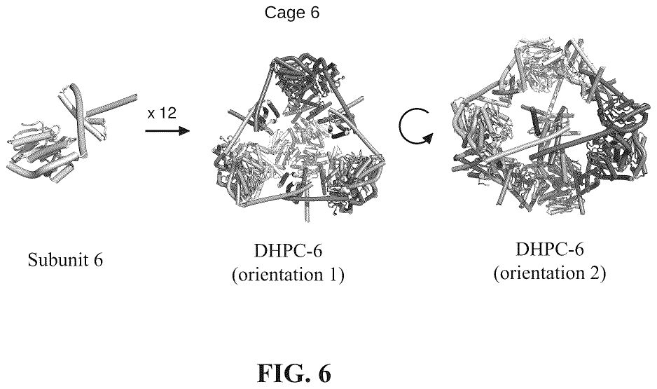

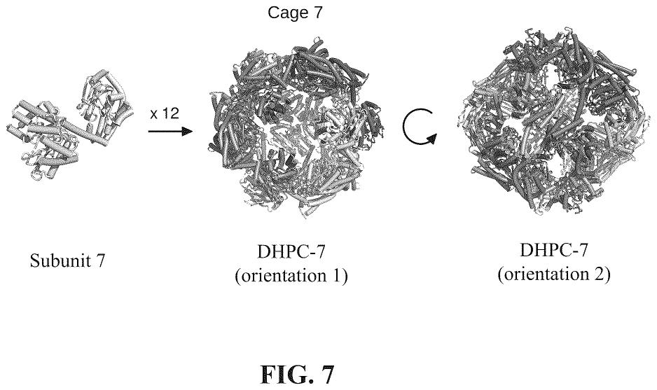

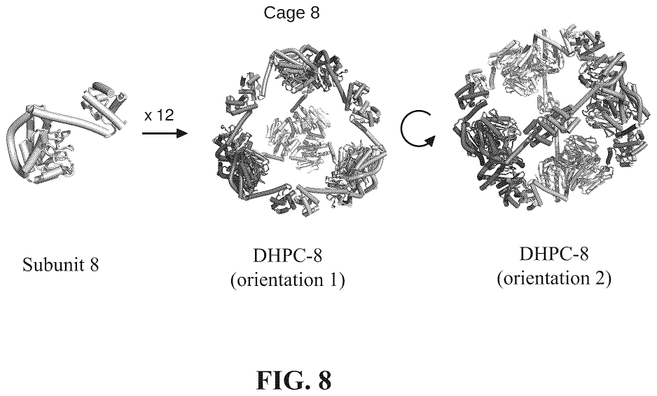

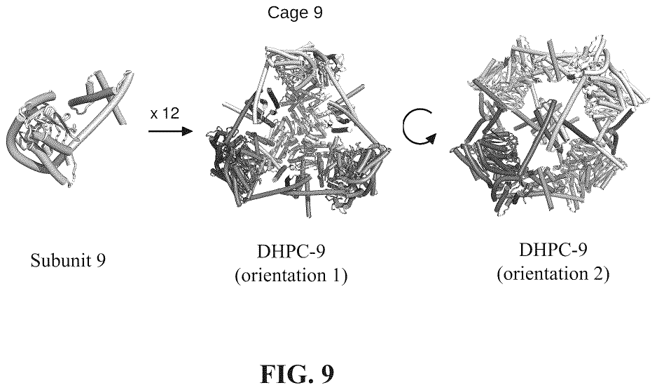

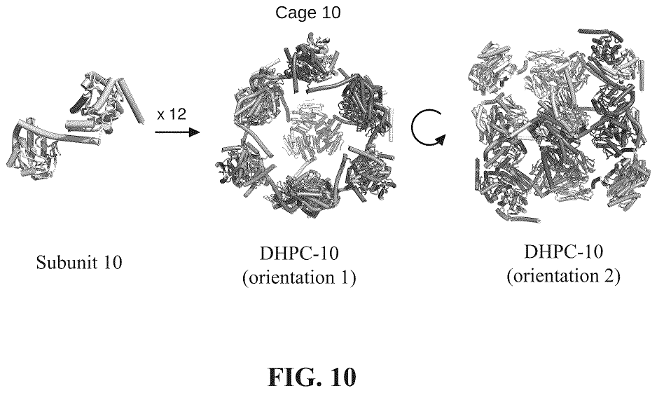

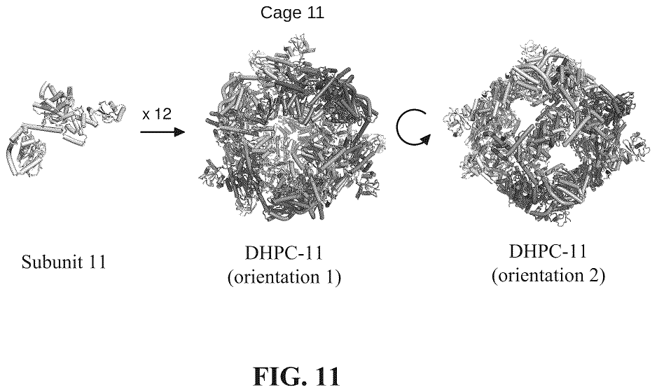

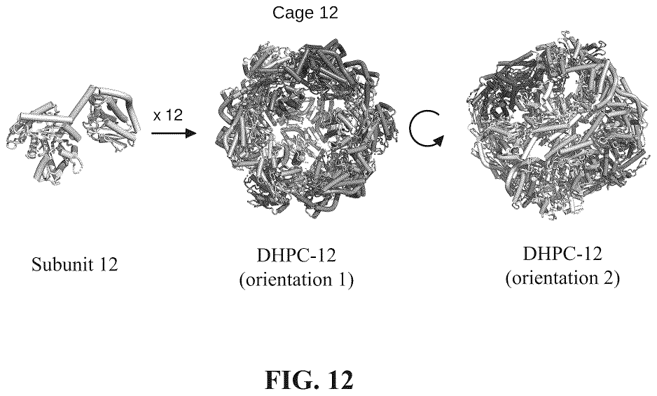

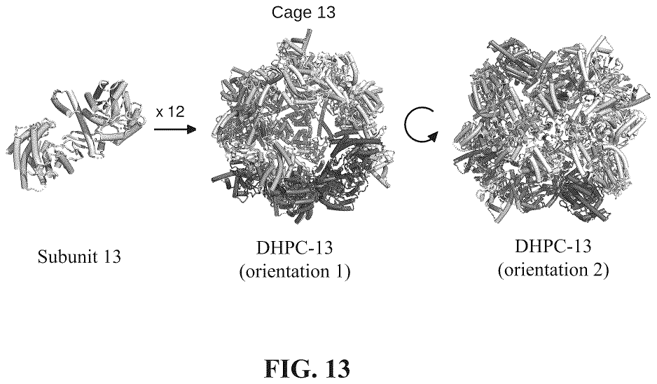

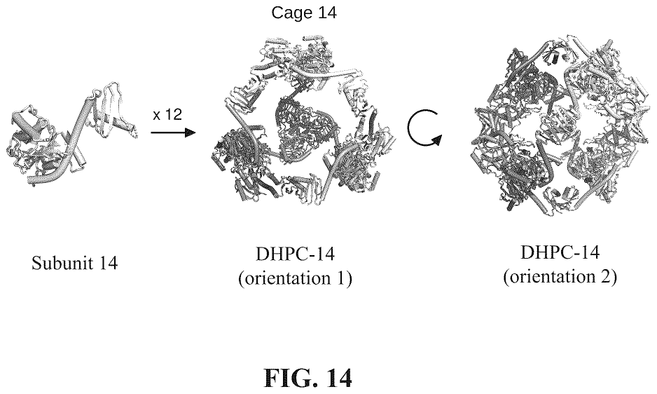

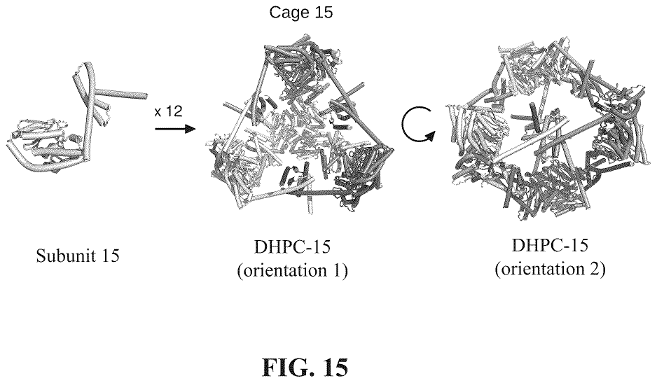

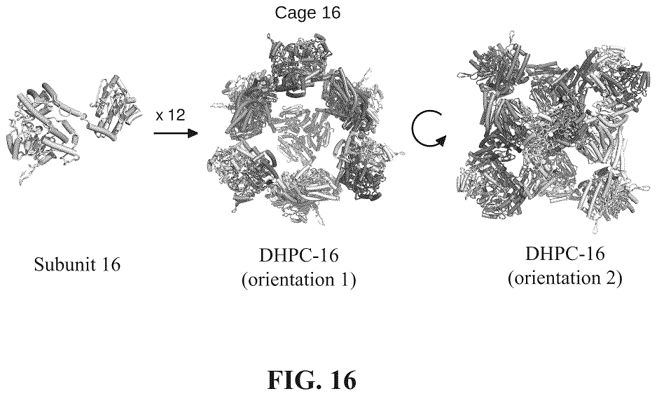

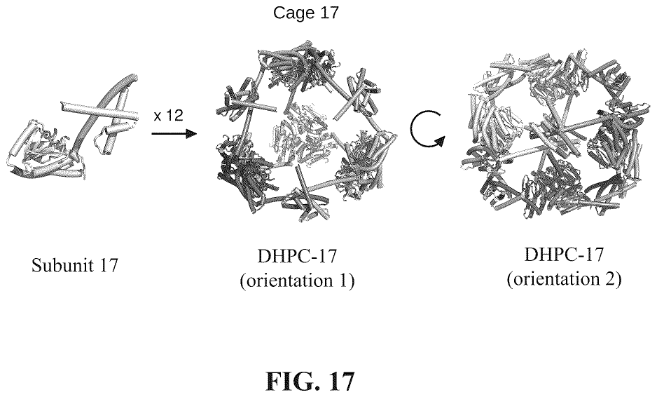

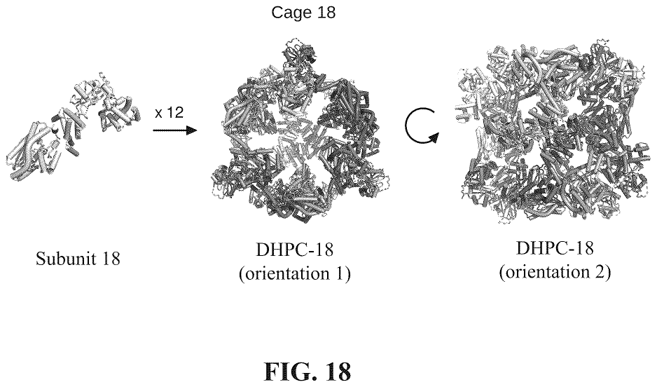

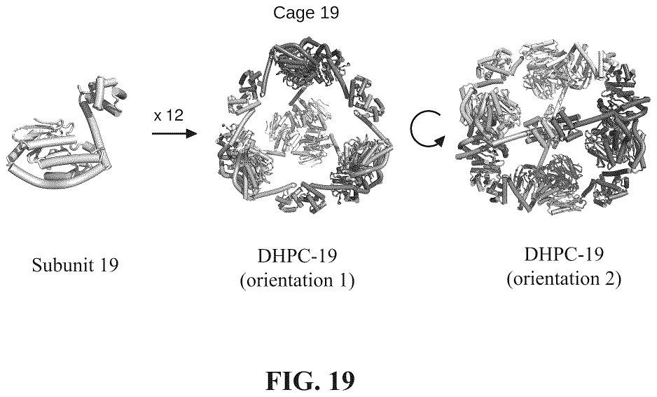

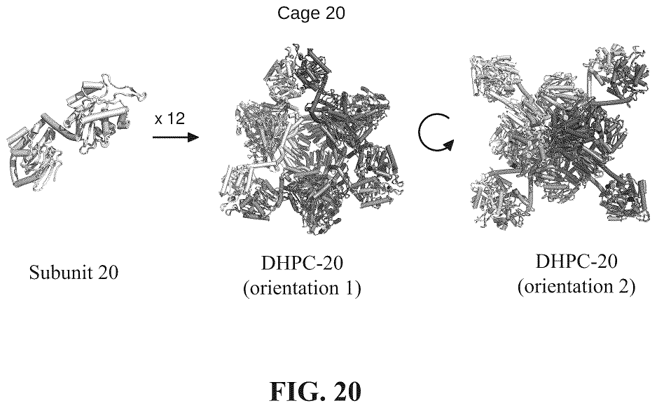

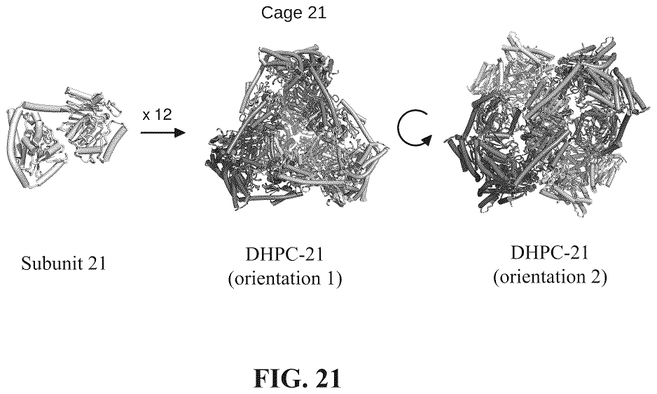

Twenty-one (21) human protein cages were designed, each of which self-assemble from 12 identical copies of a human fusion protein subunits. The 21 human protein cages are illustrated in FIGS. 1-21. The individual fusion protein subunits (Subunits 1-21, which correspond to SEQ ID NOS 1-21, respectively) are disclosed in detail in Table 10 below, in which the amino acid sequence of each fusion protein subunit is included. As indicated below, the residues of the protein from which the N-domain (underlined in the sequence) and C-domain (italicized in the sequence) of the individual fusion protein subunits is derived is identified by name and UniProt Accession No., and the number of linker residues is identified and indicated in bold in the sequence. Table 10 also indicates whether the N-terminal and/or the C-terminal is exposed. Where the N-terminal or C-terminal is exposed, an effector protein may be fused to the terminal.

TABLE 10
Human Fusion Protein Subunits

| SEQ | | | | |
|---|---|---|---|---|
| ID | Sequence of | N-Domain | Linker | |
| NO. | Human Fusion Protein Subunit | (underlined) | (bold) | C-Domain (italics) |
| 1 | YTYEDYKNTAEWLLSHTKHRPQVAIICGS | Purine nucleoside | 6 | Ragulator complex |
| | GLGGLTDKLTQAQIFDYGEIPNFPRSTVPG | phosphorylase | inserted | protein LAMTOR3 |
| | HAGRLVFGFLNGRACVMMQGRFHMYEG | (UniProtKB/Swiss- | helix | (UniProtKB/Swiss- |
| | YPLWKVTFPVRVFHLLGVDTLVVTNAAG | Prot No. Accession | residues | Prot No. Q9UHA4, |
| | GLNPKFEVGDIMLIRDHINLPGFSGQNPLR | No. P00491, | | residues 4-122) |
| | GPNDERFGDRFPAMSDAYDRTMRQRALS | Residues 5-280) | | C-terminal exposure: |
| | TWKQMGEQRELQEGTYVMVAGPSFETVA | N-terminal | | No |
| | ECRVLQKLGADAVGMSTVPEVIVARHCG | exposure: Yes | | |
| | LRVFGFSLITNKVIMDYESLEKANHEEVLA | | | |
| | AGKQAAQKLEQFVSILMAXXXXXXDLKR | | | |
| | FLYKKLPSVEGLHAIVVSDRDGVPVIKVAND | | | |
| | NAPEHALRPGFLSTFALATDQGSKLGLSKNK | | | |
| | SIICYYNTYQVVQFNRLPLVVSFIASSSANTGLI | | | |
| | VSLEKELAPLFEELRQVVE (Subunit 1 | | | |
| | length: 401 amino acid residues) | | | |
| 2 | YTYEDYKNTAEWLLSHTKHRPQVAIICGS | Purine nucleoside | 0 | N-acylglucosamine |
| | GLGGLTDKLTQAQIFDYGEIPNFPRSTVPG | phosphorylase | inserted | 2-epimerase (Renin |
| | HAGRLVFGFLNGRACVMMQGRFHMYEG | (UniProtKB/Swiss- | helix | Binding Protein) |
| | YPLWKVTFPVRVFHLLGVDTLVVTNAAG | Prot No. P00491, | residues | (UniProtKB/Swiss- |
| | GLNPKFEVGDIMLIRDHINLPGFSGQNPLR | Residues 5-280) | | Prot No. P51606, |
| | GPNDERFGDRFPAMSDAYDRTMRQRALS | N-terminal | | residues 15-410) |
| | TWKQMGEQRELQEGTYVMVAGPSFETVA | exposure: No | | C-terminal exposure: |
| | ECRVLQKLGADAVGMSTVPEVIVARHCG | | | Yes |
| | LRVFGFSLITNKVIMDYESLEKANHEEVLA | | | |
| | AGKQAAQKLEQFVSILMARETLQAWKERV | | | |
| | GQELDRVVAFWMEHSHDQEHGGFFTCLGR | | | |
| | EGRVYDDLKYVWLQGRQVWMYCRLYRTFER | | | |
| | FRHAQLLDAAKAGGEFLLRYARVAPPGKKC | | | |
| | AFVLTRDGRPVKVQRTIFSECFYTMAMNELW | | | |
| | RATGEVRYQTEAVEMMDQIVHWVQEDASGL | | | |
| | GRPQLQGAPAAEPMAVPMMLLNLVEQLGE | | | |
| | ADEELAGKYAELGDWCARRILQHVQRDGQA | | | |
| | VLENVSEGGKELPGCLGRQQNPGHTLEAGW | | | |
| | FLLRHCIRKGDPELRAHVIDKFLLLPFHSGW | | | |
| | DPDHGGLFYFQDADNFCPTQLEWAMKLW | | | |
| | WPHSEAMIAFLMGYSDSGDPVLLRLFYQVAE | | | |
| | YTFRQFRDPEYGEWFGYLSREGKVALSIKGG | | | |
| | PFKGCFHVPRCLAMCEEMLGALLSR | | | |
| | (Subunit 2 length: 672 amino | | | |
| | acid residues) | | | |
| 3 | RFGSQRVLVEPDAGAGVAVMKFKNPPVN | Enoyl-CoAdelta | 5 | Farnesyl |
| | SLSLEFLTELVISLEKLENDKSFRGVILTSD | isomerase 1- | inserted | pyrophosphate |
| | RPGVFSAGLDLTEMCGRSPAHYAGYWKA | mitochondrial | helix | synthase |
| | VQELWLRLYQSNLVLVSAINGACPAGGCL | (UniProtKB/Swiss- | residues | (UniProtKB/Swiss- |
| | VALTCDYRILADNPRYCIGLNETQLGIIAPF | Prot No. P42126, | | Prot No. P14324, |
| | WLKDTLENTIGHRAAERALQLGLLFPPAE | residues 41-297) | | residues 76-417) |
| | ALQVGIVDQVVPEEQVQSTALSAIAQWM | N-terminal | | C-terminal exposure: |
| | AIPDHARQLTKAMMRKATASRLVTQRDA | exposure: Yes | | No |
| | DVQNFVSFISKDSIQKSLQMYLERLXXXX | | | |
| | XYAQEKQDFVQHFSQIVRVLTEDEMGHPEI | | | |
| | GDAIARLKEVLEYNAIGGKYNRGLTVVVAFR | | | |
| | ELVEPRKQDADSLQRAWTVGWCVELLQAFF | | | |
| | LVADDIMDSSLTRRGQICWYQKPGVGLDAIN | | | |
| | DANLLEACIYRLLKLYCREQPYYLNLIELFLQS | | | |
| | SYQTEIGQTLDLLTAPQGNVDLVRFTEKRYK | | | |
| | SIVKYKTAFYSFYLPIAAAMYMAGIDGEKEHA | | | |
| | NAKKILLEMGEFFQIQDDYLDLFGDPSVTGK | | | |
| | IGTDIQDNKCSWLVVQCLQRATPEQYQILKE | | | |
| | NYGQKEAEKVARVKALYEELDLPAVFLQYEE | | | |
| | DSYSHIMALIEQYAAPLPPAVFLGLARKIYKR | | | |
| | (Subunit 3 length: 604 amino | | | |
| | acid residues) | | | |
| 4 | FETLVVTSEDGITKIMFNRPKKKNAINTEM | Enoyl-CoAdelta | 0 | Ran-binding protein |
| | YHEIMRALKAASKDDSIITVLTGNGDYYSS | isomerase 2 | inserted | 10 |
| | GNDLTNFTDIPPGGVEEKAKNNAVLLREF | (UniProtKB/Swiss- | helix | (UniProtKB/Swiss- |
| | VGCFIDFPKPLIAVVNGPAVGISVTLLGLF | Prot No. 075521, | residues | Prot No. Q6VN20, |
| | DAVYASDRATFHTPFSHLGQSPEGCSSYTF | residues 139-388) | | residues 32-613) |
| | PKIMSPAKATEMLIFGKKLTAGEACAQGL | N-terminal | | C-terminal exposure: |
| | VTEVFPDSTFQKEVWTRLKAFAKLPPNAL | exposure: Yes | | No |
| | RISKEVIRKREREKLHAVNAEECNVLQGR | | | |
| | WLSDECTNAVVNFLEQELSRRLQRLYPAVN | | | |
| | QQETPLPRSWSPKDKYNYIGLSQGNLRVHYK | | | |
| | GHGKNHKDAASVRATHPIPAACGIYYFEVKI | | | |
| | VSKGRDGYMGIGLSAQGVNMNRLPGWDKH | | | |
| | SYGYHGDDGHSFCSSGTGQPYGPTFTTGDVI | | | |
| | GCCVNLINGTCFYTKNGHSLGIAFTDLPANL | | | |
| | YPTVGLQTPGEIVDANFGQQPFLFDIEDYMR | | | |
| | EWRAKVQGTVHCFPSIARLGEWQAVLQNMV | | | |
| | SSYLVHHGYCATATAFARMTETPIQEEQASIK | | | |
| | NRQKIQKLVLEGRVGEAIETTQRFYPGLLEH | | | |
| | NPNLLFMLKCRQFVEMVNGTDSEVRSLSSRS | | | |
| | PKSQDSYPGSPSLSPRHGPSSSHMHNTGADS | | | |
| | PSCSNGVASTKSKQNHSKYPAPSSSSSSSSSSS | | | |
| | SSSPSSVNYSESNSTDSTKSQHHSSTSNQETSD | | | |
| | SEMEMEAEHYPNGVLGSMSTRIVNGAYKHE | | | |
| | DLQTDESSMDDRHPRRQLCGGNQAATERIIL | | | |
| | FGRELQALSEQLGREYGKNLAHTEMLQDAF | | | |
| | SLLAYSDPWSCPVGQQLDPIQREPVCAALNS | | | |
| | AILESQNLPKQPPLMLALGQASECLRLMARA | | | |
| | GLGSCSFA(Subunit 4 length: | | | |
| | 832 amino acid residues) | | | |
| 5 | KYIPRRAVLYVPGNDEKKIKKIPSLNVDCA | Citramalyl-CoA | 5 | NTF2-related export |
| | VLDCEDGVAANKKNEARLRIVKTLEDIDL | lyase- | inserted | protein 1 |
| | GPTEKCVRVNSVSSGLAEEDLETLLQSRV | mitochondrial | helix | (UniProtKB/Swiss- |
| | LPSSLMLPKVESPEEIQWFADKFSFHLKGR | (UniProtKB/Swiss- | residues | Prot No. Q9UKK6, |
| | KLEQPMNLIPFVETAMGLLNFKAVCEETL | Prot No. Q8NOX4, | | residues 7-136) |
| | KVGPQVGLFLDAVVFGGEDFRASIGATSS | residues 41-337) | | C-terminal exposure: |
| | KETLDILYARQKIVVIAKAFGLQAIDLVYI | N-terminal | | Yes |
| | DFRDGAGLLRQSREGAAMGFTGKQVIHP | exposure: Yes | | |
| | NQIAVVQEQFSPSPEKIKWAEELIAAFKEH | | | |
| | QQLGKGAFTFQGSMIDMPLLKQAQNTVT | | | |
| | LATSIXXXXXKTYVDQACRAAEEFVNVYYTT | | | |
| | MDKRRRLLSRLYMGTATLVWNGNAVSGQES | | | |
| | LSEFFEMLPSSEFQISVVDCQPVHDEATPSQ | | | |
| | TTVLVVICGSVKFEGNKQRDFNQNFILTAQA | | | |
| | SPSNTVWKIASDCFRFQ (Fusion protein | | | |
| | length: 432 amino acid residues) | | | |
| 6 | RFGSQRVLVEPDAGAGVAVMKFKNPPVN | Enoyl-CoAdelta | 1 | Centromere protein S |
| | SLSLEFLTELVISLEKLENDKSFRGVILTSD | isomerase 1- | inserted | (UniProtKB/Swiss- |
| | RPGVFSAGLDLTEMCGRSPAHYAGYWKA | mitochondrial | helix | Prot No. Q8N2Z9, |
| | VQELWLRLYQSNLVLVSAINGACPAGGCL | (UniProtKB/Swiss- | residues | residues 4-117) |
| | VALTCDYRILADNPRYCIGLNETQLGIIAPF | Prot No. P42126, | | C-terminal exposure: |
| | WLKDTLENTIGHRAAERALQLGLLFPPAE | residues 41-297) | | Yes |
| | ALQVGIVDQVVPEEQVQSTALSAIAQWM | N-terminal | | |
| | AIPDHARQLTKAMMRKATASRLVTQRDA | exposure: Yes | | |
| | DVQNFVSFISKDSIQKSLQMYLERLXEAET | | | |
| | EEQQRFSYQQRLKAAVHYTVGCLCEEVALD | | | |
| | KEMQFSKQTIAAISELTFRQCENFAKDLEMF | | | |
| | ARHAKRTTINTEDVKLLARRSNSLLKYITDKS | | | |
| | EEIAQINLERKAQKKKK (Fusion protein | | | |
| | length: 372 amino acid residues) | | | |
| 7 | FETLVVTSEDGITKIMFNRPKKKNAINTEM | Enoyl-CoAdelta | 1 | Fructose- |
| | YHEIMRALKAASKDDSIITVLTGNGDYYSS | isomerase 2 | inserted | bisphosphate aldolase |
| | GNDLTNFTDIPPGGVEEKAKNNAVLLREF | (UniProtKB/Swiss- | helix | B (UniProtKB/Swiss- |
| | VGCFIDFPKPLIAVVNGPAVGISVTLLGLF | Prot No. 075521, | residues | Prot No. P05062, |
| | DAVYASDRATFHTPFSHLGQSPEGCSSYTF | residues 139-388) | | residues 10-358) |
| | PKIMSPAKATEMLIFGKKLTAGEACAQGL | N-terminal | | C-terminal exposure: |
| | VTEVFPDSTFQKEVWTRLKAFAKLPPNAL | exposure: Yes | | No |
| | RISKEVIRKREREKLHAVNAEECNVLQGR | | | |
| | WLSDECTNAVVNFLXQEQKKELSEIAQSIV | | | |
| | ANGKGILAADESVGTMGNRLQRIKVENTEEN | | | |
| | RRQFREILFSVDSSINQSIGGVILFHETLYQKD | | | |
| | SQGKLFRNILKEKGIVVGIKLDQGGAPLAGT | | | |
| | NKETTIQGLDGLSERCAQYKKDGVDFGKWR | | | |
| | AVLRIADQCPSSLAIQENANALARYASICQQN | | | |
| | GLVPIVEPEVIPDGDHDLEHCQYVTEKVLAA | | | |
| | VYKALNDHHVYLEGTLLKPNMVTAGHACTK | | | |
| | KYTPEQVAMATVTALHRTVPAAVPGICFLSG | | | |
| | GMSEEDATLNLNAINLCPLPKPWKLSFSYGR | | | |
| | ALQASALAAWGGKAANKEATQEAFMKRAMA | | | |
| | NCQAAKGQYVHTGSSGAASTQSLFTACYTY | | | |
| | (Fusion protein length: 600 | | | |
| | amino acid residues) | | | |
| 8 | RFGSQRVLVEPDAGAGVAVMKFKNPPVN | Enoyl-CoAdelta | 6 | Barrier-to- |
| | SLSLEFLTELVISLEKLENDKSFRGVILTSD | isomerase 1- | inserted | autointegration |
| | RPGVFSAGLDLTEMCGRSPAHYAGYWKA | mitochondrial | helix | factor-like protein |
| | VQELWLRLYQSNLVLVSAINGACPAGGCL | (UniProtKB/Swiss- | residues | (UniProtKB/Swiss- |
| | VALTCDYRILADNPRYCIGLNETQLGIIAPF | Prot No. P42126, | | Prot No. Q9H503, |
| | WLKDTLENTIGHRAAERALQLGLLFPPAE | residues 41-297) | | residues 6-90) |
| | ALQVGIVDQVVPEEQVQSTALSAIAQWM | N-terminal | | C-terminal exposure: |
| | AIPDHARQLTKAMMRKATASRLVTQRDA | exposure: Yes | | No |
| | DVQNFVSFISKDSIQKSLQMYLERLXXXX | | | |
| | XXARLRAFLSEPIGEKDVCWVDGISHELAIN | | | |
| | LVTKGINKAYILLGQFLLMHKNEAEFQRWLI | | | |
| | CCFGATECEAQQTSHCLKEWCACFL(Fusion | | | |
| | protein length: 348 amino | | | |
| | acid residues) | | | |
| 9 | FETLVVTSEDGITKIMFNRPKKKNAINTEM | Enoyl-CoAdelta | 0 | Centromere protein S |
| | YHEIMRALKAASKDDSIITVLTGNGDYYSS | isomerase 2 | inserted | (UniProtKB/Swiss- |
| | GNDLTNFTDIPPGGVEEKAKNNAVLLREF | (UniProtKB/Swiss- | helix | Prot No. Q8N2Z9, |
| | VGCFIDFPKPLIAVVNGPAVGISVTLLGLF | Prot No. 075521, | residues | residues 4-117) |
| | DAVYASDRATFHTPFSHLGQSPEGCSSYTE | residues 139-388) | | C-terminal exposure: |
| | PKIMSPAKATEMLIFGKKLTAGEACAQGL | N-terminal | | Yes |
| | VTEVFPDSTFQKEVWTRLKAFAKLPPNAL | exposure: Yes | | |
| | RISKEVIRKREREKLHAVNAEECNVLQGR | | | |
| | WLSDECTNAVVNFLEAETEEQQRFSYQQR | | | |
| | LKAAVHYTVGCLCEEVALDKEMQFSKQTIAA | | | |
| | ISELTFRQCENFAKDLEMFARHAKRTTINTE | | | |
| | DVKLLARRSNSLLKYITDKSEEIAQINLERKAQ | | | |
| | KKKK (Fusion protein length: | | | |
| | 364 amino acid residues) | | | |
| 10 | RFGSQRVLVEPDAGAGVAVMKFKNPPVN | Enoyl-CoAdelta | 2 | Retinol |
| | SLSLEFLTELVISLEKLENDKSFRGVILTSD | isomerase 1- | inserted | dehydrogenase 10 |
| | RPGVFSAGLDLTEMCGRSPAHYAGYWKA | mitochondrial | helix | (UniProtKB/Swiss- |
| | VQELWLRLYQSNLVLVSAINGACPAGGCL | (UniProtKB/Swiss- | residues | Prot No. Q8IZV5, |
| | VALTCDYRILADNPRYCIGLNETQLGIIAPF | Prot No. P42126, | | residues 4-332) |
| | WLKDTLENTIGHRAAERALQLGLLFPPAE | residues 41-297) | | C-terminal exposure: |
| | ALQVGIVDQVVPEEQVQSTALSAIAQWM | N-terminal | | Yes |
| | AIPDHARQLTKAMMRKATASRLVTQRDA | exposure: Yes | | |
| | DVQNFVSFISKDSIQKSLQMYLERLXXVVE | | | |
| | FFVVTFKVLWAFVLAAARWLVRPKEKSVAG | | | |
| | QVCLITGAGSGLGRLFALEFARRRALLVLWDI | | | |
| | NTQSNEETAGMVRHIYRDLEAADAAALQAG | | | |
| | NGEEEILPHCNLQVFTYTCDVGKRENVYLTA | | | |
| | ERVRKEVGEVSVLVNNAGVVSGHHLLECPD | | | |
| | ELIERTMMVNCHAHFWTTKAFLPTMLEINH | | | |
| | GHIVTVASSLGLFSTAGVEDYCASKFGVVGF | | | |
| | HESLSHELKAAEKDGIKTTLVCPYLVDTGMF | | | |
| | RGCRIRKEIEPFLPPLKPDYCVKQAMKAILT | | | |
| | DQPMICTPRLMYIVTFMKSILPFEAVVCMYR | | | |
| | FLGADKCMYPFIAQRKQAT (Fusion protein | | | |
| | length: 588 amino acid residues) | | | |
| 11 | RFGSQRVLVEPDAGAGVAVMKFKNPPVN | Enoyl-CoAdelta | 5 | Tyrosine-tRNA |
| | SLSLEFLTELVISLEKLENDKSFRGVILTSD | isomerase 1- | inserted | ligase- |
| | RPGVFSAGLDLTEMCGRSPAHYAGYWKA | mitochondrial | helix | mitochondrial |
| | VQELWLRLYQSNLVLVSAINGACPAGGCL | (UniProtKB/Swiss- | residues | (UniProtKB/Swiss- |
| | VALTCDYRILADNPRYCIGLNETQLGIIAPF | Prot No. P42126, | | Prot No. Q9Y2Z4, |
| | WLKDTLENTIGHRAAERALQLGLLFPPAE | residues 41-297) | | residues 39-476) |
| | ALQVGIVDQVVPEEQVQSTALSAIAQWM | N-terminal | | C-terminal exposure: |
| | AIPDHARQLTKAMMRKATASRLVTQRDA | exposure: Yes | | Yes |
| | DVQNFVSFISKDSIQKSLQMYLERLXXXX | | | |
| | XLAAQKARGLFKDFFPETGTKIELPELFDRG | | | |
| | TASFPQTIYCGFDPTADSLHVGHLLALLGLF | | | |
| | HLQRAGHNVIALVGGATARLGDPSGRTKERE | | | |
| | ALETERVRANARALRLGLEALAANHQQLFTD | | | |
| | GRSWGSFTVLDNSAWYQKQHLVDFLAAVGG | | | |
| | HFRMGTLLSRQSVQLRLKSPEGMSLAEFFYQ | | | |
| | VLQAYDFYYLFQRYGCRVQLGGSDQLGNIM | | | |
| | SGYEFINKLTGEDVFGITVPLITSTTGAKLGKS | | | |
| | AGNAVWLNRDKTSPFELYQFFVRQPDDSVE | | | |
| | RYLKLFTFLPLPEIDHIMQLHVKEPERRGPQ | | | |
| | KRLAAEVTKLVHGREGLDSAKRCTQALYHSS | | | |
| | IDALEVMSDQELKELFKEAPFSEFFLDPGTS | | | |
| | VLDTCRKANAIPDGPRGYRMITEGGVSINHQ | | | |
| | QVTNPESVLIVGQHILKNGLSLLKIGKRNFYII | | | |
| | KWLQ (Fusion protein length: | | | |
| | 700 amino acid residues) | | | |
| 12 | RFGSQRVLVEPDAGAGVAVMKFKNPPVN | Enoyl-CoAdelta | 0 | Fructose- |
| | SLSLEFLTELVISLEKLENDKSFRGVILTSD | isomerase 1- | inserted | bisphosphate aldolase |
| | RPGVFSAGLDLTEMCGRSPAHYAGYWKA | mitochondrial | helix | B (UniProtKB/Swiss- |
| | VQELWLRLYQSNLVLVSAINGACPAGGCL | (UniProtKB/Swiss- | residues | Prot No. P05062, |
| | VALTCDYRILADNPRYCIGLNETQLGIIAPF | Prot No. P42126, | | residues 12-358) |
| | WLKDTLENTIGHRAAERALQLGLLFPPAE | residues 41-297) | | C-terminal exposure: |
| | ALQVGIVDQVVPEEQVQSTALSAIAQWM | N-terminal | | Yes |
| | AIPDHARQLTKAMMRKATASRLVTQRDA | exposure: Yes | | |
| | DVQNFVSFISKDSIQKSLQMYLERLQKKEL | | | |
| | SEIAQSIVANGKGILAADESVGTMGNRLQRIK | | | |
| | VENTEENRRQFREILFSVDSSINQSIGGVILFH | | | |
| | ETLYQKDSQGKLFRNILKEKGIVVGIKLDQG | | | |
| | GAPLAGTNKETTIQGLDGLSERCAQYKKDG | | | |
| | VDFGKWRAVLRIADQCPSSLAIQENANALAR | | | |
| | YASICQQNGLVPIVEPEVIPDGDHDLEHCQY | | | |
| | VTEKVLAAVYKALNDHHVYLEGTLLKPNMVT | | | |
| | AGHACTKKYTPEQVAMATVTALHRTVPAAVP | | | |
| | GICFLSGGMSEEDATLNLNAINLCPLPKPWK | | | |
| | LSFSYGRALQASALAAWGGKAANKEATQEAF | | | |
| | MKRAMANCQAAKGQYVHTGSSGAASTQSLF | | | |
| | TACYTY (Fusion protein length: | | | |
| | 604 amino acid residues) | | | |
| 13 | KYIPRRAVLYVPGNDEKKIKKIPSLNVDCA | Citramalyl-CoA | 3 | Choline/ethanolamine |
| | VLDCEDGVAANKKNEARLRIVKTLEDIDL | lyase- | inserted | kinase |
| | GPTEKCVRVNSVSSGLAEEDLETLLQSRV | mitochondrial | helix | (UniProtKB/Swiss- |
| | LPSSLMLPKVESPEEIQWFADKFSFHLKGR | (UniProtKB/Swiss- | residues | Prot No. Q9Y259, |
| | KLEQPMNLIPFVETAMGLLNFKAVCEETL | Prot No. Q8NOX4, | | residues 48-389) |
| | KVGPQVGLFLDAVVFGGEDFRASIGATSS | residues 41-337) | | C-terminal exposure: |
| | KETLDILYARQKIVVIAKAFGLQAIDLVYI | N-terminal | | Yes |
| | DFRDGAGLLRQSREGAAMGFTGKQVIHP | exposure: Yes | | |
| | NQIAVVQEQFSPSPEKIKWAEELIAAFKEH | | | |
| | QQLGKGAFTFQGSMIDMPLLKQAQNTVT | | | |
| | LATSIXXXRAYQWCREYLGGAWRRVQPEEL | | | |
| | RVYPVSGGLSNLLFRCSLPDHLPSVGEEPRE | | | |
| | VLLRLYGAILQGVDSLVLESVMFAILAERSLG | | | |
| | PQLYGVFPEGRLEQYIPSRPLKTQELREPVLS | | | |
| | AAIATKMAQFHGMEMPFTKEPHWLFGTME | | | |
| | RYLKQIQDLPPTGLPEMNLLEMYSLKDEMG | | | |
| | NLRKLLESTPSPVVFCHNDIQEGNILLLSEPE | | | |
| | NADSLMLVDFEYSSYNYRGFDIGNHFCEWVY | | | |
| | DYTHEEWPFYKARPTDYPTQEQQLHFIRHYL | | | |
| | AEAKKGETLSQEEQRKLEEDLLVEVSRYALA | | | |
| | SHFFWGLWSILQASMSTIEFGYLDYAQSRFQ | | | |
| | FYFQQKGQLT (Fusion protein length: | | | |
| | 642 amino acid residues) | | | |
| 14 | YTYEDYKNTAEWLLSHTKHRPQVAIICGS | Purine nucleoside | 6 | Ragulator complex |
| | GLGGLTDKLTQAQIFDYGEIPNFPRSTVPG | phosphorylase | inserted | protein LAMTOR5 |
| | HAGRLVFGFLNGRACVMMQGRFHMYEG | (UniProt Accession | helix | (UniProtKB/Swiss- |
| | YPLWKVTFPVRVFHLLGVDTLVVTNAAG | No. P00491, | residues | Prot No. 043504, |
| | GLNPKFEVGDIMLIRDHINLPGFSGQNPLR | Residues 5-280) | | residues 4-90) |
| | GPNDERFGDRFPAMSDAYDRTMRQRALS | N-terminal | | C-terminal exposure: |
| | TWKQMGEQRELQEGTYVMVAGPSFETVA | exposure: Yes | | Yes |
| | ECRVLQKLGADAVGMSTVPEVIVARHCG | | | |
| | LRVFGFSLITNKVIMDYESLEKANHEEVLA | | | |
| | AGKQAAQKLEQFVSILMAXXXXXXTLEQ | | | |
| | HLEDTMKNPSIVGVLCTDSQGLNLGCRGTLS | | | |
| | DEHAGVISVLAQQAAKLTSDPTDIPVVCLESD | | | |
| | NGNIMIQKHDGITVAVHKMA(Fusion protein | | | |
| | length: 369 amino acid residues) | | | |
| 15 | FETLVVTSEDGITKIMFNRPKKKNAINTEM | Enoyl-CoAdelta | 4 | Centromere protein S |
| | YHEIMRALKAASKDDSIITVLTGNGDYYSS | isomerase 2 | inserted | (UniProtKB/Swiss- |
| | GNDLTNFTDIPPGGVEEKAKNNAVLLREF | (UniProtKB/Swiss- | helix | Prot No. Q8N2Z9, |
| | VGCFIDFPKPLIAVVNGPAVGISVTLLGLF | Prot No. 075521, | residues | residues 4-117) |
| | DAVYASDRATFHTPFSHLGQSPEGCSSYTF | residues 139-388) | | C-terminal exposure: |
| | PKIMSPAKATEMLIFGKKLTAGEACAQGL | N-terminal | | No |
| | VTEVFPDSTFQKEVWTRLKAFAKLPPNAL | exposure: Yes | | |
| | RISKEVIRKREREKLHAVNAEECNVLQGR | | | |
| | WLSDECTNAVVNFLXXXXEAETEEQQRFS | | | |
| | YQQRLKAAVHYTVGCLCEEVALDKEMQFSK | | | |
| | QTIAAISELTFRQCENFAKDLEMFARHAKRT | | | |
| | TINTEDVKLLARRSNSLLKYITDKSEEIAQINL | | | |
| | ERKAQKKKK (Fusion protein length: | | | |
| | 368 amino acid residues) | | | |
| 16 | FETLVVTSEDGITKIMFNRPKKKNAINTEM | Enoyl-CoAdelta | 0 | Serine |
| | YHEIMRALKAASKDDSIITVLTGNGDYYSS | isomerase 2 | inserted | hydroxymethyltrans- |
| | GNDLTNFTDIPPGGVEEKAKNNAVLLREF | (UniProtKB/Swiss- | helix | ferase-cytosolic- |
| | VGCFIDFPKPLIAVVNGPAVGISVTLLGLF | Prot No. 075521, | residues | pdb/gene-to- |
| | DAVYASDRATFHTPFSHLGQSPEGCSSYTE | residues 139-388) | | fusion residue |
| | PKIMSPAKATEMLIFGKKLTAGEACAQGL | N-terminal | | mapping: 15-481 to |
| | VTEVFPDSTFQKEVWTRLKAFAKLPPNAL | exposure: Yes | | 251-717 |
| | RISKEVIRKREREKLHAVNAEECNVLQGR | | | C-terminal exposure: |
| | WLSDECTNAVVNFLWSSHDKMLAQPLKDS | | | No |
| | DVEVYNIIKKESNRQRVGLELIASENFASRAVL | | | |
| | EALGSCLNNKYSEGYPGQRYYGGTEFIDELE | | | |
| | TLCQKRALQAYKLDPQCWGVNVQPYSGSPA | | | |
| | NFAVYTALVEPHGRIMGLDLPDGGHLTHGF | | | |
| | MTDKKKISATSIFFESMPYKVNPDTGYINYDQ | | | |
| | LEENARLFHPKLIIAGTSCYSRNLEYARLRKIA | | | |
| | DENGAYLMADMAHISGLVAAGVVPSPFEHC | | | |
| | HVVTTTTHKTLRGCRAGMIFYRKGVKSVDPK | | | |
| | TGKEILYNLESLINSAVFPGLQGGPHNHAIAG | | | |
| | VAVALKQAMTLEFKVYQHQVVANCRALSEAL | | | |
| | TELGYKIVTGGSDNHLILVDLRSKGTDGGRA | | | |
| | EKVLEACSIACNKNTCPGDRSALRPSGLRLG | | | |
| | TPALTSRGLLEKDFQKVAHFIHRGIELTLQIQ | | | |
| | SDTGVRATLKEFKERLAGDKYQAAVQALREE | | | |
| | VESFASLFPLPGLP (Fusion protein | | | |
| | length: 717 amino acid residues) | | | |
| 17 | FETLVVTSEDGITKIMFNRPKKKNAINTEM | Enoyl-CoAdelta | 2 | Centromere protein S |
| | YHEIMRALKAASKDDSIITVLTGNGDYYSS | isomerase 2 | inserted | (UniProtKB/Swiss- |
| | GNDLTNFTDIPPGGVEEKAKNNAVLLREF | (UniProtKB/Swiss- | helix | Prot No. Q8N2Z9, |
| | VGCFIDFPKPLIAVVNGPAVGISVTLLGLF | Prot No. 075521, | residues | residues 4-117) |
| | DAVYASDRATFHTPFSHLGQSPEGCSSYTF | residues 139-388) | | C-terminal exposure: |
| | PKIMSPAKATEMLIFGKKLTAGEACAQGL | N-terminal | | Yes |
| | VTEVFPDSTFQKEVWTRLKAFAKLPPNAL | exposure: Yes | | |
| | RISKEVIRKREREKLHAVNAEECNVLQGR | | | |
| | WLSDECTNAVVNFLXXEAETEEQQRFSYQ | | | |
| | QRLKAAVHYTVGCLCEEVALDKEMQFSKQTI | | | |
| | AAISELTFRQCENFAKDLEMFARHAKRTTINT | | | |
| | EDVKLLARRSNSLLKYITDKSEEIAQINLERKA | | | |
| | QKKKK (Fusion protein length: | | | |
| | 366 amino acid residues) | | | |
| 18 | RFGSQRVLVEPDAGAGVAVMKFKNPPVN | Enoyl-CoAdelta | 0 | Malonyl-CoA |
| | SLSLEFLTELVISLEKLENDKSFRGVILTSD | isomerase 1- | inserted | decarboxylase- |
| | RPGVFSAGLDLTEMCGRSPAHYAGYWKA | mitochondrial | helix | mitochondrial |
| | VQELWLRLYQSNLVLVSAINGACPAGGCL | (UniProtKB/Swiss- | residues | (UniProtKB/Swiss- |
| | VALTCDYRILADNPRYCIGLNETQLGIIAPF | Prot No. P42126, | | Prot No. 095822, |
| | WLKDTLENTIGHRAAERALQLGLLFPPAE | residues 41-297) | | residues 39-489) |
| | ALQVGIVDQVVPEEQVQSTALSAIAQWM | N-terminal | | C-terminal exposure: |
| | AIPDHARQLTKAMMRKATASRLVTQRDA | exposure: Yes | | No |
| | DVQNFVSFISKDSIQKSLQMYLERLAMDEL | | | |
| | LRRAVPPTPAYELREKTPAPAEGQCADFVSF | | | |
| | YGGLAETAQRAELLGRLARGFGVDHGQVAE | | | |
| | QSAGVLHLRQQQREAAVLLQAEDRLRYALVP | | | |
| | RYRGLFHHISKLDGGVRFLVQLRADLLEAQA | | | |
| | LKLVEGPDVREMNGVLKGMLSEWFSSGFLN | | | |
| | LERVTWHSPCEVLQKISEAEAVHPVKNWMD | | | |
| | MKRRVGPYRRCYFFSHCSTPGEPLVVLHVAL | | | |
| | TGDISSNIQAIVKEHPPSETEEKNKITAAIFYSI | | | |
| | SLTQQGLQGVELGTFLIKRVVKELQREFPHL | | | |
| | GVFSSLSPIPGFTKWLLGLLNSQTKEHGRNE | | | |
| | LFTDSECKEISEITGGPINETLKLLLSSSEWVQ | | | |
| | SEKLVRALQTPLMRLCAWYLYGEKHRGYALN | | | |
| | PVANFHLQNGAVLWRINWMADVSLRGITGS | | | |
| | CGLMANYRYFLEETGPNSTSYLGSKIIKASEQ | | | |
| | VLSLVAQFQK (Fusion protein length: | | | |
| | 708 amino acid residues) | | | |
| 19 | RFGSQRVLVEPDAGAGVAVMKFKNPPVN | Enoyl-CoAdelta | 6 | Barrier-to- |
| | SLSLEFLTELVISLEKLENDKSFRGVILTSD | isomerase 1- | inserted | autointegration factor |
| | RPGVFSAGLDLTEMCGRSPAHYAGYWKA | mitochondrial | helix | (UniProtKB/Swiss- |
| | VQELWLRLYQSNLVLVSAINGACPAGGCL | (UniProtKB/Swiss- | residues | Prot No. 075531, |
| | VALTCDYRILADNPRYCIGLNETQLGIIAPF | Prot No. P42126, | | residues 5-89) |
| | WLKDTLENTIGHRAAERALQLGLLFPPAE | residues 41-297) | | C-terminal exposure: |
| | ALQVGIVDQVVPEEQVQSTALSAIAQWM | N-terminal | | No |
| | AIPDHARQLTKAMMRKATASRLVTQRDA | exposure: Yes | | |
| | DVQNFVSFISKDSIQKSLQMYLERLXXXX | | | |
| | XXQKHRDFVAEPMGEKPVGSLAGIGEVLGK | | | |
| | KLEERGFDKAYVVLGQFLVLKKDEDLFREW | | | |
| | LKDTCGANAKQSRDCFGCLREWCDAFL | | | |
| | (Fusion protein length: 348 | | | |
| | amino acid residues) | | | |
| 20 | RFGSQRVLVEPDAGAGVAVMKFKNPPVN | Enoyl-CoAdelta | 1 | Cytosolic Fe-S |
| | SLSLEFLTELVISLEKLENDKSFRGVILTSD | isomerase 1- | inserted | cluster assembly |
| | RPGVFSAGLDLTEMCGRSPAHYAGYWKA | mitochondrial | helix | factor NUBP1 |
| | VQELWLRLYQSNLVLVSAINGACPAGGCL | (UniProtKB/Swiss- | residues | (UniProtKB/Swiss- |
| | VALTCDYRILADNPRYCIGLNETQLGIIAPF | Prot No. P42126, | | Prot No. P53384, |
| | WLKDTLENTIGHRAAERALQLGLLFPPAE | residues 41-297) | | residues 43-309) |
| | ALQVGIVDQVVPEEQVQSTALSAIAQWM | N-terminal | | C-terminal exposure: |
| | AIPDHARQLTKAMMRKATASRLVTQRDA | exposure: Yes | | Yes |
| | DVQNFVSFISKDSIQKSLQMYLERLXIEEIK | | | |
| | EKMKTVKHKILVLSGKGGVGKSTFSAHLAH | | | |
| | GLAEDENTQIALLDIDICGPSIPKIMGLEGEQ | | | |
| | VHQSGSGWSPVYVEDNLGVMSVGFLLSSPD | | | |
| | DAVIWRGPKKNGMIKQFLRDVDWGEVDYLI | | | |
| | VDTPPGTSDEHLSVVRYLATAHIDGAVIITTP | | | |
| | QEVSLQDVRKEINFCRKVKLPIIGVVENMSG | | | |
| | FICPKCKKESQIFPPTTGGAELMCQDLEVPL | | | |
| | LGRVPLDPLIGKNCDKGQSFFIDAPDSPATL | | | |
| | AYRSIIQRIQEFCNL(Fusion protein | | | |
| | length: 525 amino acid residues) | | | |
| 21 | FETLVVTSEDGITKIMFNRPKKKNAINTEM | Enoyl-CoAdelta | 6 | C-domain: Inositol |
| | YHEIMRALKAASKDDSIITVLTGNGDYYSS | isomerase 2 | inserted | monophosphatase |
| | GNDLTNFTDIPPGGVEEKAKNNAVLLREF | (UniProtKB/Swiss- | helix | (UniProtKB/Swiss- |
| | VGCFIDFPKPLIAVVNGPAVGISVTLLGLF | Prot No. 075521, | residues | Prot No. P29218, |
| | DAVYASDRATFHTPFSHLGQSPEGCSSYTF | residues 139-388) | | residues 6-276) |
| | PKIMSPAKATEMLIFGKKLTAGEACAQGL | N-terminal | | C-terminal exposure: |
| | VTEVFPDSTFQKEVWTRLKAFAKLPPNAL | exposure: Yes | | No |
| | RISKEVIRKREREKLHAVNAEECNVLQGR | | | |
| | WLSDECTNAVVNFLXXXXXXQECMDYAV | | | |
| | TLARQAGEVVCEAIKNEMNVMLKSSPVDLVT | | | |
| | ATDQKVEKMLISSIKEKYPSHSFIGEESVAAG | | | |
| | EKSILTDNPTWIIDPIDGTTNFVHRFPFVAVSI | | | |
| | GFAVNKKIEFGVVYSCVEGKMYTARKGKGA | | | |
| | FCNGQKLQVSQQEDITKSLLVTELGSSRTPET | | | |
| | VRMVLSNMEKLFCIPVHGIRSVGTAAVNMCL | | | |
| | VATGGADAYYEMGIHCWDVAGAGIIVTEAG | | | |
| | GVLMDVTGGPFDLMSRRVIAANNRILAERIA | | | |
| | KEIQVIPLQRDDE (Fusion protein | | | |
| | length: 527 amino acid residues) | | | |

### Example 2: Mammalian Fusion Protein Subunits

Twenty-one (42) mammalian protein cages were designed as homologous to the human protein cages disclosed in Example 1 above. Each of the mammalian protein cages self-assembles from 12 identical copies of a mammalian fusion protein subunit. The individual fusion protein subunits (Subunits 1-21) for cyno monkey and mouse homologues are disclosed in detail in Table 11 below, in which the Subunit number corresponds to that of the human fusion protein subunit above, and the amino acid sequence of each fusion protein subunit is included. As indicated below, the residues of the protein from which the N-domain is underlined in the sequence, the C-domain is italicized in the sequence) and the number of linker residues is identified and indicated in bold in the sequences.

TABLE 11
Mammalian Fusion Protein Subunits

| Fusion | | |
|---|---|---|
| Protein | Sequence of Cyno Monkey Fusion | Sequence of Mouse Fusion |
| Subunit | Protein Subunit | Protein Subunit |
| Subunit 1 | YTYEDYKNTAEWLLSHTKHRPQVAIICGSGL | FTYEDYETTAKWLLQHTEYRPQVAVICGS |
| | GGLTDKLTQAQIFDYSEIPNFPQSTVPGHVGR | GLGGLTAHLKEAQIFDYNEIPNFPQSTVQ |
| | LVFGFLNGRACVIMQGRFHMYEGYSLWKVT | GHAGRLVFGLLNGRCCVMMQGRFHMYE |
| | FPVRVFHLLGVDTLVVTNAAGGLNPEFEVG | GYSLSKVTFPVRVFHLLGVETLVVTNAAG |
| | DIMLIRDHINLPGFSGQNPLRGPNDERFGVRF | GLNPNFEVGDIMLIRDHINLPGFCGQNPLR |
| | PAMSDAYDRTMRQRALSMWKQMGEQREL | GPNDERFGVRFPAMSDAYDRDMRQKAFS |
| | QEGTYVMLAGPSFETVAESRLLQKLGADAV | AWKQMGEQRKLQEGTYVMLAGPNFETV |
| | GMSTVPEVIVARHCGLRVFGFSLITNKVIMD | AESRLLKMLGADAVGMSTVPEVIVARHC |
| | YESLEKANHEEVLAAGKQAAQKLEQLVSIL | GLRVFGFSLITNKVVMDYENLEKANHME |
| | MAXXXXXXDLKRFLYKKLPSVEGLHAIVVSDR | VLDAGKAAAQTLERFVSILMEXXXXXX |
| | DGVPVIKVANDNAPEHALRPGFLSTFALATDQ | DLKRFLYKKLPSVEGLHAIVVSDRDGVPVIK |
| | GSKLGLSKNKSIICYYNTYQVVQFNRLPLVVSFIA | VANDSAPEHALRPGFLSTFALATDQGSKLGL |
| | SSSANTGLIVSLEKELAPLFEELRQVVE (SEQ ID | SKNKSIICYYNTYQVVQFNRLPLVVSFIASSSA |
| | NO: 22) | NTGLIVSLEKELAPLFEELIKVVE (SEQ ID |
| | | NO: 23) |
| Subunit 2 | YTYEDYKNTAEWLLSHTKHRPQVAIICGSGL | FTYEDYETTAKWLLQHTEYRPQVAVICGS |
| | GGLTDKLTQAQIFDYSEIPNFPQSTVPGHVGR | GLGGLTAHLKEAQIFDYNEIPNFPQSTVQ |
| | LVFGFLNGRACVIMQGRFHMYEGYSLWKVT | GHAGRLVFGLLNGRCCVMMQGRFHMYE |
| | FPVRVFHLLGVDTLVVTNAAGGLNPEFEVG | GYSLSKVTFPVRVFHLLGVETLVVTNAAG |
| | DIMLIRDHINLPGFSGQNPLRGPNDERFGVRF | GLNPNFEVGDIMLIRDHINLPGFCGQNPLR |
| | PAMSDAYDRTMRQRALSMWKQMGEQREL | GPNDERFGVRFPAMSDAYDRDMRQKAFS |
| | QEGTYVMLAGPSFETVAESRLLQKLGADAV | AWKQMGEQRKLQEGTYVMLAGPNFETV |
| | GMSTVPEVIVARHCGLRVFGFSLITNKVIMD | AESRLLKMLGADAVGMSTVPEVIVARHC |
| | YESLEKANHEEVLAAGKQAAQKLEQLVSIL | GLRVFGFSLITNKVVMDYENLEKANHME |
| | MARETLQAWKERVAQELDRVVAFWMEHSHD | VLDAGKAAAQTLERFVSILMERETLQVWK |
| | QEHGGFFTCLGREGRVYDDLKYVWLQGRQVW | KRVEQELDRVIAFWMEHSHDQEHGGFFTC |
| | MYCRLYRTFQRFHHSQLLNAAKAGGEFLLRYA | LGRDGKVYDHLKYVWLQGRQVWMYCRLYR |
| | RLAPPGKKCAFVLTRDGRPVKVQRTIFSECFYT | SFERFRRVELLDAARAGGEFLLRYARVAPPG |
| | MAMNELWRATGEVQYQTEAVEMMDQIVHWVR | KKCAFVLTRDGRPVKVQRTIFSECFYTMAM |
| | EDASGLGRPQLQGAPTAEPMAVPMMLLNLVE | NELWKVTGEVRYQSEAIEMMDQIIHWVRED |
| | QLGEADEELAGKYTELGDWCAQRILQHVQRD | PAGLGRPQLSGALATEPMAVPMMLLSLVEQ |
| | GQAVLENVSEDGKELSGCLGRQQNPGHALEA | LGEEDEELTNMYAELGDWCVHRILQHVQR |
| | GWFLLRHCNRKGDPELRAHVVDKFLLLPFRSG | DGQVVLENVSEDGKELPGCLGRHQNPGHT |
| | WDPDHGGLFYFQDADDFCPTQLEWAMKLWW | LEAGWFLLQYALRKGDPKLRMHIIDKFLLLP |
| | PHSEAMIAFLMGYSESGDPVLLRLFYQVAEYTF | FHSGWDPEHGGLFYFQDADGLCPTQLEWN |
| | RQFRDPEYGEWFGYLSREGKVALSIKGGPFKG | MKLWWPHSEAMIAFLMGYSDSGDPALLHL |
| | CFHVPRCLAMCEEMLGALLSR (SEQ ID NO: 24) | FYKVAEYTFRQFRDPEYGEWFGYLNQEGKV |
| | | ALTIKGGPFKGCFHVPRCLAMCEQILGALL |
| | | QR (SEQ ID NO: 25) |
| Subunit 3 | RFGSQRVLVEPDAGAGVAVMKLKNPPVNTL | RFANKRVLVETEGPAGVAVMKLRNPPVN |
| | SLEFLTELVICLEKLENDKSFRGVVLTSDCPG | SLSLECLTEFTISLEKLENDKSIRGVILTSE |
| | VFSAGLDLTEMCGKSPAHYAEYWKAVQEL | CPGIFSAGLDLLEMYGRNPAHYAEYWKN |
| | WLQFYQSNLVLVSAINGACPAGGCLMTLTC | VQELWLRLYTSNMILVSAINGASPAGGCL |
| | DYRVLADNPRYCIGLNETQLGIVAPFWFKDT | LALCCDYRVMADNPKYTIGLNESLLGIVA |
| | LENVIGHRAAERALQLGLLFPPAEALQVGIV | PFWFKDMYVNTIGHREAERALQLGTLFSP |
| | DQVVPEEQVQSTALSAIAQWMTIPDHARQLT | AEALKVGVVDEVVPEDQVHSKARSVMT |
| | KAMMRKATASRLITQRDADVQNFVSFISKDS | KWLAIPDHSRQLTKNMMRKATADNLIKQ |
| | IQKSLQLYLERLXXXXXYAQEKQDFVQHFSQ | READIQNFTSFISKDSIQKSLHMYLEKLXX |
| | IVRVLTEDEMGHPETGDAIARLKEVLEYNAIGG | XXXYNQEKQNFIQHFSQIVKVLTEKELGHP |
| | KYHRGLTVVVAFRELVEPRKQDADSLQRAWTV | EIGDAIARLKEVLEYNALGGKYNRGLTVVQA |
| | GWCVELLQAFFLVTDDIMDSSLTRRGQICWYQ | FQELVEPKKQDAESLQRALTVGWCVELLQA |
| | KPGVGLDAINDAILLEACIYRLLKLYCREQPYYL | FFLVSDDIMDSSLTRRGQICWYQKPGIGLDA |
| | NLIELFLQSSYQTEIGQTLDLITAPQGNVDLGRF | INDALLLEASIYRLLKFYCREQPYYLNLLELFL |
| | TEKRYKSIVKYKTAFYSFYLPIAAAMYMAGIDGE | QSSYQTEIGQTLDLMTAPQGHVDLGRYTEK |
| | KEHANAKKILLEMGEFFQIQDDYLDLFGDPSV | RYKSIVKYKTAFYSFYLPIAAAMYMAGIDGEK |
| | TGKVGTDIQDNKCSWLVVQCLQRATPEQYQIL | EHANALKILMEMGEFFQVQDDYLDLFGDP |
| | KENYGQKEAEKVARVKALYEELDLQAVFLQYE | SVTGKVGTDIQDNKCSWLVVQCLLRASPQQ |
| | EDSYSHIMALIEQYAAPLPPAIFLGLARKIYKR | RQILEENYGQKDPEKVARVKALYEALDLQSA |
| | (SEQ ID NO: 26) | FFKYEEDSYNRLKSLIEQCSAPLPPSIFMELA |
| | | NKIYKR (SEQ ID NO: 27) |
| Subunit 4 | YETLMVTSEDGITKIMLNRPTKKNALNVEM | ILVTSEDGITKITFNRPTKKNAISFQMYRDI |
| | YHEIMHALKAASKDDSAITVLTGNGDYYSS | ILALKNASTDNTVMAVFTGTGDYYCSGN |
| | GNDLTNFTDIPPGGVEEKAKNGAILLREFVG | DLTNFTSATGGIEEAASNGAVLLRDFVNS |
| | CFIDFPKPLIAVVNGPAVGISVTLLGLEDAVY | FIDFPKPLVAVVNGPAVGISVTLLGLEDAV |
| | ASDRATFHTPFSHLGQSPEGCSSYTFPKIMSP | FASDRATFHTPFSQLGQSPEACSSYTFPKM |
| | AKATEMLVFGKKLTAGEACAQGLVTEVFPD | MGSAKAAEMLLFGKKLTAREAWAQGLV |
| | STFQKEVWTRLKAFAKLPPNAMRISKEIIRNR | TEVFPESTFETEVWTRLKTYAKLPPNAMR |
| | EKEKLHAVNAEECSVLQGRWLSDECMNAV | ISKELIRKNEKEKLYAVNAEECTTLQARW |
| | VNFLEQELSRRLQRLYPAVNQQETPLPRSWSPK | LSEECMNAIMSFVEQELSRRLQRLYPAVNQ |
| | DKYNYIGLSQGNLRVHYKGHGKNHKDAASVRA | HETPLPRSWSPKDKYNYIGLSQGNLRVHYK |
| | THPIPAACGIYYFEVKIVSKGRDGYMGIGLSAQ | GHGKNHKDAASVRATHPIPAACGIYYFEVKI |
| | GVNMNRLPGWDKHSYGYHGDDGHSFCSSGTG | VSKGRDGYMGIGLSAQGVNMNRLPGWDKH |
| | QPYGPTFTTGDVIGCCVNLINGTCFYTKNGHSL | SYGYHGDDGHSFCSSGTGQPYGPTFTTGDV |
| | GIAFTDLPANLYPTVGLQTPGEIVDANFGQQPF | IGCCVNLINGTCFYTKNGHSLGIAFTDLPAN |
| | LFDIEDYMREWRAKVQGTVHCFPISARLGEWQ | LYPTVGLQTPGEIVDANFGQQPFLFDIEDY |
| | AVLQNMVSSYLVHHGYCATATAFARMTETPIQE | MREWRAKVQGTVHGFPISARLGEWQAVLQ |
| | EQASIKNRQKIQKLVLEGRVGEAIETTQRFYPGL | NMVSSYLVHHGYCSTATAFARMTETPIQEEQ |
| | LEHNPNLLFMLKCRQFVEMVNGTDSEVRSLSS | ASIKNRQKIQKLVLEGRVGEAIETTQRFYPGL |
| | RSPKSQDSYPGSPSLSPRHGPSSSHMHNTGADS | LEHNPNLLFMLKCRQFVEMVNGTDSEVRSL |
| | PSCSNGVVSTKSKQNHSKYPAPSSSSSSSSSSSSSS | SSRSPKSQDSYPGSPSLSPRHGPSSSHIHNTG |
| | PSSINYSESNSTDSTKSQPHSSTSNQETSDSEME | ADSPSCSNGVASTKNKQNHSKYPAPSSSSSSS |
| | MEAEHYPNGVLGSMSTRIVNGAYKHEDLQTDE | SSSSSSSPSSVNYSESNSTDSTKSQPHSSTSNQ |
| | SSMDDGHPRRQLCGGNQAATERIILFGRELQA | ETSDSEMEMEAEHYPNGVLESVSTRIVNGAY |
| | LSEQLGREYGNNLAHTEMLQDAFSLLAYSDPW | KHDDLQTDESSMDDGHPRRQLCGGNQAAT |
| | SCPVGQQLDPIQREPVCAALNSAILESQNLPKQ | ERIILFGRELQALSEQLGREYGKNLAHTEML |
| | PPLMLALGQASECLRLMARAGLGSCSFA (SEQ | QDAFSLLAYSDPWSCPVGHQLDPIQREPVC |
| | ID NO: 28) | AALNSAILESQNLPKQPPLMLALGQASECLR |
| | | LMARAGLGSCSFA (SEQ ID NO: 29) |
| Subunit 5 | KYIPRRAVLYVPGNDEKKIKKIPSLNVDCAV | KYVPRRAVLYVPGNDEKKIRKIPSLKVDC |
| | LDCEDGVAANKKNEARLRIVKTLEDFDLGPT | AVLDCEDGVAENKKNEARLRIAKTLEDF |
| | EKCVRVNSVSSGLAEEDLETLLQSRVLPSSL | DLGTTEKCVRINSVSSGLAEVDLETFLQA |
| | MLPKVESPEEIQWFADKFSFHLKGRKLEQPM | RVLPSSLMLPKVEGPEEIRWFSDKFSLHLK |
| | NLIPFVETAMGLLNFKAVCEETLKIGPQVGLF | GRKLEQPMNLIPFVETAMGLLNFKAVCEE |
| | LDAVVFGGEDFRASIGATSSKETLDILYARQ | TLKTGPQVGLCLDAVVFGGEDFRASIGAT |
| | KIVVIAKAFGLQAIDLVYIDFRDGAGLLRQSR | SNKDTQDILYARQKVVVTAKAFGLQAID |
| | EGAAMGFTGKQVIHPNQIAVVQEQFSPSPEKI | LVYIDFRDEDGLLRQSREAAAMGFTGKQ |
| | KWAEELIAAFKEHQQLGKGAFTFQGSMIDM | VIHPNQIAVVQEQFTPTPEKIQWAEELIAA |
| | PLLKQAQNIVTLATSIXXXXXKTYVDQACRAA | FKEHQQLGKGAFTFRGSMIDMPLLKQAQ |
| | EEFVNVYYTTMDKRRRLLSRLYMGTATLVWNG | NIVTLATSIXXXXXKTYVDQACRAAEEFVN |
| | NAVSGQESLSEFFEMLPSSEFQISVVDCQPVHD | VYYTTMDKRRRLLSRLYMGTATLVWNGNAV |
| | EATPSQTTVLVVICGSVKFEGNKQRDFNQNFIL | SGQESLSEFFEMLPSSEFQISVVDCQPVHDD |
| | TAQASPSNTVWKIASDCFRFQ (SEQ ID NO: 30) | ATPSQTTVLVVICGTVKFEGNKQRDFNQNFI |
| | | LTAQASPSNTVWKIASDCFRFQ (SEQ ID |
| | | NO: 31) |
| Subunit 6 | RFGSQRVLVEPDAGAGVAVMKLKNPPVNTL | RFANKRVLVETEGPAGVAVMKLRNPPVN |
| | SLEFLTELVICLEKLENDKSFRGVVLTSDCPG | SLSLECLTEFTISLEKLENDKSIRGVILTSE |
| | VFSAGLDLTEMCGKSPAHYAEYWKAVQEL | CPGIFSAGLDLLEMYGRNPAHYAEYWKN |
| | WLQFYQSNLVLVSAINGACPAGGCLMTLTC | VQELWLRLYTSNMILVSAINGASPAGGCL |
| | DYRVLADNPRYCIGLNETQLGIVAPFWFKDT | LALCCDYRVMADNPKYTIGLNESLLGIVA |
| | LENVIGHRAAERALQLGLLFPPAEALQVGIV | PFWFKDMYVNTIGHREAERALQLGTLFSP |
| | DQVVPEEQVQSTALSAIAQWMTIPDHARQLT | AEALKVGVVDEVVPEDQVHSKARSVMT |
| | KAMMRKATASRLITQRDADVQNFVSFISKDS | KWLAIPDHSRQLTKNMMRKATADNLIKQ |
| | IQKSLQLYLERLXEAETEDQQRFSYQQRLKAA | READIQNFTSFISKDSIQKSLHMYLEKLXE |
| | VHYTVGCLCEEVALDKEMQFSKQTIAAISELTF | VEAEEPQEFSHRQRLKAAVHYTVGCLCQEV |
| | RQCENFAKDLEMFARHAKRTTINTEDVKLLAR | TLNKQVNFSKQTIAAISEVTFRQCENFAKDL |
| | RSNSLLKYITDKSEEIAQINLERKAQKKKK (SEQ | EMFARHAKRSTVTTEDVKLLARRNNSLLKYI |
| | ID NO: 32) | TEKNEEIAQLNLKGKAKKKRKPEDESRSSRE |
| | | SMAEELDGAEELQSES (SEQ ID NO: 33) |
| Subunit 7 | YETLMVTSEDGITKIMLNRPTKKNALNVEM | ILVTSEDGITKITFNRPTKKNAISFQMYRDI |
| | YHEIMHALKAASKDDSAITVLTGNGDYYSS | ILALKNASTDNTVMAVFTGTGDYYCSGN |
| | GNDLTNFTDIPPGGVEEKAKNGAILLREFVG | DLTNFTSATGGIEEAASNGAVLLRDFVNS |
| | CFIDFPKPLIAVVNGPAVGISVTLLGLEDAVY | FIDFPKPLVAVVNGPAVGISVTLLGLFDAV |
| | ASDRATFHTPFSHLGQSPEGCSSYTFPKIMSP | FASDRATFHTPFSQLGQSPEACSSYTFPKM |
| | AKATEMLVFGKKLTAGEACAQGLVTEVFPD | MGSAKAAEMLLFGKKLTAREAWAQGLV |
| | STFQKEVWTRLKAFAKLPPNAMRISKEIIRNR | TEVFPESTFETEVWTRLKTYAKLPPNAMR |
| | EKEKLHAVNAEECSVLQGRWLSDECMNAV | ISKELIRKNEKEKLYAVNAEECTTLQARW |
| | VNFLXQEQKKELSEIAHSIVANGKGILAADESV | LSEECMNAIMSFVXXEQKKELSEIAQRIVA |
| | GTMGNRLQRIKVENTEENRRQFREILFSVDSSIN | NGKGILAADESVGTMGNRLQRIKVENTEEN |
| | QSIGGVILFHETLYQKDSQGKLFRNILKEKGIVV | RRQFRELLFSVDNSISQSIGGVILFHETLYQK |
| | GIKLDQGGAPLAGTNKETTIQGLDGLSERCAQY | DSQGNLFRNVLKEKGIVVGIKLDQGGAPLA |
| | KKDGVDFGKWRAVLRIADQCPSSLAIQENANA | GTNKETTIQGLDGLSERCAQYKKDGVDFGK |
| | LARYASICQQNGLVPIVEPEVIPDGDHDLEHCQ | WRAVLRIADQCPSSLAIQENANALARYASICQ |
| | YVTEKVLAAVYKALNDHHVYLEGTLLKPNMVTA | QNGLVPIVEPEVLPDGDHDLEHCQYVSEKV |
| | GHACTKKYTPEQVAMATVTALHRTVPAAVPGIC | LAAVYKALNDHHVYLEGTLLKPNMVTAGHA |
| | FLSGGMSEEDATLNLNAINLCPLPKPWKLSFSY | CTKKYTPEQVAMATVTALHRTVPAAVPGICF |
| | GRALQASALAAWGGKAANKEATQEAFMKRAVA | LSGGMSEEDATLNLNAINRCPLPRPWKLSFS |
| | NCQAAKGQYIHTGSSGAASTQSLFTACYTY | YGRALQASALAAWGGKAANKKATQEAFMK |
| | (SEQ ID NO: 34) | RAMANCQAAQGQYVHTGSSGAAATQSLFTA |
| | | SYTY (SEQ ID NO: 35) |
| Subunit 8 | RFGSQRVLVEPDAGAGVAVMKLKNPPVNTL | RFANKRVLVETEGPAGVAVMKLRNPPVN |
| | SLEFLTELVICLEKLENDKSFRGVVLTSDCPG | SLSLECLTEFTISLEKLENDKSIRGVILTSE |
| | VFSAGLDLTEMCGKSPAHYAEYWKAVQEL | CPGIFSAGLDLLEMYGRNPAHYAEYWKN |
| | WLQFYQSNLVLVSAINGACPAGGCLMTLTC | VQELWLRLYTSNMILVSAINGASPAGGCL |
| | DYRVLADNPRYCIGLNETQLGIVAPFWFKDT | LALCCDYRVMADNPKYTIGLNESLLGIVA |
| | LENVIGHRAAERALQLGLLFPPAEALQVGIV | PFWFKDMYVNTIGHREAERALQLGTLFSP |
| | DQVVPEEQVQSTALSAIAQWMTIPDHARQLT | AEALKVGVVDEVVPEDQVHSKARSVMT |
| | KAMMRKATASRLITQRDADVQNFVSFISKDS | KWLAIPDHSRQLTKNMMRKATADNLIKQ |
| | IQKSLQLYLERLXXXXXXXRLRAFLSEPIGEK | READIQNFTSFISKDSIQKSLHMYLEKLXX |
| | DVCWVDGISHELAINLVTKGINKAYILLGQFLL | XXXXXRLRAFLSEPIGEKDVAWVDGISREL |
| | MHKNEAEFQRWLICCCGATECEAQQSSNCLKE | AINLVTKGFNKAYVLLGQFLLMHKNEAEFQ |
| | WCACFL (SEQ ID NO: 36) | RWIICCCGATECEARQSSTCLKEWCSCFL |
| | | (SEQ ID NO: 37) |
| Subunit 9 | YETLMVTSEDGITKIMLNRPTKKNALNVEM | ILVTSEDGITKITFNRPTKKNAISFQMYRDI |
| | YHEIMHALKAASKDDSAITVLTGNGDYYSS | ILALKNASTDNTVMAVFTGTGDYYCSGN |
| | GNDLTNFTDIPPGGVEEKAKNGAILLREFVG | DLTNFTSATGGIEEAASNGAVLLRDFVNS |
| | CFIDFPKPLIAVVNGPAVGISVTLLGLEDAVY | FIDFPKPLVAVVNGPAVGISVTLLGLFDAV |
| | ASDRATFHTPFSHLGQSPEGCSSYTFPKIMSP | FASDRATFHTPFSQLGQSPEACSSYTFPKM |
| | AKATEMLVFGKKLTAGEACAQGLVTEVFPD | MGSAKAAEMLLFGKKLTAREAWAQGLV |
| | STFQKEVWTRLKAFAKLPPNAMRISKEIIRNR | TEVFPESTFETEVWTRLKTYAKLPPNAMR |
| | EKEKLHAVNAEECSVLQGRWLSDECMNAV | ISKELIRKNEKEKLYAVNAEECTTLQARW |
| | VNFLEAETEDQQRFSYQQRLKAAVHYTVGCLC | LSEECMNAIMSFVEVEAEEPQEFSHRQRLK |
| | EEVALDKEMQFSKQTIAAISELTFRQCENFAKD | AAVHYTVGCLCQEVTLNKQVNFSKQTIAAIS |
| | LEMFARHAKRTTINTEDVKLLARRSNSLLKYITD | EVTFRQCENFAKDLEMFARHAKRSTVTTED |
| | KSEEIAQINLERKAQKKKK (SEQ ID NO: 38) | VKLLARRNNSLLKYITEKNEEIAQLNLKGKA |
| | | KKKRKPEDESRSSRESMAEELDGAEELQSES |
| | | (SEQ ID NO: 39) |
| Subunit 10 | RFGSQRVLVEPDAGAGVAVMKLKNPPVNTL | RFANKRVLVETEGPAGVAVMKLRNPPVN |
| | SLEFLTELVICLEKLENDKSFRGVVLTSDCPG | SLSLECLTEFTISLEKLENDKSIRGVILTSE |
| | VFSAGLDLTEMCGKSPAHYAEYWKAVQEL | CPGIFSAGLDLLEMYGRNPAHYAEYWKN |
| | WLQFYQSNLVLVSAINGACPAGGCLMTLTC | VQELWLRLYTSNMILVSAINGASPAGGCL |
| | DYRVLADNPRYCIGLNETQLGIVAPFWFKDT | LALCCDYRVMADNPKYTIGLNESLLGIVA |
| | LENVIGHRAAERALQLGLLFPPAEALQVGIV | PFWFKDMYVNTIGHREAERALQLGTLFSP |
| | DQVVPEEQVQSTALSAIAQWMTIPDHARQLT | AEALKVGVVDEVVPEDQVHSKARSVMT |
| | KAMMRKATASRLITQRDADVQNFVSFISKDS | KWLAIPDHSRQLTKNMMRKATADNLIKQ |
| | IQKSLQLYLERLXXVVEFFVVTFKVLWAFVLA | READIQNFTSFISKDSIQKSLHMYLEKLXX |
| | AARWLVRPKEKSVAGQVCLITGAGSGLGRLFAL | VVEFFVVTFKVLWAFVLAAARWLVRPKEKS |
| | EFARRRALLVLWDINTQSNEETAGMVRHIYRDL | VAGQVCLITGAGSGLGRLFALEFARRRALLV |
| | EAADAAALQAGNGEEEILPHCNLQVFTYTCDV | LWDINTQSNEETAGMVRHIYRDLEAADAAAL |
| | GKRENVYLTAERVRKEVGEVSVLVNNAGVVSG | QAGKGEEEILPPCNLQVFTYTCDVGKRENV |
| | HHLLECPDELIERTMMVNCHAHFWTTKAFLPT | YLTAERVRKEVGEVSVLVNNAGVVSGHHLLE |
| | MLEINHGHIVTVASSLGLFSTAGVEDYCASKFG | CPDELIERTMMVNCHAHFWTTKAFLPTMLE |
| | VVGFHESLSHELKAAEKDGIKTTLVCPYLVDTG | INHGHIVTVASSLGLFSTAGVEDYCASKFGV |
| | MFRGCRIRKEIEPFLPPLKPDYCVKQAMKAILT | VGFHESLSHELKAAEKDGIKTTLVCPYLVDT |
| | DQPMICTPRLMYIVTFMKSILPFEAVVCMYRFL | GMFRGCRIRKEIEPFLPPLKPDYCVKQAMR |
| | GADKCMYPFIAQRKQAT (SEQ ID NO: 40) | AILTDQPMVCTPRLMYIVTFMKSILPFEAVV |
| | | CMYRFLGADKCMYPFIAQRKQAT (SEQ ID |
| | | NO: 41) |
| Subunit 11 | RFGSQRVLVEPDAGAGVAVMKLKNPPVNTL | RFANKRVLVETEGPAGVAVMKLRNPPVN |
| | SLEFLTELVICLEKLENDKSFRGVVLTSDCPG | SLSLECLTEFTISLEKLENDKSIRGVILTSE |
| | VFSAGLDLTEMCGKSPAHYAEYWKAVQEL | CPGIFSAGLDLLEMYGRNPAHYAEYWKN |
| | WLQFYQSNLVLVSAINGACPAGGCLMTLTC | VQELWLRLYTSNMILVSAINGASPAGGCL |
| | DYRVLADNPRYCIGLNETQLGIVAPFWFKDT | LALCCDYRVMADNPKYTIGLNESLLGIVA |
| | LENVIGHRAAERALQLGLLFPPAEALQVGIV | PFWFKDMYVNTIGHREAERALQLGTLFSP |
| | DQVVPEEQVQSTALSAIAQWMTIPDHARQLT | AEALKVGVVDEVVPEDQVHSKARSVMT |
| | KAMMRKATASRLITQRDADVQNFVSFISKDS | KWLAIPDHSRQLTKNMMRKATADNLIKQ |
| | IQKSLQLYLERLXXXXXLAAQKARGLFKDFF | READIQNFTSFISKDSIQKSLHMYLEKLXX |
| | PETGTKIELPELFDRGTASFPQTIYCGFDPTADS | XXXLVAPRARGLFKEFFPESGTKTELPELF |
| | LHVGHLLALLGLFHLQRAGHNVIALVGGATARL | DRRRAGSSPQTVYCGFDPTGDSLHVGHLLT |
| | GDPSGRTKEREALETERVRANARALRLGLEALA | LLGLFHFQRAGHNVIALVGGSTALLGDPSG |
| | ANHQQLFTDGRSWGSFTVLDNSAWYQKQHLV | RTKGREALSAECVRANAHALRRGLEALAAN |
| | DFLAAVGGHFRMGTLLSRQSVQLRLKSAEGMS | HARLFADGRPWGSFTVLDNAAWFQEQHLV |
| | LAEFFYQVLQAYDFYYLFQRYGCRVQLGGSDQ | DFLATVGGHFRMGTLLSRLSVQSRLKSPEG |
| | LGNIMSGYEFINKLTGEDVFGITVPLITSTTGAK | MSLAEFFYQVLQAYDFYYLFQHYGCRVQLG |
| | LGKSAGNAVWLNRDKTSPFELYQFFVRQPDDS | GSDQLGNIMSGYEFIHKLTGEDVFGITVPLIT |
| | VERYLKLFTFLPLPEIDHIMQLHVKEPERRGAQ | STTGAKLGKSAGNAVWLNREKTSPFELYQFF |
| | KRLAAEVTKLVHGREGLDSAKRCTQALYHSSID | VRQQDDSVERYLKLFTFLPLPEIDHIMQLHV |
| | ALEVMSDQELKELFKEAPFSELFLDPGTSVLDT | KEPEKRVAQKRLAAEVTKLVHGQEGLDSAK |
| | CRKANAIPDGPRGYRMITEGGVSINHRQVTNPE | RCTQALYHSSIEALEVMSDQELKELFKEASF |
| | SVLVVGQHILKNGLSLLKIGKRNFYIIKWLQ | SELVLDPGTSVIDTCRKANAIPDGPRGYRMI |
| | (SEQ ID NO: 42) | TEGGVSINHRQVTNPESVLVIGQHILKNGLSL |
| | | LKIGKRNFYIIKWLQ (SEQ ID NO: 43) |
| Subunit 12 | RFGSQRVLVEPDAGAGVAVMKLKNPPVNTL | RFANKRVLVETEGPAGVAVMKLRNPPVN |
| | SLEFLTELVICLEKLENDKSFRGVVLTSDCPG | SLSLECLTEFTISLEKLENDKSIRGVILTSE |
| | VFSAGLDLTEMCGKSPAHYAEYWKAVQEL | CPGIFSAGLDLLEMYGRNPAHYAEYWKN |
| | WLQFYQSNLVLVSAINGACPAGGCLMTLTC | VQELWLRLYTSNMILVSAINGASPAGGCL |
| | DYRVLADNPRYCIGLNETQLGIVAPFWFKDT | LALCCDYRVMADNPKYTIGLNESLLGIVA |
| | LENVIGHRAAERALQLGLLFPPAEALQVGIV | PFWFKDMYVNTIGHREAERALQLGTLFSP |
| | DQVVPEEQVQSTALSAIAQWMTIPDHARQLT | AEALKVGVVDEVVPEDQVHSKARSVMT |
| | KAMMRKATASRLITQRDADVQNFVSFISKDS | KWLAIPDHSRQLTKNMMRKATADNLIKQ |
| | IQKSLQLYLERLQKKELSEIAHSIVANGKGILAA | READIQNFTSFISKDSIQKSLHMYLEKLQK |
| | DESVGTMGNRLQRIKVENTEENRRQFREILFSV | KELSEIAQRIVANGKGILAADESVGTMGNRL |
| | DSSINQSIGGVILFHETLYQKDSQGKLFRNILKE | QRIKVENTEENRRQFRELLFSVDNSISQSIGG |
| | KGIVVGIKLDQGGAPLAGTNKETTIQGLDGLSE | VILFHETLYQKDSQGNLFRNVLKEKGIVVGI |
| | RCAQYKKDGVDFGKWRAVLRIADQCPSSLAIQ | KLDQGGAPLAGTNKETTIQGLDGLSERCAQ |
| | ENANALARYASICQQNGLVPIVEPEVIPDGDHD | YKKDGVDFGKWRAVLRIADQCPSSLAIQEN |
| | LEHCQYVTEKVLAAVYKALNDHHVYLEGTLLKP | ANALARYASICQQNGLVPIVEPEVLPDGDHD |
| | NMVTAGHACTKKYTPEQVAMATVTALHRTVPA | LEHCQYVSEKVLAAVYKALNDHHVYLEGTLL |
| | AVPGICFLSGGMSEEDATLNLNAINLCPLPKPW | KPNMVTAGHACTKKYTPEQVAMATVTALHR |
| | KLSFSYGRALQASALAAWGGKAANKEATQEAF | TVPAAVPGICFLSGGMSEEDATLNLNAINRC |
| | MKRAVANCQAAKGQYIHTGSSGAASTQSLFTAC | PLPRPWKLSFSYGRALQASALAAWGGKAAN |
| | YTY (SEQ ID NO: 44) | KKATQEAFMKRAMANCQAAQGQYVHTGSS |
| | | GAAATQSLFTASYTY (SEQ ID NO: 45) |
| Subunit 13 | KYIPRRAVLYVPGNDEKKIKKIPSLNVDCAV | KYVPRRAVLYVPGNDEKKIRKIPSLKVDC |
| | LDCEDGVAANKKNEARLRIVKTLEDFDLGPT | AVLDCEDGVAENKKNEARLRIAKTLEDF |
| | EKCVRVNSVSSGLAEEDLETLLQSRVLPSSL | DLGTTEKCVRINSVSSGLAEVDLETFLQA |
| | MLPKVESPEEIQWFADKFSFHLKGRKLEQPM | RVLPSSLMLPKVEGPEEIRWFSDKFSLHLK |
| | NLIPFVETAMGLLNFKAVCEETLKIGPQVGLF | GRKLEQPMNLIPFVETAMGLLNFKAVCEE |
| | LDAVVFGGEDFRASIGATSSKETLDILYARQ | TLKTGPQVGLCLDAVVFGGEDFRASIGAT |
| | KIVVIAKAFGLQAIDLVYIDFRDGAGLLRQSR | SNKDTQDILYARQKVVVTAKAFGLQAID |
| | EGAAMGFTGKQVIHPNQIAVVQEQFSPSPEKI | LVYIDFRDEDGLLRQSREAAAMGFTGKQ |
| | KWAEELIAAFKEHQQLGKGAFTFQGSMIDM | VIHPNQIAVVQEQFTPTPEKIQWAEELIAA |
| | PLLKQAQNIVTLATSIXXXRAYQWCREYLGGA | FKEHQQLGKGAFTFRGSMIDMPLLKQAQ |
| | WRRVQPEELRVYPVSGGLSNLLFRCSLPDHLPS | NIVTLATSIXXXRAYQWCREYLGGAWRRAR |
| | VGEEPREVLLRLYGAILQGVDSLVLESVMFAILA | PEELSVCPVSGGLSNLLFRCSLPNHVPSVGG |
| | ERSLGPQLYGVFPEGRLEQYIPSRPLKTQELRE | EPREVLLRLYGAILQGVDSLVLESVMFAILAE |
| | PVLSAAIATKMAQFHGMEMPFTKEPHWLFGT | RSLGPQLYGVFPEGRLEQYLPSRPLKTQELR |
| | MERYLKQIQDLPPTGLPEMNLLEMYSLKDEMG | DPVLSGAIATRMARFHGMEMPFTKEPRWLF |
| | NLRKLLESTPSPVVFCHNDIQEGNILLLSEPENA | GTMERYLKQIQDLPSTSLPQMNLVEMYSLK |
| | DSLMLVDFEYSSYNYRGFDIGNHFCEWVYDYT | DEMNSLRKLLDDTPSPVVFCHNDIQEGNILL |
| | HEEWPFYKARPTDYPTQGQQLHFIRHYLAEAK | LSEPDSDDNLMLVDFEYSSYNYRGFDIGNHF |
| | KGETLSQEEQRKLEEDLLVEVSRYALASHFFWG | CEWVYDYTYEEWPFYKARPTDYPTREQQLH |
| | LWSILQASMSTIEFGYLDYAQSRFQFYFQQKGQ | FIRHYLAEVQKGEILSEEEQKKREEELLLEIS |
| | LT (SEQ ID NO: 46) | RYSLASHFFWGLWSTLQASMSTIEFGYLEYA |
| | | QSRFQFYFQQKGQLT (SEQ ID NO: 47) |
| Subunit 14 | YTYEDYKNTAEWLLSHTKHRPQVAIICGSGL | FTYEDYETTAKWLLQHTEYRPQVAVICGS |
| | GGLTDKLTQAQIFDYSEIPNFPQSTVPGHVGR | GLGGLTAHLKEAQIFDYNEIPNFPQSTVQ |
| | LVFGFLNGRACVIMQGRFHMYEGYSLWKVT | GHAGRLVFGLLNGRCCVMMQGRFHMYE |
| | FPVRVFHLLGVDTLVVTNAAGGLNPEFEVG | GYSLSKVTFPVRVFHLLGVETLVVTNAAG |
| | DIMLIRDHINLPGFSGQNPLRGPNDERFGVRF | GLNPNFEVGDIMLIRDHINLPGFCGQNPLR |
| | PAMSDAYDRTMRQRALSMWKQMGEQREL | GPNDERFGVRFPAMSDAYDRDMRQKAFS |
| | QEGTYVMLAGPSFETVAESRLLQKLGADAV | AWKQMGEQRKLQEGTYVMLAGPNFETV |
| | GMSTVPEVIVARHCGLRVFGFSLITNK | AESRLLKMLGADAVGMSTVPEVIVARHC |
| | VIMDYESLEKANHEEVLAAGKQAAQKLEQL | GLRVFGFSLITNKVVMDYENLEKANHME |
| | VSILMAXXXXXXTLEQHLEDTMKNPSIVGVL | VLDAGKAAAQTLERFVSILMEXXXXXXT |
| | CTDSQGLNLGCRGTLSDEHAGVISVLAQQAAKL | LEQHLEDTMKNPSIVGVLCTDSQGLNLGCR |
| | TSDPTDIPVVCLESDNGNIMIQKHDGITVAVHK | GTLSDEHAGVISVLAQQAARLTSDPTDIPVV |
| | MA (SEQ ID NO: 48) | CLESDNGNIMIQKHDGITVAVHKMA (SEQ |
| | | ID NO: 49) |
| Subunit 15 | YETLMVTSEDGITKIMLNRPTKKNALNVEM | ILVTSEDGITKITFNRPTKKNAISFQMYRDI |
| | YHEIMHALKAASKDDSAITVLTGNGDYYSS | ILALKNASTDNTVMAVFTGTGDYYCSGN |
| | GNDLTNFTDIPPGGVEEKAKNGAILLREFVG | DLTNFTSATGGIEEAASNGAVLLRDFVNS |
| | CFIDFPKPLIAVVNGPAVGISVTLLGLEDAVY | FIDFPKPLVAVVNGPAVGISVTLLGLFDAV |
| | ASDRATFHTPFSHLGQSPEGCSSYTFPKIMSP | FASDRATFHTPFSQLGQSPEACSSYTFPKM |
| | AKATEMLVFGKKLTAGEACAQGLVTEVFPD | MGSAKAAEMLLFGKKLTAREAWAQGLV |
| | STFQKEVWTRLKAFAKLPPNAMRISKEIIRNR | TEVFPESTFETEVWTRLKTYAKLPPNAMR |
| | EKEKLHAVNAEECSVLQGRWLSDECMNAV | ISKELIRKNEKEKLYAVNAEECTTLQARW |
| | VNFLXXXXEAETEDQQRFSYQQRLKAAVHYTV | LSEECMNAIMSFVXXXXEVEAEEPQEFSH |
| | GCLCEEVALDKEMQFSKQTIAAISELTFRQCEN | RQRLKAAVHYTVGCLCQEVTLNKQVNFSKQ |
| | FAKDLEMFARHAKRTTINTEDVKLLARRSNSLL | TIAAISEVTFRQCENFAKDLEMFARHAKRST |
| | KYITDKSEEIAQINLERKAQKKKK (SEQ ID | VTTEDVKLLARRNNSLLKYITEKNEEIAQLNL |
| | NO: 50) | KGKAKKKRKPEDESRSSRESMAEELDGAEE |
| | | LQSES (SEQ ID NO: 51) |
| Subunit 16 | YETLMVTSEDGITKIMLNRPTKKNALNVEM | ILVTSEDGITKITFNRPTKKNAISFQMYRDI |
| | YHEIMHALKAASKDDSAITVLTGNGDYYSS | ILALKNASTDNTVMAVFTGTGDYYCSGN |
| | GNDLTNFTDIPPGGVEEKAKNGAILLREFVG | DLTNFTSATGGIEEAASNGAVLLRDFVNS |
| | CFIDFPKPLIAVVNGPAVGISVTLLGLFDAVY | FIDFPKPLVAVVNGPAVGISVTLLGLEDAV |
| | ASDRATFHTPFSHLGQSPEGCSSYTFPKIMSP | FASDRATFHTPFSQLGQSPEACSSYTFPKM |
| | AKATEMLVFGKKLTAGEACAQGLVTEVFPD | MGSAKAAEMLLFGKKLTAREAWAQGLV |
| | STFQKEVWTRLKAFAKLPPNAMRISKEIIRNR | TEVFPESTFETEVWTRLKTYAKLPPNAMR |
| | EKEKLHAVNAEECSVLQGRWLSDECMNAV | ISKELIRKNEKEKLYAVNAEECTTLQARW |
| | VNFLWSSHDKMLAQPLKDSDVEVYNIIKKESNR | LSEECMNAIMSFVWASHEKMLSQPLKDSD |
| | QRVGLELIASENFASRAVLEALGSCLNNKYSEGY | AEVYSIIKKESNRQRVGLELIASENFASRAVLE |
| | PGQRYYGGTEFIDELETLCQKRALQAYKLDPQ | ALGSCLNNKYSEGYPGQRYYGGTEFIDELE |
| | CWGVNVQPYSGSPANFAVYTALVEPHGRIMGL | MLCQKRALQAYHLDPQCWGVNVQPYSGSP |
| | DLPDGGHLTHGFMTDKKKISATSIFFESMPYKV | ANFAVYTALVEPHGRIMGLDLPDGGHLTHG |
| | NPDTGYINYDQLEENARLFHPKLIIAGTSCYSRN | FMTDKKKISATSIFFESMPYKVYPETGYINYD |
| | LDYARLRKIADENGAYLMADMAHISGLVVAGVV | QLEENASLFHPKLIIAGTSCYSRNLDYARLRK |
| | PSPFEHCHVVTTTTHKTLRGCRAGMIFYRKGV | IADDNGAYLMADMAHISGLVAAGVVPSPFE |
| | KNVDPKTGKEILYNLESLINSAVFPGLQGGPHN | HCHVVTTTTHKTLRGCRAGMIFYRKGVRSV |
| | HAIAGVAVALKQAMTLEFKVYQHQVVANCRAL | DPKTGKETYYELESLINSAVFPGLQGGPHNH |
| | SEALMELGYKIVTGGSDNHLILVDLRSKGTDGG | AIAGVAVALKQAMTTEFKIYQLQVLANCRAL |
| | RAEKVLEACSIACNKNTCPGDRSALRPSGLRLG | SDALTELGYKIVTGGSDNHLILMDLRSKGTD |
| | TPALTSRGLLEKDFQKVAQFIHRGIELTLQIQSD | GGRAEKVLEACSIACNKNTCPGDKSALRPSG |
| | IGVRATLKEFKERLAGDKYQGAVQALREEVESF | LRLGTPALTSRGLLEEDFQKVAHFIHRGIEL |
| | ASLFPLPGLP (SEQ ID NO: 52) | TLQIQSHMATKATLKEFKEKLAGDEKIQSAV |
| | | ATLREEVENFASNFSLPGLP (SEQ ID NO: 53) |
| Subunit 17 | YETLMVTSEDGITKIMLNRPTKKNALNVEM | ILVTSEDGITKITFNRPTKKNAISFQMYRDI |
| | YHEIMHALKAASKDDSAITVLTGNGDYYSS | ILALKNASTDNTVMAVFTGTGDYYCSGN |
| | GNDLTNFTDIPPGGVEEKAKNGAILLREFVG | DLTNFTSATGGIEEAASNGAVLLRDFVNS |
| | CFIDFPKPLIAVVNGPAVGISVTLLGLFDAVY | FIDFPKPLVAVVNGPAVGISVTLLGLFDAV |
| | ASDRATFHTPFSHLGQSPEGCSSYTFPKIMSP | FASDRATFHTPFSQLGQSPEACSSYTFPKM |
| | AKATEMLVFGKKLTAGEACAQGLVTEVFPD | MGSAKAAEMLLFGKKLTAREAWAQGLV |
| | STFQKEVWTRLKAFAKLPPNAMRISKEIIRNR | TEVFPESTFETEVWTRLKTYAKLPPNAMR |
| | EKEKLHAVNAEECSVLQGRWLSDECMNAV | ISKELIRKNEKEKLYAVNAEECTTLQARW |
| | VNFLXXEAETEDQQRFSYQQRLKAAVHYTVGC | LSEECMNAIMSFVXXEVEAEEPQEFSHRQ |
| | LCEEVALDKEMQFSKQTIAAISELTFRQCENFA | RLKAAVHYTVGCLCQEVTLNKQVNFSKQTIA |
| | KDLEMFARHAKRTTINTEDVKLLARRSNSLLKYI | AISEVTFRQCENFAKDLEMFARHAKRSTVTT |
| | TDKSEEIAQINLERKAQKKKK (SEQ ID NO: 54) | EDVKLLARRNNSLLKYITEKNEEIAQLNLKG |
| | | KAKKKRKPEDESRSSRESMAEELDGAEELQ |
| | | SES (SEQ ID NO: 55) |
| Subunit 18 | RFGSQRVLVEPDAGAGVAVMKLKNPPVNTL | RFANKRVLVETEGPAGVAVMKLRNPPVN |
| | SLEFLTELVICLEKLENDKSFRGVVLTSDCPG | SLSLECLTEFTISLEKLENDKSIRGVILTSE |
| | VFSAGLDLTEMCGKSPAHYAEYWKAVQEL | CPGIFSAGLDLLEMYGRNPAHYAEYWKN |
| | WLQFYQSNLVLVSAINGACPAGGCLMTLTC | VQELWLRLYTSNMILVSAINGASPAGGCL |
| | DYRVLADNPRYCIGLNETQLGIVAPFWFKDT | LALCCDYRVMADNPKYTIGLNESLLGIVA |
| | LENVIGHRAAERALQLGLLFPPAEALQVGIV | PFWFKDMYVNTIGHREAERALQLGTLFSP |
| | DQVVPEEQVQSTALSAIAQWMTIPDHARQLT | AEALKVGVVDEVVPEDQVHSKARSVMT |
| | KAMMRKATASRLITQRDADVQNFVSFISKDS | KWLAIPDHSRQLTKNMMRKATADNLIKQ |
| | IQKSLQLYLERLAMDELLRRAVPPTPAYELREK | READIQNFTSFISKDSIQKSLHMYLEKLAM |
| | TPAPAEGQCADFVSFYGGLAETAERAELLGRLA | DELLRRAVPPTPAYELREKTPAPAEGQCADF |
| | RGFGVDHGQVAEQSAGVLQLRQQQREAAVLL | VSFYGGLAEASQRAELLGRLAQGFGVDHGQ |
| | QAEDRLRYALVPRYRGLFHHISKLDGGVRFLVQ | VAEQSAGVLQLRQQAREAAVLLQAEDRLRY |
| | LRADLLEAQALKLVEGPDVREMNGVLKGMLSE | ALVPRYRGLFHHISKLDGGVRFLVQLRADLL |
| | WFSSGFLNLERVTWHSPCEVLQKISEAEAVHPV | EAQALKLVEGPHVREMNGVLKSMLSEWFSS |
| | KNWMDMKRRVGPYRRCYFFSHCSTPGEPLVVL | GFLNLERVTWHSPCEVLQKISECEAVHPVK |
| | HVALTGDISSNIQAIVKECPPSETEEKNTIAAAIF | NWMDMKRRVGPYRRCYFFSHCSTPGEPLV |
| | YSISLTQQGLQGVELGTFLIKRVVKELQREFPHL | VLHVALTGDISNNIQGIVKECPPTETEERNRI |
| | GVFSSLSPIPGFTKWLLGLLNSQVKDHGRNELF | AAAIFYSISLTQQGLQGVELGTFLIKRVVKEL |
| | TDSECKEISEITGGPTNETLKLLLSSSEWVQSETL | QKEFPQLGAFSSLSPIPGFTKWLLGLLNVQG |
| | VRALQTPLMRLCAWYLYGEKHRGYALNPVANF | KEHGRNELFTDSECQEISAVTGNPVHESLKG |
| | HLQNGAVLWRINWMADVSLKGITGSCGLMANY | FLSSGEWVKSEKLTQALQGPLMRLCAWYLY |
| | RYFLEETGPNSTSYLSSKNIKASEQVLSLVAQFQ | GEKHRGYALNPVANFHLQNGAVMWRINWM |
| | K (SEQ ID NO: 56) | ADSSLKGLTSSCGLMVNYRYYLEETGPNSISY |
| | | LGSKNIKASEQILSLVAQFQN (SEQ ID |
| | | NO: 57) |
| Subunit 19 | RFGSQRVLVEPDAGAGVAVMKLKNPPVNTL | RFANKRVLVETEGPAGVAVMKLRNPPVN |
| | SLEFLTELVICLEKLENDKSFRGVVLTSDCPG | SLSLECLTEFTISLEKLENDKSIRGVILTSE |
| | VFSAGLDLTEMCGKSPAHYAEYWKAVQEL | CPGIFSAGLDLLEMYGRNPAHYAEYWKN |
| | WLQFYQSNLVLVSAINGACPAGGCLMTLTC | VQELWLRLYTSNMILVSAINGASPAGGCL |
| | DYRVLADNPRYCIGLNETQLGIVAPFWFKDT | LALCCDYRVMADNPKYTIGLNESLLGIVA |
| | LENVIGHRAAERALQLGLLFPPAEALQVGIV | PFWFKDMYVNTIGHREAERALQLGTLFSP |
| | DQVVPEEQVQSTALSAIAQWMTIPDHARQLT | AEALKVGVVDEVVPEDQVHSKARSVMT |
| | KAMMRKATASRLITQRDADVQNFVSFISKDS | KWLAIPDHSRQLTKNMMRKATADNLIKQ |
| | IQKSLQLYLERLXXXXXXQKHRDFVAEPMGE | READIQNFTSFISKDSIQKSLHMYLEKLXX |
| | KPVGSLAGIGEVLGKKLEERGFDKAYVVLGQF | XXXXQKHRDFVAEPMGEKPVGSLAGIGDV |
| | LVLKKDEDLFREWLKDTCGANAKQSRDCFGC | LSKRLEERGFDKAYVVLGQFLVLKKDEDLF |
| | LREWCDAFL (SEQ ID NO: 58) | REWLKDTCGANAKQSRDCFGCLREWCDAF |
| | | L (SEQ ID NO: 59) |
| Subunit 20 | RFGSQRVLVEPDAGAGVAVMKLKNPPVNTL | RFANKRVLVETEGPAGVAVMKLRNPPVN |
| | SLEFLTELVICLEKLENDKSFRGVVLTSDCPG | SLSLECLTEFTISLEKLENDKSIRGVILTSE |
| | VFSAGLDLTEMCGKSPAHYAEYWKAVQEL | CPGIFSAGLDLLEMYGRNPAHYAEYWKN |
| | WLQFYQSNLVLVSAINGACPAGGCLMTLTC | VQELWLRLYTSNMILVSAINGASPAGGCL |
| | DYRVLADNPRYCIGLNETQLGIVAPFWFKDT | LALCCDYRVMADNPKYTIGLNESLLGIVA |
| | LENVIGHRAAERALQLGLLFPPAEALQVGIV | PFWFKDMYVNTIGHREAERALQLGTLFSP |
| | DQVVPEEQVQSTALSAIAQWMTIPDHARQLT | AEALKVGVVDEVVPEDQVHSKARSVMT |
| | KAMMRKATASRLITQRDADVQNFVSFISKDS | KWLAIPDHSRQLTKNMMRKATADNLIKQ |
| | IQKSLQLYLERLXIEEIKEKMKTVKHKILVLSG | READIQNFTSFISKDSIQKSLHMYLEKX |
| | KGGVGKSTFSAHLAHGLAEDENTQIALLDIDIC | VEEIREKMKTVRHKLLVLSGKGGVGKSTFSA |
| | GPSIPKIMGLEGEQVHQSGSGWSPVYVEDNLG | HLAHGLAEDGDTQVALLDIDICGPSIPKIMG |
| | VMSVGFLLSSPDDAVIWRGPKKNGMIKQFLRD | LEGEQVHQSGSGWSPVYVDDNLGVMSVGF |
| | VDWGEVDYLIVDTPPGTSDEHLSVVQYLAAARI | LLSSPDDAVIWRGPKKNGMIKQFLRDVDWG |
| | DGAVIITTPQEVSLQDVRKEINFCRKVKLPIIGV | DVDYLIVDTPPGTSDEHLSVVQYLAAAHIDG |
| | VENMSGFICPKCKKESQIFPPTTGGAELMCQD | AVILTTPQEVALQDVRKEISFCHKVKLPIIGV |
| | LEVPLLGRVPLDPLIGKNCDKGQSFFIDAPDSP | VENMSGFICPKCKKESQIFPPTTGGAEAMC |
| | ATLAYRSIIQRIQEFCDL (SEQ ID NO: 60) | QDLRIPLLGKVPLDPHIGKSCDKGQSFFVEA |
| | | PDSPATAAYRSIIQRIRDFCN (SEQ ID |
| | | NO: 61) |
| Subunit 21 | YETLMVTSEDGITKIMLNRPTKKNALNVEM | ILVTSEDGITKITFNRPTKKNAISFQMYRDI |
| | YHEIMHALKAASKDDSAITVLTGNGDYYSS | ILALKNASTDNTVMAVFTGTGDYYCSGN |
| | GNDLTNFTDIPPGGVEEKAKNGAILLREFVG | DLTNFTSATGGIEEAASNGAVLLRDFVNS |
| | CFIDFPKPLIAVVNGPAVGISVTLLGLEDAVY | FIDFPKPLVAVVNGPAVGISVTLLGLEDAV |
| | ASDRATFHTPFSHLGQSPEGCSSYTFPKIMSP | FASDRATFHTPFSQLGQSPEACSSYTFPKM |
| | AKATEMLVFGKKLTAGEACAQGLVTEVFPD | MGSAKAAEMLLFGKKLTAREAWAQGLV |
| | STFQKEVWTRLKAFAKLPPNAMRISKEIIRNR | TEVFPESTFETEVWTRLKTYAKLPPNAMR |
| | EKEKLHAVNAEECSVLQGRWLSDECMNAV | ISKELIRKNEKEKLYAVNAEECTTLQARW |
| | VNFLXXXXXXQECMDYAVTLARQAGEVVCEA | LSEECMNAIMSFVXXXXXXQECMDYAVIL |
| | LKNEMNVMLKSSPVDLVTATDQKVEKMLISSIK | ARQAGEMIREALKNEMDVMIKSSPADLVTV |
| | EKYPSHSFIGEESVAAGEKSILTNNPTWIIDPIDG | TDQKVEKMLMSSIKEKYPCHSFIGEESVAAG |
| | TTNFVHRFPFVAVSIGFAVNKKIEFGVVYSCVE | EKTVFTEQPTWVIDPIDGTTNFVHRFPFVAV |
| | DKMYTARKGKGAFCNGQKLHVSQQEDITKSLL | SIGFLVNKEMEFGIVYSCVEDKMYTGRKGK |
| | VTELGSSRTPETVRIVLSNMEKLFCIPVHGIRSV | GAFCNGQKLQVSQQEDITKSLLVTELGSSRK |
| | GTAAVNMCLVATGGADAYYEMGIHCWDVAGA | PETLRIVLSNMEKLCSIPIHGIRSVGTAAVNM |
| | GIIVTEAGGVLMDVTGGPFDLMSRRVIAANNRI | CLVATGGADAYYEMGIHCWDMAGAGIIVTE |
| | LAERIAKEIQVIPLQRDDE (SEQ ID NO: 62) | AGGVLMDVTGGPFDLMSRRIIAANSITLAKRI |
| | | AKEIEIIPLQRDDE (SEQ ID NO: 63) |

### Example 3: Human Protein Cages

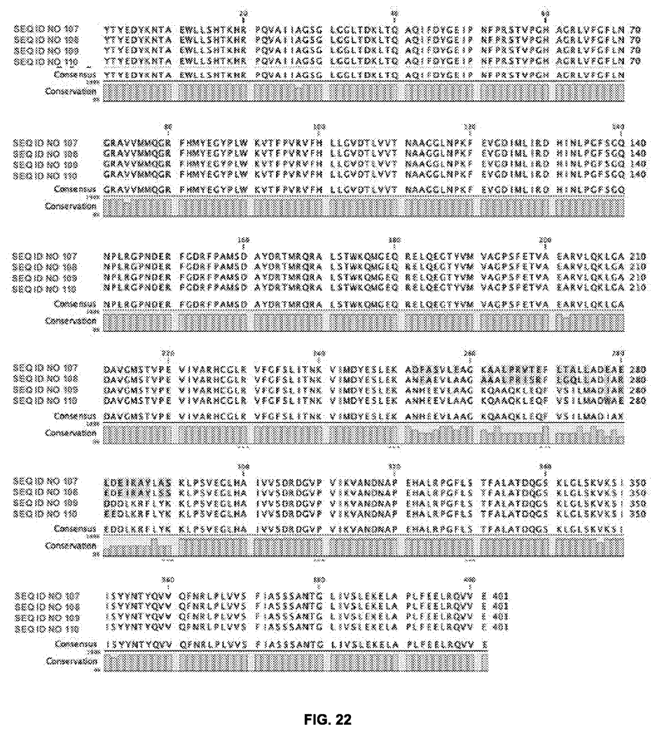

Variations for the twenty-one (21) human protein cages, as discussed in Example 1 above, were designed. 3 to 6 sequence variants of each of the 21 human protein cages were expressed by modifying residues mainly in the helical linker and adjoining parts of the protein structure. The individual variants are shown in detail in Table 12 below, in which the amino acid sequence of each variant is included. FIG. 22 further illustrates a multiple sequence alignment that highlights sequence variations SEQ ID NO 107-110, related to SEQ ID 1.

TABLE 12
Human Fusion Protein Subunit Variants

| | | | Sequence of |
|---|---|---|---|
| SEQ ID NO | Example 1 SEQ ID NO | Design Region | Human Fusion Protein Subunit |
| 64 | 17 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSPEFQAAVAAKLAAL |
| | | | AAKAAEAAAAARQARLAAVREAVTALLEKLAKEKEM |
| | | | QFSKQTIAAISELTFRQAENFAKDLEMFARHAKRTTI |
| | | | NTEDVKLLARRSNSLLKYITDKSEEIAQINLERKAQK |
| | | | KKK |
| 65 | 17 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSQEFKDAVAKVLAKI |
| | | | KAEEEKKKEEERKKKLEEVLKKVEEKLKKLAKEKEM |
| | | | QFSKQTIAAISELTFRQAENFAKDLEMFARHAKRTTI |
| | | | NTEDVKLLARRSNSLLKYITDKSEEIAQINLERKAQK |
| | | | KKK |
| 66 | 17 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVNFLKKQ |
| | | | KETEEQQRFSYQQRLKAAVHYTVGKLMEEVALDKE |
| | | | MQFSKQTIAAISELTFRQAENFAKDLEMFARHAKRT |
| | | | TINTEDVKLLARRSNSLLKYITDKSEEIAQINLERKAQ |
| | | | KKKK |
| 67 | 17 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVNFLKKIK |
| | | | ETEEQQRFSYQQRLKAAVHYTVGKLMEEVALDKEM |
| | | | QFSKQTIAAISELTFRQAENFAKDLEMFARHAKRTTI |
| | | | NTEDVKLLARRSNSLLKYITDKSEEIAQINLERKAQK |
| | | | KKK |
| 68 | 9 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSEEFKNAIAKLFEKIE |
| | | | KKKKEEEELEKRKKKVLEEVTKLLKKLAKEKEMQFS |
| | | | KQTIAAISELTFRQAENFAKDLEMFARHAKRTTINTE |
| | | | DVKLLARRSNSLLKYITDKSEEIAQINLERKAQKKKK |
| 69 | 9 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSKEFKDAVAALFKKIE |
| | | | EEKKKEEERKKKYEEVLEKVTKELEKLAKEKEMQFS |
| | | | KQTIAAISELTFRQAENFAKDLEMFARHAKRTTINTE |
| | | | DVKLLARRSNSLLKYITDKSEEIAQINLERKAQKKKK |
| 70 | 9 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVAKLKKE |
| | | | TEEQQRFSYQQRLKAAVHYTVGKLMEEVALDKEM |
| | | | QFSKQTIAAISELTFRQAENFAKDLEMFARHAKRTTI |
| | | | NTEDVKLLARRSNSLLKYITDKSEEIAQINLERKAQK |
| | | | KKK |
| 71 | 9 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVALKKKE |
| | | | TEEQQRFSYQQRLKAAVHYTVGKLMEEVALDKEM |
| | | | QFSKQTIAAISELTFRQAENFAKDLEMFARHAKRTTI |
| | | | NTEDVKLLARRSNSLLKYITDKSEEIAQINLERKAQK |
| | | | KKK |
| 72 | 6 | Extended | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQARLA |
| | | | AFLAAVAAKAAAAAAAAAARQKELEAVRAKVEEKM |
| | | | KKIAKEKEMQFSKQTIAAISELTFRQAENFAKDLEMF |
| | | | ARHAKRTTINTEDVKLLARRSNSLLKYITDKSEEIAQI |
| | | | NLERKAQKKKK |
| 73 | 6 | Extended | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQAALA |
| | | | AFLAAVAAARAAAAAAAAAREARKEAVLAAVTALLE |
| | | | KLAKEKEMQFSKQTIAAISELTFRQAENFAKDLEMF |
| | | | ARHAKRTTINTEDVKLLARRSNSLLKYITDKSEEIAQI |
| | | | NLERKAQKKKK |
| 74 | 6 | Helix | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISKDSIQKSLQ |
| | | | MYLEELKKKETEEQQRFSYQQRLKAAVHYTVGKLM |
| | | | EEVALDKEMQFSKQTIAAISELTFRQAENFAKDLEM |
| | | | FARHAKRTTINTEDVKLLARRSNSLLKYITDKSEEIA |
| | | | QINLERKAQKKKK |
| 75 | 6 | Helix | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISKDSIQKSLQ |
| | | | MYLEKVKQKETEEQQRFSYQQRLKAAVHYTVGKL |
| | | | MEEVALDKEMQFSKQTIAAISELTFRQAENFAKDLE |
| | | | MFARHAKRTTINTEDVKLLARRSNSLLKYITDKSEEI |
| | | | AQINLERKAQKKKK |
| 76 | 16 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSPEFQAAVAKVLAKL |
| | | | EKLKAEMPLALRDPEAYAALQAARAARRVGLELIAS |
| | | | ENFASRAVLEALGSDLNNKYSEGYPGQRYYGGTEF |
| | | | IDELETLAQKRALQAYKLDPQKWGVNVQPYSGSPA |
| | | | NFAVYTALVEPHGRIMGLDLPDGGHLTHGFMTDKK |
| | | | KISATSIFFESMPYKVNPDTGYINYDQLEENARLFHP |
| | | | KLIIAGTSSYSRNLEYARLRKIADENGAYLMADMAHI |
| | | | SGLVAAGVVPSPFEHAHVVTTTTHKTLRGPRAGMIF |
| | | | YRKGVKSVDPKTGKEILYNLESLINSAVFPGLQGGP |
| | | | HNHAIAGVAVALKQAMTLEFKVYQHQVVANTRALS |
| | | | EALTELGYKIVTGGSDNHLILVDLRSKGTDGGRAEK |
| | | | VLEACSIACNKNTVPGDRSALRPSGLRLGTPALTSR |
| | | | GLLEKDFQKVAHFIHRGIELTLQIQSDTGVRATLKEF |
| | | | KERLAGDKYQAAVQALREEVESFASLFPLPGLP |
| 77 | 16 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFKAAVAKVLAKA |
| | | | AAAAAAAPLAVRDPEAAAALQAARERRRVGLELIAS |
| | | | ENFASRAVLEALGSDLNNKYSEGYPGQRYYGGTEF |
| | | | IDELETLAQKRALQAYKLDPQKWGVNVQPYSGSPA |
| | | | NFAVYTALVEPHGRIMGLDLPDGGHLTHGFMTDKK |
| | | | KISATSIFFESMPYKVNPDTGYINYDQLEENARLFHP |
| | | | KLIIAGTSSYSRNLEYARLRKIADENGAYLMADMAHI |
| | | | SGLVAAGVVPSPFEHAHVVTTTTHKTLRGPRAGMIF |
| | | | YRKGVKSVDPKTGKEILYNLESLINSAVFPGLQGGP |
| | | | HNHAIAGVAVALKQAMTLEFKVYQHQVVANTRALS |
| | | | EALTELGYKIVTGGSDNHLILVDLRSKGTDGGRAEK |
| | | | VLEACSIACNKNTVPGDRSALRPSGLRLGTPALTSR |
| | | | GLLEKDFQKVAHFIHRGIELTLQIQSDTGVRATLKEF |
| | | | KERLAGDKYQAAVQALREEVESFASLFPLPGLP |
| 78 | 16 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVKVLKKS |
| | | | HDKMLAQPLKDSDVEVYNIIKKESNRQRVGLELIAS |
| | | | ENFASRAVLEALGSDLNNKYSEGYPGQRYYGGTEF |
| | | | IDELETLAQKRALQAYKLDPQKWGVNVQPYSGSPA |
| | | | NFAVYTALVEPHGRIMGLDLPDGGHLTHGFMTDKK |
| | | | KISATSIFFESMPYKVNPDTGYINYDQLEENARLFHP |
| | | | KLIIAGTSSYSRNLEYARLRKIADENGAYLMADMAHI |
| | | | SGLVAAGVVPSPFEHAHVVTTTTHKTLRGPRAGMIF |
| | | | YRKGVKSVDPKTGKEILYNLESLINSAVFPGLQGGP |
| | | | HNHAIAGVAVALKQAMTLEFKVYQHQVVANTRALS |
| | | | EALTELGYKIVTGGSDNHLILVDLRSKGTDGGRAEK |
| | | | VLEACSIACNKNTVPGDRSALRPSGLRLGTPALTSR |
| | | | GLLEKDFQKVAHFIHRGIELTLQIQSDTGVRATLKEF |
| | | | KERLAGDKYQAAVQALREEVESFASLFPLPGLP |
| 79 | 16 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVGVLKKS |
| | | | HDKMLAQPLKDSDVEVYNIIKKESNRQRVGLELIAS |
| | | | ENFASRAVLEALGSDLNNKYSEGYPGQRYYGGTEF |
| | | | IDELETLAQKRALQAYKLDPQKWGVNVQPYSGSPA |
| | | | NFAVYTALVEPHGRIMGLDLPDGGHLTHGFMTDKK |
| | | | KISATSIFFESMPYKVNPDTGYINYDQLEENARLFHP |
| | | | KLIIAGTSSYSRNLEYARLRKIADENGAYLMADMAHI |
| | | | SGLVAAGVVPSPFEHAHVVTTTTHKTLRGPRAGMIF |
| | | | YRKGVKSVDPKTGKEILYNLESLINSAVFPGLQGGP |
| | | | HNHAIAGVAVALKQAMTLEFKVYQHQVVANTRALS |
| | | | EALTELGYKIVTGGSDNHLILVDLRSKGTDGGRAEK |
| | | | VLEACSIACNKNTVPGDRSALRPSGLRLGTPALTSR |
| | | | GLLEKDFQKVAHFIHRGIELTLQIQSDTGVRATLKEF |
| | | | KERLAGDKYQAAVQALREEVESFASLFPLPGLP |
| 80 | 7 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSEEFKKAVAAKFEEA |
| | | | KKKELAAIANKIVANGKGILAADESVGTMGNRLQRIK |
| | | | VENTEENRRQFREILFSVDSSISQSIGGVILFHETLY |
| | | | QKDSQGKLFRNILKEKGIVVGIKLDQGGAPLAGTNK |
| | | | ETTIQGLDGLSERCAQYKKDGVDFGKWRAVLRIAD |
| | | | QSPSSLAIQENANALARYASICQQNGLVPIVEPEVIP |
| | | | DGDHDLEHHQYVTEKVLAAVYKALNDHHVYLEGTL |
| | | | LKPNMVTAGHAHTKKYTPEQVAMATVTALHRTVPA |
| | | | AVPGIAFLSGGMSEEDATLNLNAINLNPLPKPWKLS |
| | | | FSYGRALQASALAAWGGKAANKEATQEAFMKRAM |
| | | | ANSQAAKGQYVHTGSSGAASTQSLFTACYTY |
| 81 | 7 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSEEHKKAIAALKEQA |
| | | | KKDELAAIANKIVANGKGILAADESVGTMGNRLQRIK |
| | | | VENTEENRRQFREILFSVDSSISQSIGGVILFHETLY |
| | | | QKDSQGKLFRNILKEKGIVVGIKLDQGGAPLAGTNK |
| | | | ETTIQGLDGLSERCAQYKKDGVDFGKWRAVLRIAD |
| | | | QSPSSLAIQENANALARYASICQQNGLVPIVEPEVIP |
| | | | DGDHDLEHHQYVTEKVLAAVYKALNDHHVYLEGTL |
| | | | LKPNMVTAGHAHTKKYTPEQVAMATVTALHRTVPA |
| | | | AVPGIAFLSGGMSEEDATLNLNAINLNPLPKPWKLS |
| | | | FSYGRALQASALAAWGGKAANKEATQEAFMKRAM |
| | | | ANSQAAKGQYVHTGSSGAASTQSLFTACYTY |
| 82 | 7 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVNLFKEQ |
| | | | QKKELSEIAQSIVANGKGILAADESVGTMGNRLQRIK |
| | | | VENTEENRRQFREILFSVDSSISQSIGGVILFHETLY |
| | | | QKDSQGKLFRNILKEKGIVVGIKLDQGGAPLAGTNK |
| | | | ETTIQGLDGLSERCAQYKKDGVDFGKWRAVLRIAD |
| | | | QSPSSLAIQENANALARYASICQQNGLVPIVEPEVIP |
| | | | DGDHDLEHHQYVTEKVLAAVYKALNDHHVYLEGTL |
| | | | LKPNMVTAGHAHTKKYTPEQVAMATVTALHRTVPA |
| | | | AVPGIAFLSGGMSEEDATLNLNAINLNPLPKPWKLS |
| | | | FSYGRALQASALAAWGGKAANKEATQEAFMKRAM |
| | | | ANSQAAKGQYVHTGSSGAASTQSLFTACYTY |
| 83 | 7 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVNAFKEA |
| | | | QKKELSEIAQSIVANGKGILAADESVGTMGNRLQRIK |
| | | | VENTEENRRQFREILFSVDSSISQSIGGVILFHETLY |
| | | | QKDSQGKLFRNILKEKGIVVGIKLDQGGAPLAGTNK |
| | | | ETTIQGLDGLSERCAQYKKDGVDFGKWRAVLRIAD |
| | | | QSPSSLAIQENANALARYASICQQNGLVPIVEPEVIP |
| | | | DGDHDLEHHQYVTEKVLAAVYKALNDHHVYLEGTL |
| | | | LKPNMVTAGHAHTKKYTPEQVAMATVTALHRTVPA |
| | | | AVPGIAFLSGGMSEEDATLNLNAINLNPLPKPWKLS |
| | | | FSYGRALQASALAAWGGKAANKEATQEAFMKRAM |
| | | | ANSQAAKGQYVHTGSSGAASTQSLFTACYTY |
| 84 | 18 | Extended | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQAALA |
| | | | AYLRGLEIDALLARAVPPTPAYELREKTPAPAEGQA |
| | | | ADFVSFYGGLAETAQRAELLGRLARGFGVDHGQVA |
| | | | EQSAGVLHLRQQQREAAVLLQAEDRLRYALVPRYR |
| | | | GLFHHISKLDGGVRFLVQLRADLLEAQALKLVEGPD |
| | | | VREMNGVLKGMLSEWFSSGFLNLERVTWHSPREV |
| | | | LQKISEAEAVHPVKNWMDMKRRVGPYRRCYFFSH |
| | | | RSTPGEPLVVLHVALTGDISSNIQAIVKEHPPSETEE |
| | | | KNKITAAIFYSISLTQQGLQGVELGTFLIKRVVKELQR |
| | | | EFPHLGVFSSLSPIPGFTKWLLGLLNSQTKEHGRNE |
| | | | LFTDSERKEISEITGGPIRETLKLLLSSSEWVQSEKL |
| | | | VRALQTPLMRLAAWYLYGEKHRGYALNPVANFHLQ |
| | | | NGAVLWRINWMADVSLRGITGSAGLMANYRYFLEE |
| | | | TGPNSTSYLGSKIIKASEQVLSLVAQFQK |
| 85 | 18 | Extended | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQAALA |
| | | | AFLRGLEIDALLAAAVPPTPAYELREKTPAPAEGQA |
| | | | ADFVSFYGGLAETAQRAELLGRLARGFGVDHGQVA |
| | | | EQSAGVLHLRQQQREAAVLLQAEDRLRYALVPRYR |
| | | | GLFHHISKLDGGVRFLVQLRADLLEAQALKLVEGPD |
| | | | VREMNGVLKGMLSEWFSSGFLNLERVTWHSPREV |
| | | | LQKISEAEAVHPVKNWMDMKRRVGPYRRCYFFSH |
| | | | RSTPGEPLVVLHVALTGDISSNIQAIVKEHPPSETEE |
| | | | KNKITAAIFYSISLTQQGLQGVELGTFLIKRVVKELQR |
| | | | EFPHLGVFSSLSPIPGFTKWLLGLLNSQTKEHGRNE |
| | | | LFTDSERKEISEITGGPIRETLKLLLSSSEWVQSEKL |
| | | | VRALQTPLMRLAAWYLYGEKHRGYALNPVANFHLQ |
| | | | NGAVLWRINWMADVSLRGITGSAGLMANYRYFLEE |
| | | | TGPNSTSYLGSKIIKASEQVLSLVAQFQK |
| 86 | 18 | Helix | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISKDSIQKSLQ |
| | | | MYLFGLEADELLRRAVPPTPAYELREKTPAPAEGQ |
| | | | AADFVSFYGGLAETAQRAELLGRLARGFGVDHGQV |
| | | | AEQSAGVLHLRQQQREAAVLLQAEDRLRYALVPRY |
| | | | RGLFHHISKLDGGVRFLVQLRADLLEAQALKLVEGP |
| | | | DVREMNGVLKGMLSEWFSSGFLNLERVTWHSPRE |
| | | | VLQKISEAEAVHPVKNWMDMKRRVGPYRRCYFFS |
| | | | HRSTPGEPLVVLHVALTGDISSNIQAIVKEHPPSETE |
| | | | EKNKITAAIFYSISLTQQGLQGVELGTFLIKRVVKELQ |
| | | | REFPHLGVFSSLSPIPGFTKWLLGLLNSQTKEHGRN |
| | | | ELFTDSERKEISEITGGPIRETLKLLLSSSEWVQSEK |
| | | | LVRALQTPLMRLAAWYLYGEKHRGYALNPVANFHL |
| | | | QNGAVLWRINWMADVSLRGITGSAGLMANYRYFLE |
| | | | ETGPNSTSYLGSKIIKASEQVLSLVAQFQK |
| 87 | 18 | Helix | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISKDSIQKSLQ |
| | | | MYLRGLEIDELLRRAVPPTPAYELREKTPAPAEGQA |
| | | | ADFVSFYGGLAETAQRAELLGRLARGFGVDHGQVA |
| | | | EQSAGVLHLRQQQREAAVLLQAEDRLRYALVPRYR |
| | | | GLFHHISKLDGGVRFLVQLRADLLEAQALKLVEGPD |
| | | | VREMNGVLKGMLSEWFSSGFLNLERVTWHSPREV |
| | | | LQKISEAEAVHPVKNWMDMKRRVGPYRRCYFFSH |
| | | | RSTPGEPLVVLHVALTGDISSNIQAIVKEHPPSETEE |
| | | | KNKITAAIFYSISLTQQGLQGVELGTFLIKRVVKELQR |
| | | | EFPHLGVFSSLSPIPGFTKWLLGLLNSQTKEHGRNE |
| | | | LFTDSERKEISEITGGPIRETLKLLLSSSEWVQSEKL |
| | | | VRALQTPLMRLAAWYLYGEKHRGYALNPVANFHLQ |
| | | | NGAVLWRINWMADVSLRGITGSAGLMANYRYFLEE |
| | | | TGPNSTSYLGSKIIKASEQVLSLVAQFQK |
| 88 | 12 | Extended | RFGSQRVLVEPDAGAGVAVMKFKNPPVNSLSLEFL |
| | | | TELVISLEKLENDKSFRGVILTSDRPGVFSAGLDLTE |
| | | | MVGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQARLA |
| | | | AYLEELKKKELKAIAEKIVANGKGILAADESVGTMGN |
| | | | RLQRIKVENTEENRRQFREILFSVDSSISQSIGGVILF |
| | | | HETLYQKDSQGKLFRNILKEKGIVVGIKLDQGGAPL |
| | | | AGTNKETTIQGLDGLSERCAQYKKDGVDFGKWRAV |
| | | | LRIADQSPSSLAIQENANALARYASICQQNGLVPIVE |
| | | | PEVIPDGDHDLEHHQYVTEKVLAAVYKALNDHHVYL |
| | | | EGTLLKPNMVTAGHAHTKKYTPEQVAMATVTALHR |
| | | | TVPAAVPGIAFLSGGMSEEDATLNLNAINLNPLPKP |
| | | | WKLSFSYGRALQASALAAWGGKAANKEATQEAFM |
| | | | KRAMANSQAAKGQYVHTGSSGAASTQSLFTACYT |
| | | | Y |
| 89 | 12 | Extended | RFGSQRVLVEPDAGAGVAVMKFKNPPVNSLSLEFL |
| | | | TELVISLEKLENDKSFRGVILTSDRPGVFSAGLDLTE |
| | | | MVGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQAALK |
| | | | AFLEQLKKKELKEIANKIVANGKGILAADESVGTMGN |
| | | | RLQRIKVENTEENRRQFREILFSVDSSISQSIGGVILF |
| | | | HETLYQKDSQGKLFRNILKEKGIVVGIKLDQGGAPL |
| | | | AGTNKETTIQGLDGLSERCAQYKKDGVDFGKWRAV |
| | | | LRIADQSPSSLAIQENANALARYASICQQNGLVPIVE |
| | | | PEVIPDGDHDLEHHQYVTEKVLAAVYKALNDHHVYL |
| | | | EGTLLKPNMVTAGHAHTKKYTPEQVAMATVTALHR |
| | | | TVPAAVPGIAFLSGGMSEEDATLNLNAINLNPLPKP |
| | | | WKLSFSYGRALQASALAAWGGKAANKEATQEAFM |
| | | | KRAMANSQAAKGQYVHTGSSGAASTQSLFTACYT |
| | | | Y |
| 90 | 12 | Extended | RFGSQRVLVEPDAGAGVAVMKFKNPPVNSLSLEFL |
| | | | TELVISLEKLENDKSFRGVILTSDRPGVFSAGLDLTE |
| | | | MVGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQARLA |
| | | | AFLEELRKKELAAIANKIVANGKGILAADESVGTMGN |
| | | | RLQRIKVENTEENRRQFREILFSVDSSISQSIGGVILF |
| | | | HETLYQKDSQGKLFRNILKEKGIVVGIKLDQGGAPL |
| | | | AGTNKETTIQGLDGLSERCAQYKKDGVDFGKWRAV |
| | | | LRIADQSPSSLAIQENANALARYASICQQNGLVPIVE |
| | | | PEVIPDGDHDLEHHQYVTEKVLAAVYKALNDHHVYL |
| | | | EGTLLKPNMVTAGHAHTKKYTPEQVAMATVTALHR |
| | | | TVPAAVPGIAFLSGGMSEEDATLNLNAINLNPLPKP |
| | | | WKLSFSYGRALQASALAAWGGKAANKEATQEAFM |
| | | | KRAMANSQAAKGQYVHTGSSGAASTQSLFTACYT |
| | | | Y |
| 91 | 12 | Helix | RFGSQRVLVEPDAGAGVAVMKFKNPPVNSLSLEFL |
| | | | TELVISLEKLENDKSFRGVILTSDRPGVFSAGLDLTE |
| | | | MVGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISKDSIQKSLQ |
| | | | MYLEELKKKELSEIAQSIVANGKGILAADESVGTMG |
| | | | NRLQRIKVENTEENRRQFREILFSVDSSISQSIGGVIL |
| | | | FHETLYQKDSQGKLFRNILKEKGIVVGIKLDQGGAPL |
| | | | AGTNKETTIQGLDGLSERCAQYKKDGVDFGKWRAV |
| | | | LRIADQSPSSLAIQENANALARYASICQQNGLVPIVE |
| | | | PEVIPDGDHDLEHHQYVTEKVLAAVYKALNDHHVYL |
| | | | EGTLLKPNMVTAGHAHTKKYTPEQVAMATVTALHR |
| | | | TVPAAVPGIAFLSGGMSEEDATLNLNAINLNPLPKP |
| | | | WKLSFSYGRALQASALAAWGGKAANKEATQEAFM |
| | | | KRAMANSQAAKGQYVHTGSSGAASTQSLFTACYT |
| | | | Y |
| 92 | 12 | Helix | RFGSQRVLVEPDAGAGVAVMKFKNPPVNSLSLEFL |
| | | | TELVISLEKLENDKSFRGVILTSDRPGVFSAGLDLTE |
| | | | MVGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISKDSIQKSLQ |
| | | | MYLEELRKKELSEIAQSIVANGKGILAADESVGTMG |
| | | | NRLQRIKVENTEENRRQFREILFSVDSSISQSIGGVIL |
| | | | FHETLYQKDSQGKLFRNILKEKGIVVGIKLDQGGAPL |
| | | | AGTNKETTIQGLDGLSERCAQYKKDGVDFGKWRAV |
| | | | LRIADQSPSSLAIQENANALARYASICQQNGLVPIVE |
| | | | PEVIPDGDHDLEHHQYVTEKVLAAVYKALNDHHVYL |
| | | | EGTLLKPNMVTAGHAHTKKYTPEQVAMATVTALHR |
| | | | TVPAAVPGIAFLSGGMSEEDATLNLNAINLNPLPKP |
| | | | WKLSFSYGRALQASALAAWGGKAANKEATQEAFM |
| | | | KRAMANSQAAKGQYVHTGSSGAASTQSLFTACYT |
| | | | Y |
| 93 | 4 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSKEFQDAVKAAFQK |
| | | | QYDARLKALYPAVNQQETPLPRSWSPKDKYNYIGL |
| | | | SQGNLRVHYKGHGKNHKDAASVRATHPIPAACGIY |
| | | | YFEVKIVSKGRDGYMGIGLSAQGVNMNRLPGWDK |
| | | | HSYGYHGDDGHSFDSSGTGQPYGPTFTTGDVIGV |
| | | | VVNLIAGTLFYTKNGHSLGIAFTDLPANLYPTVGLQT |
| | | | PGEIVDANFGQQPFLFDIEDYMREWRAKVQGTVHL |
| | | | FPISARLGEWQAVLQNMVSSYLVHHGYVATATAFA |
| | | | RMTETPIQEEQASIKNRQKIQKLVLEGRVGEAIETTQ |
| | | | RFYPGLLEHNPNLLFMLKSRQFVEMVNGTDSEVRS |
| | | | LSSRSPKSQDSYPGSPSLSPRHGPSSSHMHNTGA |
| | | | DSPSASNGVASTKSKQNHSKYPAPSSSSSSSSSSS |
| | | | SSSPSSVNYSESNSTDSTKSQHHSSTSNQETSDSE |
| | | | MEMEAEHYPNGVLGSMSTRIVNGAYKHEDLQTDES |
| | | | SMDDRHPRRQLLGGNQAATERIILFGRELQALSEQL |
| | | | GREYGKNLAHTEMLQDAFSLLAYSDPWSAPVGQQ |
| | | | LDPIQREPVCAALNSAILESQNLPKQPPLMLALGQA |
| | | | SECLRLMARAGLGSCSFA |
| 94 | 4 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSPEHQAAIAALFQKQ |
| | | | YDARLKKLYPAVNQQETPLPRSWSPKDKYNYIGLS |
| | | | QGNLRVHYKGHGKNHKDAASVRATHPIPAACGIYY |
| | | | FEVKIVSKGRDGYMGIGLSAQGVNMNRLPGWDKH |
| | | | SYGYHGDDGHSFDSSGTGQPYGPTFTTGDVIGVVV |
| | | | NLIAGTLFYTKNGHSLGIAFTDLPANLYPTVGLQTPG |
| | | | EIVDANFGQQPFLFDIEDYMREWRAKVQGTVHLFPI |
| | | | SARLGEWQAVLQNMVSSYLVHHGYVATATAFARM |
| | | | TETPIQEEQASIKNRQKIQKLVLEGRVGEAIETTQRF |
| | | | YPGLLEHNPNLLFMLKSRQFVEMVNGTDSEVRSLS |
| | | | SRSPKSQDSYPGSPSLSPRHGPSSSHMHNTGADS |
| | | | PSASNGVASTKSKQNHSKYPAPSSSSSSSSSSSSS |
| | | | SPSSVNYSESNSTDSTKSQHHSSTSNQETSDSEME |
| | | | MEAEHYPNGVLGSMSTRIVNGAYKHEDLQTDESSM |
| | | | DDRHPRRQLLGGNQAATERIILFGRELQALSEQLGR |
| | | | EYGKNLAHTEMLQDAFSLLAYSDPWSAPVGQQLDP |
| | | | IQREPVCAALNSAILESQNLPKQPPLMLALGQASEC |
| | | | LRLMARAGLGSCSFA |
| 95 | 4 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVALFEQE |
| | | | LSRRLQRLYPAVNQQETPLPRSWSPKDKYNYIGLS |
| | | | QGNLRVHYKGHGKNHKDAASVRATHPIPAACGIYY |
| | | | FEVKIVSKGRDGYMGIGLSAQGVNMNRLPGWDKH |
| | | | SYGYHGDDGHSFDSSGTGQPYGPTFTTGDVIGVVV |
| | | | NLIAGTLFYTKNGHSLGIAFTDLPANLYPTVGLQTPG |
| | | | EIVDANFGQQPFLFDIEDYMREWRAKVQGTVHLFPI |
| | | | SARLGEWQAVLQNMVSSYLVHHGYVATATAFARM |
| | | | TETPIQEEQASIKNRQKIQKLVLEGRVGEAIETTQRF |
| | | | YPGLLEHNPNLLFMLKSRQFVEMVNGTDSEVRSLS |
| | | | SRSPKSQDSYPGSPSLSPRHGPSSSHMHNTGADS |
| | | | PSASNGVASTKSKQNHSKYPAPSSSSSSSSSSSSS |
| | | | SPSSVNYSESNSTDSTKSQHHSSTSNQETSDSEME |
| | | | MEAEHYPNGVLGSMSTRIVNGAYKHEDLQTDESSM |
| | | | DDRHPRRQLLGGNQAATERIILFGRELQALSEQLGR |
| | | | EYGKNLAHTEMLQDAFSLLAYSDPWSAPVGQQLDP |
| | | | IQREPVCAALNSAILESQNLPKQPPLMLALGQASEC |
| | | | LRLMARAGLGSCSFA |
| 96 | 4 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVAKFEEE |
| | | | LSRRLQRLYPAVNQQETPLPRSWSPKDKYNYIGLS |
| | | | QGNLRVHYKGHGKNHKDAASVRATHPIPAACGIYY |
| | | | FEVKIVSKGRDGYMGIGLSAQGVNMNRLPGWDKH |
| | | | SYGYHGDDGHSFDSSGTGQPYGPTFTTGDVIGVVV |
| | | | NLIAGTLFYTKNGHSLGIAFTDLPANLYPTVGLQTPG |
| | | | EIVDANFGQQPFLFDIEDYMREWRAKVQGTVHLFPI |
| | | | SARLGEWQAVLQNMVSSYLVHHGYVATATAFARM |
| | | | TETPIQEEQASIKNRQKIQKLVLEGRVGEAIETTQRF |
| | | | YPGLLEHNPNLLFMLKSRQFVEMVNGTDSEVRSLS |
| | | | SRSPKSQDSYPGSPSLSPRHGPSSSHMHNTGADS |
| | | | PSASNGVASTKSKQNHSKYPAPSSSSSSSSSSSSS |
| | | | SPSSVNYSESNSTDSTKSQHHSSTSNQETSDSEME |
| | | | MEAEHYPNGVLGSMSTRIVNGAYKHEDLQTDESSM |
| | | | DDRHPRRQLLGGNQAATERIILFGRELQALSEQLGR |
| | | | EYGKNLAHTEMLQDAFSLLAYSDPWSAPVGQQLDP |
| | | | IQREPVCAALNSAILESQNLPKQPPLMLALGQASEC |
| | | | LRLMARAGLGSCSFA |
| 97 | 21 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVKKLLEKE |
| | | | KYIEERLEESIKLAKEAGEIVREAMKNEMNVMLKSS |
| | | | PVDLVTATDQKVEKMLISSIKEKYPSHSFIGEESVAA |
| | | | GEKSILTDNPTWIIDPIDGTTNFVHRFPFVAVSIGFAV |
| | | | NKKIEFGVVYSVVEGKMYTARKGKGAFLNGQKLQV |
| | | | SQQEDITKSLLVTELGSSRTPETVRMVLSNMEKLFKI |
| | | | PVHGIRSVGTAAVNMCLVATGGADAYYEMGIHSWD |
| | | | VAGAGIIVTEAGGVLMDVTGGPFDLMSRRVIAANNR |
| | | | ILAERIAKEIQVIPLQRDDE |
| 98 | 21 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVKKKLAEE |
| | | | KYLEERLEDAIKLAKEAGEIVREAMKNEMNVMLKSS |
| | | | PVDLVTATDQKVEKMLISSIKEKYPSHSFIGEESVAA |
| | | | GEKSILTDNPTWIIDPIDGTTNFVHRFPFVAVSIGFAV |
| | | | NKKIEFGVVYSVVEGKMYTARKGKGAFLNGQKLQV |
| | | | SQQEDITKSLLVTELGSSRTPETVRMVLSNMEKLFKI |
| | | | PVHGIRSVGTAAVNMCLVATGGADAYYEMGIHSWD |
| | | | VAGAGIIVTEAGGVLMDVTGGPFDLMSRRVIAANNR |
| | | | ILAERIAKEIQVIPLQRDDE |
| 99 | 21 | Extended | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVKKKLAKE |
| | | | KELEERYELAVKLAKEAGEIVREAMKNEMNVMLKS |
| | | | SPVDLVTATDQKVEKMLISSIKEKYPSHSFIGEESVA |
| | | | AGEKSILTDNPTWIIDPIDGTTNFVHRFPFVAVSIGFA |
| | | | VNKKIEFGVVYSVVEGKMYTARKGKGAFLNGQKLQ |
| | | | VSQQEDITKSLLVTELGSSRTPETVRMVLSNMEKLF |
| | | | KIPVHGIRSVGTAAVNMCLVATGGADAYYEMGIHS |
| | | | WDVAGAGIIVTEAGGVLMDVTGGPFDLMSRRVIAA |
| | | | NNRILAERIAKEIQVIPLQRDDE |
| 100 | 21 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVNFLKRE |
| | | | KYLQERMDYAVTLARQAGEVVREAIKNEMNVMLKS |
| | | | SPVDLVTATDQKVEKMLISSIKEKYPSHSFIGEESVA |
| | | | AGEKSILTDNPTWIIDPIDGTTNFVHRFPFVAVSIGFA |
| | | | VNKKIEFGVVYSVVEGKMYTARKGKGAFLNGQKLQ |
| | | | VSQQEDITKSLLVTELGSSRTPETVRMVLSNMEKLF |
| | | | KIPVHGIRSVGTAAVNMCLVATGGADAYYEMGIHS |
| | | | WDVAGAGIIVTEAGGVLMDVTGGPFDLMSRRVIAA |
| | | | NNRILAERIAKEIQVIPLQRDDE |
| 101 | 21 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVNFLKEE |
| | | | KYLQERMDYAVTLARQAGEVVREAIKNEMNVMLKS |
| | | | SPVDLVTATDQKVEKMLISSIKEKYPSHSFIGEESVA |
| | | | AGEKSILTDNPTWIIDPIDGTTNFVHRFPFVAVSIGFA |
| | | | VNKKIEFGVVYSVVEGKMYTARKGKGAFLNGQKLQ |
| | | | VSQQEDITKSLLVTELGSSRTPETVRMVLSNMEKLF |
| | | | KIPVHGIRSVGTAAVNMCLVATGGADAYYEMGIHS |
| | | | WDVAGAGIIVTEAGGVLMDVTGGPFDLMSRRVIAA |
| | | | NNRILAERIAKEIQVIPLQRDDE |
| 102 | 21 | Helix | FETLVVTSEDGITKIMFNRPKKKNAINTEMYHEIMRA |
| | | | LKAASKDDSIITVLTGNGDYYSSGNDLTNFTDIPPGG |
| | | | VEEKAKNNAVLLREFVGAFIDFPKPLIAVVNGPAVGI |
| | | | SVTLLGLFDAVYASDRATFHTPFSHLGQSPEGCSS |
| | | | YTFPKIMSPAKATEMLIFGKKLTAGEAQAQGLVTEV |
| | | | FPDSTFQKEVWTRLKAFAKLPPNALRISKEVIRKRE |
| | | | REKLHAVNAEEANVLQGRWLSDEFTNAVVNFLKRE |
| | | | KFLQERMDYAVTLARQAGEVVREAIKNEMNVMLKS |
| | | | SPVDLVTATDQKVEKMLISSIKEKYPSHSFIGEESVA |
| | | | AGEKSILTDNPTWIIDPIDGTTNFVHRFPFVAVSIGFA |
| | | | VNKKIEFGVVYSVVEGKMYTARKGKGAFLNGQKLQ |
| | | | VSQQEDITKSLLVTELGSSRTPETVRMVLSNMEKLF |
| | | | KIPVHGIRSVGTAAVNMCLVATGGADAYYEMGIHS |
| | | | WDVAGAGIIVTEAGGVLMDVTGGPFDLMSRRVIAA |
| | | | NNRILAERIAKEIQVIPLQRDDE |
| 103 | 8 | Extended | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQAALA |
| | | | RFLAALRAAAAEARLKKFLSAPIGDRDVIWVDGISHE |
| | | | LAINLVTKGINKAYILLGQFLLMHKNEAEFQRWLIEN |
| | | | FGATEEEAQQTSHALKEWCAKFL |
| 104 | 8 | Extended | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQARLA |
| | | | AFLAALAERRRQERLEKFLSEPIGDRDVIWVDGISH |
| | | | ELAINLVTKGINKAYILLGQFLLMHKNEAEFQRWLIE |
| | | | NFGATEEEAQQTSHALKEWCAKFL |
| 105 | 8 | Helix | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISKDSIQKSLQ |
| | | | MYLERLKEAAREARLRAFLSEPIGEKDVIWVDGISH |
| | | | ELAINLVTKGINKAYILLGQFLLMHKNEAEFQRWLIE |
| | | | NFGATEEEAQQTSHALKEWCAKFL |
| 106 | 8 | Helix | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISKDSIQKSLQ |
| | | | MYLERLAQARREARLRAFLSEPIGEKDVIWVDGISH |
| | | | ELAINLVTKGINKAYILLGQFLLMHKNEAEFQRWLIE |
| | | | NFGATEEEAQQTSHALKEWCAKFL |
| 107 | 1 | Extended | YTYEDYKNTAEWLLSHTKHRPQVAIIAGSGLGGLTD |
| | | | KLTQAQIFDYGEIPNFPRSTVPGHAGRLVFGFLNGR |
| | | | AVVMMQGRFHMYEGYPLWKVTFPVRVFHLLGVDT |
| | | | LVVTNAAGGLNPKFEVGDIMLIRDHINLPGFSGQNP |
| | | | LRGPNDERFGDRFPAMSDAYDRTMRQRALSTWKQ |
| | | | MGEQRELQEGTYVMVAGPSFETVAEARVLQKLGA |
| | | | DAVGMSTVPEVIVARHCGLRVFGFSLITNKVIMDYE |
| | | | SLEKADFASVLEAGKAALPRVTEFLTALLADEAELD |
| | | | EIRAYLASKLPSVEGLHAIVVSDRDGVPVIKVANDNA |
| | | | PEHALRPGFLSTFALATDQGSKLGLSKVKSIISYYNT |
| | | | YQVVQFNRLPLVVSFIASSSANTGLIVSLEKELAPLF |
| | | | EELRQVVE |
| 108 | 1 | Extended | YTYEDYKNTAEWLLSHTKHRPQVAIIAGSGLGGLTD |
| | | | KLTQAQIFDYGEIPNFPRSTVPGHAGRLVFGFLNGR |
| | | | AVVMMQGRFHMYEGYPLWKVTFPVRVFHLLGVDT |
| | | | LVVTNAAGGLNPKFEVGDIMLIRDHINLPGFSGQNP |
| | | | LRGPNDERFGDRFPAMSDAYDRTMRQRALSTWKQ |
| | | | MGEQRELQEGTYVMVAGPSFETVAEARVLQKLGA |
| | | | DAVGMSTVPEVIVARHCGLRVFGFSLITNKVIMDYE |
| | | | SLEKANFAEVLAAGAAALPRISRFLGQLLADIAREDE |
| | | | IRAYLSSKLPSVEGLHAIVVSDRDGVPVIKVANDNAP |
| | | | EHALRPGFLSTFALATDQGSKLGLSKVKSIISYYNTY |
| | | | QVVQFNRLPLVVSFIASSSANTGLIVSLEKELAPLFE |
| | | | ELRQVVE |
| 109 | 1 | Helix | YTYEDYKNTAEWLLSHTKHRPQVAIIAGSGLGGLTD |
| | | | KLTQAQIFDYGEIPNFPRSTVPGHAGRLVFGFLNGR |
| | | | AVVMMQGRFHMYEGYPLWKVTFPVRVFHLLGVDT |
| | | | LVVTNAAGGLNPKFEVGDIMLIRDHINLPGFSGQNP |
| | | | LRGPNDERFGDRFPAMSDAYDRTMRQRALSTWKQ |
| | | | MGEQRELQEGTYVMVAGPSFETVAEARVLQKLGA |
| | | | DAVGMSTVPEVIVARHCGLRVFGFSLITNKVIMDYE |
| | | | SLEKANHEEVLAAGKQAAQKLEQFVSILMADIARDD |
| | | | DLKRFLYKKLPSVEGLHAIVVSDRDGVPVIKVANDN |
| | | | APEHALRPGFLSTFALATDQGSKLGLSKVKSIISYYN |
| | | | TYQVVQFNRLPLVVSFIASSSANTGLIVSLEKELAPL |
| | | | FEELRQVVE |
| 110 | 1 | Helix | YTYEDYKNTAEWLLSHTKHRPQVAIIAGSGLGGLTD |
| | | | KLTQAQIFDYGEIPNFPRSTVPGHAGRLVFGFLNGR |
| | | | AVVMMQGRFHMYEGYPLWKVTFPVRVFHLLGVDT |
| | | | LVVTNAAGGLNPKFEVGDIMLIRDHINLPGFSGQNP |
| | | | LRGPNDERFGDRFPAMSDAYDRTMRQRALSTWKQ |
| | | | MGEQRELQEGTYVMVAGPSFETVAEARVLQKLGA |
| | | | DAVGMSTVPEVIVARHCGLRVFGFSLITNKVIMDYE |
| | | | SLEKANHEEVLAAGKQAAQKLEQFVSILMADWAEE |
| | | | EDLKRFLYKKLPSVEGLHAIVVSDRDGVPVIKVAND |
| | | | NAPEHALRPGFLSTFALATDQGSKLGLSKVKSIISYY |
| | | | NTYQVVQFNRLPLVVSFIASSSANTGLIVSLEKELAP |
| | | | LFEELRQVVE |
| 111 | 5 | Extended | KYIPRRAVLYVPGNDEKKIKKIPSLNVDVAVLDTEDG |
| | | | VAANKKNEARLRIVKTLEDIDLGPTEKMVRVNSVSS |
| | | | GLAEEDLETLLQSRVLPSSLMLPKVESPEEIQWFAD |
| | | | KFSFHLKGRKLEQPMNLIPFVETAMGLLNFKAVCEE |
| | | | TLKVGPQVGLFLDAVVFGGEDFRASIGATSSKETLD |
| | | | ILYARQKIVVIAKAFGLQAIDLVYIDFRDGAGLLRQSR |
| | | | EGAAMGFTGKQVIHPNQIAVVQEQFSPSPEKIKWA |
| | | | EELIAAFKEHQQLGKGAFTFQGSMINQADIDEAKAIV |
| | | | EEAKAIQERKEKRAQALAEKVAQEFVETYFTTFDKR |
| | | | RRLLSRLYMGTATLVWNGNAVSGQESLSEFFEMLP |
| | | | SSEFQISVVDSQPVHDEATPSQTTVLVVIKGSVKFE |
| | | | GNKQRDFNQNFILTAQASPSNTVWKIASDDFRFQ |
| 112 | 5 | Extended | KYIPRRAVLYVPGNDEKKIKKIPSLNVDVAVLDTEDG |
| | | | VAANKKNEARLRIVKTLEDIDLGPTEKMVRVNSVSS |
| | | | GLAEEDLETLLQSRVLPSSLMLPKVESPEEIQWFAD |
| | | | KFSFHLKGRKLEQPMNLIPFVETAMGLLNFKAVCEE |
| | | | TLKVGPQVGLFLDAVVFGGEDFRASIGATSSKETLD |
| | | | ILYARQKIVVIAKAFGLQAIDLVYIDFRDGAGLLRQSR |
| | | | EGAAMGFTGKQVIHPNQIAVVQEQFSPSPEKIKWA |
| | | | EELIAAFKEHQQLGKGAFTFQGSMIDDAKIAEAEAIV |
| | | | KEAKAIKEREEARKQALAEQAAAAFVEEYFTTFDKR |
| | | | RRLLSRLYMGTATLVWNGNAVSGQESLSEFFEMLP |
| | | | SSEFQISVVDSQPVHDEATPSQTTVLVVIKGSVKFE |
| | | | GNKQRDFNQNFILTAQASPSNTVWKIASDDFRFQ |
| 113 | 5 | Helix | KYIPRRAVLYVPGNDEKKIKKIPSLNVDVAVLDTEDG |
| | | | VAANKKNEARLRIVKTLEDIDLGPTEKMVRVNSVSS |
| | | | GLAEEDLETLLQSRVLPSSLMLPKVESPEEIQWFAD |
| | | | KFSFHLKGRKLEQPMNLIPFVETAMGLLNFKAVCEE |
| | | | TLKVGPQVGLFLDAVVFGGEDFRASIGATSSKETLD |
| | | | ILYARQKIVVIAKAFGLQAIDLVYIDFRDGAGLLRQSR |
| | | | EGAAMGFTGKQVIHPNQIAVVQEQFSPSPEKIKWA |
| | | | EELIAAFKEHQQLGKGAFTFQGSMIDMPLLKQAQNT |
| | | | VTLATSIKERKEKTYVDQAKRAAEEFVNVYYTTMDK |
| | | | RRRLLSRLYMGTATLVWNGNAVSGQESLSEFFEML |
| | | | PSSEFQISVVDSQPVHDEATPSQTTVLVVIKGSVKF |
| | | | EGNKQRDFNQNFILTAQASPSNTVWKIASDDFRFQ |
| 114 | 20 | Extended | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQARLA |
| | | | AFLEGLEIAEIAERMKTVKHKILVLSGKGGVGKSTFS |
| | | | AHLAHGLAEDENTQIALLDIDICGPSIPKIMGLEGEQ |
| | | | VHQSGSGWSPVYVEDNLGVMSVGFLLSSPDDAVI |
| | | | WRGPKKNGMIKQFLRDVDWGEVDYLIVDTPPGTSD |
| | | | EHLSVVRYLATAHIDGAVIITTPQEVSLQDVRKEINF |
| | | | DRKVKLPIIGVVENMSGFITPKCKKESQIFPPTTGGA |
| | | | ELMNQDLEVPLLGRVPLDPLIGKNCDKGQSFFIDAP |
| | | | DSPATLAYRSIIQRIQEFCNL |
| 115 | 20 | Extended | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQARLK |
| | | | AFLEEQEIAKIAERMKTVKHKILVLSGKGGVGKSTFS |
| | | | AHLAHGLAEDENTQIALLDIDICGPSIPKIMGLEGEQ |
| | | | VHQSGSGWSPVYVEDNLGVMSVGFLLSSPDDAVI |
| | | | WRGPKKNGMIKQFLRDVDWGEVDYLIVDTPPGTSD |
| | | | EHLSVVRYLATAHIDGAVIITTPQEVSLQDVRKEINF |
| | | | DRKVKLPIIGVVENMSGFITPKCKKESQIFPPTTGGA |
| | | | ELMNQDLEVPLLGRVPLDPLIGKNCDKGQSFFIDAP |
| | | | DSPATLAYRSIIQRIQEFCNL |
| 116 | 20 | Extended | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQAALA |
| | | | AFLEGLEIAEIAERMKTVKHKILVLSGKGGVGKSTFS |
| | | | AHLAHGLAEDENTQIALLDIDICGPSIPKIMGLEGEQ |
| | | | VHQSGSGWSPVYVEDNLGVMSVGFLLSSPDDAVI |
| | | | WRGPKKNGMIKQFLRDVDWGEVDYLIVDTPPGTSD |
| | | | EHLSVVRYLATAHIDGAVIITTPQEVSLQDVRKEINF |
| | | | DRKVKLPIIGVVENMSGFITPKCKKESQIFPPTTGGA |
| | | | ELMNQDLEVPLLGRVPLDPLIGKNCDKGQSFFIDAP |
| | | | DSPATLAYRSIIQRIQEFCNL |
| 117 | 20 | Helix | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISKDSIQKSLQ |
| | | | MYLEQLEIEEIKEKMKTVKHKILVLSGKGGVGKSTFS |
| | | | AHLAHGLAEDENTQIALLDIDICGPSIPKIMGLEGEQ |
| | | | VHQSGSGWSPVYVEDNLGVMSVGFLLSSPDDAVI |
| | | | WRGPKKNGMIKQFLRDVDWGEVDYLIVDTPPGTSD |
| | | | EHLSVVRYLATAHIDGAVIITTPQEVSLQDVRKEINF |
| | | | DRKVKLPIIGVVENMSGFITPKCKKESQIFPPTTGGA |
| | | | ELMNQDLEVPLLGRVPLDPLIGKNCDKGQSFFIDAP |
| | | | DSPATLAYRSIIQRIQEFCNL |
| 118 | 20 | Helix | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISKDSIQKSLQ |
| | | | MYLEGLEIAEIKEKMKTVKHKILVLSGKGGVGKSTFS |
| | | | AHLAHGLAEDENTQIALLDIDICGPSIPKIMGLEGEQ |
| | | | VHQSGSGWSPVYVEDNLGVMSVGFLLSSPDDAVI |
| | | | WRGPKKNGMIKQFLRDVDWGEVDYLIVDTPPGTSD |
| | | | EHLSVVRYLATAHIDGAVIITTPQEVSLQDVRKEINF |
| | | | DRKVKLPIIGVVENMSGFITPKCKKESQIFPPTTGGA |
| | | | ELMNQDLEVPLLGRVPLDPLIGKNCDKGQSFFIDAP |
| | | | DSPATLAYRSIIQRIQEFCNL |
| 119 | 13 | Extended | KYIPRRAVLYVPGNDEKKIKKIPSLNVDVAVLDLEDG |
| | | | VAANKKNEARLRIVKTLEDIDLGPTEKMVRVNSVSS |
| | | | GLAEEDLETLLQSRVLPSSLMLPKVESPEEIQWFAD |
| | | | KFSFHLKGRKLEQPMNLIPFVETAMGLLNFKAVCEE |
| | | | TLKVGPQVGLFLDAVVFGGEDFRASIGATSSKETLD |
| | | | ILYARQKIVVIAKAFGLQAIDLVYIDFRDGAGLLRQSR |
| | | | EGAAMGFTGKQVIHPNQIAVVQEQFSPSPEKIKWA |
| | | | EELIAAFKEHQQLGKGAFTFQGSMVTQADIDKAKAII |
| | | | EQAEAELRRFAARWCSELLGGAWRRVQPEELRVY |
| | | | PVSGGLSNLLFRVSLPDHLPSVGEEPREVLLRLYGA |
| | | | ILQGVDSLVLESVMFAILAERSLGPQLYGVFPEGRL |
| | | | EQYIPSRPLKTQELREPVLSAAIATKMAQFHGMEMP |
| | | | FTKEPHWLFGTMERYLKQIQDLPPTGLPEMNLLEM |
| | | | YSLKDEMGNLRKLLESTPSPVVFCHNDIQEGNILLLS |
| | | | EPENADSLMLVDFEYSSYNYRGFDIGNHFNEWVYD |
| | | | YTHEEWPFYKARPTDYPTQEQQLHFIRHYLAEAKK |
| | | | GETLSQEEQRKLEEDLLVEVSRYALASHFFWGLWS |
| | | | ILQASMSTIEFGYLDYAQSRFQFYFQQKGQLT |
| 120 | 13 | Extended | KYIPRRAVLYVPGNDEKKIKKIPSLNVDVAVLDLEDG |
| | | | VAANKKNEARLRIVKTLEDIDLGPTEKMVRVNSVSS |
| | | | GLAEEDLETLLQSRVLPSSLMLPKVESPEEIQWFAD |
| | | | KFSFHLKGRKLEQPMNLIPFVETAMGLLNFKAVCEE |
| | | | TLKVGPQVGLFLDAVVFGGEDFRASIGATSSKETLD |
| | | | ILYARQKIVVIAKAFGLQAIDLVYIDFRDGAGLLRQSR |
| | | | EGAAMGFTGKQVIHPNQIAVVQEQFSPSPEKIKWA |
| | | | EELIAAFKEHQQLGKGAFTFQGSMVDEAKIAEAKAI |
| | | | VEEAEALLRRFAARWCSEHLGGAWRRVQPEELRV |
| | | | YPVSGGLSNLLFRVSLPDHLPSVGEEPREVLLRLYG |
| | | | AILQGVDSLVLESVMFAILAERSLGPQLYGVFPEGR |
| | | | LEQYIPSRPLKTQELREPVLSAAIATKMAQFHGMEM |
| | | | PFTKEPHWLFGTMERYLKQIQDLPPTGLPEMNLLE |
| | | | MYSLKDEMGNLRKLLESTPSPVVFCHNDIQEGNILL |
| | | | LSEPENADSLMLVDFEYSSYNYRGFDIGNHFNEWV |
| | | | YDYTHEEWPFYKARPTDYPTQEQQLHFIRHYLAEA |
| | | | KKGETLSQEEQRKLEEDLLVEVSRYALASHFFWGL |
| | | | WSILQASMSTIEFGYLDYAQSRFQFYFQQKGQLT |
| 121 | 13 | Helix | KYIPRRAVLYVPGNDEKKIKKIPSLNVDVAVLDLEDG |
| | | | VAANKKNEARLRIVKTLEDIDLGPTEKMVRVNSVSS |
| | | | GLAEEDLETLLQSRVLPSSLMLPKVESPEEIQWFAD |
| | | | KFSFHLKGRKLEQPMNLIPFVETAMGLLNFKAVCEE |
| | | | TLKVGPQVGLFLDAVVFGGEDFRASIGATSSKETLD |
| | | | ILYARQKIVVIAKAFGLQAIDLVYIDFRDGAGLLRQSR |
| | | | EGAAMGFTGKQVIHPNQIAVVQEQFSPSPEKIKWA |
| | | | EELIAAFKEHQQLGKGAFTFQGSMIDMPLLKQAQNT |
| | | | VTLATSLLELKAYQWAREYLGGAWRRVQPEELRVY |
| | | | PVSGGLSNLLFRVSLPDHLPSVGEEPREVLLRLYGA |
| | | | ILQGVDSLVLESVMFAILAERSLGPQLYGVFPEGRL |
| | | | EQYIPSRPLKTQELREPVLSAAIATKMAQFHGMEMP |
| | | | FTKEPHWLFGTMERYLKQIQDLPPTGLPEMNLLEM |
| | | | YSLKDEMGNLRKLLESTPSPVVFCHNDIQEGNILLLS |
| | | | EPENADSLMLVDFEYSSYNYRGFDIGNHFNEWVYD |
| | | | YTHEEWPFYKARPTDYPTQEQQLHFIRHYLAEAKK |
| | | | GETLSQEEQRKLEEDLLVEVSRYALASHFFWGLWS |
| | | | ILQASMSTIEFGYLDYAQSRFQFYFQQKGQLT |
| 122 | 13 | Helix | KYIPRRAVLYVPGNDEKKIKKIPSLNVDVAVLDLEDG |
| | | | VAANKKNEARLRIVKTLEDIDLGPTEKMVRVNSVSS |
| | | | GLAEEDLETLLQSRVLPSSLMLPKVESPEEIQWFAD |
| | | | KFSFHLKGRKLEQPMNLIPFVETAMGLLNFKAVCEE |
| | | | TLKVGPQVGLFLDAVVFGGEDFRASIGATSSKETLD |
| | | | ILYARQKIVVIAKAFGLQAIDLVYIDFRDGAGLLRQSR |
| | | | EGAAMGFTGKQVIHPNQIAVVQEQFSPSPEKIKWA |
| | | | EELIAAFKEHQQLGKGAFTFQGSMIDMPLLKQAQNT |
| | | | VTLATSLLERKAYQWAREYLGGAWRRVQPEELRVY |
| | | | PVSGGLSNLLFRVSLPDHLPSVGEEPREVLLRLYGA |
| | | | ILQGVDSLVLESVMFAILAERSLGPQLYGVFPEGRL |
| | | | EQYIPSRPLKTQELREPVLSAAIATKMAQFHGMEMP |
| | | | FTKEPHWLFGTMERYLKQIQDLPPTGLPEMNLLEM |
| | | | YSLKDEMGNLRKLLESTPSPVVFCHNDIQEGNILLLS |
| | | | EPENADSLMLVDFEYSSYNYRGFDIGNHFNEWVYD |
| | | | YTHEEWPFYKARPTDYPTQEQQLHFIRHYLAEAKK |
| | | | GETLSQEEQRKLEEDLLVEVSRYALASHFFWGLWS |
| | | | ILQASMSTIEFGYLDYAQSRFQFYFQQKGQLT |
| 123 | 13 | Helix | KYIPRRAVLYVPGNDEKKIKKIPSLNVDVAVLDLEDG |
| | | | VAANKKNEARLRIVKTLEDIDLGPTEKMVRVNSVSS |
| | | | GLAEEDLETLLQSRVLPSSLMLPKVESPEEIQWFAD |
| | | | KFSFHLKGRKLEQPMNLIPFVETAMGLLNFKAVCEE |
| | | | TLKVGPQVGLFLDAVVFGGEDFRASIGATSSKETLD |
| | | | ILYARQKIVVIAKAFGLQAIDLVYIDFRDGAGLLRQSR |
| | | | EGAAMGFTGKQVIHPNQIAVVQEQFSPSPEKIKWA |
| | | | EELIAAFKEHQQLGKGAFTFQGSMIDMPLLKQAQNT |
| | | | VTLATSLLERRAYQWAREYLGGAWRRVQPEELRV |
| | | | YPVSGGLSNLLFRVSLPDHLPSVGEEPREVLLRLYG |
| | | | AILQGVDSLVLESVMFAILAERSLGPQLYGVFPEGR |
| | | | LEQYIPSRPLKTQELREPVLSAAIATKMAQFHGMEM |
| | | | PFTKEPHWLFGTMERYLKQIQDLPPTGLPEMNLLE |
| | | | MYSLKDEMGNLRKLLESTPSPVVFCHNDIQEGNILL |
| | | | LSEPENADSLMLVDFEYSSYNYRGFDIGNHFNEWV |
| | | | YDYTHEEWPFYKARPTDYPTQEQQLHFIRHYLAEA |
| | | | KKGETLSQEEQRKLEEDLLVEVSRYALASHFFWGL |
| | | | WSILQASMSTIEFGYLDYAQSRFQFYFQQKGQLT |
| 124 | 3 | Extended | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQAALA |
| | | | AFLAAAAAAAERAALLAEFLAHFEQVIRDLTEDEMG |
| | | | HPEIGDAIARLKEVLEYNAIGGKYNRGLTVVVAFREL |
| | | | VEPRKQDADSLQRAWTVGWCVELLQAFFLVADDIM |
| | | | DSSLTRRGQIAWYQKPGVGLDAINDANLLEASIYRL |
| | | | LKLYCREQPYYLNLIELFLQSSYQTEIGQTLDLLTAP |
| | | | QGNVDLVRFTEKRYKSIVKYKTAFYSFYLPIAAAMY |
| | | | MAGIDGEKEHANAKKILLEMGEFFQIQDDYLDLFGD |
| | | | PSVTGKIGTDIQDNKLSWLVVQALQRATPEQYQILK |
| | | | ENYGQKEAEKVARVKALYEELDLPAVFLQYEEDSY |
| | | | SHIMALIEQYAAPLPPAVFLGLARKIYKR |
| 125 | 3 | Extended | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISQPEVQAALA |
| | | | AFLAALAAAAEEAALLAEFLAHFETIIKDLTEDEMGH |
| | | | PEIGDAIARLKEVLEYNAIGGKYNRGLTVVVAFRELV |
| | | | EPRKQDADSLQRAWTVGWCVELLQAFFLVADDIMD |
| | | | SSLTRRGQIAWYQKPGVGLDAINDANLLEASIYRLL |
| | | | KLYCREQPYYLNLIELFLOSSYQTEIGQTLDLLTAPQ |
| | | | GNVDLVRFTEKRYKSIVKYKTAFYSFYLPIAAAMYM |
| | | | AGIDGEKEHANAKKILLEMGEFFQIQDDYLDLFGDP |
| | | | SVTGKIGTDIQDNKLSWLVVQALQRATPEQYQILKE |
| | | | NYGQKEAEKVARVKALYEELDLPAVELQYEEDSYS |
| | | | HIMALIEQYAAPLPPAVFLGLARKIYKR |
| 126 | 3 | Helix | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISKDSIQKSLQ |
| | | | MYLERLAAEAAYAQEKQDFVQHFSQIVRVLTEDEM |
| | | | GHPEIGDAIARLKEVLEYNAIGGKYNRGLTVVVAFR |
| | | | ELVEPRKQDADSLQRAWTVGWCVELLQAFFLVAD |
| | | | DIMDSSLTRRGQIAWYQKPGVGLDAINDANLLEASI |
| | | | YRLLKLYCREQPYYLNLIELFLOSSYQTEIGQTLDLL |
| | | | TAPQGNVDLVRFTEKRYKSIVKYKTAFYSFYLPIAAA |
| | | | MYMAGIDGEKEHANAKKILLEMGEFFQIQDDYLDLF |
| | | | GDPSVTGKIGTDIQDNKLSWLVVQALQRATPEQYQI |
| | | | LKENYGQKEAEKVARVKALYEELDLPAVELQYEEDS |
| | | | YSHIMALIEQYAAPLPPAVFLGLARKIYKR |
| 127 | 3 | Helix | RFGSQRVLVEPDAGAGVAVMKFKNPPVNSLSLEFL |
| | | | TELVISLEKLENDKSFRGVILTSDRPGVFSAGLDLTE |
| | | | MTGRSPAHYAGYWKAVQELWLRLYQSNLVLVSAIN |
| | | | GACPAGGALVALTCDYRILADNPRYPIGLNETQLGII |
| | | | APFWLKDTLENTIGHRAAERALQLGLLFPPAEALQV |
| | | | GIVDQVVPEEQVQSTALSAIAQWMAIPDHARQLTKA |
| | | | MMRKATASRLVTQRDADVQNFVSFISKDSIQKSLQ |
| | | | MYLERLAAAAAYAQEKQDFVQHFSQIVRVLTEDEM |
| | | | GHPEIGDAIARLKEVLEYNAIGGKYNRGLTVVVAFR |
| | | | ELVEPRKQDADSLQRAWTVGWCVELLQAFFLVAD |
| | | | DIMDSSLTRRGQIAWYQKPGVGLDAINDANLLEASI |
| | | | YRLLKLYCREQPYYLNLIELFLOSSYQTEIGQTLDLL |
| | | | TAPQGNVDLVRFTEKRYKSIVKYKTAFYSFYLPIAAA |
| | | | MYMAGIDGEKEHANAKKILLEMGEFFQIQDDYLDLF |
| | | | GDPSVTGKIGTDIQDNKLSWLVVQALQRATPEQYQI |
| | | | LKENYGQKEAEKVARVKALYEELDLPAVFLQYEEDS |
| | | | YSHIMALIEQYAAPLPPAVFLGLARKIYKR |

### Example 4: Small-Scale Production of Human Protein Cages

### Cloning and Expression

DNA encoding each recombinant construct was synthesized with codon optimization for mammalian expression encoding a single polypeptide chain as enumerated in Table 12 above. The DNA fragment was cloned into the pcDNA3.4 vector backbone using the restriction sites EcoRI (G/AATTC) and HindIII (A/AGCTT). The resulting expression plasmid encoded the target protein with a histidine tag suitable for affinity purification. The plasmid was introduced into CHO-S cells (suspension culture) using a TurboCHO-HT 2.0 expression system. Expression was performed in a volume of 10 ml and incubated for 5 days under standard culture conditions. At the end of the incubation, the culture supernatant was harvested as starting material for purification.

### Purification

Proteins were purified from clarified supernatant by a multi-step Ni-affinity chromatography method. The supernatant was equilibrated into a loading buffer comprising 30 mM Tris pH 8.0, 300 mM NaCl, 20 mM imidazole, and 0.05% LDAO. The mixture was applied to a nickel-chelating resin, and the bound protein was subsequently washed with 10 column volumes of 30 mM Tris pH 8.0, 300 mM NaCl, and 20 mM imidazole in order to remove nonspecific binders.

Bound proteins were eluted in a stepwise fashion using 30 mM Tris pH 8.0 and 300 mM NaCl supplemented with either first 250 mM imidazole and then 500 mM imidazole as a second step. Eluted fractions were further processed separately by size exclusion chromatography (SEC) and SDS-PAGE analysis after buffer exchange into a formulation buffer consisting of 20 mM Tris pH 8.0, 150 mM NaCl, and 5 mM EDTA to remove imidazole, excess detergent, and small molecule contaminants. The resulting protein solutions typically had concentrations ~1 mg/ml.

As part of quality control, endotoxin levels were assessed by randomly selecting at least 5% of the total samples produced (minimum of 4 per batch). The target acceptance criterion was an endotoxin value of <1 EU/mg of purified protein.

### Analytical Characterization

The purified protein was subjected to size exclusion chromatography-high performance liquid chromatography (SEC-HPLC) to assess monodispersity and aggregation. Analysis was carried out using a Waters ACQUITY UPLC H-Class Bio system (System ID GR23010364) equipped with a Phenomenex HPLC Biozen column. Protein samples were loaded at 40 μL volumes in a mobile phase containing 0.1 M sodium carbonate and 0.118 M phosphate buffer, pH 6.7. Retention times of sample peaks were compared to standard molecular weight markers of 670 kDa, 158 kDa, 44 kDa, 17 kDa, and 1.4 kDa. The protein eluted at a retention time corresponding to its expected molecular weight, confirming the absence of significant aggregation.

Further confirmation of purity and molecular identity was achieved using sodium dodecyl sulfate polyacrylamide gel electrophoresis (SDS-PAGE). Samples were prepared under both reducing and non-reducing conditions and analyzed alongside molecular weight standards.

Table 13 below shows the protein production quantities of the human protein cages with SEQ ID NO: 64-127.

TABLE 13

| SEQ | 10 ml Production |
|---|---|
| ID NO | CHO-S (mgs) |
| 64 | 0.01 |
| 65 | 0.01 |
| 66 | 0.01 |
| 67 | 0.01 |
| 68 | 0.01 |
| 69 | 0.01 |
| 70 | 0.01 |
| 71 | 0.07 |
| 72 | 0.01 |
| 73 | 0.01 |
| 74 | 0.04 |
| 75 | 0.03 |
| 76 | 0.08 |
| 77 | 0.01 |
| 78 | 0.05 |
| 79 | 0.12 |
| 80 | 0.04 |
| 81 | 0.01 |
| 82 | 0.01 |
| 83 | 0.01 |
| 84 | 0.09 |
| 85 | 0.06 |
| 86 | 0.06 |
| 87 | 0.06 |
| 88 | 0.07 |
| 89 | 0.04 |
| 90 | 0.01 |
| 91 | 0.07 |
| 92 | 0.08 |
| 93 | 0.01 |
| 94 | 0.01 |
| 95 | 0.01 |
| 96 | 0.19 |
| 97 | 0.05 |
| 98 | 0.04 |
| 99 | 0.1 |
| 100 | 0.01 |
| 101 | 0.01 |
| 102 | 0.08 |
| 103 | 0.01 |
| 104 | 0.01 |
| 105 | 0.01 |
| 106 | 0.01 |
| 107 | 0.2 |
| 108 | 0.24 |
| 109 | 0.22 |
| 110 | 0.01 |
| 111 | 0.01 |
| 112 | 0.01 |
| 113 | 0.01 |
| 114 | 0.01 |
| 115 | 0.01 |
| 116 | 0.01 |
| 117 | 0.01 |
| 118 | 0.01 |
| 119 | 0.01 |
| 120 | 0.01 |
| 121 | 0.01 |
| 122 | 0.01 |
| 123 | 0.01 |
| 124 | 0.01 |
| 125 | 0.01 |
| 126 | 0.01 |
| 127 | 0.01 |

### Example 5: Scale-Up Production of Human Protein Cages

To support determination of cryoEM, thermal stability, BLI as well as accurate titer determination, constructs showing production and assembly in small scale productions were scaled to 100 ml CHO production and purified.

### Cloning and Expression

DNA encoding each recombinant construct was synthesized with codon optimization for mammalian expression encoding a single polypeptide chain as enumerated in Table 12 above. The DNA fragment was cloned into the pcDNA3.4 vector backbone using the restriction sites EcoRI (G/AATTC) and HindIII (A/AGCTT). The resulting expression plasmid encoded the target protein with a histidine tag suitable for affinity purification. The plasmid was introduced into CHO-S cells (suspension culture) using a TurboCHO-HT 2.0 expression system. Expression was performed in a volume of 10 ml and incubated for 5 days under standard culture conditions. At the end of the incubation, the culture supernatant was harvested as starting material for purification.

### Purification

Proteins were purified from clarified supernatant by a multi-step Ni-affinity chromatography method. The supernatant was equilibrated into a loading buffer comprising 30 mM Tris pH 8.0, 300 mM NaCl, 20 mM imidazole, and 0.05% LDAO. The mixture was applied to a nickel-chelating resin, and the bound protein was subsequently washed with 10 column volumes of 30 mM Tris pH 8.0, 300 mM NaCl, and 20 mM imidazole in order to remove nonspecific binders.

Bound proteins were eluted in a stepwise fashion using 30 mM Tris pH 8.0 and 300 mM NaCl supplemented with either first 250 mM imidazole and then 500 mM imidazole as a second step. Eluted fractions were further analyzed by size exclusion chromatography (SEC) and SDS-PAGE analysis after buffer exchange into a formulation buffer consisting of 20 mM Histidine pH 6.0, 150 mM NaCl, and 5 mM EDTA to remove imidazole, excess detergent, and small molecule contaminants. The resulting protein solutions typically had concentrations ~1 mg/ml.

As part of quality control, endotoxin levels were assessed by randomly selecting at least 5% of the total samples produced (minimum of 4 per batch). The target acceptance criterion was an endotoxin value of <1 EU/mg of purified protein.

### Analytical Characterization

The purified protein was subjected to size exclusion chromatography-high performance liquid chromatography (SEC-HPLC) to assess monodispersion and aggregation. Analysis was carried out using a Waters Arc UPLC H-Class Bio system equipped with a Superdex 200 Increase 5/150 column. Protein samples were loaded at 6 μL volumes in a mobile phase containing 0.1 mol/L arginine in 2×PBS, pH 10.55. Retention times of sample peaks were compared to standard molecular weight markers of 670 kDa, 158 kDa, 44 kDa, 17 kDa, and 1.4 kDa. The protein eluted at a retention time corresponding to its expected molecular weight, confirming the absence of significant aggregation.

Further confirmation of purity and molecular identity was achieved using sodium dodecyl sulfate polyacrylamide gel electrophoresis (SDS-PAGE). Samples were prepared under both reducing and non-reducing conditions and analyzed alongside molecular weight standards. Under reducing conditions, electrophoresis revealed a primary band corresponding to the expected molecular weight, while under non-reducing conditions, the protein displayed a similar migration pattern, indicating a predominantly monomeric, non-aggregated state.

100 ml production in CHO cells, followed by Ni column purification via his-tags, yielded purified protein that was buffer exchanged into Histidine pH 6.0, 150 mM NaCl, and 5 mM EDTA and analyzed for endotoxin, concentration and titer (based on predicted extinction coefficient based on amino acid composition) and purity (based on SEC and SDS-PAGE gel).

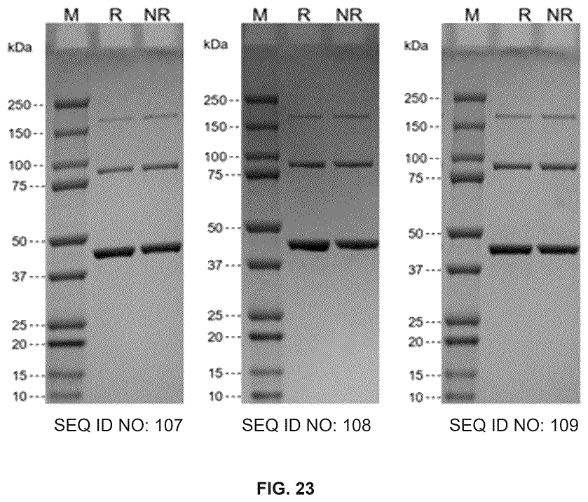

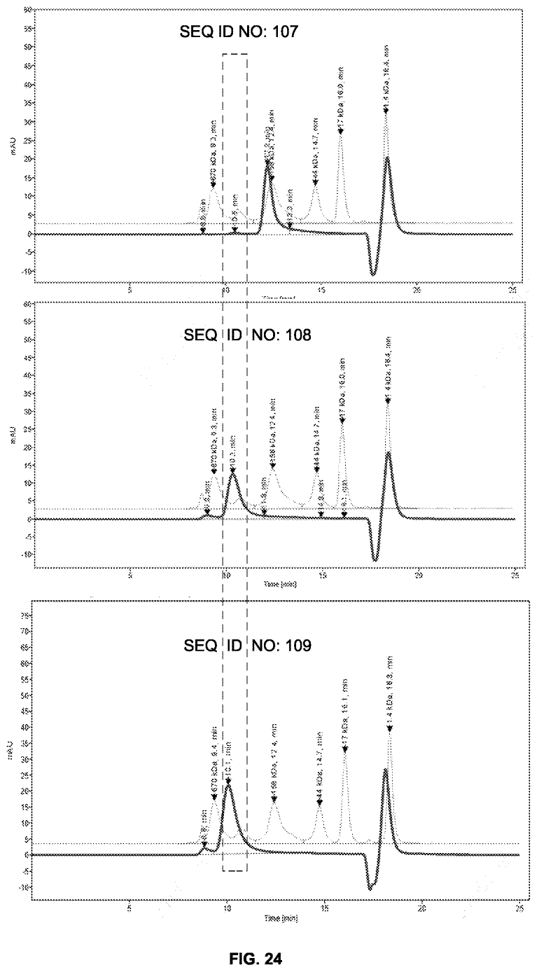

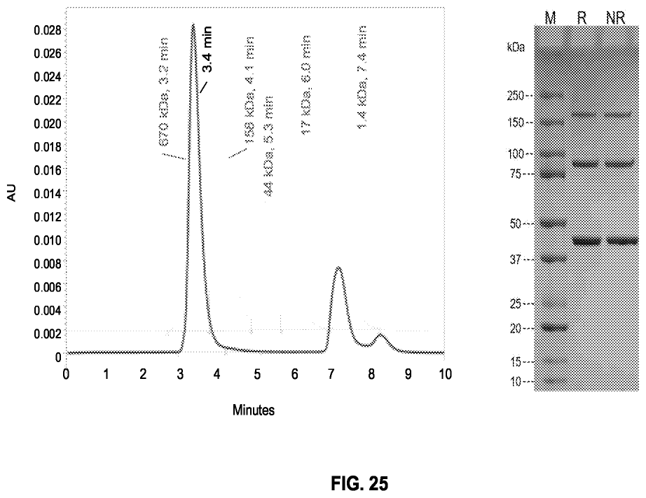

Proteins were produced at titers equivalent to antibodies, with levels as high as 100 mg/L post his-tag purification. SDS-PAGE gels of each construct under reducing and non-reducing conditions show a band at the correct molecular weight, as well as bands at 2×, 3×, etc the weight of the monomer MW. FIG. 23 illustrates the SDS gel for human protein cages with SEQ ID NO: 107, 108, and 109. Size exclusion chromatography of the three constructs (i.e., human protein cages with SEQ ID NO: 107, 108, and 109) under native conditions show particles for each with a molecular ~540 kD, as expected for an assembly of 12 monomers of molecular weight 45 kD. The size exclusion chromatography results are illustrated in FIG. 24, showing favorable production of constructs with SEQ ID NO: 108 and 109. FIG. 25 illustrates the size exclusion chromatography and SDS gel for construct with SEQ ID NO: 109 that was re-runed after a first size exclusion chromatography step.

Table 14 below shows the yield and endotoxin of the construct with SEQ ID NO: 107, 108, and 109.

TABLE 14

| | Endotoxin | Conc | Purity by | Purity by | Total | Titer |
|---|---|---|---|---|---|---|
| SEQ_ID_NO | (EU/mg) | (mg/ml) | SDS-PAGE (%) | SEC-HPLC (%) | (mg) | (mg/L) |
| 107 | 0.155 | 0.64 | 70 | 82 | 7.04 | 70.4 |
| 108 | 0.048 | 1.14 | 65 | 79 | 12.54 | 125.4 |
| 109 | 0.063 | 0.8 | 60 | 95 | 8.8 | 88 |

### Example 7: Thermal Stability Analysis for Human Protein Cages

### Methods

Protein thermal stability of the protein cages, that is the temperature at which they begin to unfolded, was assessed using both endogenous fluorescence detection and exogenous fluorescence detection methods. Differential Scanning Fluorimetry (DSF) and Static Light Scattering (SLS) were employed to monitor protein structural stability during thermal denaturation.

DSF measures conformational changes of a protein by monitoring temperature-dependent changes in fluorescence intensity. Fluorescence was derived either from an extrinsic fluorophore (SYPRO® Orange). SLS was performed by irradiating a laser in a heterogeneous medium, enabling determination of aggregate size via angle-dependent scattered light intensity measurements.

A Protein Thermal Stability Analyzer (Unchained Labs, Product No. RSP24110640) was used for DSF assays, and a qPCR instrument (Applied Biosystems 7500, Product No. RSP24110624) was used for exogenous fluorescence detection. Reference positive and negative control monoclonal antibodies (mAbs) were included at 1 mg/mL to verify quality control of results.

The endogenous fluorescence detection method (DSF) was employed as the primary analytical approach. When the Tm value could not be determined by endogenous fluorescence, the exogenous fluorescence detection method was used as a complementary assay.

Exogenous fluorescence detection (SLS) was carried out using the Protein Thermal Stability Analyzer. Samples were heated from 25 °C to 95 °C with a heating rate of either 0.4 °C/min or 0.8 °C/min. Each sample was equilibrated for 180 seconds before the temperature ramp was initiated, with no additional plate hold applied. Each sample was measured in duplicate to assess reproducibility of the melting temperature (Tm).

Differential Scanning Fluorimetry was performed using the qPCR instrument. Samples were heated from 25 °C to 95 °C at a constant rate of 0.96 °C/min. Prior to heating, samples were incubated for 120 seconds to allow fluorophore binding, after which they were subjected to the thermal gradient. Each sample was tested in triplicate to ensure consistency of the observed unfolding profile.

The exogenous fluorescence detection method is a complementary method to the endogenous fluorescence detection method, and the endogenous fluorescence detection method is preferred, and when the endogenous fluorescence detection method is unable to obtain the Tm value of this sample, the exogenous fluorescence detection method is used instead.

Two samples were measured by each method, and each sample was measured twice, providing 4 data points per construct per method. Results are shown in Table 15 below. All samples showed robust estimates of temperature induced unfolded, with values for each method and construct typically >65 C.

TABLE 15

| | DFS | DFS | SLS | SLS | SLS | SLS |
|---|---|---|---|---|---|---|
| SEQ_ID_NO | Tm (°C) | STDEV | Tonset (°C) | STDEV | Tagg (°C) | STDEV |
| 107 | 73.8 | 1.0 | 66.8 | 2.0 | 61.5 | 2.0 |
| 108 | 71.9 | 0.6 | 63.7 | 1.1 | 65.9 | 1.0 |
| 109 | 72.9 | 2.5 | 65.1 | 1.7 | 69.4 | 1.8 |

### Example 8: Electron Microscopy on Construct with SEQ ID NO: 109

### Methods

### Negative Stain EM:

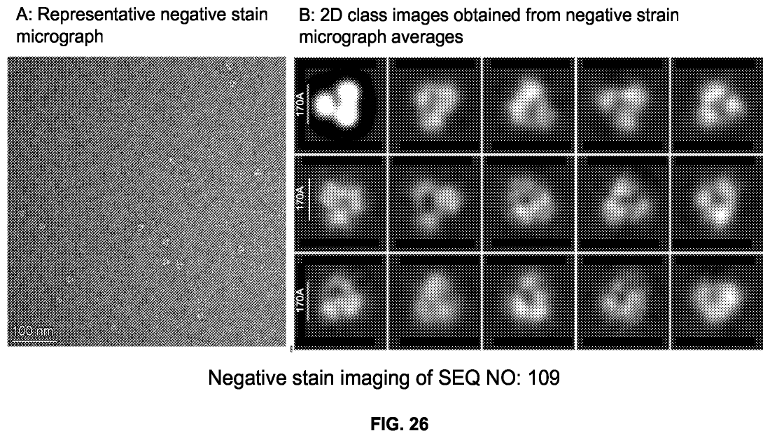

The sample was prepared by applying the sample at a concentration of 0.0315 mg/mL. The sample was diluted in a buffer consisting of 20 mM Tris pH 8.0, 5 mM EDTA, 150 mM NaCl to a final concentration of 0.0315 mg/mL. A small aliquot of the prepared sample was deposited onto a glow-discharged EM grid and allowed to adsorb for a suitable duration before excess liquid was blotted away. The grid was then stained with uranyl acetate to enhance contrast. Imaging was performed on a Talos F200S transmission electron microscope operated at 200 kV. Micrographs were acquired at a magnification of 94,000×, corresponding to a particle size range of approximately 13.4-13.8 nm. To check for correct assembly, 407 particles from 30 micrographs were subjected to 2D classification. FIG. 26 shows the negative stain imaging.

### CryoEM Grid Preparation and Data Collection:

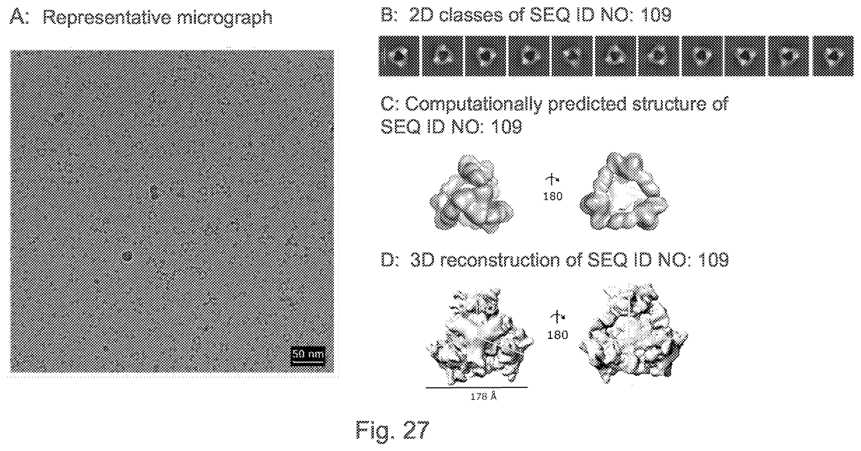

For cryo-electron microscopy (cryo-EM) sample preparation, the protein was diluted to a concentration of 0.63 mg/mL in buffer containing 20 mM Tris-HCl, 5 mM EDTA, 150 mM NaCl, pH 8.0. A 3 μL aliquot of the sample was applied to a glow-discharged Quantifoil R2/1, Au, 200 mesh grid. The grid was blotted for 3 seconds using force 3, followed by a 10-second wait time to allow for proper cryo-protection. The grid was then plunge-frozen in liquid ethane using a Vitrobot under optimal conditions to ensure vitreous ice formation. Data collection was performed on a Glacios 200 kV transmission electron microscope equipped with a direct electron detector. Micrographs were recorded at a magnification of 120,000×, with a pixel size of 1.2 Å. The defocus range was set from −1.4 μm to-2.4 μm, with a spherical aberration coefficient of 2.7 mm. The total electron dose was 50 e−/Å², fractionated into 32 frames per exposure to facilitate motion correction. A total of 1,110 micrographs were collected for subsequent image processing and analysis. The cryo-EM imaging is shown in FIG. 27.

### Data Processing:

Following data collection, 995 micrographs out of the total 1,110 were selected for further analysis after performing motion correction and contrast transfer function (CTF) estimation. Particle picking was conducted using the Template Picker and Inspect Picks tools, resulting in the identification of 847,833 particles. These particles were subjected to 2D classification to assess their quality and conformational states. Two distinct classes of particles were identified during this process. All subsequent analyses and reconstructions were performed assuming no symmetry (C1), with a total of 14,746 particles included in the final dataset for high-resolution 3D reconstruction.

### Example 9: Production of Human Protein Cages Fused to Effector Proteins as Payloads

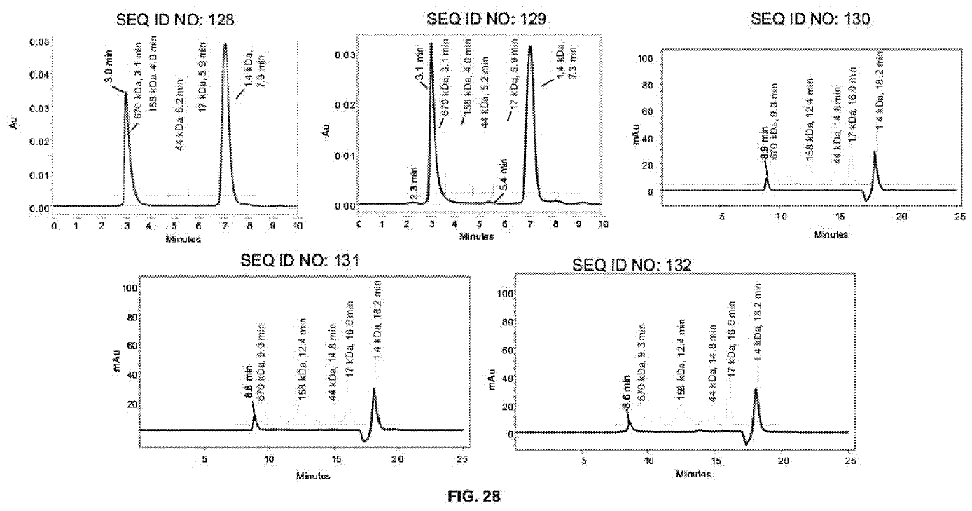

The two best-performing human protein cages (SEQ ID NO: 108 and 109) were tested as the basis for fusing to various effector proteins or payloads. SEQ ID NO: 128 and 129 are fusions of the two core cage variants to the PDL1 protein ligand, whose natural function is to bind the PD1 receptor. SEQ ID NO: 130 and 131 are fusions of the two core cage variants to a single-chain triple domain repeat version of the TNF protein (denoted scTNF), whose natural receptor is TNFR2. As a control, a fusion of the cage cores to an established single-chain antibody against the hen egg white lysozyme (HYHEL) protein was produced (SEQ ID NO: 132). Details regarding the purification and characterization of the proteins is shown in FIG. 28 and the sequence is shown in Table 16 below.

TABLE 16

| SEQ ID | | | | Purifi- | PAY- | MONOMER | CAGE MW | |
|---|---|---|---|---|---|---|---|---|
| NO | Target | Production | Scale | cation | LOAD | MW (kD) | (kD) | SEQUENCE |
| 128 | PD1 | Turbo | 100 | His | PDL1 | 71.1 | 853.2 | MGWSCIILFLVATATGVHSHHHHHHGSSAFT |
| | | CHO | ml | Tag | | | | VTVPKDLYVVEYGSNMTIECKFPVEKQLDLA |
| | | | | | | | | ALIVYWEMEDKNIIQFVHGEEDLKVQHSSYR |
| | | | | | | | | QRARLLKDQLSLGNAALQITDVKLQDAGVYR |
| | | | | | | | | CMISYGGADYKRITVKVNAPYNKINQRILVV |
| | | | | | | | | DPVTSEHELTCQAEGYPKAEVIWTSSDHQVL |
| | | | | | | | | SGKTTTTNSKREEKLFNVTSTLRINTTTNEI |
| | | | | | | | | FYCTFRRLDPEENHTAELVIPELPLAHPPNE |
| | | | | | | | | RTGGGSGGSGYTYEDYKNTAEWLLSHTKHRP |
| | | | | | | | | QVAIIAGSGLGGLTDKLTQAQIFDYGEIPNF |
| | | | | | | | | HPRSTVPGAGRLVFGFLNGRAVVMMQGRFHM |
| | | | | | | | | YEGYPLWKVTFPVRVFHLLGVDTLVVTNAAG |
| | | | | | | | | GLNPKFEVGDIMLIRDHINLPGFSGQNPLRG |
| | | | | | | | | PNDERFGDRFPAMSDAYDRTMRQRALSTWKQ |
| | | | | | | | | MGEQRELQEGTYVMVAGPSFETVAEARVLQK |
| | | | | | | | | VLGADAGMSTVPEVIVARHCGLRVFGFSLIT |
| | | | | | | | | NKVIMDYESLEKANFAEVLAAGAAALPRISR |
| | | | | | | | | FLGQLLADIAREDEIRAYLSSKLPSVEGLHA |
| | | | | | | | | PIVVSDRDGVVIKVANDNAPEHALRPGFLST |
| | | | | | | | | FALATDQGSKLGLSKVKSIISYYNTYQVVQF |
| | | | | | | | | NRLPLVVSFIASSSANTGLIVSLEKELAPLF |
| | | | | | | | | EELRQVVE |
| 129 | PD1 | Turbo | 100 | His | PDL1 | 71.4 | 856.8 | MGWSCIILFLVATATGVHSHHHHHHGSSAFT |
| | | CHO | ml | Tag | | | | VTVPKDLYVVEYGSNMTIECKFPVEKQLDLA |
| | | | | | | | | ALIVYWEMEDKNIIQFVHGEEDLKVQHSSYR |
| | | | | | | | | QRARLLKDQLSLGNAALQITDVKLQDAGVYR |
| | | | | | | | | CMISYGGADYKRITVKVNAPYNKINQRILVV |
| | | | | | | | | DPVTSEHELTCQAEGYPKAEVIWTSSDHQVL |
| | | | | | | | | SGKTTTTNSKREEKLFNVTSTLRINTTTNEI |
| | | | | | | | | FYCTFRRLDPEENHTAELVIPELPLAHPPNE |
| | | | | | | | | RTGGGSGGSGYTYEDYKNTAEWLLSHTKHRP |
| | | | | | | | | QVAIIAGSGLGGLTDKLTQAQIFDYGEIPNF |
| | | | | | | | | PRSTVPGHAGRLVFGFLNGRAVVMMQGRFHM |
| | | | | | | | | WYEGYPLKVTFPVRVFHLLGVDTLVVTNAAG |
| | | | | | | | | EGLNPKFVGDIMLIRDHINLPGFSGQNPLRG |
| | | | | | | | | DPNDERFGRFPAMSDAYDRTMRQRALSTWKQ |
| | | | | | | | | MGEQRELQEGTYVMVAGPSFETVAEARVLQK |
| | | | | | | | | VLGADAGMSTVPEVIVARHCGLRVFGFSLIT |
| | | | | | | | | NKVIMDYESLEKANHEEVLAAGKQAAQKLEQ |
| | | | | | | | | FVSILMADIARDDDLKRFLYKKLPSVEGLHA |
| | | | | | | | | IVVSDRDGVPVIKVANDNAPEHALRPGFLST |
| | | | | | | | | FALATDQGSKLGLSKVKSIISYYNTYQVVQF |
| | | | | | | | | NRLPLVVSFIASSSANTGLIVSLEKELAPLF |
| | | | | | | | | EELRQVVE |
| 130 | TNF | Turbo | 100 | His | sxTNFa | 98 | 1176 | MGWSCIILFLVATATGVHSHHHHHHGSSVRS |
| | R2 | CHO | ml | Tag | | | | SSRTPSDKPVAHVVANPQAEGQLQWLNRRAN |
| | | | | | | | | ALLANGVELRDNQLVVPSEGLYLIYSQVLFK |
| | | | | | | | | GQGCPSTHVLLTHTISRIAVSYQTKVNLLSA |
| | | | | | | | | IKSPCQRETPEGAEAKPWYEPIYLGGVFQLE |
| | | | | | | | | KGDRLSAEINRPDYLNFRESGQVYFGIIALG |
| | | | | | | | | GGGSSSRTPSDKPVAHVVANPQAEGQLQWLN |
| | | | | | | | | RRANALLANGVELRDNQLVVPSEGLYLIYSQ |
| | | | | | | | | VLFKGQGCPSTHVLLTHTISRIAVSYQTKVN |
| | | | | | | | | LLSAIKSPCQRETPEGAEAKPWYEPIYLGGV |
| | | | | | | | | FQLEKGDRLSAEINRPDYLNFRESGQVYFGI |
| | | | | | | | | SIALGGGGSSRTPSDKPVAHVVANPQAEGQL |
| | | | | | | | | QWLNRRANALLANGVELRDNQLVVPSEGLYL |
| | | | | | | | | IYSQVLFKGQGCPSTHVLLTHTISRIAVSYQ |
| | | | | | | | | TKVNLLSAIKSPCQRETPEGAEAKPWYEPIY |
| | | | | | | | | LGGVFQLEKGDRLSAEINRPDYLNFRESGQV |
| | | | | | | | | YFGIIALSGGGSGGSGYTYEDYKNTAEWLLS |
| | | | | | | | | HTKHRPQVAIIAGSGLGGLTDKLTQAQIFDY |
| | | | | | | | | GEIPNFPRSTVPGHAGRLVFGFLNGRAVVMM |
| | | | | | | | | QGRFHMYEGYPLWKVTFPVRVFHLLGVDTLV |
| | | | | | | | | VTNAAGGLNPKFEVGDIMLIRDHINLPGFSG |
| | | | | | | | | QNPLRGPNDERFGDRFPAMSDAYDRTMRQRA |
| | | | | | | | | LSTWKQMGEQRELQEGTYVMVAGPSFETVAE |
| | | | | | | | | ARVLQKLGADAVGMSTVPEVIVARHCGLRVF |
| | | | | | | | | GFSLITNKVIMDYESLEKANFAEVLAAGAAA |
| | | | | | | | | LPRISRFLGQLLADIAREDEIRAYLSSKLPS |
| | | | | | | | | VVEGLHAIVVSDRDGVPIKVANDNAPEHALR |
| | | | | | | | | PGFLSTFALATDOGSKLGLSKVKSIISYYNT |
| | | | | | | | | YQVVQFNRLPLVVSFIASSSANTGLIVSLEK |
| | | | | | | | | ELAPLFEELRQVVE |
| 131 | TNF | Turbo | 100 | His | scTNFa | 98.3 | 1179.6 | MGWSCIILFLVATATGVHSHHHHHHGSSVRS |
| | R2 | CHO | ml | Tag | | | | SSRTPSDKPVAHVVANPQAEGQLQWLNRRAN |
| | | | | | | | | ALLANGVELRDNQLVVPSEGLYLIYSQVLFK |
| | | | | | | | | GQGCPSTHVLLTHTISRIAVSYQTKVNLLSA |
| | | | | | | | | IKSPCQRETPEGAEAKPWYEPIYLGGVFQLE |
| | | | | | | | | KGDRLSAEINRPDYLNFRESGQVYFGIIALG |
| | | | | | | | | GGGSSSRTPSDKPVAHVVANPQAEGQLQWLN |
| | | | | | | | | RRANALLANGVELRDNQLVVPSEGLYLIYSQ |
| | | | | | | | | VLFKGQGCPSTHVLLTHTISRIAVSYQTKVN |
| | | | | | | | | LLSAIKSPCQRETPEGAEAKPWYEPIYLGGV |
| | | | | | | | | FQLEKGDRLSAEINRPDYLNFRESGQVYFGI |
| | | | | | | | | IALGGGGSSSRTPSDKPVAHVVANPQAEGQL |
| | | | | | | | | QWLNRRANALLANGVELRDNQLVVPSEGLYL |
| | | | | | | | | QIYSQVLFKGGCPSTHVLLTHTISRIAVSYQ |
| | | | | | | | | PTKVNLLSAIKSCQRETPEGAEAKPWYEPIY |
| | | | | | | | | RLGGVFQLEKGDLSAEINRPDYLNFRESGQV |
| | | | | | | | | YFGIIALSGGGSGGSGYTYEDYKNTAEWLLS |
| | | | | | | | | HTKHRPQVAIIAGSGLGGLTDKLTQAQIFDY |
| | | | | | | | | GEIPNFPRSTVPGHAGRLVFGFLNGRAVVMM |
| | | | | | | | | QGRFHMYEGYPLWKVTFPVRVFHLLGVDTLV |
| | | | | | | | | VTNAAGGLNPKFEVGDIMLIRDHINLPGFSG |
| | | | | | | | | QNPLRGPNDERFGDRFPAMSDAYDRTMRQRA |
| | | | | | | | | LSTWKQMGEQRELQEGTYVMVAGPSFETVAE |
| | | | | | | | | ARVLQKLGADAVGMSTVPEVIVARHCGLRVF |
| | | | | | | | | GFSLITNKVIMDYESLEKANHEEVLAAGKQA |
| | | | | | | | | AQKLEQFVSILMADIARDDDLKRFLYKKLPS |
| | | | | | | | | VEGLHAIVVSDRDGVPVIKVANDNAPEHALR |
| | | | | | | | | PGFLSTFALATDQGSKLGLSKVKSIISYYNT |
| | | | | | | | | YQVVQFNRLPLVVSFIASSSANTGLIVSLEK |
| | | | | | | | | ELAPLFEELRQVVE |
| 132 | HYHEL | Turbo | 100 | His | scFv | 71.4 | 856.8 | MGWSCIILFLVATATGVHSHHHHHHGSSDIV |
| | | CHO | ml | Tag | | | | LTQSPATLSVTPGNSVSLSCRASQSIGNNLH |
| | | | | | | | | WYQQKSHESPRLLIKYASQSISGIPSRFSGS |
| | | | | | | | | GSGTDFTLSINSVETEDFGMYFCQQSNSWPY |
| | | | | | | | | TFGGGTKLEIKRGGGGSGGGGSGGGGSGGGG |
| | | | | | | | | SDVQLQESGPSLVKPSQTLSLTCSVTGDSIT |
| | | | | | | | | SDYWSWIRKFPGNRLEYMGYVSYSGSTYYNP |
| | | | | | | | | SLKSRISITRDTSKNQYYLDLNSVTTEDTAT |
| | | | | | | | | YYCANWDGDYWGQGTLVTVSGGGSGGSGYTY |
| | | | | | | | | EDYKNTAEWLLSHTKHRPQVAIIAGSGLGGL |
| | | | | | | | | TDKLTQAQIFDYGEIPNFPRSTVPGHAGRLV |
| | | | | | | | | FGFLNGRAVVMMQGRFHMYEGYPLWKVTFPV |
| | | | | | | | | RVFHLLGVDTLVVTNAAGGLNPKFEVGDIML |
| | | | | | | | | IRDHINLPGFSGQNPLRGPNDERFGDRFPAM |
| | | | | | | | | SDAYDRTMRQRALSTWKQMGEQRELQEGTYV |
| | | | | | | | | VMVAGPSFETAEARVLQKLGADAVGMSTVPE |
| | | | | | | | | VIVARHCGLRVFGFSLITNKVIMDYESLEKA |
| | | | | | | | | NFAEVLAAGAAALPRISRFLGQLLADIARED |
| | | | | | | | | EIRAYLSSKLPSVEGLHAIVVSDRDGVPVIK |
| | | | | | | | | PVANDNAPEHALRGFLSTFALATDOGSKLGL |
| | | | | | | | | SKVKSIISYYNTYQVVQFNRLPLVVSFIASS |
| | | | | | | | | SANTGLIVSLEKELAPLFEELRQVVE |

### Example 10: Binding of Cages Fused to Effector Protein Payloads to their Cognate Receptor Proteins

### Methods

Binding kinetics were evaluated using biolayer interferometry (BLI) to determine the binding affinity of histidine-tagged protein cage payload fusions (SEQ ID NO: 129 and 130) to their intended receptor proteins (PD-1 for SEQ ID NO 129 and TNER2 for SEQ ID NO 130). The assays were performed on an Octet RED384 instrument.

### First Binding Assay:

Nanocage variants SEQ ID NO: 129 and 130 were each separately immobilized onto an anti-histidine tag sensor surface. The running buffer in all steps consisted of phosphate-buffered saline (PBS) containing 0.03% Tween-20, adjusted to pH 7.2 (GenScript, Lot No. 20250916027). Assays were conducted at 30 °C with constant orbital shaking at 1000 rpm.

The histidine-tagged protein nanocages SEQ ID NO: 129 and 130 were immobilized onto Ni-NTA biosensors (Cat No. 18-5101, Lot No. 2502004411). Following baseline stabilization, cognate receptor proteins, prepared as Fc fusion constructs, were introduced as analytes. Human Fc, PD1-Fc, and TNER2-Fc were presented at 1000 nM. Association kinetics were recorded for 120 seconds, followed by a dissociation phase of 300 seconds. Binding was observed for PD1-Fc and TNFR2-Fc to their respective protein cages under these conditions.

As a negative control, 41BB-Fc antigen was assessed under identical conditions at 1 μM concentration. No binding was observed in either association or dissociation phases, confirming the specificity of the binding interactions between nanocages (SEQ ID NO: 129 and 130) and their intended target receptor proteins, PD-1 and TNER2.

### Second Binding Assay:

A reciprocal assay was performed with identical experimental parameters (30 °C, 1000 rpm). Fc-tagged receptor proteins (PD-1 or TNFR2) were immobilized onto Protein A (ProA) biosensors, and histidine-tagged nanocage proteins SEQ ID NO: 129 and 130 were introduced as analytes at 1000 nM. Association and dissociation were monitored for 120 seconds and 300 seconds, respectively.

Specific binding between immobilized receptors and nanocages was observed. In contrast, when 41BB-Fc was used as analyte under identical conditions, no measurable binding signal was detected, serving as an internal negative control for nonspecific interactions.

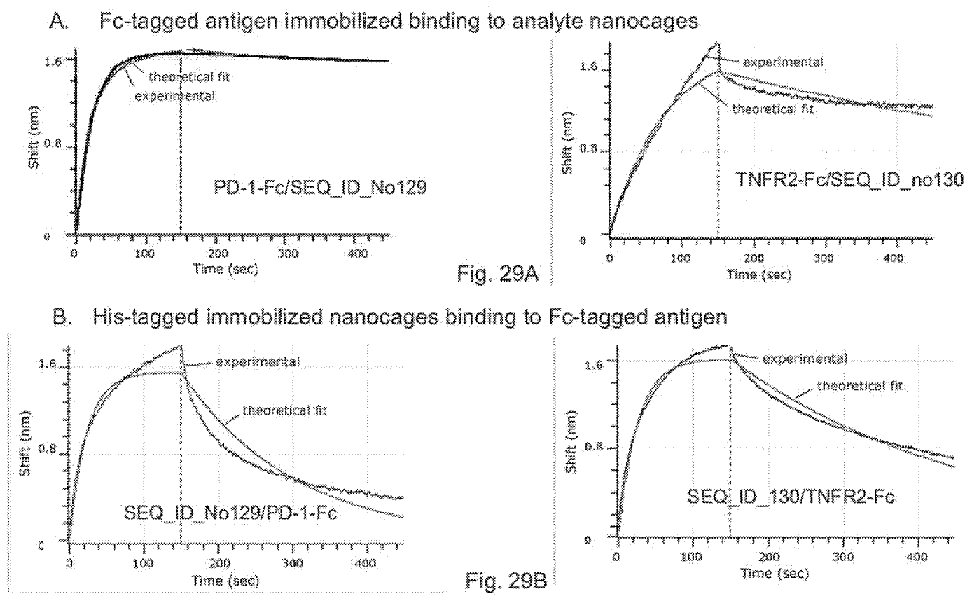

FIG. 29 illustrates the BLI binding curves showing specific binding of cage constructs plus effector payload proteins to their intended receptor proteins. More specifically, FIG. 29A shows the Fc tagged antigen immobilized binding to analyte nanocages. FIG. 29B shows the His tag immobilized nanocages binding to Fc tagged antigen.

### REFERENCES

The following references are herein incorporated by reference in their entirety with the exception that, should the scope and meaning of a term conflict with a definition explicitly set forth herein, the definition explicitly set forth herein controls:
Yeates T O. Geometric Principles for Designing Highly Symmetric Self-Assembling Protein Nanomaterials. Annu Rev Biophys. 2017 May 22; 46:23-42
C. Spiess, Q. Zhai, P. J. Carter, Alternative molecular formats and therapeutic applications for bispecific antibodies. Mol. Immunol. 67, 95 106 (2015).
D. M. Ecker, S. D. Jones, H. L. Levine, The therapeutic monoclonal antibody market. MAbs 7, 9 14 (2015).
D. W. LaFleur et al., Monoclonal antibody therapeutics with up to five specificities: functional enhancement through fusion of target specific peptides. MAbs 5, 208 218 (2013).
H. Byrne, P. J. Conroy, J. C. Whisstock, R. J. O'Kennedy, A tale of two specificities: bispecific antibodies for therapeutic and diagnostic applications. Trends Biotechnol. 31, 621 632 (2013).
H. J. Kang et al., Developing an antibody binding protein cage as a molecular recognition drug modular nanoplatform. Biomaterials 33, 5423 5430 (2012).
J. E. Padilla, C. Colovos, T. O. Yeates, Nanohedra: using symmetry to design self assembling protein cages, layers, crystals, and filaments. Proc. Natl. Acad. Sci. U.S.A 98, 2217 2221 (2001).
J. Stieglmaier, J. Benjamin, D. Nagorsen, Utilizing the BiTE (bispecific T cell engager) platform for immunotherapy of cancer. Expert Opin Biol Ther 15, 1093 1099 (2015).
N. Dimasi et al., Development of a Trispecific Antibody Designed to Simultaneously and Efficiently Target Three Different Antigens on Tumor Cells. Mol. Pharm. 12, 3490 3501 (2015).
T. O. Yeates, Y. Liu, J. Laniado. The design of symmetric protein nanomaterials comes of age in theory and practice. Curr Opin Struct Biol. 39, 134-143 (2016).
W. Choe, T. A. Durgannavar, S. J. Chung, Fc Binding Ligands of Immunoglobulin G: An Overview of High Affinity Proteins and Peptides. Materials (Basel) 9, (2016).
Y. J. Kang et al., Polyvalent display of monosaccharides on ferritin protein cage nanoparticles for the recognition and binding of cell surface lectins. Macromol. Biosci. 14, 619 625 (2014).
Y. T. Lai, D. Cascio, T. O. Yeates, Structure of a 16 nm cage designed by using protein oligomers. Science 336, 1129 (2012).
Y. T. Lai, K. L. Tsai, M. R. Sawaya, F. J. Asturias, T. O. Yeates, Structure and flexibility of nanoscale protein cages designed by symmetric self assembly. J. Am. Chem. Soc. 135, 7738 7743 (2013).
S. A. McConnell, K. A. Cannon, C. Morgan, R. McAllister, B. R. Amer, R. T. Clubb, T. O. Yeates. Designed Protein Cages as Scaffolds for Building Multienzyme Materials. ACS Synth Biol. 9, 381-391 (2020).
U.S. Pat. No. 6,756,039.
U.S. Pat. No. 8,969,521
U.S. Pat. No. 11,911,482
U.S. Patent Application Publication No. US20070218547
U.S. Patent Application Publication No. US20190341124
U.S. Patent Application, International Application Number PCT/US2020/030142.

1. A fusion protein subunit comprising:
an N-terminal domain comprising a first mammalian or human homo-oligomeric protein or portion thereof; and
a C-terminal domain comprising a second mammalian or human homo-oligomeric protein or portion thereof;
wherein the fusion protein subunit is capable of self-assembling into a three-dimensional multi-subunit structure.

2. (canceled)

3. (canceled)

4. The fusion protein subunit of claim 1, wherein the C-terminus of the N-terminal domain is fused to the N-terminus of C-terminal domain directly or via a linker, wherein the linker is 1, 2, 3, 4, 5, or 6 amino acids in length.

5. (canceled)

6. (canceled)

7. The fusion protein subunit of claim 1, wherein the N-terminal domain comprises a portion of Purine nucleoside phosphorylase, Enoyl-CoA delta isomerase 1—mitochondrial, Enoyl-CoA delta isomerase 2, or Citramalyl-CoA lyase—mitochondrial.

8. The fusion protein subunit of claim 1, wherein the C-terminal domain comprises a portion of Ragulator complex protein LAMTOR3, N-acylglucosamine 2-epimerase (Renin Binding Protein), Farnesyl pyrophosphate synthase, Ran-binding protein 10, NTF2-related export protein 1, Centromere protein S, Fructose-bisphosphate aldolase B, Barrier-to-autointegration factor-like protein, Retinol dehydrogenase 10, Tyrosine—tRNA ligase—mitochondrial, Fructose-bisphosphate aldolase B, Choline/ethanolamine kinase, Serine hydroxymethyltransferase—cytosolic, Malonyl-CoA decarboxylase—mitochondrial, Barrier-to-autointegration factor, Cytosolic Fe—S cluster assembly factor NUBP1, or Inositol monophosphatase 1.

9. The fusion protein subunit of claim 1 comprising an amino acid sequence that has at least 98% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO: 13, SEQ ID NO:14, SEQ ID NO:15, SEQ ID NO: 16, SEQ ID NO: 17, SEQ ID NO:18, SEQ ID NO: 19, SEQ ID NO:20, and SEQ ID NO:21.

10. (canceled)

11. (canceled)

12. (canceled)

13. The fusion protein subunit of claim 1, wherein the fusion protein subunit comprises:
an amino acid sequence that has at least 98% sequence identity to SEQ ID NO:1, SEQ ID NO:3, SEQ ID NO:4, SEQ ID NO:5, SEQ ID NO: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO: 15, SEQ ID NO: 16, SEQ ID NO:17, SEQ ID NO:18, SEQ ID NO: 19, SEQ ID NO:20, and SEQ ID NO: 21, further comprising an effector protein fused to the N-terminus of the first mammalian or human homo-oligomeric protein or portion thereof via a linker; or
an amino acid sequence selected that has at least 98% sequence identity to SEQ ID NO:2, SEQ ID NO:5, SEQ ID NO:6, SEQ ID NO:9, SEQ ID NO:10, SEQ ID NO: 11, SEQ ID NO:12, SEQ ID NO:13, SEQ ID NO:14, SEQ ID NO:17, and SEQ ID NO:20, further comprising an effector protein fused to the C-terminus of the second mammalian or human homo-oligomeric protein or portion thereof via a linker.

14. (canceled)

15. The fusion protein subunit of claim 13, wherein the effector protein is a cytokine, a cytokine receptor ligand, a chemokine, a chemokine receptor ligand, a growth factor, a hormone, a neuropeptide, a gastrointestinal or metabolic peptide, a cardiovascular peptide, or any other molecule that acts to modulate a therapeutic target, such as a receptor or cell surface protein on a target cell.

16. The fusion protein subunit of claim 15, wherein the effector protein is selected from Tables 1 to 9.

17. (canceled)

18. (canceled)

19. The fusion protein subunit of claim 13, wherein the effector protein is an antibody or antigenic fragment thereof.

20. The fusion protein subunit of claim 1, wherein the first mammalian or human homo-oligomeric protein or portion thereof and the second mammalian human homo-oligomeric protein or portion thereof are human proteins.

21. The fusion protein subunit of claim 1, wherein the first mammalian or human homo-oligomeric protein or portion thereof and the second mammalian or human homo-oligomeric protein or portion thereof are mouse or macaque proteins.

22. The fusion protein subunit of claim 1, wherein the first mammalian or human homo-oligomeric protein or portion thereof and/or the second mammalian or human homo-oligomeric protein or portion thereof comprise one or more amino acid substitutions to prevent or minimize chemical modifications, by either (i) substituting one or more cysteine residues with one or more of an alanine, serine or threonine residue and/or (ii) substituting one or more asparagine residues with one or more of an alanine, glutamine or aspartic acid residue.

23. (canceled)

24. The fusion protein subunit of claim 1, wherein the first mammalian or human homo-oligomeric protein or portion thereof and/or the second mammalian or human homo-oligomeric protein or portion thereof is a non-enzymatic variant comprising one or more amino acid substitutions to attenuate endogenous enzymatic activity.

25. (canceled)

26. (canceled)

27. The fusion protein subunit of claim 1 comprising an amino acid sequence that has at least 98% sequence identity to SEQ ID NO: 64-127.

28. (canceled)

29. The fusion protein subunit of claim 1, comprising an amino acid sequence that has at least 98% sequence identity to SEQ ID NO: 107-109.

30. (canceled)

31. (canceled)

32. (canceled)

33. (canceled)

34. The fusion protein subunit of claim 1, wherein the fusion protein subunit comprises an amino acid sequence having at least 98% sequence identity to SEQ ID NO: 22-63.

35. (canceled)

36. (canceled)

37. (canceled)

38. (canceled)

39. The fusion protein subunit of claim 1, wherein the fusion protein subunit comprises an amino acid sequence s is selected from SEQ ID NO: 107-109.

40. The fusion protein subunit of claim 1, wherein the fusion protein subunit is further fused by a linker to an Fc-binding sequence that is substantially identical to SEQ ID NO 133.

41. The fusion protein subunit of claim 1, wherein the fusion protein subunit comprises an amino acid sequence selected from SEQ ID NO: 107-109 and is further fused by a linker to an Fc-binding sequence that is substantially identical to SEQ ID NO 133.

42. A polypeptide, wherein the polypeptide comprises a sequence having at least 98% sequence identity to SEQ ID NO: 128-131.

43. (canceled)

44. The polypeptide of claim 42, wherein the polypeptide comprises a sequence having 100% sequence identity to SEQ ID NO: 128-131.

45. A protein cage comprising twelve copies of the fusion protein subunit of claim 1, wherein the twelve copies are assembled to form the protein cage.

46. A method of producing the fusion protein subunit of claim 1, the method comprising
expressing the fusion protein subunit; and
purifying the fusion protein subunit.

47. The method of claim 46, wherein the fusion protein subunit is expressed in CHO cells.

48. (canceled)

49. (canceled)

50. (canceled)
